(19) United States
(12) Patent Application Publication
(10) Pub. No.: US 2022/0183964 A1
(43) Pub. Date: Jun. 16, 2022

(54) IMPLANTABLE POLYMER DEPOTS FOR THE CONTROLLED, SUSTAINED RELEASE OF THERAPEUTIC AGENTS

(71) Applicant: Foundry Therapeutics, Inc., Menlo Park, CA (US)

(72) Inventors: Karun D. Naga, Los Altos, CA (US); Hanson S. Gifford, III, Woodside, CA (US); Mark Deem, Portola Valley, CA (US); Stephen W. Boyd, San Francisco, CA (US); Nassireddin Mokarram-Dorri, Campbell, CA (US); Koon Kiat Teu, Singapore (SG); Daniel Boon Lim Seet, Singapore (SG); Wei Li Lee, Singapore (SG); Honglei Wang, Singapore (SG)

(21) Appl. No.: 17/594,247

(22) PCT Filed: Apr. 11, 2020
(86) PCT No.: PCT/US2020/027852

§ 371 Date: Oct. 7, 2021

Related U.S. Application Data
(60) Provisional application No. 62/832,552, filed on Apr. 11, 2019.
(60) Provisional application No. 62/832,510, filed on Apr. 11, 2019.
(60) Provisional application No. 62/832,482, filed on Apr. 11, 2019.
(60) Provisional application No. 62/832,742, filed on Apr. 11, 2019.
(60) Provisional application No. 62/832,570, filed on Apr. 11, 2019.
(60) Provisional application No. 62/832,730, filed on Apr. 11, 2019.
(60) Provisional application No. 62/832,841, filed on Apr. 11, 2019.
(60) Provisional application No. 62/832,650, filed on Apr. 11, 2019.

(51) Int. Cl. A61K 9/00; A61K 31/155; A61K 47/26; A61K 38/17

(52) U.S. Cl. CPC A61K 9/0024; A61K 38/1796; A61K 47/26; A61K 31/155

(57) ABSTRACT

The present technology relates to depots for the treatment of select symptoms via sustained, controlled release of a therapeutic agent. In some embodiments, the depot may comprise a therapeutic region comprising the therapeutic agent, and a control region comprising a bioresorbable polymer and a releasing agent mixed with the polymer. The releasing agent may be configured to dissolve when the depot is placed in vivo to form diffusion openings in the control region. The depot may be configured to be implanted at a treatment site in vivo and, while implanted, release the therapeutic agent at the treatment site for an extended period of time.

### CROSS-REFERENCE TO RELATED APPLICATIONS

The present application claims the benefit of priority to U.S. Patent Application No. 62/832,482, filed Apr. 11, 2019, U.S. Patent Application No. 62/832,510, filed Apr. 11, 2019, U.S. Patent Application No. 62/832,552, filed Apr. 11, 2019, U.S. Patent Application No. 62/832,742, filed Apr. 11, 2019, U.S. Patent Application No. 62/832,570, filed Apr. 11, 2019, U.S. Patent Application No. 62/832,730, filed Apr. 11, 2019, U.S. Patent Application No. 62/832,650, filed Apr. 11, 2019, and U.S. Patent Application No. 62/832,841, filed Apr. 11, 2019, each of which is incorporated by reference herein in its entirety.

The present application also incorporates by reference each of the following applications in its entirety: International Application No. PCT/US2019/048437, filed Aug. 27, 2019; International Application No. PCT/US2019/048386, filed Aug. 27, 2019; International Application No. PCT/US2019/012795, filed Jan. 8, 2019; International Application No. PCT/US2018/054777, filed Oct. 6, 2018; U.S. Application No. 62/723,478, filed Aug. 28, 2018; U.S. Application No. 62/670,721, filed May 12, 2018; U.S. Application No. 62/640,571, filed Mar. 8, 2018; U.S. Application No. 62/614,884, filed Jan. 8, 2018; U.S. Patent Application No. 62/742,357, filed October 6, 2018; and U.S. Application No. 62/569,349, filed Oct. 6, 2017.

### TECHNICAL FIELD

The present technology relates to implants for controlled, sustained release of therapeutic agents in vivo.

### BACKGROUND OF THE INVENTION

Implantable systems for the controlled release of therapeutic agents offer advantages over other drug delivery methods, such as oral or parenteral methods. Devices comprised of biocompatible and/or biodegradable polymers and therapeutic agents can be implanted in clinically desirable anatomic locations, thereby providing localized delivery of select agents. This localized delivery enables a substantial proportion of the agent to reach the intended target and undesirable systemic side effects can be avoided. However, these systems often suffer from a lack of a true controlled release mechanism in that they typically provide a burst of drug upon contact with surrounding physiologic fluids followed by a residual release of drug.

In order to improve drug release in certain polymer carriers, hydrophilic polymers, such as polysorbate, have been added to these carriers as wetting agents to accelerate or to enhance drug release from biocompatible polymers such polyethylene glycol (PEG) in oral formulations (Akbari, J., et al., ADV. PHARM. BULL., 2015, 5(3): 435-441). However, these formulations are intended to provide an immediate release of a hydrophobic drug into a hydrophilic environment (the in vivo physiologic fluid), where a substantial portion of the entire drug payload is immediately or aggressively released, not a variable or sustained control release.

While these drug release kinetics may be desirable in some clinical applications, a controlled, sustained release of a therapeutic agent can be of clinical benefit in certain circumstances. In particular, it may be desirable to implant a biodegradable carrier holding a large dose of a therapeutic agent for a controlled, sustained release over time.

Thus, a need exists for biocompatible implantable systems capable of providing a highly controlled release of drug.

### SUMMARY

The present technology relates to implants for controlled release of a therapeutic agent to treat a medical condition and associated systems and methods. In particular, the present technology relates to implants for sustained and/or local release of a therapeutic agent at a surgical or interventional site and associated systems and methods.

The subject technology is illustrated, for example, according to various aspects described below, including with reference to FIGS. 1-59. Various examples of aspects of the subject technology are described as numbered clauses (1, 2, 3, etc.) for convenience. These are provided as examples and do not limit the subject technology.

1. A depot for the controlled, sustained release of a therapeutic agent, comprising:
a therapeutic region comprising the therapeutic agent, the therapeutic region elongated along a first axis; and
a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region;
wherein the depot is configured to be implanted at a treatment site in vivo and, while implanted, release the therapeutic agent at the treatment site for a period of time.

2. The depot of any one of the clauses herein, wherein the depot is at least 5 times longer along the first axis than a maximum transverse dimension along a second axis orthogonal to the first.

3. The depot of any one of the clauses herein, wherein the depot is at least 10 times longer along the first axis than a maximum transverse dimension along a second axis orthogonal to the first.

4. The depot of any one of the clauses herein, wherein the depot is substantially columnar.

5. The depot of any one of the clauses herein, wherein the depot is substantially cylindrical.

6. The depot of any one of the clauses herein, wherein the depot is configured to be injected or inserted via a needle that is no greater than 14, 16, 18, 20, or 22 gauge in size.

7. The depot of any one of the clauses herein, wherein the therapeutic region is substantially cylindrical.

8. The depot of any one of the clauses herein, further comprising at least one opening extending through the therapeutic region.

9. The depot of any one of the clauses herein, wherein the opening forms a cylindrical lumen extending parallel to the first axis.

10. The depot of any one of the clauses herein, wherein the opening comprises a lumen extending along a second axis substantially perpendicular to the first axis.

11. The depot of any one of the clauses herein, further comprising a plurality of elongated openings extending parallel to the second axis.

12. The depot of any one of the clauses herein, wherein the therapeutic region comprises a plurality of separate elongated sub-regions extending substantially parallel to the first axis.

13. The depot of any one of the clauses herein, wherein each of the elongated sub-regions is substantially cylindrical.

14. The depot of any one of the clauses herein, wherein each of the elongated sub-regions are radially separated from one another by the control region.

15. The depot of any one of the clauses herein, wherein a radially outermost dimension of the depot varies along the first axis.

16. The depot of any one of the clauses herein, wherein a radially outermost dimension of the therapeutic region varies along the first axis.

17. The depot of any one of the clauses herein, wherein the therapeutic region is a series of separate regions, covered by and connected by a continuous control region.

18. The depot of any one of the clauses herein, wherein the control region is narrower in the regions without an internal therapeutic region.

19. The depot of any one of the clauses herein, wherein the control region is designed to bend or break during or after delivery.

20. The depot of any one of the clauses herein, wherein the control region has a variable thickness along a length of the depot along the first axis.

21. The depot of any one of the clauses herein, wherein the control region has a thickness that varies radially around the first axis.

22. The depot of any one of the clauses herein, wherein the variable thickness of the control region causes the depot to curve or bend when deployed in vivo.

23. The depot of any one of the clauses herein, wherein the depot is configured to curve or bend preferentially when placed in contact with physiological fluids in vivo.

24. The depot of any one of the clauses herein, wherein the depot comprises an elongated polymer strip having a length between its longitudinal ends and a width between lateral edges, the length greater than the width, and wherein the depot has a preset shape in an expanded configuration in which the strip is curled about an axis with the width of the strip facing the axis, thereby forming a ring-like shape.

25. The depot of any one of the clauses herein, wherein the depot forms an annular or semi-annular shape.

26. The depot of any one of the clauses herein, wherein the depot has a first region and a second region, each extending longitudinally and coextensive with one another over all or a portion of their respective lengths, the first region having a first elasticity and the second region having a second elasticity less than the first elasticity.

27. The depot of any one of the clauses herein, wherein the depot has been stretched beyond the elastic hysteresis point of the second region such that, when released from a delivery device, the depot transitions from a straightened state to a curved state in which the second region pulls the depot into the curved shape.

28. The depot of any one of the clauses herein, wherein the depot has a first region and a second region, each extending longitudinally and coextensive with one another over all or a portion of their respective lengths, the first region being more hydrophilic than the second region.

29. The depot of any one of the clauses herein, wherein, when released from a delivery device, the depot transitions from a straightened state to a curved state in which the second region pulls the depot into the curved shape.

30. The depot of any one of the clauses herein, wherein the control region has first and second portions having a first thickness, the first and second portions separated along the first axis by a third portion having a second thickness different from the first.

31. The depot of any one of the clauses herein, wherein the depot extends along the first axis from a first end to a second end, and wherein the control region has a thickness that increases from the first end to the second end.

32. The depot of any one of the clauses herein, wherein the depot extends along the first axis from a first end to a second end, and wherein the control region does not cover the therapeutic region at the first end of the depot.

33. The depot of any one of the clauses herein, wherein the depot extends along the first axis from a first end to a second end, and wherein the control region does not cover the therapeutic region at the first end or the second end.

34. The depot of any one of the clauses herein, wherein the control region has a plurality of discrete openings formed therein.

35. The depot of any one of the clauses herein, wherein the control region has an opening elongated along the first axis.

36. The depot of any one of the clauses herein, wherein the elongated opening in the control region extends along the entire length of the depot.

37. The depot of any one of the clauses herein, wherein the control region comprises a plurality of circular apertures formed therein.

38. The depot of any one of the clauses herein, wherein the therapeutic region is a first therapeutic region, the depot further comprising a second therapeutic region, each of the first and second therapeutic regions being elongated along the first axis, wherein the first and second therapeutic regions are configured to release the therapeutic agent at different rates.

39. The depot of any one of the clauses herein, wherein the therapeutic region is a first therapeutic region, the depot further comprising a second therapeutic region, each of the first and second therapeutic regions being elongated along the first axis, wherein the first and second therapeutic regions comprise different therapeutic agents.

40. The depot of any one of the clauses herein, wherein the first and second therapeutic regions are coaxially aligned.

41. The depot of any one of the clauses herein, wherein the first and second therapeutic regions extend parallel to one another along a length of the depot.

42. The depot of any one of the clauses herein, further comprising a barrier region configured to dissolve in vivo more slowly than the control region or the therapeutic region.

43. The depot of any one of the clauses herein, further comprising a barrier region configured to slow the passage of physiological fluids in vivo therethrough to the control region or the therapeutic region.

44. The depot of any one of the clauses herein, wherein the barrier region is disposed coaxially with the therapeutic region, such that the control region at least partially surrounds both the therapeutic region and the barrier region.

45. The depot of any one of the clauses herein, wherein the barrier region is a first barrier region, the depot further comprising a second barrier region, the first and second barrier regions separated axially from one another by the therapeutic region.

46. The depot of any one of the clauses herein, wherein the first and second barrier regions have different dimensions.

47. The depot of any one of the clauses herein, wherein the barrier region is disposed coaxially with the control region, such that the control region and barrier region together at least partially surround the therapeutic region.

48. The depot of any one of the clauses herein, wherein the first and second barrier regions are separated axially from one another by the control region.

49. The depot of any one of the clauses herein, wherein the depot extends along the first axis from a first end to a second end, and wherein the barrier region is disposed over the first end of the depot.

50. The depot of any one of the clauses herein, wherein the depot extends along the first axis from a first end to a second end, and wherein the barrier region comprises a first end cap disposed over the first end of the depot and a second end cap disposed over the second end of the depot.

51. The depot of any one of the clauses herein, wherein the therapeutic region comprises a covered portion and an exposed portion, wherein the covered portion is covered by the control region such that, when the depot is initially positioned at the treatment site in vivo, the control region is between the covered portion of the therapeutic region and physiologic fluids at the treatment site and the exposed portion of the therapeutic region is exposed to the physiologic fluids.

52. The depot of any one of the clauses herein, wherein the therapeutic agent in the therapeutic region comprises at least 50% of the total weight of the depot.

53. The depot of any one of the clauses herein, wherein the period of time is not less not less than 7 days, than 15 days, not less than 30 days, not less than 45 days, not less than 60 days, or not less than 90 days.

54. The depot of any one of the clauses herein, wherein about 40% to about 60% of the therapeutic agent in the therapeutic region is released in the first half of the period of time.

55. The depot of any one of the clauses herein, wherein at least 90% of the therapeutic agent in the therapeutic region is released within the period of time.

56. The depot of any one of the clauses herein, wherein the depot is configured to release about 2 μg to about 5 mg of the therapeutic agent to the treatment site per day.

57. The depot of any one of the clauses herein, wherein the depot is configured to release the therapeutic agent at the treatment site in vivo for no less than 1 day, no less than 2 days, no less than 3 days, no less than 4 days, no less than 5 days, no less than 6 days, no less than 7 days, no less than 8 days, no less than 9 days, no less than 10 days, no less than 11 days, no less than 12 days, no less than 13 days, no less than 14 days, no less than 15 days, no less than 16 days, no less than 17 days, no less than 18 days, no less than 19 days, no less than 20 days, no less than 21 days, no less than 22 days, no less than 23 days, no less than 24 days, no less than 25 days, no less than 26 days, no less than 27 days, no less than 28 days, no less than 29 days, no less than 30 days, no less than 40 days, no less than 50 days, no less than 60 days, no less than 70 days, no less than 90 days, no less than 100 days, no less than 200 days, no less than 300 days, or no less than 365 days.

58. The depot of any one of the clauses herein, wherein the therapeutic agent is released at a substantially steady state rate throughout the period of time.

59. The depot of any one of the clauses herein, wherein,
the depot has a total surface area comprising the exposed surface area of the control region plus the exposed surface area of the therapeutic region, and
when the depot is initially positioned at the treatment site in vivo, a ratio of the exposed surface area of the therapeutic region to the exposed surface area of the control region is from about 5% to about 20%, or from about 5% to about 15%, or from about 5% to about 10%.

60. The depot of any one of the clauses herein, wherein the exposed surface area of the control region is less than the exposed surface area of the therapeutic region.

61. The depot of any one of the clauses herein, wherein the exposed surface area of the control region is greater than the exposed surface area of the therapeutic region.

62. The depot of any one of the clauses herein, wherein the control region is a first control region, and wherein the depot comprises a second control region.

63. The depot of any one of the clauses herein, wherein the first control region is disposed at a first side of the therapeutic region and the second control region is disposed at a second side of the therapeutic region opposite the first side.

64. The depot of any one of the clauses herein, wherein the depot comprises a plurality of control regions and a plurality of therapeutic regions, and wherein each of the therapeutic regions is separated from an adjacent one of the therapeutic regions by one or more control regions.

65. The depot of any one of the clauses herein, wherein the depot comprises from about 2 to about 10 therapeutic regions.

66. The depot of any one of the clauses herein, wherein the control region comprises a first control layer and a second control layer.

67. The depot of any one of the clauses herein, wherein the second control layer is adjacent to the therapeutic region and the first control layer encapsulates/encloses the therapeutic region and the second control layer.

68. The depot of any one of the clauses herein, wherein the first control layer and the second control layer together enclose the therapeutic region.

69. The depot of any one of the clauses herein, wherein the first control layer comprises a first plurality of sub-layers and the second control layer comprises a second plurality of sub-layers.

70. The depot of any one of the clauses herein, wherein the first control layer includes a first amount of the releasing agent and the second control layer includes a second amount of the releasing agent different than the first amount.

71. The depot of any one of the clauses herein, wherein the second control layer is positioned between the first control layer and the therapeutic region, and wherein the first control layer includes a first concentration of the releasing agent and the second control layer includes a second concentration of the releasing agent greater than the first concentration.

72. The depot of any one of the clauses herein, wherein the second control layer is positioned between the first control layer and the therapeutic region, and wherein the first control layer includes a first concentration of the releasing agent and the second control layer includes a second concentration of the releasing agent less than the first concentration.

73. The depot of any one of the clauses herein, wherein the second control layer is positioned between the first control layer and the therapeutic region, and wherein
the first control layer includes up to 5% by weight of the releasing agent, up to 10% by weight of the releasing agent, up to 15% by weight of the releasing agent, up to 20% by weight of the releasing agent, up to 25% by weight of the releasing agent, up to 30% by weight of the releasing agent, up to 35% by weight of the releasing agent, up to 40% by weight of the releasing agent, up to 45% by weight of the releasing agent, or 50% by weight of the releasing agent; and
the second control layer includes up to 5% by weight of the releasing agent, up to 10% by weight of the releasing agent, up to 15% by weight of the releasing agent, up to 20% by weight of the releasing agent, up to 25% by weight of the releasing agent, up to 30% by weight of the releasing agent, up to 35% by weight of the releasing agent, up to 40% by weight of the releasing agent, up to 45% by weight of the releasing agent, or up to 50% by weight of the releasing agent.

74. The depot of any one of the clauses herein, wherein the second control layer is positioned between the first control layer and the therapeutic region, and wherein the first control layer includes a first amount of the releasing agent and the second control layer includes a second amount of the releasing agent, the second amount being at least 2×, at least 3×, at least 4×, or at least 5× the first amount.

75. The depot of any one of the clauses herein, wherein a thickness of the control region is less than or equal to 1/10, 1/15, 1/20, 1/25, 1/30, 1/35, 1/40, 1/45, 1/50, 1/75, or 1/100 of a thickness of the therapeutic region.

76. The depot of any one of the clauses herein, wherein the depot comprises an elongate columnar structure configured to be implanted in a patient.

77. The depot of any one of the clauses herein, wherein the depot comprises one of a plurality of beads or microspheres.

78. The depot of any one of the clauses herein, wherein the beads or microspheres have varying release profiles.

79. The depot of any one of the clauses herein, wherein the beads or microspheres comprise varying amounts of therapeutic agent.

80. The depot of any one of the clauses herein, wherein the beads or microspheres comprise varying thicknesses of their respective control regions.

81. The depot of any one of the clauses herein, wherein the beads of microspheres have varying dimensions.

82. The depot of any one of the clauses herein, wherein the depot comprises one of a plurality of pellets.

83. The depot of any one of the clauses herein, wherein the pellets have varying release profiles.

84. The depot of any one of the clauses herein, wherein the pellets comprise varying amounts of therapeutic agent.

85. The depot of any one of the clauses herein, wherein the pellets comprise varying thicknesses of their respective control regions.

86. The depot of any one of the clauses herein, wherein the pellets have varying dimensions.

87. The depot of any one of the clauses herein, wherein the pellets are substantially cylindrical.

88. The depot of any one of the clauses herein, wherein the depot comprises a plurality of substantially cylindrical beads, each comprising a therapeutic region and control region and wherein the plurality of beads are substantially aligned along a common longitudinal axis.

89. The depot of any one of the clauses herein, wherein the depot is biodegradable and/or bioerodible.

90. The depot of any one of the clauses herein, wherein the depot is a flexible solid that is structurally capable of being handled by a clinician during the normal course of a surgery without breaking into multiple pieces and/or losing its general shape.

91. The depot of any one of the clauses herein, wherein the depot is configured to be subcutaneously placed within a patient and release the therapeutic agent in vivo for up to 7 days without breaking into multiple pieces.

92. The depot of any one of the clauses herein, wherein the depot has a surface area and a volume, and wherein a ratio of the surface area to volume is at least 1.

93. The depot of any one of the clauses herein, wherein the diffusion openings include at least one or more pores and/or one or more channels.

94. The depot of any one of the clauses herein, wherein dissolution of the releasing agent following in vivo placement in the treatment site causes the control region and the therapeutic region to transition from a state of lesser porosity to a state of greater porosity to facilitate the release of the therapeutic agent from the depot.

95. The depot of any one of the clauses herein, wherein the releasing agent is a first releasing agent and the therapeutic region includes a second releasing agent mixed with the therapeutic agent.

96. The depot of any one of the clauses herein, wherein the releasing agent is a first releasing agent and the polymer is a first polymer, and the therapeutic region includes a second releasing agent and a second polymer mixed with the therapeutic agent.

97. The depot of any one of the clauses herein, wherein the first releasing agent is the same as the second releasing agent.

98. The depot of any one of the clauses herein, wherein the first releasing agent is the different than the second releasing agent.

99. The depot of any one of the clauses herein, wherein a concentration of the first releasing agent within the control region is the greater than a concentration of the second releasing agent within the therapeutic region.

100. The depot of any one of the clauses herein, wherein a concentration of the first releasing agent within the control region is the less than a concentration of the second releasing agent within the therapeutic region.

101. The depot of any one of the clauses herein, wherein a concentration of the first releasing agent within the control region is the same as a concentration of the second releasing agent within the therapeutic region.

102. The depot of any one of the clauses herein, wherein a concentration of the first releasing agent within the control region is different than a concentration of the second releasing agent within the therapeutic region.

103. The depot of any one of the clauses herein, wherein the therapeutic region includes a plurality of microlayers.

104. The depot of any one of the clauses herein, wherein the mass of the therapeutic agent comprises at least 50% of the mass of the depot.

105. The depot of any one of the clauses herein, wherein the ratio of the mass of the therapeutic agent in the depot to the depot polymer mass is at least at least 1:1, at least 2:1, 3:1, at least 4:1, at least 5:1, at least 6:1, at least 7:1, at least 8:1, at least 9:1, at least 10:1, or at least 16:1.

106. The depot of any one of the clauses herein, wherein the therapeutic region comprises a bioresorbable polymer and the therapeutic agent.

107. The depot of any one of the clauses herein, wherein the therapeutic region includes at least 40% by weight of the therapeutic agent, at least 50% by weight of the therapeutic agent, at least 60% by weight of the therapeutic agent, 60% by weight of therapeutic agent, at least 70% by weight of the therapeutic agent, at least 80% by weight of the therapeutic agent, at least 90% by weight of the therapeutic agent, or 100% by weight of the therapeutic agent.

108. The depot of any one of the clauses herein, wherein the depot includes at least 15% by weight of the therapeutic agent, at least 20% by weight of the therapeutic agent, at least 30% by weight of the therapeutic agent, at least 40% by weight of the therapeutic agent, at least 50% by weight of the therapeutic agent, at least 60% by weight of the therapeutic agent, at least 70% by weight of the therapeutic agent, at least 80% by weight of the therapeutic agent, at least 90% by weight of the therapeutic agent, 99% by weight of the therapeutic agent, or 99.99% by weight of the therapeutic agent.

109. The depot of any one of the clauses herein, wherein the releasing agent is a non-ionic surfactant.

110. The depot of any one of the clauses herein, wherein the releasing agent has hydrophilic properties.

111. The depot of any one of the clauses herein, wherein the releasing agent is a polysorbate.

112. The depot of any one of the clauses herein, wherein the releasing agent is Tween 20.

113. The depot of any one of the clauses herein, wherein the releasing agent is Tween 80.

114. The depot of any one of the clauses herein, wherein the releasing agent is non-polymeric.

115. The depot of any one of the clauses herein, wherein the releasing agent is not a plasticizer.

116. The depot of any one of the clauses herein, wherein the polymer is configured to degrade only after substantially all of the therapeutic agent has been released from the depot.

117. The depot of any one of the clauses herein, wherein the polymer is a copolymer.

118. The depot of any one of the clauses herein, wherein the polymer is a terpolymer.

119. The depot of any one of the clauses herein, wherein the polymer includes at least one of polyglycolide (PGA), polycaprolactone (PCL), poly(DL-lactic acid) (PLA), poly(alpha-hydroxy acids), poly(lactide-co-glycolide)(PLGA or DLG), poly(DL-lactide-co-caprolactone) (DL-PLCL), poly(trimethylene carbonate) (PTMC), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (such as hyaluronic acid, chitosan and starch), proteins (such as gelatin and collagen) or PEG derivatives, polyaspirins, polyphosphagenes, pre-gelatinized starch, hyaluronic acid, chitosans, gelatin, alginates, albumin, fibrin, vitamin E analogs, such as alpha tocopheryl acetate, d-alpha tocopheryl succinate, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate)hydroxypropyl cellulose, hydroxypropyl methylcellulose, hydroxyethyl methylcellulose, carboxymethylcellulose or salts thereof, Carbopol®, poly(hydroxyethylmethacrylate), poly(methoxyethylmethacrylate), poly(methoxyethoxy-ethylmethacrylate), polymethylmethacrylate (PMMA), polyvinyl alcohols, propylene glycol, and poly(DL-lactide-co-glycolide-co-caprolactone).

120. The depot of any one of the clauses herein, wherein the polymer is one of poly(DL-lactide-co-glycolide-co-caprolactone) and poly(DL-lactide-co-glycolide)(PLGA).

121. The depot of any one of the clauses herein, wherein the polymer is poly(DL-lactide-co-glycolide-co-caprolactone) in a molar ratio of about 60:30:10.

122. The depot of any one of the clauses herein, wherein the polymer is poly(DL-lactide-co-glycolide)(PLGA) in a molar ratio of between about 10:90 and about 90:10.

123. The depot of any one of the clauses herein, wherein the polymer is poly(DL-lactide-co-glycolide)(PLGA) in a molar ratio of about 50:50.

124. The depot of any one of the clauses herein, wherein the polymer is ester-terminated.

125. The depot of any one of the clauses herein, wherein the polymer is a terpolymer that includes three polymers selected from the following: polyglycolide (PGA), polycaprolactone (PCL), poly(L-lactic acid) (PLA), poly(DL-lactic acid) (PLA), poly(trimethylene carbonate) (PTMC), polydioxanone (PDO), poly(-hydroxy butyrate) (PHB), polyhydroxyalkanoates (PHA), poly(phosphazene), and polyethylene glycol.

126. The depot of any one of the clauses herein, wherein the polymer is a first polymer, and the therapeutic region includes a second polymer mixed with the therapeutic agent.

127. The depot of any one of the clauses herein, wherein the first polymer and/or the second polymer include at least one of polyglycolide (PGA), polycaprolactone (PCL), poly(DL-lactic acid) (PLA), poly(alpha-hydroxy acids), poly(lactide-co-glycolide)(PLGA or DLG), poly(DL-lactide-co-caprolactone) (DL-PLCL), poly(trimethylene carbonate) (PTMC), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (such as hyaluronic acid, chitosan and starch), proteins (such as gelatin and collagen) or PEG derivatives, polyaspirins, polyphosphagenes, pre-gelatinized starch, hyaluronic acid, chitosans, gelatin, alginates, albumin, fibrin, vitamin E analogs, such as alpha tocopheryl acetate, d-alpha tocopheryl succinate, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate)hydroxypropyl cellulose, hydroxypropyl methylcellulose, hydroxyethyl methylcellulose, carboxymethylcellulose or salts thereof, Carbopol®, poly(hydroxyethylmethacrylate), poly(methoxyethylmethacrylate), poly(methoxyethoxy-ethylmethacrylate), polymethylmethacrylate (PMMA), polyvinyl alcohols, propylene glycol, poly(DL-lactide-co-glycolide-co-caprolactone).

128. The depot of any one of the clauses herein, wherein the first polymer and/or the second polymer selected from the following: poly(DL-lactide-co-glycolide-co-caprolactone) and poly(DL-lactide-co-glycolide)(PLGA).

129. The depot of any one of the clauses herein, wherein the first polymer and/or the second polymer is poly(DL-lactide-co-glycolide-co-caprolactone) and has a molar ratio of about 60:30:10.

130. The depot of any one of the clauses herein, wherein the first polymer and/or the second polymer is poly(DL-lactide-co-glycolide) and has a molar ratio of about 50:50.

131. The depot of any one of the clauses herein, wherein the first polymer and/or the second polymer is ester-terminated.

132. The depot of any one of the clauses herein, wherein the first polymer and/or the second polymer is a terpolymer that includes three polymers selected from the following: polyglycolide (PGA), polycaprolactone (PCL), poly(L-lactic acid) (PLA), poly(trimethylene carbonate) (PTMC), polydioxanone (PDO), poly(4-hydroxy butyrate) (PHB), polyhydroxyalkanoates (PHA), poly(phosphazene), and polyethylene glycol.

133. The depot of any one of the clauses herein, wherein the ratio of the polymer to the releasing agent in the control region is at least 1:1, at least 2:1, at least 3:1, at least 4:1, at least 5:1, at least 6:1, at least 7:1, at least 8:1, at least 9:1, at least 10:1, or at least 15:1.

134. The depot of any one of the clauses herein, wherein the releasing agent is configured to dissolve when the depot is placed in contact with phosphate buffered saline to form diffusion openings.

135. The depot of any one of the clauses herein, wherein the exposed surface area of the control region is less than the exposed surface area of the therapeutic region.

136. The depot of any one of the clauses herein, wherein the exposed surface area of the control region is greater than the exposed surface area of the therapeutic region.

137. The depot of any one of the clauses herein, wherein the control region is a first control region, and wherein the depot comprises a second control region.

138. The depot of any one of the clauses herein, wherein the first control region is disposed at a first side of the therapeutic region and the second control region is disposed at a second side of the therapeutic region opposite the first side.

139. The depot of any one of the clauses herein, wherein the depot comprises a plurality of control regions and a plurality of therapeutic regions, and wherein each of the therapeutic regions is separated from an adjacent one of the therapeutic regions by one or more control regions.

140. The depot of any one of the clauses herein, wherein each of the therapeutic regions and each of the control regions is a micro-thin layer.

141. The depot of any one of the clauses herein, wherein the depot comprises from about 2 to about 100 therapeutic regions.

142. The depot of any one of the clauses herein, wherein the depot comprises from about 2 to about 50 therapeutic regions.

143. The depot of any one of the clauses herein, wherein the depot comprises from about 2 to about 10 therapeutic regions.

144. The depot of any one of the clauses herein, wherein the therapeutic region is enclosed by the control region such that, when the depot is positioned in vivo, the control region is between the therapeutic region and in vivo physiologic fluids.

145. The depot of any one of the clauses herein, wherein the control region comprises a first control layer and a second control layer.

146. The depot of any one of the clauses herein, wherein the second control layer is adjacent to the therapeutic region and the first control layer encapsulates/encloses the therapeutic region and the second control layer.

147. The depot of any one of the clauses herein, wherein the first control layer and the second control layer together enclose the therapeutic region.

148. The depot of any one of the clauses herein, wherein the first control layer is disposed at a first side of the therapeutic region and the second control layer is disposed at a second side of the therapeutic region opposite the first side.

149. The depot of any one of the clauses herein, wherein the first control layer comprises a first plurality of sub-layers and the second control layer comprises a second plurality of sub-layers.

150. The depot of any one of the clauses herein, wherein the first control layer includes a first amount of the releasing agent and the second control layer includes a second amount of the releasing agent different than the first amount.

151. The depot of any one of the clauses herein, wherein the second control layer is positioned between the first control layer and the therapeutic region, and wherein the first control layer includes a first concentration of the releasing agent and the second control layer includes a second concentration of the releasing agent greater than the first concentration.

152. The depot of any one of the clauses herein, wherein the second control layer is positioned between the first control layer and the therapeutic region, and wherein the first control layer includes a first concentration of the releasing agent and the second control layer includes a second concentration of the releasing agent less than the first concentration.

153. The depot of any one of the clauses herein, wherein the second control layer is positioned between the first control layer and the therapeutic region, and wherein:
the first control layer includes up to 5% by weight of the releasing agent, up to 10% by weight of the releasing agent, up to 15% by weight of the releasing agent, up to 20% by weight of the releasing agent, up to 25% by weight of the releasing agent, up to 30% by weight of the releasing agent, up to 35% by weight of the releasing agent, up to 40% by weight of the releasing agent, up to 45% by weight of the releasing agent, or 50% by weight of the releasing agent, and
the second control layer includes up to 5% by weight of the releasing agent, up to 10% by weight of the releasing agent, up to 15% by weight of the releasing agent, up to 20% by weight of the releasing agent, up to 25% by weight of the releasing agent, up to 30% by weight of the releasing agent, up to 35% by weight of the releasing agent, up to 40% by weight of the releasing agent, up to 45% by weight of the releasing agent, or up to 50% by weight of the releasing agent.

154. The depot of any one of the clauses herein, wherein the second control layer is positioned between the first control layer and the therapeutic region, and wherein the first control layer includes a first amount of the releasing agent and the second control layer includes a second amount of the releasing agent, the second amount being at least 2×, at least 3×, at least 4×, or at least 5× the first amount.

155. The depot of any one of the clauses herein, wherein a thickness of the control region is less than or equal to 1/50, 1/75, or 1/100 of a thickness of the therapeutic region.

156. The depot of any one of the clauses herein, wherein the depot is a flexible solid that is structurally capable of being handled by a clinician during the normal course of a surgery without breaking into multiple pieces and/or losing its general shape.

157. The depot of any one of the clauses herein, wherein the depot is configured to be subcutaneously placed within a patient and release the therapeutic agent in vivo for up to 7 days without breaking into multiple pieces.

158. The depot of any one of the clauses herein, wherein the depot has a width and a thickness, and wherein a ratio of the width to the thickness is 21 or greater, at least 30 or greater, or at least 40 or greater.

159. The depot of any one of the clauses herein, wherein the depot has a surface area and a volume, and wherein a ratio of the surface area to volume is at least 1.

160. The depot of any one of the clauses herein, wherein the diffusion openings include at least one or more pores and/or one or more channels.

161. The depot of any one of the clauses herein, wherein the two or more micro-thin layers of the bioresorbable polymer are bonded via heat compression to form the therapeutic region.

162. The depot of any one of the clauses herein, wherein the control region and the therapeutic region are bonded via heat compression.

163. The depot of any one of the clauses herein, wherein the control region and the therapeutic region are thermally bonded.

164. The depot of any one of the clauses herein, wherein dissolution of the releasing agent following in vivo placement causes the control region and the therapeutic region to transition from a state of lesser porosity to a state of greater porosity to facilitate the release of the therapeutic agent from the depot.

165. The depot of any one of the clauses herein, wherein the releasing agent is a first releasing agent and the therapeutic region includes a second releasing agent mixed with the therapeutic agent.

166. The depot of any one of the clauses herein, wherein the releasing agent is a first releasing agent and the polymer is a first polymer, and the therapeutic region includes a second releasing agent and a second polymer mixed with the therapeutic agent.

167. The depot of any one of the clauses herein, wherein the first releasing agent is the same as the second releasing agent.

168. The depot of any one of the clauses herein, wherein the first releasing agent is the different than the second releasing agent.

169. The depot of any one of the clauses herein, wherein a concentration of the first releasing agent within the control region is the greater than a concentration of the second releasing agent within the therapeutic region.

170. The depot of any one of the clauses herein, wherein a concentration of the first releasing agent within the control region is the less than a concentration of the second releasing agent within the therapeutic region.

171. The depot of any one of the clauses herein, wherein a concentration of the first releasing agent within the control region is the same as a concentration of the second releasing agent within the therapeutic region.

172. The depot of any one of the clauses herein, wherein a concentration of the first releasing agent within the control region is different than a concentration of the second releasing agent within the therapeutic region.

173. The depot of any one of the clauses herein, wherein the therapeutic region includes a plurality of microlayers.

174. The depot of any one of the clauses herein, wherein the mass of the therapeutic agent comprises at least 50% of the mass of the depot.

175. The depot of any one of the clauses herein, wherein the ratio of the mass of the therapeutic agent in the depot to the depot polymer mass is at least 1:1, at least 2:1, at least 3:1, at least 4:1, at least 5:1, at least 6:1, at least 7:1, at least 8:1, at least 9:1, at least 10:1, or at least 16:1.

176. The depot of any one of the clauses herein, wherein the therapeutic region includes at least 50% by weight of the therapeutic agent, 60% by weight of the therapeutic agent, at least 70% by weight of the therapeutic agent, at least 80% by weight of the therapeutic agent, or at least 90% by weight of the therapeutic agent.

177. The depot of any one of the clauses herein, wherein the depot includes at least 15% by weight of the therapeutic agent, at least 20% by weight of the therapeutic agent, at least 30% by weight of the therapeutic agent, at least 40% by weight of the therapeutic agent, at least 50% by weight of the therapeutic agent, at least 60% by weight of the therapeutic agent, at least 70% by weight of the therapeutic agent, at least 80% by weight of the therapeutic agent, or at least 90% by weight of the therapeutic agent.

178. The depot of any one of the clauses herein, wherein the releasing agent is a non-ionic surfactant.

179. The depot of any one of the clauses herein, wherein the releasing agent has hydrophilic properties.

180. The depot of any one of the clauses herein, wherein the releasing agent is a polysorbate.

181. The depot of any one of the clauses herein, wherein the releasing agent is Tween 20.

182. The depot of any one of the clauses herein, wherein the releasing agent is Tween 80.

183. The depot of any one of the clauses herein, wherein the releasing agent is non-polymeric.

184. The depot of any one of the clauses herein, wherein the releasing agent is not a plasticizer.

185. The depot of any one of the clauses herein, wherein the polymer is configured to degrade only after substantially all of the therapeutic agent has been released from the depot.

186. The depot of any one of the clauses herein, wherein the polymer is a copolymer.

187. The depot of any one of the clauses herein, wherein the polymer is a terpolymer.

188. The depot of any one of the clauses herein, wherein the polymer includes at least one of polyglycolide (PGA), polycaprolactone (PCL), poly(DL-lactic acid) (PLA), poly(alpha-hydroxy acids), poly(lactide-co-glycolide)(PLGA or DLG), poly(DL-lactide-co-caprolactone) (DL-PLCL), poly(trimethylene carbonate) (PTMC), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (such as hyaluronic acid, chitosan and starch), proteins (such as gelatin and collagen) or PEG derivatives, polyaspirins, polyphosphagenes, pre-gelatinized starch, hyaluronic acid, chitosans, gelatin, alginates, albumin, fibrin, vitamin E analogs, such as alpha tocopheryl acetate, d-alpha tocopheryl succinate, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate)hydroxypropyl cellulose, hydroxypropyl methylcellulose, hydroxyethyl methylcellulose, carboxymethylcellulose or salts thereof, Carbopol®, poly(hydroxyethylmethacrylate), poly(methoxyethylmethacrylate), poly(methoxyethoxy-ethylmethacrylate), polymethylmethacrylate (PMMA), polyvinyl alcohols, propylene glycol, and poly(DL-lactide-co-glycolide-co-caprolactone).

189. The depot of any one of the clauses herein, wherein the polymer is one of poly (DL-lactide-co-glycolide-co-caprolactone) and poly(DL-lactide-co-glycolide)(PLGA).

190. The depot of any one of the clauses herein, wherein the polymer is poly (DL-lactide-co-glycolide-co-caprolactone) in a molar ratio of about 60:30:10.

191. The depot of any one of the clauses herein, wherein the polymer is poly (DL-lactide-co-glycolide)(PLGA) in a molar ratio of about 50:50.

192. The depot of any one of the clauses herein, wherein the polymer is ester-terminated.

193. The depot of any one of the clauses herein, wherein the polymer is a terpolymer that includes three polymers selected from the following: polyglycolide (PGA), polycaprolactone (PCL), poly(L-lactic acid) (PLA), poly(DL-lactic acid) (PLA), poly(trimethylene carbonate) (PTMC), polydioxanone (PDO), poly(-hydroxy butyrate) (PHB), polyhydroxyalkanoates (PHA), poly(phosphazene), and polyethylene glycol.

194. The depot of any one of the clauses herein, wherein the polymer is a first polymer, and the therapeutic region includes a second polymer mixed with the therapeutic agent.

195. The depot of any one of the clauses herein, wherein the first polymer and/or the second polymer include at least one of polyglycolide (PGA), polycaprolactone (PCL), poly(DL-lactic acid) (PLA), poly(alpha-hydroxy acids), poly(lactide-co-glycolide)(PLGA or DLG), poly(DL-lactide-co-caprolactone) (DL-PLCL), poly(trimethylene carbonate) (PTMC), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (such as hyaluronic acid, chitosan and starch), proteins (such as gelatin and collagen) or PEG derivatives, polyaspirins, polyphosphagenes, pre-gelatinized starch, hyaluronic acid, chitosans, gelatin, alginates, albumin, fibrin, vitamin E analogs, such as alpha tocopheryl acetate, d-alpha tocopheryl succinate, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate)hydroxypropyl cellulose, hydroxypropyl methylcellulose, hydroxyethyl methylcellulose, carboxymethylcellulose or salts thereof, Carbopol®, poly(hydroxyethylmethacrylate), poly(methoxyethylmethacrylate), poly(methoxyethoxy-ethylmethacrylate), polymethylmethacrylate (PMMA), polyvinyl alcohols, propylene glycol, poly(DL-lactide-co-glycolide-co-caprolactone).

196. The depot of any one of the clauses herein, wherein the first polymer and/or the second polymer selected from the following: poly (DL-lactide-co-glycolide-co-caprolactone) and poly(DL-lactide-co-glycolide)(PLGA).

197. The depot of any one of the clauses herein, wherein the first polymer and/or the second polymer is poly (DL-lactide-co-glycolide-co-caprolactone) and has a molar ratio of about 60:30:10.

198. The depot of any one of the clauses herein, wherein the first polymer and/or the second polymer is poly (DL-lactide-co-glycolide) and has a molar ratio of about 50:50.

199. The depot of any one of the clauses herein, wherein the first polymer and/or the second polymer is ester-terminated.

200. The depot of any one of the clauses herein, wherein the first polymer and/or the second polymer is a terpolymer that includes three polymers selected from the following: polyglycolide (PGA), polycaprolactone (PCL), poly(L-lactic acid) (PLA), poly(trimethylene carbonate) (PTMC), polydioxanone (PDO), poly(4-hydroxy butyrate) (PHB), polyhydroxyalkanoates (PHA), poly(phosphazene), and polyethylene glycol.

201. The depot of any one of the clauses herein, wherein the ratio of the releasing agent to the polymer in the control region is at least 1:1, at least 2:1, at least 3:1, at least 4:1, at least 5:1, at least 6:1, at least 7:1, at least 8:1, at least 9:1, at least 10:1, or at least 15:1.

202. The depot of any one of the clauses herein, wherein:

the polymer is a first polymer and the therapeutic region further includes a second polymer,

the depot has a depot polymer mass equivalent to a mass of the first polymer plus a mass of the second polymer, and

a ratio of a mass of the therapeutic agent in the depot to the depot polymer mass is approximately 1:1.

203. The depot of any one of the clauses herein, wherein the first polymer is the same as the second polymer.

204. The depot of any one of the clauses herein, wherein the first polymer is different than the second polymer.

205. The depot of any one of the clauses herein, wherein the ratio of the mass of the therapeutic agent in the depot to the depot polymer mass is at least 2:1, at least 3:1, at least 4:1, at least 5:1, at least 6:1, at least 7:1, at least 8:1, at least 9:1, at least 10:1, or at least 16:1 206. The depot of any one of the clauses herein, wherein the releasing agent is configured to dissolve when the depot is placed in contact with phosphate buffered saline to form diffusion openings.

207. The depot of any one of the clauses herein, wherein the depot comprises a rolled film configured to be implanted in a patient.

208. The depot of any one of the clauses herein, wherein the depot comprises an elongate columnar structure configured to be implanted in a patient.

209. The depot of any one of the clauses herein, wherein the depot comprises a dumpling configuration.

210. The depot of any one of the clauses herein, wherein the depot comprises a plurality of discrete microdepots.

211. The depot of any one of the clauses herein, wherein the depot comprises one of a plurality of beads.

212. The depot of any one of the clauses herein, wherein the depot comprises one of a plurality of microspheres.

213. The depot of any one of the clauses herein, wherein the depot comprises one of a plurality of microcylinders.

214. The depot of any one of the clauses herein, wherein the depot is configured to be subcutaneously implanted.

215. The depot of any one of the clauses herein, wherein the depot is configured to be intramuscularly implanted.

216. The depot of any one of the clauses herein, wherein the depot is implanted at a treatment site.

217. The depot of any one of the clauses herein, wherein the treatment site comprises an area at or proximate to the abdomen, deltoid, gluteal, arm, or thigh.

218. The depot of any one of the clauses herein, wherein the treatment site comprises a fatty layer between a patient's dermis and muscle.

219. A system for delivering a therapeutic agent to a treatment site, the system comprising:
a shaft having a lumen;
a pusher operatively coupled to the lumen; and
a depot disposed within the lumen and configured to be displaced from the shaft via activation of the pusher, the depot comprising:
   a therapeutic region comprising the therapeutic agent, the therapeutic region elongated along a first axis;
   a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region; and
   wherein the depot is configured to be implanted at a treatment site in vivo and, while implanted, release the therapeutic agent at the treatment site for a period of time not less than 3 days.

220. The system of any one of the clauses herein, wherein the depot comprises the depot of any one of the clauses herein.

221. The system of any one of the clauses herein, wherein the shaft comprises a needle, and wherein the pusher comprises a plunger.

222. The system of any one of the clauses herein, wherein the needle is no greater than 14, 16, 18, 20, or 22 gauge in size.

223. A system for delivering a therapeutic agent to a treatment site, the system comprising:
an expandable member configured to be expanded from a reduced-volume configuration for delivery to an expanded-volume configuration for deployment at the treatment site; and
a depot carried by the expandable member, the depot comprising:
   a therapeutic region comprising the therapeutic agent, the therapeutic region elongated along a first axis;
   a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region; and
   wherein the depot is configured to be implanted at a treatment site in vivo and, while implanted, release the therapeutic agent at the treatment site for a period of time not less than 3 days.

224. The system of any one of the clauses herein, wherein the depot comprises the depot of any one of the clauses herein.

225. The system of any one of the clauses herein, wherein the expandable member comprises a stent.

226. The system of any one of the clauses herein, wherein the expandable member comprises a spherical, semi-spherical, ellipsoid, or semi-ellipsoid structure.

227. The system of any one of the clauses herein, wherein the expandable member comprises a curved outer surface, and wherein the depot is disposed over the curved outer surface.

228. The system of any one of the clauses herein, wherein the depot substantially covers at least one surface of the expandable member.

229. The system of any one of the clauses herein, wherein the expandable member comprises a shape-memory material.

230. A system for delivering a therapeutic agent to a treatment site, the system comprising:
a delivery device; and
a depot configured to be delivered to a treatment site via the delivery device, the depot comprising:
   a therapeutic region comprising the therapeutic agent;
   a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region; and
   wherein the depot is configured to be implanted at a treatment site in vivo and, while implanted, release the therapeutic agent at the treatment site for a period of time not less than 3 days.

231. The system of any one of the clauses herein, wherein the depot is disposed in a lubricious coating and wherein the lubricious coating comprises a hydrogel.

232. The system of any one of the clauses herein, wherein the exposed surface area of the control region is less than the exposed surface area of the therapeutic region.

233. The system of any one of the clauses herein, wherein the exposed surface area of the control region is greater than the exposed surface area of the therapeutic region.

234. The system of any one of the clauses herein, wherein the control region is a first control region, and wherein the depot comprises a second control region.

235. The system of any one of the clauses herein, wherein the first control region is disposed at a first side of the therapeutic region and the second control region is disposed at a second side of the therapeutic region opposite the first side.

236. The system of any one of the clauses herein, wherein the depot comprises a plurality of control regions and a plurality of therapeutic regions, and wherein each of the therapeutic regions is separated from an adjacent one of the therapeutic regions by one or more control regions.

237. The system of any one of the clauses herein, wherein each of the therapeutic regions and each of the control regions is a micro-thin layer.

238. The system of any one of the clauses herein, wherein the depot comprises from about 2 to about 100 therapeutic regions, from about 2 to about 50 therapeutic regions, or from about 2 to about 10 therapeutic regions.

239. The system of any one of the clauses herein, wherein the therapeutic region is enclosed by the control region such that, when the depot is positioned in vivo, the control region is between the therapeutic region and in vivo physiologic fluids.

240. The system of any one of the clauses herein, wherein the control region comprises a first control layer and a second control layer.

241. The system of any one of the clauses herein, wherein the second control layer is adjacent to the therapeutic region and the first control layer encapsulates/encloses the therapeutic region and the second control layer.

242. The system of any one of the clauses herein, wherein the first control layer and the second control layer together enclose the therapeutic region.

243. The system of any one of the clauses herein, wherein the first control layer is disposed at a first side of the therapeutic region and the second control layer is disposed at a second side of the therapeutic region opposite the first side.

244. The system of any one of the clauses herein, wherein the first control layer comprises a first plurality of sub-layers and the second control layer comprises a second plurality of sub-layers.

245. The system of any one of the clauses herein, wherein the first control layer includes a first amount of the releasing agent and the second control layer includes a second amount of the releasing agent different than the first amount.

246. The system of any one of the clauses herein, wherein the second control layer is positioned between the first control layer and the therapeutic region, and wherein the first control layer includes a first concentration of the releasing agent and the second control layer includes a second concentration of the releasing agent greater than the first concentration.

247. The system of any one of the clauses herein, wherein the second control layer is positioned between the first control layer and the therapeutic region, and wherein the first control layer includes a first concentration of the releasing agent and the second control layer includes a second concentration of the releasing agent less than the first concentration.

248. The system of any one of the clauses herein, wherein the second control layer is positioned between the first control layer and the therapeutic region, and wherein:
the first control layer includes up to 5% by weight of the releasing agent, up to 10% by weight of the releasing agent, up to 15% by weight of the releasing agent, up to 20% by weight of the releasing agent, up to 25% by weight of the releasing agent, up to 30% by weight of the releasing agent, up to 35% by weight of the releasing agent, up to 40% by weight of the releasing agent, up to 45% by weight of the releasing agent, or 50% by weight of the releasing agent, and
the second control layer includes up to 5% by weight of the releasing agent, up to 10% by weight of the releasing agent, up to 15% by weight of the releasing agent, up to 20% by weight of the releasing agent, up to 25% by weight of the releasing agent, up to 30% by weight of the releasing agent, up to 35% by weight of the releasing agent, up to 40% by weight of the releasing agent, up to 45% by weight of the releasing agent, or up to 50% by weight of the releasing agent.

249. The system of any one of the clauses herein, wherein the second control layer is positioned between the first control layer and the therapeutic region, and wherein the first control layer includes a first amount of the releasing agent and the second control layer includes a second amount of the releasing agent, the second amount being at least 2×, at least 3×, at least 4×, or at least 5× the first amount.

250. The system of any one of the clauses herein, wherein a thickness of the control region is less than or equal to 1/50, 1/75, or 1/100 of a thickness of the therapeutic region.

251. The system of any one of the clauses herein, wherein the depot is a flexible solid that is structurally capable of being handled by a clinician during the normal course of a surgery without breaking into multiple pieces and/or losing its general shape.

252. The system of any one of the clauses herein, wherein the depot is configured to be subcutaneously placed within a patient and release the therapeutic agent in vivo for up to 7 days without breaking into multiple pieces.

253. The system of any one of the clauses herein, wherein the depot has a width and a thickness, and wherein a ratio of the width to the thickness is 21 or greater, 30 or greater, or 40 or greater.

254. The system of any one of the clauses herein, wherein the depot has a surface area and a volume, and wherein a ratio of the surface area to volume is at least 1.

255. The system of any one of the clauses herein, wherein the diffusion openings include at least one or more pores and/or one or more channels.

256. The system of any one of the clauses herein, wherein the two or more micro-thin layers of the bioresorbable polymer are bonded via heat compression to form the therapeutic region.

257. The system of any one of the clauses herein, wherein the control region and the therapeutic region are bonded via heat compression.

258. The system of any one of the clauses herein, wherein the control region and the therapeutic region are thermally bonded.

259. The system of any one of the clauses herein, wherein dissolution of the releasing agent following in vivo placement causes the control region and the therapeutic region to transition from a state of lesser porosity to a state of greater porosity to facilitate the release of the therapeutic agent from the depot.

260. The system of any one of the clauses herein, wherein the releasing agent is a first releasing agent and the therapeutic region includes a second releasing agent mixed with the therapeutic agent.

261. The system of any one of the clauses herein, wherein the releasing agent is a first releasing agent and the polymer is a first polymer, and the therapeutic region includes a second releasing agent and a second polymer mixed with the therapeutic agent.

262. The system of any one of the clauses herein, wherein the first releasing agent is the same as the second releasing agent.

263. The system of any one of the clauses herein, wherein the first releasing agent is the different than the second releasing agent.

264. The system of any one of the clauses herein, wherein a concentration of the first releasing agent within the control region is the greater than a concentration of the second releasing agent within the therapeutic region.

265. The system of any one of the clauses herein, wherein a concentration of the first releasing agent within the control region is the less than a concentration of the second releasing agent within the therapeutic region.

266. The system of any one of the clauses herein, wherein a concentration of the first releasing agent within the control region is the same as a concentration of the second releasing agent within the therapeutic region.

267. The system of any one of the clauses herein, wherein a concentration of the first releasing agent within the control region is different than a concentration of the second releasing agent within the therapeutic region.

268. The system of any one of the clauses herein, wherein the therapeutic region includes a plurality of microlayers.

269. The system of any one of the clauses herein, wherein the mass of the therapeutic agent comprises at least 50% of the mass of the depot.

270. The system of any one of the clauses herein, wherein the ratio of the mass of the therapeutic agent in the depot to the depot polymer mass is at least 3:1, at least 4:1, at least 5:1, at least 6:1, at least 7:1, at least 8:1, at least 9:1, at least 10:1, or at least 16:1.

271. The system of any one of the clauses herein, wherein the therapeutic region includes at least 50% by weight of the therapeutic agent, 60% by weight of the therapeutic agent, at least 70% by weight of the therapeutic agent, at least 80% by weight of the therapeutic agent, or at least 90% by weight of the therapeutic agent.

272. The system of any one of the clauses herein, wherein the depot includes at least 15% by weight of the therapeutic agent, at least 20% by weight of the therapeutic agent, at least 30% by weight of the therapeutic agent, at least 40% by weight of the therapeutic agent, at least 50% by weight of the therapeutic agent, at least 60% by weight of the therapeutic agent, at least 70% by weight of the therapeutic agent, at least 80% by weight of the therapeutic agent, or at least 90% by weight of the therapeutic agent.

273. The system of any one of the clauses herein, wherein the releasing agent is a non-ionic surfactant.

274. The system of any one of the clauses herein, wherein the releasing agent has hydrophilic properties.

275. The system of any one of the clauses herein, wherein the releasing agent is a polysorbate.

276. The system of any one of the clauses herein, wherein the releasing agent is Tween 20.

277. The system of any one of the clauses herein, wherein the releasing agent is Tween 80.

278. The system of any one of the clauses herein, wherein the releasing agent is non-polymeric.

279. The system of any one of the clauses herein, wherein the releasing agent is not a plasticizer.

280. The system of any one of the clauses herein, wherein the polymer is configured to degrade only after substantially all of the therapeutic agent has been released from the depot.

281. The system of any one of the clauses herein, wherein the polymer is a copolymer.

282. The system of any one of the clauses herein, wherein the polymer is a terpolymer.

283. The system of any one of the clauses herein, wherein the polymer includes at least one of polyglycolide (PGA), polycaprolactone (PCL), poly(DL-lactic acid) (PLA), poly(alpha-hydroxy acids), poly(lactide-co-glycolide)(PLGA or DLG), poly(DL-lactide-co-caprolactone) (DL-PLCL), poly(trimethylene carbonate) (PTMC), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (such as hyaluronic acid, chitosan and starch), proteins (such as gelatin and collagen) or PEG derivatives, polyaspirins, polyphosphagenes, pre-gelatinized starch, hyaluronic acid, chitosans, gelatin, alginates, albumin, fibrin, vitamin E analogs, such as alpha tocopheryl acetate, d-alpha tocopheryl succinate, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAM (sucrose acetate isobutyrate)hydroxypropyl cellulose, hydroxypropyl methylcellulose, hydroxyethyl methylcellulose, carboxymethylcellulose or salts thereof, Carbopol®, poly(hydroxyethylmethacrylate), poly(methoxyethylmethacrylate), poly(methoxyethoxy-ethylmethacrylate), polymethylmethacrylate (PMMA), polyvinyl alcohols, propylene glycol, and poly(DL-lactide-co-glycolide-co-caprolactone).

284. The system of any one of the clauses herein, wherein the polymer is one of poly (DL-lactide-co-glycolide-co-caprolactone) and poly(DL-lactide-co-glycolide)(PLGA).

285. The system of any one of the clauses herein, wherein the polymer is poly (DL-lactide-co-glycolide-co-caprolactone) in a molar ratio of about 60:30:10.

286. The system of any one of the clauses herein, wherein the polymer is poly (DL-lactide-co-glycolide)(PLGA) in a molar ratio of about 50:50.

287. The system of any one of the clauses herein, wherein the polymer is ester-terminated.

288. The system of any one of the clauses herein, wherein the polymer is a terpolymer that includes three polymers selected from the following: polyglycolide (PGA), polycaprolactone (PCL), poly(L-lactic acid) (PLA), poly(DL-lactic acid) (PLA), poly(trimethylene carbonate) (PTMC), polydioxanone (PDO), poly(-hydroxy butyrate) (PHB), polyhydroxyalkanoates (PHA), poly(phosphazene), and polyethylene glycol.

289. The system of any one of the clauses herein, wherein the polymer is a first polymer, and the therapeutic region includes a second polymer mixed with the therapeutic agent.

290. The system of any one of the clauses herein, wherein the first polymer and/or the second polymer include at least one of polyglycolide (PGA), polycaprolactone (PCL), poly(DL-lactic acid) (PLA), poly(alpha-hydroxy acids), poly(lactide-co-glycolide)(PLGA or DLG), poly(DL-lactide-co-caprolactone) (DL-PLCL), poly(trimethylene carbonate) (PTMC), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (such as hyaluronic acid, chitosan and starch), proteins (such as gelatin and collagen) or PEG derivatives, polyaspirins, polyphosphagenes, pre-gelatinized starch, hyaluronic acid, chitosans, gelatin, alginates, albumin, fibrin, vitamin E analogs, such as alpha tocopheryl acetate, d-alpha tocopheryl succinate, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate)hydroxypropyl cellulose, hydroxypropyl methylcellulose, hydroxyethyl methylcellulose, carboxymethylcellulose or salts thereof, Carbopol®, poly(hydroxyethylmethacrylate), poly(methoxyethylmethacrylate), poly(methoxyethoxy-ethylmethacrylate), polymethylmethacrylate (PMMA), polyvinyl alcohols, propylene glycol, poly(DL-lactide-co-glycolide-co-caprolactone).

291. The system of any one of the clauses herein, wherein the first polymer and/or the second polymer selected from the following: poly (DL-lactide-co-glycolide-co-caprolactone) and poly(DL-lactide-co-glycolide)(PLGA).

292. The system of any one of the clauses herein, wherein the first polymer and/or the second polymer is poly (DL-lactide-co-glycolide-co-caprolactone) and has a molar ratio of about 60:30:10.

293. The system of any one of the clauses herein, wherein the first polymer and/or the second polymer is poly (DL-lactide-co-glycolide) and has a molar ratio of about 50:50.

294. The system of any one of the clauses herein, wherein the first polymer and/or the second polymer is ester-terminated.

295. The system of any one of the clauses herein, wherein the first polymer and/or the second polymer is a terpolymer that includes three polymers selected from the following: polyglycolide (PGA), polycaprolactone (PCL), poly(L-lactic acid) (PLA), poly(trimethylene carbonate) (PTMC), polydioxanone (PDO), poly(4-hydroxy butyrate) (PHB), polyhydroxyalkanoates (PHA), poly(phosphazene), and polyethylene glycol.

296. The system of any one of the clauses herein, wherein the ratio of the releasing agent to the polymer in the control region is at least 1:1, at least 2:1, at least 3:1, at least 4:1, at least 5:1, at least 6:1, at least 7:1, at least 8:1, at least 9:1, at least 10:1, or at least 15:1.

297. The system of any one of the clauses herein, wherein:

the polymer is a first polymer and the therapeutic region further includes a second polymer,

the depot has a depot polymer mass equivalent to a mass of the first polymer plus a mass of the second polymer, and

a ratio of a mass of the therapeutic agent in the depot to the depot polymer mass is approximately 1:1.

298. The system of any one of the clauses herein, wherein the first polymer is the same as the second polymer.

299. The system of any one of the clauses herein, wherein the first polymer is different than the second polymer.

300. The system of any one of the clauses herein, wherein the ratio of the mass of the therapeutic agent in the depot to the depot polymer mass is at least 2:1, at least 3:1, at least 4:1, at least 5:1, at least 6:1, at least 7:1, at least 8:1, at least 9:1, at least 10:1, or at least 16:1.

301. The system of any one of the clauses herein, wherein the releasing agent is configured to dissolve when the depot is placed in contact with phosphate buffered saline to form diffusion openings.

302. A method for delivering a therapeutic agent to a treatment site within a body:
positioning a depot at a treatment site in vivo having physiologic fluids, the depot comprising:
   a therapeutic region comprising the therapeutic agent, the therapeutic region elongated along a first axis;
   a control region at least partially surrounding the therapeutic region and elongated along the first axis, the control region comprising a bioresorbable polymer and a releasing agent mixed with the polymer; and
allowing the releasing agent to dissolve at the treatment site to form diffusion openings in the control region, thereby releasing the therapeutic agent from the depot for a period of time.

303. The method of any one of the clauses herein, wherein the depot comprises the depot of any one of the clauses herein.

304. The method of any one of the clauses herein, wherein positioning the depot comprises inserting the depot subcutaneously at the treatment site via a needle.

305. The method of any one of the clauses herein, wherein the needle is no greater than 14, 16, 18, 20, or 22 gauge in size.

306. The method of any one of the clauses herein, wherein positioning the depot comprises positioning the depot proximate to a nerve bundle at the treatment site.

307. The method of any one of the clauses herein, further comprising dissolving the releasing agent at a first rate and degrading the polymer at a second rate, wherein the first rate is greater than the second rate.

308. The method of any one of the clauses herein, further comprising dissolving the releasing agent in response to contact between the control region and the physiologic fluids at the treatment site.

309. The method of any one of the clauses herein, further comprising creating diffusion openings in the control region via the dissolution of the releasing agent in response to physiologic fluids at the treatment site.

310. The method of any one of the clauses herein, wherein the releasing agent is a first releasing agent and the therapeutic region includes a second releasing agent, and wherein the method further comprises creating microchannels in the therapeutic region and the control region via dissolution of the first and/or second releasing agents.

311. The method of any one of the clauses herein, wherein at least some of the microchannels penetrate both the therapeutic region and the control region.

312. The method of any one of the clauses herein, further including increasing a porosity of the depot via dissolution of the releasing agent.

313. The method of any one of the clauses herein, wherein the therapeutic agent is released one or more times in substantially discrete doses after implantation.

314. The method of any one of the clauses herein, wherein the therapeutic agent is released at a substantially steady state rate for the period of time.

315. The method of any one of the clauses herein, wherein the period of time is not less than 8 days, no less than 9 days, no less than 10 days, no less than 11 days, no less than 12 days, no less than 13 days, no less than 14 days, no less than 15 days, no less than 16 days, no less than 17 days, no less than 18 days, no less than 19 days, no less than 20 days, no less than 21 days, no less than 22 days, no less than 23 days, no less than 24 days, no less than 25 days, no less than 26 days, no less than 27 days, no less than 28 days, no less than 29 days, no less than 30 days, no less than 40 days, no less than 50 days, no less than 60 days, no less than 70 days, no less than 90 days, no less than 100 days, no less than 200 days, no less than 300 days, or no less than 365 days.

316. The method of any one of the clauses herein, wherein the depot is a first depot and the method further comprises positioning a second depot at the treatment site.

317. A depot for the treatment of symptoms associated with type II diabetes, comprising:
a therapeutic region comprising a therapeutic agent, the therapeutic agent including a glucagon-like peptide-1 (GLP-1) receptor agonist; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted in vivo and, while implanted, release the GLP-1 receptor agonist for a period of time.

318. The depot of any one of the clauses herein, wherein the GLP-1 receptor agonist comprises exenatide.

319. The depot of any one of the clauses herein, wherein the GLP-1 receptor agonist comprises liraglutide.

320. The depot of any one of the clauses herein, wherein the GLP-1 receptor agonist comprises exenatide, liraglutide, albiglutide, dulaglutide, lixisenatide, semaglutide, derivatives thereof, or combinations thereof. 321. The depot of any one of the clauses herein, wherein the therapeutic agent is a first therapeutic agent, the therapeutic region further comprising a second therapeutic agent comprising metformin.

322. The depot of any one of the clauses herein, wherein the first therapeutic agent is released before or after the second therapeutic agent.

323. The depot of any of one the clauses herein, wherein the first and second therapeutic agents are released substantially simultaneously.

324. The depot of any one of the clauses herein, wherein the GLP-1 receptor agonist in the therapeutic region comprises at least 50% of the total weight of the depot.

325. The depot of any one of the clauses herein, wherein the period of time is no less than 1 month.

326. The depot of any one of the clauses herein, wherein the period of time is no less than 2 months.

327. The depot of any one of the clauses herein, wherein the period of time is no less than 3 months.

328. The depot of any one of the clauses herein, wherein the period of time is no less than 4 months.

329. The depot of any one of the clauses herein, wherein the period of time is no less than 5 months.

330. The depot of any one of the clauses herein, wherein the period of time is no less than 6 months.

331. The depot of any one of the clauses herein, wherein the depot is biodegradable and/or bioerodible.

332. The depot of any one of the clauses herein, wherein about 40% to about 60% of the GLP-1 receptor agonist in the therapeutic region is released in the first half of the period of time.

333. The depot of any one of the clauses herein, wherein at least 90% of the GLP-1 receptor agonist in the therapeutic region is released within the period of time.

334. The depot of any one of the clauses herein, wherein the depot is configured to release about 2 μg/day to about 10 mg/day of the GLP-1 receptor agonist.

335. The depot of any one of the clauses herein, wherein the depot is configured to release less than about 10 mg/day of the GLP-1 receptor agonist.

336. The depot of any one of the clauses herein, wherein the depot is configured to release about 0.2 nmol/day to about 6 μmol/day of the GLP-1 receptor agonist.

337. The depot of any one of the clauses herein, wherein the depot is configured to release less than about 6 μmol/day of the GLP-1 receptor agonist.

338. The depot of any one of the clauses herein, wherein the depot is configured to release about 10 μg/day to about 30 μg/day of the GLP-1 receptor agonist.

339. The depot of any one of the clauses herein, wherein the depot is configured to release less than about 30 μg/day of the GLP-1 receptor agonist.

340. The depot of any one of the clauses herein, wherein the depot is configured to release about 2 nmol/day to about 10 nmol/day of GLP-1 receptor agonist.

341. The depot of any one of the clauses herein, wherein the depot is configured to release less than about 10 nmol/day of the GLP-1 receptor agonist.

342. The depot of any one of the clauses herein, wherein the depot is configured to release about 0.5 mg/day to about 5 mg/day of the GLP-1 receptor agonist.

343. The depot of any one of the clauses herein, wherein the depot is configured to release less than about 10 mg/day of the GLP-1 receptor agonist.

344. The depot of any one of the clauses herein, wherein the depot is configured to release about 0.1 μmol/day to about 0.5 μmol/day of GLP-1 receptor agonist.

345. The depot of any one of the clauses herein, wherein the depot is configured to release less than about 0.5 μmol/day of the GLP-1 receptor agonist.

346. The depot of any one of the clauses herein, wherein the depot is configured to release about 0.25 mg/day to about 1 mg/day of the GLP-1 receptor agonist.

347. The depot of any one of the clauses herein, wherein the depot is configured to release less than about 1 mg/day of the GLP-1 receptor agonist.

348. The depot of any one of the clauses herein, wherein the depot is configured to release about 0.05 μmol/day to about 0.2 μmol/day of the GLP-1 receptor agonist.

349. The depot of any one of the clauses herein, wherein the depot is configured to release less than about 0.2 μmol/day of the GLP-1 receptor agonist.

350. The depot of any one of the clauses herein, wherein no more than 400 μg/day, no more than 300 μg/day, no more than 200 μg/day, no more than 100 μg/day, no more than 75 μg/day, no more than 50 μg/day, no more than 40 μg/day, no more than 30 μg/day, no more than 20 μg/day, no more than 10 μg/day, or no more than 5 μg/day of the GLP-1 receptor agonist is released within any day of the period of time.

351. The depot of any one of the clauses herein, wherein less than about 500 nmol/day, less than about 300 nmol/day, less than about 250 nmol/day, less than about 200 nmol/day, less than about 175 nmol/day, less than about 150 nmol/day, less than about 125 nmol/day, less than about 100 nmol/day, less than about 90 nmol/day, less than about 80 nmol/day, less than about 70 nmol/day, less than about 60 nmol/day, less than about 50 nmol/day, less than about 45 nmol/day, less than about 40 nmol/day, less than about 35 nmol/day, less than about 30 nmol/day, less than about 25 nmol/day, less than about 20 nmol/day, less than about 15 nmol/day, or less than about 10 nmol/day of the GLP-1 receptor agonist is released within any day of the period of time.

352. The depot of any one of the clauses herein, wherein the period of time is no less than 1 day, no less than 2 days, no less than 3 days, no less than 4 days, no less than 5 days, no less than 6 days, no less than 7 days, no less than 8 days, no less than 9 days, no less than 10 days, no less than 11 days, no less than 12 days, no less than 13 days, no less than 14 days, no less than 15 days, no less than 16 days, no less than 17 days, no less than 18 days, no less than 19 days, no less than 20 days, no less than 21 days, no less than 22 days, no less than 23 days, no less than 24 days, no less than 25 days, no less than 26 days, no less than 27 days, no less than 28 days, no less than 29 days, no less than 30 days, no less than 40 days, no less than 50 days, no less than 60 days, no less than 70 days, no less than 90 days, no less than 100 days, no less than 200 days, no less than 300 days, or no less than 365 days.

353. The depot of any one of the clauses herein, wherein the GLP-1 receptor agonist is released at a substantially steady state rate throughout the period of time.

354. The depot of any one of the clauses herein, wherein the GLP-1 receptor agonist is continuously released throughout the period of time.

355. The depot of any one of the clauses herein, wherein the release of the GLP-1 receptor agonist decreases appetite, stimulates insulin secretion and/or slows gastric emptying.

356. The depot of any one of the clauses herein, wherein the depot further comprises a thermal stabilizer.

357. The depot of any one of the clauses herein, wherein the thermal stabilizer comprises at least one of a sugar, antioxidant or buffer.

358. The depot of any one of the clauses herein, wherein the sugar comprises at least one of trehalose, raffinose or mannitol.

359. The depot of any one of the clauses herein, wherein the antioxidant comprises at least one of methionine, ascorbic acid, sodium thiosulfate, catalase, platinum ethylenediaminetetraacetic acid (EDTA), citric acid, cysteins, thioglycerol, thioglycolic acid, thiosorbitol, butylated hydroxyanisol, butylated hydroxyltoluene, and propyl gallate.

360. The depot of any one of the clauses herein, wherein the buffer comprises at least one of citrate, histidine, succinate or tris.

361. The depot of any one of the clauses herein, wherein the GLP-1 receptor agonist comprises at least one of the sugar, antioxidant or buffer.

362. The depot of any one of the clauses herein, wherein at least one of the sugar, antioxidant or buffer at least partially encapsulates the GLP-1 receptor agonist.

363. The depot of any one of the clauses herein, wherein the depot further comprises a chemical compound configured to inhibit denaturing of the therapeutic agent in vivo.

364. A method for treating a patient having symptoms associated with diabetes, comprising:
positioning a depot at a treatment site in vivo having physiologic fluids, the depot comprising (a) a control region including a bioresorbable polymer and a releasing agent mixed with the polymer, and (b) a therapeutic region comprising at least 50% by weight of a therapeutic agent including a glucagon-like peptide-1 (GLP-1) receptor agonist; and
releasing the GLP-1 receptor agonist from the depot to the treatment site for a period of time.

365. The method of the immediately preceding clause, wherein the depot comprises the depots of any one of the clauses herein.

366. The method of any one of the clauses herein, wherein the GLP-1 receptor agonist comprises exenatide.

367. The method of any one of the clauses herein, wherein the GLP-1 receptor agonist comprises liraglutide.

368. The method of any one of the clauses herein, wherein the GLP-1 receptor agonist comprises exenatide, liraglutide, albiglutide, dulaglutide, lixisenatide, semaglutide, derivatives thereof, or combinations thereof. 369. The method of any one of the clauses herein, wherein the GLP-1 receptor agonist in the therapeutic region comprises at least 50% of the total weight of the depot.

370. The method of any one of the clauses herein, wherein the depot is biodegradable and/or bioerodible.

371. The method of any one of the clauses herein, wherein about 40% to about 60% of the GLP-1 receptor agonist in the therapeutic region is released in the first half of the period of time.

372. The method of any one of the clauses herein, wherein at least 90% of the GLP-1 receptor agonist in the therapeutic region is released within the period of time.

373. The method of any one of the clauses herein, wherein releasing the GLP-1 receptor agonist includes releasing the GLP-1 receptor agonist at a rate of about 2 μg/day to about 10 mg/day.

374. The method of any one of the clauses herein, wherein releasing the GLP-1 receptor agonist includes releasing the GLP-1 receptor agonist at a rate less than about 10 mg/day.

375. The method of any one of the clauses herein, wherein releasing the GLP-1 receptor agonist includes releasing the GLP-1 receptor agonist at a rate of about 0.2 nmol/day to about 6 μmol/day.

376. The method of any one of the clauses herein, wherein releasing the GLP-1 receptor agonist includes releasing the GLP-1 receptor agonist at a rate less than about 6 μmol/day, 377. The method of any one of the clauses herein, wherein releasing the GLP-1 receptor agonist includes releasing the GLP-1 receptor agonist at a rate of about 10 μg/day to about 30 μg/day.

378. The method of any one of the clauses herein, wherein releasing the GLP-1 receptor agonist includes releasing the GLP-1 receptor agonist at a rate less than about 30 μg/day.

379. The method of any one of the clauses herein, wherein releasing the GLP-1 receptor agonist includes releasing the GLP-1 receptor agonist at a rate of about 2 nmol/day to about 10 nmol/day of GLP-1 receptor agonist.

380. The method of any one of the clauses herein, wherein releasing the GLP-1 receptor agonist includes releasing the GLP-1 receptor agonist at a rate less than about 10 nmol/day.

381. The method of any one of the clauses herein, wherein releasing the GLP-1 receptor agonist includes releasing the GLP-1 receptor agonist at a rate of about 0.5 mg/day to about 10 mg/day.

382. The method of any one of the clauses herein, wherein releasing the GLP-1 receptor agonist includes releasing the GLP-1 receptor agonist at a rate less than about 10 mg/day.

383. The method of any one of the clauses herein, wherein releasing the GLP-1 receptor agonist includes releasing the GLP-1 receptor agonist at a rate of about 0.1 μmol/day to about 0.5 μmol/day.

384. The method of any one of the clauses herein, wherein releasing the GLP-1 receptor agonist includes releasing the GLP-1 receptor agonist at a rate less than about 0.5 μmol/day.

385. The method of any one of the clauses herein, wherein releasing the GLP-1 receptor agonist includes releasing the GLP-1 receptor agonist at a rate of about 0.25 mg/day to about 1 mg/day.

386. The method of any one of the clauses herein, wherein releasing the GLP-1 receptor agonist includes releasing the GLP-1 receptor agonist at a rate less than about 1 mg/day.

387. The method of any one of the clauses herein, wherein releasing the GLP-1 receptor agonist includes releasing the GLP-1 receptor agonist at a rate of about 0.05 μmol/day to about 0.2 μmol/day.

388. The method of any one of the clauses herein, wherein releasing the GLP-1 receptor agonist includes releasing the GLP-1 receptor agonist at a rate less than about 0.2 μmol/day.

389. The method of any one of the clauses herein, wherein releasing the GLP-1 receptor agonist includes releasing the GLP-1 receptor agonist at a rate no more than about 400 μg/day, no more than 300 μg/day, no more than 200 μg/day, no more than 100 μg/day, no more than 75 μg/day, no more than 50 μg/day, no more than 40 μg/day, no more than 30 μg/day, no more than 20 μg/day, no more than 10 μg/day, or no more than 5 μg/day within any day of the period of time.

390. The method of any one of the clauses herein, wherein the period of time is no less than 1 day, no less than 2 days, no less than 3 days, no less than 4 days, no less than 5 days, no less than 6 days, no less than 7 days, no less than 8 days, no less than 9 days, no less than 10 days, no less than 11 days, no less than 12 days, no less than 13 days, no less than 14 days, no less than 15 days, no less than 16 days, no less than 17 days, no less than 18 days, no less than 19 days, no less than 20 days, no less than 21 days, no less than 22 days, no less than 23 days, no less than 24 days, no less than 25 days, no less than 26 days, no less than 27 days, no less than 28 days, no less than 29 days, no less than 30 days, no less than 40 days, no less than 50 days, no less than 60 days, no less than 70 days, no less than 90 days, no less than 100 days, no less than 200 days, no less than 300 days, or no less than 365 days.

391. The method of any one of the clauses herein, wherein releasing the GLP-1 receptor agonist includes releasing the GLP-1 receptor agonist at a substantially steady state rate throughout the period of time.

392. The method of any one of the clauses herein, wherein the treatment site is a fat layer between a patient's dermis and muscle.

393. The method of any one of the clauses herein, wherein the depot further comprises a thermal stabilizer.

394. The method of any one of the clauses herein, wherein the thermal stabilizer comprises at least one of a sugar, antioxidant or buffer.

395. The method of any one of the clauses herein, wherein the sugar comprises at least one of trehalose, raffinose or mannitol.

396. The method of any one of the clauses herein, wherein the antioxidant comprises at least one of methionine, ascorbic acid, sodium thiosulfate, catalase, platinum ethylenediaminetetraacetic acid (EDTA), citric acid, cysteins, thioglycerol, thioglycolic acid, thiosorbitol, butylated hydroxyanisol, butylated hydroxyltoluene, and propyl gallate.

397. The method of any one of the clauses herein, wherein the buffer comprises at least one of citrate, histidine, succinate or tris.

398. The method of any one of the clauses herein, wherein the GLP-1 receptor agonist comprises at least one of the sugar, antioxidant or buffer.

399. The method of any one of the clauses herein, wherein at least one of the sugar, antioxidant or buffer at least partially encapsulates the GLP-1 receptor agonist.

400. The method of any one of the clauses herein, wherein the treatment site comprises an area at or adjacent a patient's abdominal area, gluteal area, femur or arm.

401. The method of any one of the clauses herein, wherein the treatment site is proximate or within the peritoneal cavity.

402. The method of anyone of the clauses herein, wherein positioning the depot comprises subcutaneously implanting the depot at or proximate to a patient's abdominal area, gluteal area, femur or arm.

403. The method of any one of the clauses herein, further comprising dissolving the releasing agent at a first rate and degrading the polymer at a second rate, wherein the first rate is greater than the second rate.

404. The method of any one of the clauses herein, further comprising dissolving the releasing agent in response to contact between the control region and the physiologic fluids at the treatment site.

405. The method of any one of the clauses herein, further comprising creating diffusion openings in the control region via the dissolution of the releasing agent in response to physiologic fluids at the treatment site.

406. The method of any one of the clauses herein, wherein the releasing agent is a first releasing agent and the therapeutic region includes a second releasing agent, and wherein the method further comprises creating microchannels in the therapeutic region and the control region via dissolution of the first and/or second releasing agents.

407. The method of any one of the clauses herein, wherein at least some of the microchannels penetrate both the therapeutic region and the control region.

408. The method of any one of the clauses herein, wherein the therapeutic region comprises a plurality of microlayers, and wherein at least some of the microchannels extend through consecutive microlayers.

409. The method of any one of the clauses herein, wherein the control region comprises a first plurality of microlayers and the therapeutic region comprises a second plurality of microlayers, and wherein at least some of the microchannels extend through the first and second plurality of microlayers.

410. The method of any one of the clauses herein, further comprising increasing a porosity of the depot via dissolution of the releasing agent.

411. The method of any one of the clauses herein, wherein the therapeutic agent is released one or more times in substantially discrete doses after implantation.

412. The method of any one of the clauses herein, wherein the depot is a first depot and the method further comprises positioning a second depot at the treatment site.

413. The method of any one of the clauses herein, wherein the releasing agent is configured to dissolve when the depot is placed in contact with phosphate buffered saline to form diffusion openings.

414. A system for delivering a therapeutic agent to a patient, the system comprising:
a delivery device having a distal region configured to be positioned under the skin of a patient; and
a depot positioned within the delivery device and configured to be emitted from the distal region to the patient, the depot comprising:
   a therapeutic region comprising the therapeutic agent, the therapeutic agent including glucagon-like peptide-1 (GLP-1) receptor agonist; and
   a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region;
wherein the depot is configured to be implanted in vivo and, while implanted, release the therapeutic agent for a period of time.

415. A system for delivering a therapeutic agent to a patient, the system comprising:
a needle having a lumen;
a syringe operatively coupled to the needle; and
a depot disposed within the lumen and configured to be emitted from the needle via activation of the syringe, the depot comprising:
   a therapeutic region comprising the therapeutic agent, the therapeutic agent including glucagon-like peptide-1 (GLP-1) receptor agonist; and
   a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region;
wherein the depot is configured to be implanted in vivo and, while implanted, release the therapeutic agent for a period of time.

416. The system of the immediately preceding clause, wherein the depot comprises a depot of any one of the clauses herein.

417. The system of any one of the clauses herein, wherein the needle is no greater than 14, 16, 18, 20, or 22 gauge in size.

418. A system for delivering a therapeutic agent to a patient, the system comprising:
an expandable member configured to be expanded from a reduced-volume configuration for delivery to an expanded-volume configuration for deployment; and
a depot carried by the expandable member, the depot comprising:
   a therapeutic region comprising the therapeutic agent, the therapeutic agent including a glucagon-like peptide-1 (GLP-1) receptor agonist; and
   a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region,
wherein the depot is configured to be implanted in vivo and, while implanted, release the GLP-1 receptor agonist for a period of time.

419. The system of the immediately preceding clause, wherein the depot comprises a depot of any one of the clauses herein.

420. The system of any one of the clauses herein, wherein the GLP-1 receptor agonist comprises exenatide.

421. The system of any one of the clauses herein, wherein the GLP-1 receptor agonist comprises liraglutide.

422. The system of any one of the clauses herein, wherein the GLP-1 receptor agonist comprises exenatide, liraglutide, albiglutide, dulaglutide, lixisenatide, semaglutide, derivatives thereof, or combinations thereof. 423. The system of any one of the clauses herein, wherein the GLP-1 receptor agonist in the therapeutic region comprises at least 50% of the total weight of the depot.

424. The system of any one of the clauses herein, wherein about 40% to about 60% of the GLP-1 receptor agonist in the therapeutic region is released in the first half of the period of time.

425. The system of any one of the clauses herein, wherein at least 90% of the GLP-1 receptor agonist in the therapeutic region is released within the period of time.

426. The system of any one of the clauses herein, wherein the depot is configured to release about 2μg/day to about 10 mg/day of the GLP-1 receptor agonist.

427. The system of any one of the clauses herein, wherein the depot is configured to release less than about 10 mg/day of the GLP-1 receptor agonist.

428. The system of any one of the clauses herein, wherein the depot is configured to release about 0.2 nmol/day to about 6 μmol/day of the GLP-1 receptor agonist.

429. The system of any one of the clauses herein, wherein the depot is configured to release less than about 6 μmol/day of the GLP-1 receptor agonist.

430. The system of any one of the clauses herein, wherein the depot is configured to release about 10 μg/day to about 30 μg/day of the GLP-1 receptor agonist.

431. The system of any one of the clauses herein, wherein the depot is configured to release less than about 30 μg/day of the GLP-1 receptor agonist.

432. The system of any one of the clauses herein, wherein the depot is configured to release about 2 nmol/day to about 10 nmol/day of GLP-1 receptor agonist.

433. The system of any one of the clauses herein, wherein the depot is configured to release less than about 10 nmol/day of the GLP-1 receptor agonist.

434. The system of any one of the clauses herein, wherein the depot is configured to release about 0.5 mg/day to about 10 mg/day of the GLP-1 receptor agonist.

435. The system of any one of the clauses herein, wherein the depot is configured to release less than about 10 mg/day of the GLP-1 receptor agonist.

436. The system of any one of the clauses herein, wherein the depot is configured to release about 0.1 μmol/day to about 0.5 μmol/day of GLP-1 receptor agonist.

437. The system of any one of the clauses herein, wherein the depot is configured to release less than about 0.5 μmol/day of the GLP-1 receptor agonist.

438. The system of any one of the clauses herein, wherein the depot is configured to release about 0.25 mg/day to about 1 mg/day of the GLP-1 receptor agonist.

439. The system of any one of the clauses herein, wherein the depot is configured to release less than about 1 mg/day of the GLP-1 receptor agonist.

440. The system of any one of the clauses herein, wherein the depot is configured to release about 0.05 μmol/day to about 0.2 μmol/day of the GLP-1 receptor agonist.

441. The system of any one of the clauses herein, wherein the depot is configured to release less than about 0.2 μmol/day of the GLP-1 receptor agonist.

442. The system of any one of the clauses herein, wherein no more than 400 μg/day, no more than 300 μg/day, no more than 200 μg/day, no more than 100 μg/day, no more than 75 μg/day, no more than 50 μg/day, no more than 40 μg/day, no more than 30 μg/day, no more than 20 μg/day, no more than 10 μg/day, or no more than 5 μg/day of the GLP-1 receptor agonist is released within any day of the period of time.

443. The system of any one of the clauses herein, wherein the period of time is no less than 1 day, no less than 2 days, no less than 3 days, no less than 4 days, no less than 5 days, no less than 6 days, no less than 7 days, no less than 8 days, no less than 9 days, no less than 10 days, no less than 11 days, no less than 12 days, no less than 13 days, no less than 14 days, no less than 15 days, no less than 16 days, no less than 17 days, no less than 18 days, no less than 19 days, no less than 20 days, no less than 21 days, no less than 22 days, no less than 23 days, no less than 24 days, no less than 25 days, no less than 26 days, no less than 27 days, no less than 28 days, no less than 29 days, no less than 30 days, no less than 40 days, no less than 50 days, no less than 60 days, no less than 70 days, no less than 90 days, no less than 100 days, no less than 200 days, no less than 300 days, or no less than 365 days.

444. The system of any one of the clauses herein, wherein the GLP-1 receptor agonist is released at a substantially steady state rate throughout the period of time.

445. The system of any one of the clauses herein, wherein the GLP-1 receptor agonist is released on a continuous basis throughout the period of time.

446. The system of any one of the clauses herein, wherein the depot is biodegradable and bioerodible.

447. The system of any one of the clauses herein, wherein the depot further comprises a thermal stabilizer.

448. The system of any one of the clauses herein, wherein the thermal stabilizer comprises at least one of a sugar, antioxidant or buffer.

449. The system of any one of the clauses herein, wherein the sugar comprises at least one of trehalose, raffinose or mannitol.

450. The system of any one of the clauses herein, wherein the antioxidant comprises at least one of methionine, ascorbic acid, sodium thiosulfate, catalase, platinum ethylenediaminetetraacetic acid (EDTA), citric acid, cysteins, thioglycerol, thioglycolic acid, thiosorbitol, butylated hydroxyanisol, butylated hydroxyltoluene, and propyl gallate.

451. The system of any one of the clauses herein, wherein the buffer comprises at least one of citrate, histidine, succinate or tris.

452. The system of any one of the clauses herein, wherein the GLP-1 receptor agonist comprises at least one of the sugar, antioxidant or buffer.

453. The system of any one of the clauses herein, wherein at least one of the sugar, antioxidant or buffer at least partially encapsulates the GLP-1 receptor agonist.

454. A biodegradable depot for sustained, controlled release of a therapeutic agent, the depot comprising:
a therapeutic region comprising the therapeutic agent including a glucagon-like peptide-1 (GLP-1) receptor agonist; and
a control region comprising a bioresorbable polymer and a releasing agent mixed with the polymer, wherein the releasing agent is configured to dissolve when the depot is placed in vivo to form diffusion openings in the control region,
wherein the depot is configured such that, following submersion of the depot in buffer solution for a period of time, the flexural strength of the depot decreases by no more than 75%.

455. The depot of any one of the clauses herein, wherein the depot is configured such that, following submersion of the depot in the buffer solution, the flexural strength of the depot decreases by no more than 70%, no more than 65%, no more than 60%, no more than 55%, no more than 50%, no more than 45%, no more than 40%, or no more than 30%.

456. A biodegradable depot for the treatment of symptoms associated with type II diabetes, comprising:
a therapeutic region comprising a glucagon-like peptide-1 (GLP-1) receptor agonist; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted at a treatment site in vivo and, while implanted, release the GLP-1 receptor agonist at the treatment site for no less than 3 days, and
wherein the control region does not include the GLP-1 receptor agonist at least prior to implantation of the depot at the treatment site.

457. A biodegradable depot for the treatment of symptoms associated with type II diabetes, comprising:
a therapeutic region comprising a glucagon-like peptide-1 (GLP-1) receptor agonist; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted at a treatment site in vivo and, while implanted, release the GLP-1 receptor agonist at the treatment site for no less than 3 days,
wherein the control region comprises a GLP-1 receptor agonist separate from the GLP-1 receptor agonist in the therapeutic region.

458. A biodegradable depot for the treatment of symptoms associated with type II diabetes, comprising:
a therapeutic region comprising a glucagon-like peptide-1 (GLP-1) receptor agonist; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted at a treatment site in vivo and, while implanted, release the GLP-1 receptor agonist at the treatment site for no less than 3 days, and
wherein the releasing agent is a first releasing agent and the therapeutic region includes a second releasing agent mixed with the GLP-1 receptor agonist.

459. A biodegradable depot for the treatment of symptoms associated with type II diabetes, comprising:
a therapeutic region comprising a glucagon-like peptide-1 (GLP-1) receptor agonist; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted at a treatment site in vivo and, while implanted, release the GLP-1 receptor agonist at the treatment site for no less than 3 days, and
wherein the releasing agent is a first releasing agent and the polymer is a first polymer, and the therapeutic region includes a second releasing agent and a second polymer mixed with the GLP-1 receptor agonist.

460. A biodegradable depot for the treatment of symptoms associated with type II diabetes, comprising:
a therapeutic region comprising a glucagon-like peptide-1 (GLP-1) receptor agonist; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted at a treatment site in vivo and, while implanted, release the GLP-1 receptor agonist at the treatment site for no less than 3 days, and
wherein a thickness of the control region is less than or equal to 1/50, 1/75, or 1/100 of a thickness of the therapeutic region.

461. A biodegradable depot for the treatment of symptoms associated with type II diabetes, comprising:
a therapeutic region comprising a glucagon-like peptide-1 (GLP-1) receptor agonist; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted at a treatment site in vivo and, while implanted, release the GLP-1 receptor agonist at the treatment site for no less than 3 days, and
wherein the first control layer includes a first amount of the releasing agent and the second control layer includes a second amount of the releasing agent different than the first amount.

462. A biodegradable depot for the treatment of symptoms associated with type II diabetes, comprising:
a therapeutic region comprising a glucagon-like peptide-1 (GLP-1) receptor agonist; and
a control region comprising a bioresorbable polymer and a releasing agent mixed with the polymer, wherein the releasing agent is configured to dissolve when the depot is placed in vivo to form diffusion openings in the control region;
wherein the depot is configured to be implanted at a treatment site in vivo and, while implanted, release the GLP-1 receptor agonist at the treatment site for no less than 3 days,
wherein the depot has a total surface area comprising the exposed surface area of the cover region plus the exposed surface area of the therapeutic region, and
wherein, when the depot is initially positioned at the treatment site in vivo, a ratio of the exposed surface area of the therapeutic region to the exposed surface area of the cover region is from about 5% to about 20%, or from about 5% to about 15%, or from about 5% to about 10%.

463. A depot for the treatment of symptoms associated with a mental illness, comprising:
a therapeutic region comprising a therapeutic agent;
a control region comprising a bioresorbable polymer and a releasing agent mixed with the polymer, wherein the releasing agent is configured to dissolve when the depot is placed in vivo to form diffusion openings in the control region; and
wherein the depot is configured to be implanted in vivo and, while implanted, release the therapeutic agent for a period of time.

464. The depot of the immediately preceding clause, wherein the depot comprises a depot of any one of the clauses herein.

465. The depot of any one of the clauses herein, wherein the therapeutic agent comprises an antidepressant including at least one of a selective serotonin reuptake inhibitor (SSRI), a serotonin-norepinephrine reuptake inhibitor (SNRI), a tricyclic antidepressant (TCA), a monoamine oxidase inhibitor (MAOI), atypical antidepressant, or derivatives thereof.

466. The depot of any one of the clauses herein, wherein the SSRI comprises at least one of citalopram, escitalopram, fluoxetine, fluvoxamine, fluvoxamine, paroxetine or sertraline.

467. The depot of any one of the clauses herein, wherein the SNRT comprises at least one of desvenlafaxine, duloxetine, venlafaxine, milnacipran or levomilnacipran.

468. The depot of any one of the clauses herein, wherein the TCA comprises at least one of amitriptyline, desipramine, doxepine, imipramine, nortriptyline, amoxapine, clomipramine, maprotiline, trimipramine or protriptyline.

469. The depot of any one of the clauses herein, wherein the MAOI comprises at least one of phenelzine, selegiline or tranylcypromine.

470. The depot of any one of the clauses herein, wherein the atypical antidepressant comprises at least one of bupropion, mirtazapine, nefazodone, trazodone, vilazodone or vortioxetine.

471. The depot of any one of the clauses herein, wherein the therapeutic agent comprises an antipsychotic including at least one of aripiprazole, aripirazole lauroxil, flupentixol, pipotiazine palmitate, haloperidol, asenapine, brexpiprazole, cariprazine, clozapine, iloperidone, lurasidone, olanzapine, paliperidone, quetiapine, risperidone, ziprasidone, chlorpromazine, fluphenazine, haloperidol, perphenazine, zuclopenthixol, or derivatives thereof. 472. The depot of any one of the clauses herein, wherein the therapeutic agent is configured to treat dementia and includes at least one of donepezil, galantamine, rivastigmine or memantine.

473. The depot of any one of the clauses herein, wherein the therapeutic agent in the therapeutic region comprises at least 50% of the total weight of the depot.

474. The depot of any one of the clauses herein, wherein about 40% to about 60% of the therapeutic agent in the therapeutic region is released in the first half of the period of time.

475. The depot of any one of the clauses herein, wherein at least 90% of the therapeutic agent in the therapeutic region is released within the period of time.

476. The depot of any one of the clauses herein, wherein the depot is configured to release about 0.1 mg/day to about 100 mg/day of the therapeutic agent over the period of time.

477. The depot of any one of the clauses herein, wherein the depot is configured to release less than about 100 mg/day of the therapeutic agent over the period of time.

478. The depot of any one of the clauses herein, wherein the depot is configured to release about 1 mg/day to about 30 mg/day of the therapeutic agent over the period of time.

479. The depot of any one of the clauses herein, wherein the depot is configured to release less than about 30 mg/day of the therapeutic agent over the period of time.

480. The depot of any one of the clauses herein, wherein the depot is configured to release about 0.5 mg/day to about 10 mg/day of the therapeutic agent over the period of time.

481. The depot of any one of the clauses herein, wherein the depot is configured to release less than about 10 mg/day of the therapeutic agent over the period of time.

482. The depot of any one of the clauses herein, wherein no more than 400 mg/day, no more than 300 mg/day, no more than 200 mg/day, no more than 100 mg/day, no more than 75 mg/day, no more than 50 mg/day, no more than 40 mg/day, no more than 30 mg/day, no more than 20 mg/day, no more than 10 mg/day, or no more than 5 mg/day of the therapeutic agent is released within any day of the period of time.

483. The depot of any one of the clauses herein, wherein the release of the therapeutic agent selectively blocks the reabsorption of serotonin and/or norepinephrine in the brain.

484. The depot of any one of the clauses herein, wherein the release of the therapeutic agent selectively blocks muscarinic M1, histamine H1, and/or α-adrenergic receptors.

485. The depot of any one of the clauses herein, wherein the release of the therapeutic agent selectively blocks dopamine receptors, 5-HT receptors, and/or dopaminergic pathways.

486. A method for treating a patient having symptoms associated with a mental illness, comprising:
positioning a depot at a treatment site in vivo having physiologic fluids, the depot comprising (a) a control region including a bioresorbable polymer and a releasing agent mixed with the polymer, and (b) a therapeutic region including at least 50% by weight of a therapeutic agent; and
releasing the therapeutic agent from the depot for a period of time.

487. The method of the immediately preceding clause, wherein the depot comprises a depot of any one of the clauses herein.

488. The method of any one of the clauses herein, wherein the therapeutic agent comprises an antidepressant including at least one of a selective serotonin reuptake inhibitor (SSRI), a serotonin-norepinephrine reuptake inhibitor (SNRI), a tricyclic antidepressant (TCA), a monoamine oxidase inhibitor (MAOI), atypical antidepressant, or derivatives thereof.

489. The method of any one of the clauses herein, wherein the SSRI comprises at least one of citalopram, escitalopram, fluoxetine, fluvoxamine, fluvoxamine, paroxetine or sertraline.

490. The method of any one of the clauses herein, wherein the SNRI comprises at least one of desvenlafaxine, duloxetine, venlafaxine, milnacipran or levomilnacipran.

491. The method of any one of the clauses herein, wherein the TCA comprises at least one of amitriptyline, desipramine, doxepine, imipramine, nortriptyline, amoxapine, clomipramine, maprotiline, trimipramine or protriptyline.

492. The method of any one of the clauses herein, wherein the MAOI comprises at least one of phenelzine, selegiline or tranylcypromine.

493. The method of any one of the clauses herein, wherein the atypical antidepressant comprises at least one of bupropion, mirtazapine, nefazodone, trazodone, vilazodone or vortioxetine.

494. The method of any one of the clauses herein, wherein the therapeutic agent comprises an antipsychotic including at least one of aripiprazole, aripirazole lauroxil, flupentixol, pipotiazine palmitate, haloperidol, asenapine, brexpiprazole, cariprazine, clozapine, iloperidone, lurasidone, olanzapine, paliperidone, quetiapine, risperidone, ziprasidone, chlorpromazine, fluphenazine, haloperidol, perphenazine, zuclopenthixol, or derivatives thereof.

495. The method of any one of the clauses herein, wherein the therapeutic agent is configured to treat dementia and includes at least one of donepezil, galantamine, rivastigmine or memantine.

496. The method of any one of the clauses herein, wherein releasing the therapeutic agent includes releasing the therapeutic agent such that about 40% to about 60% of the therapeutic agent in the therapeutic region is released in the first half of the period of time.

497. The method of any one of the clauses herein, wherein releasing the therapeutic agent includes releasing the therapeutic agent such that at least 90% of the therapeutic agent in the therapeutic region is released within the period of time.

498. The method of any one of the clauses herein, wherein releasing the therapeutic agent includes releasing the therapeutic agent at a rate of about 0.1 mg/day to about 100 mg/day over the period of time.

499. The method of any one of the clauses herein, wherein releasing the therapeutic agent includes releasing the therapeutic agent at a rate less than about 100 mg/day over the period of time.

500. The method of any one of the clauses herein, wherein releasing the therapeutic agent includes releasing the therapeutic agent at a rate of about 1 mg/day to about 30 mg/day over the period of time.

501. The method of any one of the clauses herein, wherein releasing the therapeutic agent includes releasing the therapeutic agent at a rate less than about 30 mg/day over the period of time.

502. The method of any one of the clauses herein, wherein releasing the therapeutic agent includes releasing the therapeutic agent at a rate of about 0.5 mg/day to about 10 mg/day of the therapeutic agent over the period of time.

503. The method of any one of the clauses herein, wherein releasing the therapeutic agent includes releasing the therapeutic agent at a rate less than about 10 mg/day over the period of time.

504. The method of any one of the clauses herein, wherein releasing the therapeutic agent includes releasing the therapeutic agent such that no more than 400 mg/day, no more than 300 mg/day, no more than 200 mg/day, no more than 100 mg/day, no more than 75 mg/day, no more than 50 mg/day, no more than 40 mg/day, no more than 30 mg/day, no more than 20 mg/day, no more than 10 mg/day, or no more than 5 mg/day of the therapeutic agent is released within any day of the period of time.

505. The method of any one of the clauses herein, wherein the period of time is no less than 1 day, no less than 2 days, no less than 3 days, no less than 4 days, no less than 5 days, no less than 6 days, no less than 7 days, no less than 8 days, no less than 9 days, no less than 10 days, no less than 11 days, no less than 12 days, no less than 13 days, no less than 14 days, no less than 15 days, no less than 16 days, no less than 17 days, no less than 18 days, no less than 19 days, no less than 20 days, no less than 21 days, no less than 22 days, no less than 23 days, no less than 24 days, no less than 25 days, no less than 26 days, no less than 27 days, no less than 28 days, no less than 29 days, no less than 30 days, no less than 40 days, no less than 50 days, no less than 60 days, no less than 70 days, no less than 90 days, no less than 100 days, no less than 200 days, no less than 300 days, or no less than 365 days.

506. A system for delivering a therapeutic agent to a patient to treat a mental illness, the system comprising:
a delivery device having a distal region configured to be positioned under the skin of a patient; and
a depot positioned within the delivery device and configured to be emitted from the distal region to the patient, the depot comprising:
   a therapeutic region comprising the therapeutic agent; and
   a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region,
   wherein the depot is configured to be implanted under the skin of a patient and, while implanted, release the therapeutic agent for a period of time not less than 7 days.

507. A system for delivering a therapeutic agent configured to treat a mental illness, the system comprising:
a needle having a lumen;
a syringe operatively coupled to the needle; and
a depot disposed within the lumen and configured to be emitted from the needle via activation of the syringe, the depot comprising:
   a therapeutic region comprising the therapeutic agent; and
   a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region,
   wherein the depot is configured to be implanted and, while implanted, release the therapeutic agent for a period of time.

508. A system for delivering a therapeutic agent to treat a mental illness, the system comprising:
an expandable member configured to be expanded from a reduced-volume configuration for delivery to an expanded-volume configuration for deployment; and
a depot carried by the expandable member, the depot comprising:
   a therapeutic region comprising the therapeutic agent; and
   a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region,
wherein the depot is configured to be implanted in vivo and, while implanted, release the therapeutic agent for a period of time.

509. The system of any one of the clauses herein, wherein the depot comprises a depot of any one of the clauses herein.

510. The system of any one of the clauses herein, wherein the therapeutic agent comprises an antidepressant including at least one of a selective serotonin reuptake inhibitor (SSRI), a serotonin-norepinephrine reuptake inhibitor (SNRI), a tricyclic antidepressant (TCA), a monoamine oxidase inhibitor (MAOI), atypical antidepressant, or derivatives thereof.

511. The system of any one of the clauses herein, wherein the SSRI comprises at least one of citalopram, escitalopram, fluoxetine, fluvoxamine, fluvoxamine, paroxetine or sertraline.

512. The system of any one of the clauses herein, wherein the SNRI comprises at least one of desvenlafaxine, duloxetine, venlafaxine, milnacipran or levomilnacipran.

513. The system of any one of the clauses herein, wherein the TCA comprises at least one of amitriptyline, desipramine, doxepine, imipramine, nortriptyline, amoxapine, clomipramine, maprotiline, trimipramine or protriptyline.

514. The system of any one of the clauses herein, wherein the MAOI comprises at least one of phenelzine, selegiline or tranylcypromine.

515. The system of any one of the clauses herein, wherein the atypical antidepressant comprises at least one of bupropion, mirtazapine, nefazodone, trazodone, vilazodone or vortioxetine.

516. The system of any one of the clauses herein, wherein the therapeutic agent comprises an antipsychotic including at least one of aripiprazole, aripirazole lauroxil, flupentixol, pipotiazine palmitate, haloperidol, asenapine, brexpiprazole, cariprazine, clozapine, iloperidone, lurasidone, olanzapine, paliperidone, quetiapine, risperidone, ziprasidone, chlorpromazine, fluphenazine, haloperidol, perphenazine, zuclopenthixol, or derivatives thereof. 517. The system of any one of the clauses herein, wherein the therapeutic agent is configured to treat dementia and includes at least one of donepezil, galantamine, rivastigmine or memantine.

518. The system of any one of the clauses herein, wherein the depot is configured to release about 0.1 mg/day to about 100 mg/day of the therapeutic agent over the period of time.

519. The system of any one of the clauses herein, wherein the depot is configured to release less than about 100 mg/day of the therapeutic agent over the period of time.

520. The system of any one of the clauses herein, wherein the depot is configured to release about 1 mg/day to about 30 mg/day of the therapeutic agent over the period of time.

521. The system of any one of the clauses herein, wherein the depot is configured to release less than about 30 mg/day of the therapeutic agent over the period of time.

522. The system of any one of the clauses herein, wherein the depot is configured to release about 0.5 mg/day to about 10 mg/day of the therapeutic agent to the treatment site over the period of time.

523. The system of any one of the clauses herein, wherein the depot is configured to release less than about 10 mg/day of the therapeutic agent to the treatment site over the period of time.

524. The system of any one of the clauses herein, wherein no more than 400 mg/day, no more than 300 mg/day, no more than 200 mg/day, no more than 100 mg/day, no more than 75 mg/day, no more than 50 mg/day, no more than 40 mg/day, no more than 30 mg/day, no more than 20 mg/day, no more than 10 mg/day, or no more than 5 mg/day of the therapeutic agent is released within any day of the period of time.

525. The system of any one of the clauses herein, wherein the release of the therapeutic agent to the treatment site selectively blocks the reabsorption of serotonin and/or norepinephrine in the brain.

526. The system of any one of the clauses herein, wherein the release of the therapeutic agent to the treatment site selectively blocks muscarinic M1, histamine H1, and/or α-adrenergic receptors.

527. The system of any one of the clauses herein, wherein the release of the therapeutic agent to the treatment site selectively blocks dopamine receptors, 5-HT receptors, and/or dopaminergic pathways.

528. A depot for the treatment of symptoms or risk factors associated with a cardiovascular disease, comprising:
a therapeutic region comprising a therapeutic agent;
a control region comprising a bioresorbable polymer and a releasing agent mixed with the polymer, wherein the releasing agent is configured to dissolve when the depot is placed in vivo to form diffusion openings in the control region; and
wherein the depot is configured to be implanted in vivo and, while implanted, release the therapeutic agent for a period of time.

529. The depot of any one of the clauses herein, wherein the therapeutic agent comprises an antihypertensive agent including at least one of a thiazide-type diuretic, angiotensin converting enzyme (ACE) inhibitor, angiotensin II receptor blocker (ARB), calcium-channel blocker, or derivatives thereof. 530. The depot of any one of the clauses herein, wherein the thiazide-type diuretic comprises at least one of chlorthalidone, hydrochlorithiazide or indapamide.

531. The depot of any one of the clauses herein, wherein the ACE inhibitor comprises at least one of benazepril, fosinopril, lisinopril, moexipril, perindopril, quinapril, ramipril or trandolapril.

532. The depot of any one of the clauses herein, wherein the ARB comprises at least one of azilsartan, candesartan, eprosartan, irbesartan, losartan, olmesartan, telmisartan or valsartan.

533. The depot of any one of the clauses herein, wherein the calcium-channel blocker comprises at least one of a dihydropyridine type blocker or a nondihydropyridine type blocker.

534. The depot of any one of the clauses herein, wherein the dihydropyridine type blocker comprises amlodipine, felodipine, isradipine, nicardipine, nifedipine, nisoldipine, bepridil, dilitiazem or nisoldipine.

535. The depot of any one of the clauses herein, wherein the nondihydropyridine type blocker comprises diltiazem or verapamil.

536. The depot of any one of the clauses herein, wherein the therapeutic agent comprises at least one of a statin, cholesterol absorption inhibitor, proprotein convertase subtilisin-kexin type 9 (PCSK9) inhibitor, nicotinic acid, fibric acid, or omega-3-fatty acids, or derivatives thereof.

537. The depot of any one of the clauses herein, wherein the statin comprises at least one of lovastatin, pravastatin, simvastatin, fluvastatin, atorvastatin, rosuvastatin or pitavastatin.

538. The depot of any one of the clauses herein, wherein the cholesterol absorption inhibitor comprises exetimibe.

539. The depot of any one of the clauses herein, wherein the PCSK9 comprises at least one of evolocumab or alirocumab.

540. The depot of any one of the clauses herein, wherein the fibric acid comprises at least one of gemfibrozil, bezafibrate, fenofibrate or fenofibric acid.

541. The depot of any one of the clauses herein, wherein the therapeutic agent is configured to treat hypercholesteremia by lowering LDL levels, increasing HDL levels, and/or lower blood triglyceride levels.

542. The depot of any one of the clauses herein, wherein the depot is configured to release about 1 mg/day to about 600 mg/day of the therapeutic agent over the period of time.

543. The depot of any one of the clauses herein, wherein the depot is configured to release less than about 600 mg/day of the therapeutic agent over the period of time.

544. The depot of any one of the clauses herein, wherein the depot is configured to release about 2 mg/day to about 40 mg/day of the therapeutic agent over the period of time.

545. The depot of any one of the clauses herein, wherein the depot is configured to release less than about 40 mg/day of the therapeutic agent over the period of time.

546. The depot of any one of the clauses herein, wherein the depot is configured to release about 20 mg/day to about 80 mg/day of the therapeutic agent over the period of time.

547. The depot of any one of the clauses herein, wherein the depot is configured to release less than about 80 mg/day of the therapeutic agent over the period of time.

548. The depot of any one of the clauses herein, wherein the depot is configured to release about 2 mg/day to about 60 mg/day of the therapeutic agent over the period of time.

549. The depot of any one of the clauses herein, wherein the depot is configured to release less than about 60 mg/day of the therapeutic agent over the period of time.

550. The depot of any one of the clauses herein, wherein the depot is configured to release about 100 mg/day to about 480 mg/day of the therapeutic agent over the period of time.

551. The depot of any one of the clauses herein, wherein the depot is configured to release less than about 480 mg/day of the therapeutic agent over the period of time.

552. The depot of any one of the clauses herein, wherein the depot is configured to release about 200 mg/day to about 600 mg/day of the therapeutic agent over the period of time.

553. The depot of any one of the clauses herein, wherein the depot is configured to release about 5 mg/day to about 80 mg/day of the therapeutic agent over the period of time.

554. The depot of any one of the clauses herein, wherein the depot is configured to release less than about 80 mg/day of the therapeutic agent over the period of time.

555. The depot of any one of the clauses herein, wherein the depot is configured to release about 70 mg/day to about 150 mg/day of the therapeutic agent over the period of time.

556. The depot of any one of the clauses herein, wherein the depot is configured to release less than about 150 mg/day of the therapeutic agent over the period of time.

557. The depot of any one of the clauses herein, wherein the depot is configured to release about 1 g/day to about 4 g/day of the therapeutic agent over the period of time.

558. The depot of any one of the clauses herein, wherein the depot is configured to release less than about 4 g/day of the therapeutic agent over the period of time.

559. The depot of any one of the clauses herein, wherein no more than about 4 g/day, no more than about 3 g/day, no more than about 2 g/day, no more than about 1 g/day, no more than about 900 mg/day, no more than about 800 mg/day, no more than about 700 mg/day, no more than about 600 mg/day, no more than about 500 mg/day, no more than about 400 mg/day, no more than about 300 mg/day, no more than about 200 mg/day, no more than about 100 mg/day, no more than about 75 mg/day, no more than about 50 mg/day, no more than about 40 mg/day, no more than about 30 mg/day, no more than about 20 mg/day, no more than about 10 mg/day, or no more than about 5 mg/day of the therapeutic agent is released within any day of the period of time.

560. The depot of any one of the clauses herein, wherein the release of the therapeutic agent decreases the ability of the kidneys to reabsorb salt and water from urine.

561. The depot of any one of the clauses herein, wherein the release of the therapeutic agent decreases the activity of the angiotensin converting enzyme.

562. The depot of any one of the clauses herein, wherein the release of the therapeutic agent inhibits the effect of angiotensin II.

563. A method for treating a patient having symptoms or risk factors associated with a cardiovascular disease, comprising:
positioning a depot at a treatment site in vivo having physiologic fluids, the depot comprising (a) a control region including a bioresorbable polymer and a releasing agent mixed with the polymer, and (b) a therapeutic region including at least 50% by weight of a therapeutic agent; and
releasing the therapeutic agent from the depot for a period of time.

564. The method of any one of the clauses herein, wherein the depot comprises the depot of any one of the clauses herein.

565. The method of any one of the clauses herein, wherein the therapeutic agent comprises an antihypertensive agent including at least one of a thiazide-type diuretic, angiotensin converting enzyme (ACE) inhibitor, angiotensin II receptor blocker (ARB), calcium-channel blocker, or derivatives thereof. 566. The method of any one of the clauses herein, wherein the thiazide-type diuretic comprises at least one of chlorthalidone, hydrochlorithiazide or indapamide.

567. The method of any one of the clauses herein, wherein the ACE inhibitor comprises at least one of benazepril, fosinopril, lisinopril, moexipril, perindopril, quinapril, ramipril or trandolapril.

568. The method of any one of the clauses herein, wherein the ARB comprises at least one of azilsartan, candesartan, eprosartan, irbesartan, losartan, olmesartan, telmisartan or valsartan.

569. The method of any one of the clauses herein, wherein the calcium-channel blocker comprises at least one of a dihydropyridine type blocker or a nondihydropyridine type blocker.

570. The method of any one of the clauses herein, wherein the dihydropyridine type blocker comprises amlodipine, felodipine, isradipine, nicardipine, nifedipine, nisoldipine, bepridil, dilitiazem or nisoldipine.

571. The method of any one of the clauses herein, wherein the nondihydropyridine type blocker comprises diltiazem or verapamil.

572. The method of any one of the clauses herein, wherein the therapeutic agent comprises at least one of a statin, cholesterol absorption inhibitor, proprotein convertase subtilisin-kexin type 9 (PCSK9) inhibitor, nicotinic acid, fibric acid, or omega-3-fatty acids, or derivatives thereof.

573. The method of any one of the clauses herein, wherein the statin comprises at least one of lovastatin, pravastatin, simvastatin, fluvastatin, atorvastatin, rosuvastatin or pitavastatin.

574. The depot of any one of the clauses herein, wherein the cholesterol absorption inhibitor comprises exetimibe.

575. The method of any one of the clauses herein, wherein the PCSK9 comprises at least one of evolocumab or alirocumab.

576. The method of any one of the clauses herein, wherein the fibric acid comprises at least one of gemfibrozil, bezafibrate, fenofibrate or fenofibric acid.

577. The method of any one of the clauses herein, wherein the therapeutic agent is configured to treat hypercholesteremia by lowering LDL levels, increasing HDL levels, and/or lower blood triglyceride levels.

578. The method of any one of the clauses herein, wherein releasing the therapeutic agent comprises releasing the therapeutic agent at about 1 mg/day to about 600 mg/day to the treatment site over the period of time.

579. The method of any one of the clauses herein, wherein releasing the therapeutic agent comprises releasing the therapeutic agent at a rate less than about 600 mg/day to the treatment site over the period of time.

580. The method of any one of the clauses herein, wherein releasing the therapeutic agent comprises releasing the therapeutic agent at about 2 mg/day to about 40 mg/day to the treatment site over the period of time.

581. The method of any one of the clauses herein, wherein releasing the therapeutic agent comprises releasing the therapeutic agent at a rate less than about 40 mg/day to the treatment site over the period of time.

582. The method of any one of the clauses herein, wherein releasing the therapeutic agent comprises releasing the therapeutic agent at about 20 mg/day to about 80 mg/day to the treatment site over the period of time.

583. The method of any one of the clauses herein, wherein releasing the therapeutic agent comprises releasing the therapeutic agent at a rate less than about 80 mg/day to the treatment site over the period of time.

584. The method of any one of the clauses herein, wherein releasing the therapeutic agent comprises releasing the therapeutic agent at about 2 mg/day to about 60 mg/day to the treatment site over the period of time.

585. The method of any one of the clauses herein, wherein releasing the therapeutic agent comprises releasing the therapeutic agent at a rate less than about 60 mg/day to the treatment site over the period of time.

586. The method of any one of the clauses herein, wherein releasing the therapeutic agent comprises releasing the therapeutic agent at about 100 mg/day to about 480 mg/day to the treatment site over the period of time.

587. The method of any one of the clauses herein, wherein releasing the therapeutic agent comprises releasing the therapeutic agent at a rate less than about 480 mg/day to the treatment site over the period of time.

588. The method of any one of the clauses herein, wherein releasing the therapeutic agent comprises releasing the therapeutic agent at about 200 mg/day to about 600 mg/day to the treatment site over the period of time.

589. The method of any one of the clauses herein, wherein releasing the therapeutic agent comprises releasing the therapeutic agent at about 5 mg/day to about 80 mg/day to the treatment site over the period of time.

590. The method of any one of the clauses herein, wherein releasing the therapeutic agent comprises releasing the therapeutic agent at about 80 mg/day to the treatment site over the period of time.

591. The method of any one of the clauses herein, wherein releasing the therapeutic agent comprises releasing the therapeutic agent at about 70 mg/day to about 150 mg/day to the treatment site over the period of time.

592. The method of any one of the clauses herein, wherein releasing the therapeutic agent comprises releasing the therapeutic agent at about 150 mg/day to the treatment site over the period of time.

593. The method of any one of the clauses herein, wherein releasing the therapeutic agent comprises releasing the therapeutic agent at about 1 g/day to about 4 g/day to the treatment site over the period of time.

594. The method of any one of the clauses herein, wherein releasing the therapeutic agent comprises releasing the therapeutic agent at about 4 g/day to the treatment site over the period of time.

595. The method of any one of the clauses herein, wherein releasing the therapeutic agent comprises releasing no more than about 4 g/day, no more than about 3 g/day, no more than about 2 g/day, no more than about 1 g/day, no more than about 900 mg/day, no more than about 800 mg/day, no more than about 700 mg/day, no more than about 600 mg/day, no more than about 500 mg/day, no more than about 400 mg/day, no more than about 300 mg/day, no more than about 200 mg/day, no more than about 100 mg/day, no more than about 75 mg/day, no more than about 50 mg/day, no more than about 40 mg/day, no more than about 30 mg/day, no more than about 20 mg/day, no more than about 10 mg/day, or no more than about 5 mg/day of the therapeutic agent within any day of the period of time.

596. A system for delivering a therapeutic agent to a patient to treat symptoms or risk factors associated with a cardiovascular disease, the system comprising:
a delivery device having a distal region configured to be positioned under the skin of a patient; and
a depot positioned within the delivery device and configured to be emitted from the distal region to the patient, the depot comprising:
   a therapeutic region comprising the therapeutic agent; and
   a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region,
   wherein the depot is configured to be implanted and, while implanted, release the therapeutic agent at the treatment site for a period of time.

597. A system for delivering a therapeutic agent configured to treat symptoms or risk factors associated with a cardiovascular disease, the system comprising:
a needle having a lumen;
a syringe operatively coupled to the needle; and
a depot disposed within the lumen and configured to be emitted from the needle via activation of the syringe, the depot comprising:
   a therapeutic region comprising the therapeutic agent; and
   a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region,
   wherein the depot is configured to be implanted in vivo and, while implanted, release the therapeutic agent for a period of time.

598. A system for delivering a therapeutic agent to treat symptoms or risk factors associated with a cardiovascular disease, the system comprising:
an expandable member configured to be expanded from a reduced-volume configuration for delivery to an expanded-volume configuration for deployment; and
a depot carried by the expandable member, the depot comprising:
   a therapeutic region comprising the therapeutic agent; and
   a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region,
   wherein the depot is configured to be implanted in vivo and, while implanted, release the therapeutic agent for a period of time.

599. The system of any one of the clauses herein, wherein the therapeutic agent comprises an antihypertensive agent including at least one of a thiazide-type diuretic, angiotensin converting enzyme (ACE) inhibitor, angiotensin II receptor blocker (ARB), calcium-channel blocker, or derivatives thereof. 600. The system of any one of the clauses herein, wherein the thiazide-type diuretic comprises at least one of chlorthalidone, hydrochlorithiazide or indapamide.

601. The system of any one of the clauses herein, wherein the ACE inhibitor comprises at least one of benazepril, fosinopril, lisinopril, moexipril, perindopril, quinapril, ramipril or trandolapril.

602. The system of any one of the clauses herein, wherein the ARB comprises at least one of azilsartan, candesartan, eprosartan, irbesartan, losartan, olmesartan, telmisartan or valsartan.

603. The system of any one of the clauses herein, wherein the calcium-channel blocker comprises at least one of a dihydropyridine type blocker or a nondihydropyridine type blocker.

604. The system of any one of the clauses herein, wherein the dihydropyridine type blocker comprises amlodipine, felodipine, isradipine, nicardipine, nifedipine, nisoldipine, bepridil, dilitiazem or nisoldipine.

605. The system of any one of the clauses herein, wherein the nondihydropyridine type blocker comprises diltiazem or verapamil.

606. The system of any one of the clauses herein, wherein the therapeutic agent comprises at least one of a statin, cholesterol absorption inhibitor, proprotein convertase subtilisin-kexin type 9 (PCSK9) inhibitor, nicotinic acid, fibric acid, or omega-3-fatty acids, or derivatives thereof.

607. The system of any one of the clauses herein, wherein the statin comprises at least one of lovastatin, pravastatin, simvastatin, fluvastatin, atorvastatin, rosuvastatin or pitavastatin.

608. The system of any one of the clauses herein, wherein the cholesterol absorption inhibitor comprises exetimibe.

609. The system of any one of the clauses herein, wherein the PCSK9 comprises at least one of evolocumab or alirocumab.

610. The system of any one of the clauses herein, wherein the fibric acid comprises at least one of gemfibrozil, bezafibrate, fenofibrate or fenofibric acid.

611. The system of any one of the clauses herein, wherein the therapeutic agent is configured to treat hypercholesteremia by lowering LDL levels, increasing HDL levels, and/or lower blood triglyceride levels.

612. The system of any one of the clauses herein, wherein the depot is configured to release about 1 mg/day to about 600 mg/day of the therapeutic agent over the period of time.

613. The system of any one of the clauses herein, wherein the depot is configured to release less than about 600 mg/day of the therapeutic agent over the period of time.

614. The system of any one of the clauses herein, wherein the depot is configured to release about 2 mg/day to about 40 mg/day of the therapeutic agent over the period of time.

615. The system of any one of the clauses herein, wherein the depot is configured to release less than about 40 mg/day of the therapeutic agent over the period of time.

616. The system of any one of the clauses herein, wherein the depot is configured to release about 20 mg/day to about 80 mg/day of the therapeutic agent over the period of time.

617. The system of any one of the clauses herein, wherein the depot is configured to release less than about 80 mg/day of the therapeutic agent over the period of time.

618. The system of any one of the clauses herein, wherein the depot is configured to release about 2 mg/day to about 60 mg/day of the therapeutic agent over the period of time.

619. The system of any one of the clauses herein, wherein the depot is configured to release less than about 60 mg/day of the therapeutic agent over the period of time.

620. The system of any one of the clauses herein, wherein the depot is configured to release about 100 mg/day to about 480 mg/day of the therapeutic agent over the period of time.

621. The system of any one of the clauses herein, wherein the depot is configured to release less than about 480 mg/day of the therapeutic agent over the period of time.

622. The system of any one of the clauses herein, wherein the depot is configured to release about 200 mg/day to about 600 mg/day of the therapeutic agent over the period of time.

623. The system of any one of the clauses herein, wherein the depot is configured to release about 5 mg/day to about 80 mg/day of the therapeutic agent over the period of time.

624. The system of any one of the clauses herein, wherein the depot is configured to release less than about 80 mg/day of the therapeutic agent over the period of time.

625. The system of any one of the clauses herein, wherein the depot is configured to release about 70 mg/day to about 150 mg/day of the therapeutic agent over the period of time.

626. The system of any one of the clauses herein, wherein the depot is configured to release less than about 150 mg/day of the therapeutic agent over the period of time.

627. The system of any one of the clauses herein, wherein the depot is configured to release about 1 g/day to about 4 g/day of the therapeutic agent over the period of time.

628. The system of any one of the clauses herein, wherein the depot is configured to release less than about 4 g/day of the therapeutic agent over the period of time.

629. The system of any one of the clauses herein, wherein no more than about 4 g/day, no more than about 3 g/day, no more than about 2 g/day, no more than about 1 g/day, no more than about 900 mg/day, no more than about 800 mg/day, no more than about 700 mg/day, no more than about

600 mg/day, no more than about 500 mg/day, no more than about 400 mg/day, no more than about 300 mg/day, no more than about 200 mg/day, no more than about 100 mg/day, no more than about 75 mg/day, no more than about 50 mg/day, no more than about 40 mg/day, no more than about 30 mg/day, no more than about 20 mg/day, no more than about 10 mg/day, or no more than about 5 mg/day of the therapeutic agent is released within any day of the period of time.

630. The system of any one of the clauses herein, wherein the release of the therapeutic agent decreases the ability of the kidneys to reabsorb salt and water from urine.

631. The system of any one of the clauses herein, wherein the release of the therapeutic agent decreases the activity of the angiotensin converting enzyme.

632. The system of any one of the clauses herein, wherein the release of the therapeutic agent inhibits the effect of angiotensin II.

633. A biodegradable depot for the treatment of symptoms associated with a cardiovascular disease, comprising:
a therapeutic region comprising a therapeutic agent; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted in vivo and, while implanted, release the therapeutic agent for no less than 3 days, and
wherein the control region does not include the therapeutic agent at least prior to implantation of the depot.

634. A biodegradable depot for the treatment of symptoms associated with a cardiovascular disease, comprising:
a therapeutic region comprising a first therapeutic agent; and
a control region comprising a bioresorbable polymer, a releasing agent mixed with the polymer, and a second therapeutic agent, wherein the releasing agent is configured to dissolve when the depot is placed in vivo to form diffusion openings in the control region,
wherein the depot is configured to be implanted in vivo and, while implanted, release the first and second therapeutic agents for a period of time no less than 30 days.

635. A biodegradable depot for the treatment of symptoms associated with a cardiovascular disease, comprising:
a therapeutic region comprising a therapeutic agent, and a first releasing agent mixed with the therapeutic agent; and
a control region comprising a bioresorbable polymer and a second releasing agent mixed with the polymer,
wherein the first and second releasing agents are configured to dissolve when the depot is placed in vivo to form diffusion openings in the control region,
wherein the depot is configured to be implanted in vivo and, while implanted, release the therapeutic agent for no less than 3 days.

636. A biodegradable depot for the treatment of symptoms associated with a cardiovascular disease, comprising:
a therapeutic region comprising a therapeutic agent; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted in vivo and, while implanted, release the therapeutic agent for no less than 3 days, and
wherein the releasing agent is a first releasing agent and the polymer is a first polymer, and the therapeutic region includes a second releasing agent and a second polymer mixed with the therapeutic agent.

637. A biodegradable depot for the treatment of symptoms associated with a cardiovascular disease, comprising:
a therapeutic region comprising a therapeutic agent; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted in vivo and, while implanted, release the therapeutic agent for no less than 3 days, and
wherein a thickness of the control region is less than or equal to 1/50of a thickness of the therapeutic region.

638. A biodegradable depot for the treatment of symptoms associated with a cardiovascular disease, comprising:
a therapeutic region comprising a therapeutic agent; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted in vivo and, while implanted, release the therapeutic agent for no less than 3 days, and
wherein the first control layer includes a first amount of the releasing agent and the second control layer includes a second amount of the releasing agent different than the first amount.

639. A biodegradable depot for the treatment of symptoms associated with a cardiovascular disease, comprising:
a therapeutic region comprising a therapeutic agent; and
a control region comprising a bioresorbable polymer and a releasing agent mixed with the polymer, wherein the releasing agent is configured to dissolve when the depot is placed in vivo to form diffusion openings in the control region;
wherein the depot is configured to be implanted in vivo and, while implanted, release the therapeutic agent at the treatment site for no less than 3 days,
wherein the depot has a total surface area comprising the exposed surface area of the cover region plus the exposed surface area of the therapeutic region, and
wherein, when the depot is initially positioned in vivo, a ratio of the exposed surface area of the therapeutic region to the exposed surface area of the cover region is from about 5% to about 20%, or from about 5% to about 15%, or from about 5% to about 10%.

640. A depot for treating or preventing of symptoms associated with HIV, comprising:
a therapeutic region comprising a therapeutic agent; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted in vivo and, while implanted, release the therapeutic agent for a period of time.

641. The depot of any one of the clauses herein, wherein the therapeutic agent comprises an antiretroviral.

642. The depot of any one of the clauses herein, wherein the antiretroviral comprises at least one of dolutegravir, cabotegravir or riplivirine.

643. The depot of any one of the clauses herein, wherein the therapeutic agent comprises at least one of an entry inhibitor, pharmacokinetic enhancer, integrase inhibitor, nucleoside or nucleotide reverse transcriptase inhibitor, non-nucleoside reverse transcriptase inhibitor or protease inhibitor.

644. The depot of any one of the clauses herein, wherein the entry inhibitor comprises at least one of enfuvirtide and maraviroc.

645. The depot of any one of the clauses herein, wherein the pharmacokinetic enhancer comprises at least one of ritonavir or cobicistat.

646. The depot of any one of the clauses herein, wherein the integrase inhibitor comprises at least one of raltegravir, dolutegravir or elvitegravir.

647. The depot of any one of the clauses herein, wherein the nucleoside or nucleotide reverse transcriptase inhibitor comprises at least one of tricitabine, lamivudine, zidovudine, didanosine, tenofovir, stavudine or abacavir.

648. The depot of any one of the clauses herein, wherein the non-nucleoside reverse transcriptase inhibitor comprises at least one of rilpivirine, etravirine, delavirdine, doravirine, efavirenz or nevirapine.

649. The depot of any one of the clauses herein, wherein the protease inhibitor comprises at least one of tipranavir, indinavir, saquinavir, lopinavir and norvir, fosamprenavir, darunavir, atazanavir or nelfinavir.

650. The depot of any one of the clauses herein, wherein the therapeutic agent comprises at least one of sustiva, viread, emtriva, bictegravir, tenofovir alagenamide, edurant, pifeltro, epivir, vitekta, tybost, tivicay, retrovir or ziagen.

651. The depot of any one of the clauses herein, wherein the therapeutic region comprises at least 10 mg, 50 mg, 100 mg, 200 mg, 300 mg, 400 mg, 500 mg, 600 mg, 700 mg, 800 mg, 900 mg, 1.0 g, 1.1 g, 1.2 g, 1.3 g, 1.4 g or 1.5 g of the therapeutic agent.

652. The depot of any one of the clauses herein, wherein the depot is configured to release the therapeutic agent through the period of time at rate of no more than 10 mg/day, no more than 1 mg/day, no more than 500 μg/day, no more than 100 μg/day, no more than 90 μg/day, no more than 80 μg/day, no more than 70 μg/day, no more than 60 μg/day, or no more than 50 μg/day.

653. The depot of any one of the clauses herein, wherein the therapeutic region is configured to release the therapeutic agent through the period of time at a rate of from about 100 μg/day to 50 mg/day, 100 μg/day to 40 mg/day, 100 μg/day to 30 mg/day, 100 μg/day to 20 mg/day, 100 μg/day to 10 mg/day, 100 μg/day to 5 mg/day, 100 μg/day to 1000 μg/day, 100 μg/day to 100 μg/day, 100 μg/day to 800 μg/day, 100 μg/day to 700 μg/day, 100 μg/day to 600 μg/day, 100 μg/day to 600 μg/day, 200 μg/day to 600 μg/day, 300 μg/day to 600 μg/day, 400 μg/day to 600 μg/day, or 400 μg/day to 500 μg/day.

654. A depot for treating or preventing of symptoms associated with malaria, comprising:
a therapeutic region comprising a therapeutic agent; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted in vivo and, while implanted, release the therapeutic agent for a period of time.

655. The depot of any one of the clauses herein, wherein the therapeutic agent comprises at least one of an antimalarial agent, artemisinin-based combination therapy, or vaccine.

656. The depot of any one of the clauses herein, wherein the antimalarial agent comprises at least one of quinine, chloroquine, amodiaquine, mefloquine, primaquine, sulfadoxine-pyrimethamine, intravenous artesunate, atovaquone-proguanil, azithromycin, ferroquine, artesunate, foxmidomycin, clindamycin, ozonide, piperaquine, sprioindolone, artesunate-amodiaquine, artesunate, coartem, eurartesim, pyramax, imidazolopiperazine, timidazole, tafenoquine or bulaquine.

657. The depot of any one of the clauses herein, wherein the vaccine comprises RTS,S.

658. The depot of any one of the clauses herein, wherein the therapeutic agent comprises chemoprophylaxis.

659. The depot of any one of the clauses herein, wherein the therapeutic agent is configured to treat or prevent infection from P. falciparum.

660. The depot of any one of the clauses herein, wherein the therapeutic agent is configured to target at least one of infected red blood cell (iRBC).

661. The depot of any one of the clauses herein, wherein the therapeutic region comprises at least 100 mg, 200 mg, 300 mg, 400 mg, 500 mg, 600 mg, 700 mg, 800 mg, 900 mg, 1.0 g, 1.5 g, 2.0 g, 3.0 g, 4.0 g, 5.0 g, 6.0 g, 7.0 g, 8.0 g, 9.0 g, or 10 g of the therapeutic agent.

662. The depot of any one of the clauses herein, wherein the depot is configured to release the therapeutic agent through the period of time at rate of no more than 400 mg/day, no more than 300 mg/day, no more than 200 mg/day, no more than 150 mg/day, no more than 100 mg/day, or no more than 50 mg/day.

663. The depot of any one of the clauses herein, wherein the therapeutic region is configured to release the therapeutic agent through the period of time at a rate of from about 10 mg/day to 400 mg/day, 10 mg/day to 350 mg/day, 10 mg/day to 300 mg/day, 10 mg/day to 250 mg/day, 50 mg/day to 250 mg/day, 100 mg/day to 250 mg/day, 150 mg/day to 250 mg/day, 200 mg/day to 250 mg/day.

664. The depot of any one of the clauses herein, wherein the period of time is no less than 1 day, no less than two days, no less than three days, no less than four days, no less than five days, no less than six days, no less than one week, no less than two weeks, no less than three weeks, no less than four weeks, no less than five weeks, no less than 8 weeks, no less than 2 months, no less than 3 months, no less than 4 months, no less than 6 months, no less than 7 months, no less than 8 months, no less than 9 months, no less than 10 months, no less than 12 months.

665. The depot of any one of the clauses herein, wherein the therapeutic region further comprises an analgesic.

666. The depot of any one of the clauses herein, wherein the analgesic includes at least one of bupivacaine, ropivacaine, mepivacaine, etidocaine, levobupivacaine, trimecaine, carticaine, articaine, lidocaine, prilocaine, benzocaine, procaine, tetracaine or chloroprocaine.

667. The depot of any one of the clauses herein, wherein the therapeutic region further comprises a chemotherapeutic agent.

668. The depot of any one of the clauses herein, wherein the chemotherapeutic agent includes at least one of antibodies, alkylating agents, angiogenesis inhibitors, antimetabolites, DNA cleavers, DNA crosslinkers, DNA intercalators, DNA minor groove binders, enediynes, heat shock protein 90 inhibitors, histone deacetylase inhibitors, immunomodulators, microtubule stabilizers, nucleoside (purine or pyrimidine) analogs, nuclear export inhibitors, proteasome inhibitors, topoisomerase (I or II) inhibitors, tyrosine kinase inhibitors, and serine/threonine kinase inhibitors. Specific therapeutic agents include, but are not limited to, adalimumab, ansamitocin P3, auristatin, bendamustine, bevacizumab, bicalutamide, bleomycin, bortezomib, busulfan, callistatin A, camptothecin, capecitabine, carboplatin, carmustine, cetuximab, cisplatin, cladribin, cytarabin, cryptophycins, dacarbazine, dasatinib, daunorubicin, docetaxel, doxorubicin, duocarmycin, dynemycin A, epothilones, etoposide, floxuridine, fludarabine, 5-fluorouracil, gefitinib, gemcitabine, ipilimumab, hydroxyurea, imatinib, infliximab, interferons, interleukins, beta-lapachone, lenalidomide, irinotecan, maytansine, mechlorethamine, melphalan, 6-mercaptopurine, methotrexate, mitomycin C, nilotinib, oxaliplatin, paclitaxel, procarbazine, suberoylanilide hydroxamic acid (SAHA), 6-thioguanidine, thiotepa, teniposide, topotecan, trastuzumab, trichostatin A, vinblastine, vincristine, vindesine or tamoxifen.

669. The depot of any one of the clauses herein, wherein the therapeutic region further comprises an anti-inflammatory agent.

670. The depot of any one of the clauses herein, wherein the anti-inflammatory agent includes at least one of prednisone, betamethasone, cortisone, dexamethasone, hydrocortisone, methylprednisolone, aspirin, Ibuprofen, naproxen sodium, diclofenac, diclofenac-misoprostol, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, mefenamic acid or COX-2 inhibitors.

671. The depot of any one of the clauses herein, wherein the therapeutic region further comprises an antibiotic and/or antimicrobial agent.

672. The depot of any one of the clauses herein, wherein the antibiotic and/or antimicrobial agent includes at least one of amoxicillin, amoxicillin/clavulanate, cephalexin, ciprofloxacin, clindamycin, metronidazole, azithromycin, levofloxacin, sulfamethoxazole/trimethoprim, tetracycline(s), minocycline, tigecycline, doxycycline, rifampin, triclosan, chlorhexidine, penicillin(s), aminoglycides, quinolones, fluoroquinolones, vancomycin, gentamycin, cephalosporin(s), carbapenems, imipenem, ertapenem, antimicrobial peptides, cecropin-mellitin, magainin, dermaseptin, cathelicidin, α-defensins or α-protegrins.

673. The depot of any one of the clauses herein, wherein the therapeutic region further comprises an antifungal agent.

674. The depot of any one of the clauses herein, wherein the antifungal agent includes at least one of ketoconazole, clortrimazole, miconazole, econazole, intraconazole, fluconazole, bifoconazole, terconazole, butaconazole, tioconazole, oxiconazole, sulconazole, saperconazole, voriconazole, terbinafine, amorolfine, naftifine, griseofulvin, haloprogin, butenafine, tolnaftate, nystatin, cyclohexamide, ciclopirox, flucytosine, terbinafine or amphotericin.

675. The depot of any one of the clauses herein, wherein the therapeutic region further comprises a steroid.

676. The depot of any one of the clauses herein, wherein the steroid includes at least one of prednisone, betamethasone, cortisone, dexamethasone, hydrocortisone or methylprednisolone.

677. The depot of any one of the clauses herein, wherein the therapeutic region further comprises an immunosuppressant.

678. The depot of any one of the clauses herein, wherein the immunosuppressant includes at least one of cyclosporine, pimecrolimus, sirolimus or tacrolimus.

679. The depot of any one of the clauses herein, wherein the therapeutic region includes a first portion and a second portion, wherein the first portion comprises the therapeutic agent and the second portion comprises at least one the immunotherapeutic agent, analgesic, anti-inflammatory agent, antibiotic agent, antifungal agent, steroid or immunosuppressant.

680. The depot of any one of the clauses herein, wherein the first portion is closer to an exterior surface of the depot than the second portion.

681. The depot of any one of the clauses herein, wherein the first portion is farther from an exterior surface of the depot than the second portion.

682. The depot of any one of the clauses herein, wherein therapeutic region is configured to release the immunotherapeutic agent, analgesic, anti-inflammatory agent, antibiotic agent, antifungal agent, steroid and/or immunosuppressant continuously for the period of time.

683. The depot of any one of the clauses herein, wherein the depot is configured to release the therapeutic agent at a first rate and the immunotherapeutic agent, analgesic, anti-inflammatory agent, antibiotic agent, antifungal agent, steroid and/or immunosuppressant at a second rate.

684. The depot of any one of the clauses herein, wherein the first rate is the same as the second rate.

685. The depot of any one of the clauses herein, wherein the first rate is different than the second rate.

686. The depot of any one of the clauses herein, wherein the first rate is greater than the second rate.

687. The depot of any one of the clauses herein, wherein the first rate is less than the second rate.

688. A system for treating or preventing HIV via the controlled, sustained release of a therapeutic agent, the system comprising:

the depot of any one of the clauses herein; and

a delivery device configured to deliver the depot beneath a patient's skin.

689. A system for treating or preventing symptoms associated with HIV, comprising:

a plurality of depots, each depot comprising a depot of any one of the clauses herein; and

a delivery device configured to position the depots beneath a patient's skin.

690. A system for treating or preventing malaria via the controlled, sustained release of a therapeutic agent, the system comprising:

the depot of any one of the clauses herein; and

a delivery device configured to deliver the depot beneath a patient's skin.

691. A system for treating or preventing symptoms associated with malaria, comprising:

a plurality of depots, each depot comprising a depot of any one of the clauses herein; and

a delivery device configured to position the depots beneath a patient's skin.

692. The system of any one of the clauses herein, wherein the delivery device is a syringe.

693. The system of any one of the clauses herein, further comprising securing the depot at or proximate to the abdomen, deltoid, gluteal, arm, and thigh/femur.

694. The system of any one of the clauses herein, wherein the period of time is no less than 1 day, no less than two days, no less than three days, no less than four days, no less than five days, no less than six days, no less than one week, no less than two weeks, no less than three weeks, no less than four weeks, no less than five weeks, no less than 8 weeks, no less than 2 months, no less than 3 months, no less than 4 months, no less than 6 months, no less than 7 months, no less than 8 months, no less than 9 months, no less than 10 months, no less than 12 months, no less than 18 months, no less than 24 months, no less than 30 months, no less than 36 months.

695. A method for treating or preventing symptoms associated with HIV via the controlled, sustained release of a therapeutic agent, the method comprising:

providing a depot of any one of the clauses herein.

696. A method for treating or preventing symptoms associated with HIV via the controlled, sustained release of a therapeutic agent, the method comprising:
positioning a depot of any one of the clauses herein at a treatment site proximate an eye of a patient; and
delivering the therapeutic agent to the patient for a period of time.

697. A method for treating or preventing symptoms associated with malaria via the controlled, sustained release of a therapeutic agent, the method comprising:

providing a depot of any one of the clauses herein.

698. A method for treating or preventing symptoms associated with malaria via the controlled, sustained release of a therapeutic agent, the method comprising:
positioning a depot of depot of any one of the clauses herein at a treatment site proximate an eye of a patient; and
delivering the therapeutic agent to the patient for a period of time.

699. The method of any one of the clauses herein, further comprising securing the depot at or proximate to the abdomen, deltoid, gluteal, arm, and thigh/femur.

700. The method of any one of the clauses herein, wherein the period of time is no less than 1 day, no less than two days, no less than three days, no less than four days, no less than five days, no less than six days, no less than one week, no less than two weeks, no less than three weeks, no less than four weeks, no less than five weeks, no less than 8 weeks, no less than 2 months, no less than 3 months, no less than 4 months, no less than 6 months, no less than 7 months, no less than 8 months, no less than 9 months, no less than 10 months, no less than 12 months, no less than 18 months, no less than 24 months, no less than 30 months, no less than 36 months.

701. An implantable medical device (IMD) cover comprising a depot configured to provide for the controlled, sustained release of a therapeutic agent, the depot comprising:
a therapeutic region comprising a therapeutic agent;
a control region comprising a bioresorbable polymer and a releasing agent mixed with the polymer, wherein the releasing agent is configured to dissolve when the depot is placed in vivo to form diffusion openings in the control region; and
wherein the cover is configured to at least partially cover the IMD and, while implanted, release the therapeutic agent for a period of time that is no less than 3 days.

702. A depot configured to be disposed along an outer portion of an implantable medical device (IMD) assembly, the depot configured to provide for the controlled, sustained release of a
therapeutic agent, the depot comprising:
a therapeutic region comprising a therapeutic agent; a control region comprising a bioresorbable polymer and a releasing agent mixed with the polymer, wherein the releasing agent is configured to dissolve when the depot is placed in vivo to form diffusion openings in the control region; and
wherein the IMD is configured to be implanted in a body of a patient and, while implanted, release the therapeutic agent for a period of time.

703. A depot configured to cover at least a portion of an implantable medical device (IMD) and provide for the controlled, sustained release of a therapeutic agent, the depot comprising:
a therapeutic region comprising a therapeutic agent; and
a control region comprising a bioresorbable polymer and a releasing agent mixed with the polymer, wherein the releasing agent is configured to dissolve when the depot is placed in vivo to form diffusion openings in the control region;
wherein the depot is configured to cover the IMD and, while implanted, release the therapeutic agent for a period of time.

704. A depot configured to provide for the controlled, sustained release of a therapeutic agent to treat or prevent infection, the depot comprising:
a therapeutic region comprising a therapeutic agent;
a control region comprising a bioresorbable polymer and a releasing agent mixed with the polymer, wherein the releasing agent is configured to dissolve when the depot is placed in vivo to form diffusion openings in the control region; and
wherein the cover is configured to at least partially cover the IMD and, while implanted, release the therapeutic agent for a period of time.

705. The depot of any one of the clauses herein, wherein the therapeutic agent comprises at least one of an antibiotic agent, an anti-biofilm agent, an anti-septic agent, or an anti-fungal agent.

706. The depot of any one of the clauses herein, wherein the therapeutic agent comprises one or more of: amoxicillin, amoxicillin/clavulanate, ampicilline, cephalexin, cefixime, ceftriaxone, ciprofloxacin, clindamycin, cloxacillin, cotrimaxazole, metronidazole, clindamycin, azithromycin, erythromycin, and clarithromycin, levofloxacin, ofloxacin, sulfamethoxazole/trimethoprim, tetracycline(s), minocycline, tigecycline, doxycycline, rifampin, triclosan, chlorhexidine, penicillin(s), aminoglycides, quinolones, fluoroquinolones,beta-lactams, rifampicin, vancomycin, daptomycin, fostomycin, gentamycin, cephalosporin(s), carbapenems, imipenem, ertapenem, antimicrobial peptides, cecropin-mellitin, magainin, dermaseptin, cathelicidin, α-defensins, α-protegrins, lactoferrin, ethylenediaminetetraacetic acid (EDTA), xylitol, gallium, dispersin B, farsenol, RNA-III inhibiting peptide (RIP), and furanone C30, lysostaphin, DNase I, V8 protease, apto-transferrin, ethylene glycol tetraacetic acid (EGTA), 1,2,3,4,6-Penta-O-galloyl-beta-D-glucopyranose (PGG), Cis-2 decenoic acid (C2DA), diarylacrylonitriles, aryl ethyl ketones, vinyl sulfones, N-acetyl-L-cysteine (NAC), ethanol, chlorohexidine, ketoconazole, clortrimazole, miconazole, econazole, intraconazole, fluconazole, bifoconazole, terconazole, butaconazole, tioconazole, oxiconazole, sulconazole, saperconazole, voriconazole, terbinafine, amorolfine, naftifine, griseofulvin, haloprogin, butenafine, tolnaftate, nystatin, cyclohexamide, ciclopirox, flucytosine, terbinafine, or amphotericin B.

707. The depot of any one of the clauses herein, wherein the IMD comprises one or more of: an intravascular IMD, a cardiovascular IMD, a neurosurgical IMD, an orthopedic IMD, a urological IMD, a gynecological IMD, an otolaryngological IMD, an ophthalmological IMD, or a dental IMD.

708. The depot of any one of the clauses herein, wherein the IMD comprises one or more of: a peripheral venous catheter, a peripheral arterial catheter, a midline catheter, a central venous catheter, a non-tunneled catheter, a tunneled catheter, a pulmonary artery catheter, a totally implanted port, a vascular access device, a mechanical heart valve, an implantable defibrillator, a vascular graft, a ventricular assist device, a coronary stent, an implantable patient monitor, a ventricular shunt, an Ommaya reservoir, an intracranial pressure device, an implantable neurological stimulators, a joint prosthesis, a reconstructive orthopedic implant, a spinal implant, a fracture-fixation device, an inflatable penile implant, an IMD, a cochlear implant, a middle-ear implant, an intra-ocular lens, a glaucoma tube, a dental prosthesis, and a dental appliance.

709. The depot of any one of the clauses herein, wherein the depot substantially encapsulates the IMD.

710. The depot of any one of the clauses herein, wherein the depot comprises an aperture configured to receive the IMD therethrough.

711. The depot of any one of the clauses herein, wherein the depot circumferentially surrounds at least a portion of the IMD.

712. The depot of any one of the clauses herein, wherein the depot forms a socket, sleeve, or band configured to at least partially surround the IMD.

713. The depot of any one of the clauses herein, wherein the depot is elastomeric and configured to stretch around at least a portion of the IMD.

714. The depot of any one of the clauses herein, wherein the therapeutic agent in the therapeutic region comprises at least 50% of the total weight of the depot.

715. The depot of any one of the clauses herein, wherein about 40% to about 60% of the therapeutic agent in the therapeutic region is released in the first half of the period of time.

716. The depot of any one of the clauses herein, wherein at least 90% of the therapeutic agent in the therapeutic region is released within the period of time.

717. The depot of any one of the clauses herein, wherein depot is configured to release the therapeutic agent at a rate of at least 100 mg per day, at least 200 mg per day, at least 300 mg per day, at least 400 mg per day, at least 500 mg per day, at least 600 mg per day, at least 700 mg per day, at least 800 mg per day, at least 900 mg per day, at least 1 g per day, at least 1.5 g per day, at least 2 g per day, at least 2.5 g per day, at least 3 g per day, at least 4 g per day, at least 5 g per day, at least 6 g per day, at least 7 g per day, at least 8 g per day, at least 9 g per day, or at least 10 g per day.

718. The depot of any one of the clauses herein, wherein depot is configured to release the therapeutic agent at a rate of no more than 100 mg per day, no more than 200 mg per day, no more than 300 mg per day, no more than 400 mg per day, no more than 500 mg per day, no more than 600 mg per day, no more than 700 mg per day, no more than 800 mg per day, no more than 900 mg per day, no more than 1 g per day, no more than 1.5 g per day, no more than 2 g per day, no more than 2.5 g per day, no more than 3 g per day, no more than 4 g per day, no more than 5 g per day, no more than 6 g per day, no more than 7 g per day, no more than 8 g per day, no more than 9 g per day, or no more than 10 g per day.

719. The depot of any one of the clauses herein, wherein the depot is configured to release the therapeutic agent to the treatment site continuously over the period of time.

720. The depot of any one of the clauses herein, wherein the depot is configured to release the therapeutic agent to the treatment site intermittently over the period of time.

721. The depot of any one of the clauses herein, wherein the therapeutic agent is released at a substantially steady state rate throughout the period of time.

722. The depot of any one of the clauses herein, wherein the period of time is no less than 8 days, no less than 9 days, no less than 10 days, no less than 11 days, no less than 12 days, no less than 13 days, no less than 14 days, no less than 15 days, no less than 16 days, no less than 17 days, no less than 18 days, no less than 19 days, no less than 20 days, no less than 21 days, no less than 22 days, no less than 23 days, no less than 24 days, no less than 25 days, no less than 26 days, no less than 27 days, no less than 28 days, no less than 29 days, no less than 30 days, no less than 40 days, no less than 50 days, no less than 60 days, no less than 70 days, no less than 90 days, no less than 100 days, no less than 200 days, no less than 300 days, or no less than 365 days.

723. The depot of any one of the clauses herein, wherein the depot is biodegradable and/or bioerodible.

724. The depot of any one of the clauses herein, wherein the therapeutic region comprises at least 10 mg, at least 20 mg, at least 30 mg, at least 40, at least 50 mg, at least 100 mg, at least 150 mg, at least 200 mg, at least 300 mg, at least 400 mg, at least 500 mg, at least 600 mg, at least 700 mg, at least 800 mg, at least 1 g, at least 1.25 g, at least 1.5 g, at least 1.75 g, at least 2.0 g, at least 2.25 g, at least 2.5 g, at least 2.75 g, at least 3.0 g, at least 3.25 g, at least 3.5 g, at least 3.75 g, at least 4.0 g, at least 4.25 g, at least 4.5 g, at least 4.75 g, or at least 5.0 g.

725. A method for treating or preventing infection associated with an implantable medical device (IMD), the method comprising:

providing an IMD; and

providing a depot of any one of the clauses herein.

726. A method for treating or preventing infection associated with an implantable medical device (IMD), the method comprising:
positioning a depot of any one of the clauses herein at a treatment site proximate an IMD implanted within a patient;
delivering the therapeutic agent to the treatment site for a period of time that is no less than 3 days.

727. The method of any one of the clauses herein, wherein positioning the depot comprises covering at least a portion of the IMD with the depot.

728. The method of any one of the clauses herein, wherein positioning the depot comprises coupling the depot to the IMD and then implanting the IMD in the patient.

729. A depot for treating an ocular condition of a patient via sustained, controlled release of a therapeutic agent to the patient, the depot comprising:
a therapeutic region comprising the therapeutic agent;
a control region comprising a bioresorbable polymer and a releasing agent mixed with the polymer, wherein the releasing agent is configured to dissolve when the depot is placed in vivo to form diffusion openings in the control region; and
wherein the depot is configured to be implanted at a treatment site at or proximate an eye of the patient and, while implanted, release the therapeutic agent at the treatment site for a period of time.

730. The depot of any one of the clauses herein, wherein the treatment site includes the conjunctiva, subconjunctival space, punctal space, cornea, sclera, pars plana, macula, vitreous cavity, choroid, suprachoroidal space, retina, posterior chamber, or anterior chamber of the eye.

731. The depot of any one of the clauses herein, wherein the depot includes a securing portion configured to adhere to a surface of at least one of the conjunctiva, subconjunctival space, punctal space, cornea, sclera, pars plana, macula, vitreous cavity, choroid, suprachoroidal space, retina, posterior chamber, or anterior chamber of the eye.

732. The depot of any one of the clauses herein, wherein the depot includes an anchor member coupled to the therapeutic region, control region, and/or base region, and wherein the anchor member is configured to self-expand into a position with at least a portion of the surface, thereby securing the depot at or within the eye.

733. The depot of any one of the clauses herein, wherein the depot includes a fixation portion configured to penetrate at least a portion of the thickness of the sclera, choroid or retina of the eye, thereby securing the depot at the eye.

734. The depot of any one of the clauses herein, wherein the ocular condition includes at least one of glaucoma, inflammation, macular degeneration, macular edema, cataracts, ocular hypertension, uveitis or dry eye.

735. The depot of any one of the clauses herein, wherein the therapeutic agent comprises at least one of a cholinergic agonist, prostaglandins analog, carbonic anhydrase inhibitor, alpha and/or beta adrenic agonist, antibody, fusion protein, peptide, chemokine, interleukin, or neuroprotective agent.

736. The depot of any one of the clauses herein, wherein the cholinergic agonist comprises at least one of pilocarpine or cevimeline.

737. The depot of any one of the clauses herein, wherein the prostaglandins analog comprises at least one of latanoprost, travoprost, bimatoprost, or unoprostine.

738. The depot of any one of the clauses herein, wherein the carbonic anhydrase inhibitor comprises at least one of methazolamide, 5-acylimino- or related iminosubstituted analogs.

739. The depot of any one of the clauses herein, wherein the alpha and/or beta adrenic agonist comprises at least one of brimonidine, brimonidine tartrate, apraclonidine, timolol, levobunalol, carteolol, metipranolol, or betaxolol.

740. The depot of any one of the clauses herein, wherein the antibody comprises at least one of adalimumab, alefacept, basiliximab, bevacizumab, certolizumab, daclizumab, efalizumab, golimumab, infliximab, natalizumab, ranibizumab, or rituximab.

741. The depot of any one of the clauses herein, wherein the fusion protein comprises at least one of abatacept, alefacept, anakinra, or etanercept.

742. The depot of any one of the clauses herein, wherein the peptide comprises at least one of antimicrobial peptides, calcitonin gene-related peptide, cell penetrating peptides, fibronectin-derived peptides, neurotransmitters, substance P, tachykinins or vasoactive intestinal peptide.

743. The depot of any one of the clauses herein, wherein the chemokine comprises C—C motif chemokine 22.

744. The depot of any one of the clauses herein, wherein the interleukin comprises at least one of IL-2, TNF or IL-Iβ.

745. The depot of any one of the clauses herein, wherein the neuroprotective agent comprises at least one of brain-derived neurotrophic factor, glial cell-line neurotrophic factor or nerve growth factor.

746. The depot of any one of the clauses herein, wherein the therapeutic agent comprises at least one of dipivefrin, carbachol, acetazolamide, dorzolamide, ethacrynic acid, mitomycin C, diclofenac, flurbiprofen, dexamethasone, coenzyme-Q10, ganciclovir, fluocinolone acetonide, triamcinolone acetonide, hydroxypropylcellulose, brinzolamide, albumin, or immunoglobulin.

747. The depot of any one of the clauses herein, wherein the therapeutic region is configured to release pilocarpine at rate of about 0.2 mg/day to about 0.8 mg/day.

748. The depot of any one of the clauses herein, wherein the therapeutic region is configured to release dexamethasone at rate of about 1 μg/day to about 100 μg/day.

749. The depot of any one of the clauses herein, wherein the therapeutic region is configured to release triamcinolone acetonide at rate of about 1 μg/day to about 100 μg/day.

750. The depot of any one of the clauses herein, wherein the therapeutic region is configured to release ganciclovir at rate of about 0.1 μg/day to about 10 μg/day.

751. The depot of any one of the clauses herein, wherein the therapeutic region is configured to release fluocinolone acetonide at rate less than about 100 ng/day.

752. The depot of any one of the clauses herein, wherein the therapeutic region is configured to release dexamethasone at rate of about 900 μg/day to about 1.2 mg/day.

753. The depot of any one of the clauses herein, wherein the therapeutic region is configured to release triamcinolone acetonide at rate of about 0.5 μg/day to about 5 μg/day.

754. The depot of any one of the clauses herein, wherein the therapeutic region is configured to release fluocinolone acetonide at rate of about 0.1 μg/day to about 0.8 μg/day.

755. The depot of any one of the clauses herein, wherein the therapeutic region is configured to release ciliary neurotropic factor at rate less than about 50 ng/day.

756. The depot of any one of the clauses herein, wherein the therapeutic region is configured to release hydroxypropylcellulose at rate of about 1 mg/day to about 10 mg/day.

757. The depot of any one of the clauses herein, wherein the period of time is no less than two weeks, no less than three weeks, no less than four weeks, no less than five weeks, no less than 8 weeks, no less than 2 months, no less than 3 months, no less than 4 months, no less than 6 months, no less than 7 months, no less than 8 months, no less than 9 months, no less than 10 months, no less than 12 months, no less than 18 months, no less than 24 months, no less than 30 months, no less than 36 months.

758. The depot of any one of the clauses herein, wherein the therapeutic agent contains at least 10 μg, 50 μg, 100 μg, 200 μg, 300 μg, 400 μg, 500 μg, 600 μg, 700 μg, 800 μg, 900 μg, 1 mg or 10 mg, of the therapeutic agent.

759. The depot of any one of the clauses herein, wherein the therapeutic region contains less than 20 mg, less than 15 mg, less than 10 mg, less than 5 mg, less than 1 mg, less than 900 μg, less than 800 μg, less than 700 μg, less than 600 μg, less than 500 μg, less than 400 μg, less than 300 μg, less than 200 μg, less than 100 μg, less than 50 μg, less than 25 μg, or less than 10 μg, of the therapeutic agent.

760. The depot of any one of the clauses herein, wherein the therapeutic region contains from 10 μg to 10 mg, 10 μg to 1000 μg, 10 μg to 900 μg, 10 μg to 800 μg, 10 μg to 700 μg, 10 μg to 600 μg, 10 μg to 500 μg, 10 μg to 400 μg, 10 μg to 300 μg, 10 μg to 200 μg, 10 μg to 100 μg, 10 μg to 75 μg, 10 μg to 50 μg, or 10 μg to 20 μg of the therapeutic agent.

761. The depot of any one of the clauses herein, wherein the therapeutic region is configured to release the therapeutic agent through the period of time at a rate of from 10 ng/day to 900 μg/day, 10 ng/day to 700 μg/day, 10 ng/day to 500 μg/day, 10 ng/day to 400 μg/day, 10 ng/day to 300 μg/day, 10 ng/day to 200 μg/day, 10 ng/day to 100 μg/day, 10 ng/day to 10 μg/day, 10 ng/day to 900 ng/day, 10 ng/day to 800 ng/day, 10 ng/day to 700 ng/day, 10 ng/day to 600 ng/day, 10 ng/day to 500 ng/day, 10 ng/day to 400 ng/day, 10 ng/day to 300 ng/day, 10 ng/day to 200 ng/day, 10 ng/day to 100 ng/day, 10 ng/day to 75 ng/day, 10 ng/day to 50 ng/day, 10 ng/day to 25 ng/day.

762. The depot of any one of the clauses herein, wherein the therapeutic region is configured to release the therapeutic agent through the period of time at a rate no more than 10 mg/day, no more than 1 mg/day, no more than 500 μg/day, no more than 100 μg/day, no more than 90 μg/day, no more than 80 μg/day, no more than 70 μg/day, no more than 60 μg/day, no more than 50 μg/day, no more than 40 μg/day, no more than 30 μg/day, no more than 20 μg/day, no more than 10 μg/day, no more than 5 μg/day, no more than 1 μg/day, no more than 900 ng/day, no more than 800 ng/day, no more than 700 ng/day, no more than 600 ng/day, no more than 500 ng/day, no more than 400 ng/day, no more than 300 ng/day, no more than 200 ng/day, no more than 100 ng/day, no more than 50 ng/day, no more than 40 ng/day, no more than 30 ng/day, no more than 20 ng/day, or no more than 10 ng/day.

763. A method for treating an ocular condition via the controlled, sustained release of a therapeutic agent, the method comprising:

providing a depot of any one of clauses herein.

764. A method for treating an ocular condition via the controlled, sustained release of a therapeutic agent, the method comprising:
positioning a depot of any one of clauses herein at a treatment site proximate an eye of a patient; and
delivering the therapeutic agent to the treatment site for a period of time.

765. The method of any one of the clauses herein, further comprising securing the depot at the conjunctiva, subconjunctival space, punctal space, cornea, sclera, pars plana, macula, vitreous cavity, choroid, suprachoroidal space, retina, posterior chamber, or anterior chamber of the eye.

766. The method of any one of the clauses herein, further comprising securing the depot to a portion of the eye.

767. The method of any one of the clauses herein, wherein the target site comprises the conjunctiva, subconjunctival space, punctal space, cornea, sclera, pars plana, macula, vitreous cavity, choroid, suprachoroidal space, retina, posterior chamber, or anterior chamber of the eye.

768. The method of any one of the clauses herein, wherein the period of time is no less than two weeks, no less than three weeks, no less than four weeks, no less than five weeks, no less than 8 weeks, no less than 2 months, no less than 3 months, no less than 4 months, no less than 6 months, no less than 7 months, no less than 8 months, no less than 9 months, no less than 10 months, no less than 12 months, no less than 18 months, no less than 24 months, no less than 30 months, no less than 36 months.

769. The method of any one of the clauses herein, wherein the depot is a first depot and the method further comprises positioning a second depot at the treatment site.

770. A system for treating symptoms associated with an ocular condition via the controlled, sustained release of a therapeutic agent, the system comprising:

the depot of any one of clauses herein; and

a delivery device configured to position the depot at a target site of a patient's eye.

771. The system of any one of the clauses herein, wherein the target site includes the conjunctiva, subconjunctival space, punctal space, cornea, sclera, pars plana, macula, vitreous cavity, choroid, suprachoroidal space, retina, posterior chamber, or anterior chamber of the eye.

772. The system of any one of the clauses herein, wherein the delivery device is a syringe and the target site is the vitreous cavity of the eye.

773. A system for treating an ocular condition, comprising:

a plurality of depots, each depot comprising a depot of any one of clauses herein; and

a delivery device configured to position the depots depot at a target site of a patient's eye.

774. A depot for treating an otolaryngologic condition of a patient via sustained, controlled release of a therapeutic agent to the patient, the depot comprising:
a therapeutic region comprising the therapeutic agent;
a control region comprising a bioresorbable polymer and a releasing agent mixed with the polymer, wherein the releasing agent is configured to dissolve when the depot is placed in vivo to form diffusion openings in the control region; and
wherein the depot is configured to be implanted at a treatment site at or proximate a nasal cavity of the patient and, while implanted, release the therapeutic agent at the treatment site for a period of time.

775. The depot of any one of the clauses herein, wherein the treatment site includes the nasal cavity, the frontal sinus, the sphenoid sinus, the ethmoid sinus, the maxillary sinus, the superior nasal concha, the middle nasal concha, the inferior nasal concha, the vestibule, or the nasopharynx.

776. The depot of any one of the clauses herein, wherein the depot includes a securing portion configured to adhere to a surface of at least one of the nasal cavity, the frontal sinus, the sphenoid sinus, the ethmoid sinus, the maxillary sinus, the superior nasal concha, the middle nasal concha, the inferior nasal concha, the vestibule, or the nasopharynx.

777. The depot of any one of the clauses herein, wherein the depot includes an anchor member coupled to the therapeutic region, control region, and/or base region, and wherein the anchor member is configured to self-expand into apposition with at least a portion of the surface, thereby securing the depot at or within the nasal cavity.

778. The depot of any one of the clauses herein, wherein the depot includes a fixation portion configured to penetrate at least a portion of the thickness of the nasal cavity wall, the superior nasal concha, the middle nasal concha, the inferior nasal concha, the vestibule, or the nasopharynx thereby securing the depot at the nasal cavity.

779. The depot of any one of the clauses herein, wherein the otolaryngologic condition includes at least one of sinusitis, allergic rhinitis, nasal infection or chronic nasal congestion.

780. The depot of any one of the clauses herein, wherein the therapeutic region is configured to release the therapeutic agent continuously at a substantially constant rate over the period of time.

781. The depot of any one of the clauses herein, wherein the therapeutic region is configured to release the therapeutic agent continuously at a rate that increases over the period of time.

782. The depot of any one of the clauses herein, wherein the period of time includes a first period of time and a second period of time after the first period of time, and wherein the therapeutic region is configured to release the therapeutic agent at a first rate during the first period of time and a second rate during the second period of time, the second rate being less than the first rate.

783. The depot of any one of the clauses herein, wherein the therapeutic agent is configured to treat rhinosinusitis.

784. The depot of any one of the clauses herein, wherein the therapeutic agent comprises at least one of a steroid, an anti-inflammatory agent or an antibiotic agent.

785. The depot of any one of the clauses herein, wherein the steroid comprises at least one of mometasone, triamcinolone, prednisone, prednisolone, methylprednisolone, ciclesonide, fluticasone furoate, fluticasone propionate, mometasone, beclomethasone, budesonide, flunisolide, or triamcinolone, cortisone, dexamethasone or hydrocortisone.

786. The depot of any one of the clauses herein, wherein the antiobiotic agent includes at least one of: amoxicillin, amoxycillin/clavunate, clindamycin, cephalexin, metronidazole/cefalexin, metronidazole/cefluroxime, metronidazole/cefprozil, moxifloxacin, levofloxacin, clarithromycin, tobramycin, cefuroxime, ceftazidime, ofloxacin, gentamycin, mupirocin, macrolides, doxycycline, ceftriaxone, femifloxacin, trimethoprim-sulfame-thoxazol, ciprofloxacin, clindamycin, metronidazole, azithromycin, levofloxacin, sulfamethoxazole/trimethoprim, tetracycline(s), minocycline, tigecycline, doxycycline, rifampin, triclosan, chlorhexidine, penicillin(s), aminoglycides, quinolones, fluoroquinolones, vancomycin, gentamycin, cephalosporin(s), carbapenems, imipenem, ertapenem, antimicrobial peptides, cecropin-mellitin, magainin, dermaseptin, cathelicidin, α-defensins or α-protegrins.

787. The depot of any one of the clauses herein, wherein the anti-inflammatory includes or more of: macrolide, erythromycin, roxithromycin, azithromycin, prednisone, betamethasone, cortisone, dexamethasone, hydrocortisone, methylprednisolone, aspirin, Ibuprofen, naproxen sodium, diclofenac, diclofenac-misoprostol, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, mefenamic acid or COX-2 inhibitors.

788. The depot of any one of the clauses herein, wherein the therapeutic region is configured to release mometasone furoate at rate of about 1μg/day to about 30 μg/day.

789. The depot of any one of the clauses herein, wherein the therapeutic region is configured to release mometasone furoate at rate of about 10 μg/day to about 20 μg/day.

790. The depot of any one of the clauses herein, wherein the therapeutic region is configured to release triamcinolone acetonide at rate of about 1 μg/day to about 100 μg/day.

791. The depot of any one of the clauses herein, wherein the therapeutic region is configured to release prednisolone at a rate of about 25 mg/day to about 75 mg/day.

792. The depot of any one of the clauses herein, wherein the period of time is no less than 3 days, no less than 4 days, no less than 5 days, no less than 6 days, no less than 7 days, no less than 8 days, no less than 9 days, no less than 10 days, no less than 11 days, no less than 12 days, no less than 13 days, no less than 2 weeks, no less than 3 weeks, no less than 4 weeks, no less than 5 weeks, no less than 8 weeks, no less than 2 months, no less than 3 months, no less than 4 months, no less than 6 months, no less than 7 months, no less than 8 months, no less than 9 months, no less than 10 months, no less than 12 months, no less than 18 months, no less than 24 months, no less than 30 months, no less than 36 months.

793. The depot of any one of the clauses herein, wherein the therapeutic agent contains at least 10 μg, 50 μg, 100 μg, 200 μg, 300 μg, 400 μg, 500 μg, 600 μg, 700 μg, 800 μg, 900 μg, 1 mg or 10 mg, of the therapeutic agent.

794. The depot of any one of the clauses herein, wherein the therapeutic region contains less than 20 mg, less than 15 mg, less than 10 mg, less than 5 mg, less than 1 mg, less than 900 μg, less than 800 μg, less than 700 μg, less than 600 μg, less than 500 μg, less than 400 μg, less than 300 μg, less than 200 μg, less than 100 μg, less than 50 μg, less than 25 μg, or less than 10 μg, of the therapeutic agent.

795. The depot of any one of the clauses herein, wherein the therapeutic region contains from 10 μg to 10 mg, 10 μg to 1000 μg, 10 μg to 900 μg, 10 μg to 800 μg, 10 μg to 700 μg, 10 μg to

600 μg, 10 μg to 500 μg, 10 μg to 400 μg, 10 μg to 300 μg, 10 μg to 200 μg, 10 μg to 100 μg, 10 μg to 75 μg, 10 μg to 50 μg, or 10 μg to 20 μg of the therapeutic agent.

796. The depot of any one of the clauses herein, wherein the therapeutic region is configured to release the therapeutic agent through the period of time at a rate of from 10 ng/day to 900 μg/day, 10 ng/day to 700 μg/day, 10 ng/day to 500 μg/day, 10 ng/day to 400 μg/day, 10 ng/day to 300 μg/day, 10 ng/day to 200 μg/day, 10 ng/day to 100 μg/day, 10 ng/day to 10 μg/day, 10 ng/day to 900 ng/day, 10 ng/day to 800 ng/day, 10 ng/day to 700 ng/day, 10 ng/day to 600 ng/day, 10 ng/day to 500 ng/day, 10 ng/day to 400 ng/day, 10 ng/day to 300 ng/day, 10 ng/day to 200 ng/day, 10 ng/day to 100 ng/day, 10 ng/day to 75 ng/day, 10 ng/day to 50 ng/day, 10 ng/day to 25 ng/day.

797. The depot of any one of the clauses herein, wherein the therapeutic region is configured to release the therapeutic agent through the period of time at a rate no more than 10 mg/day, no more than 1 mg/day, no more than 500 μg/day, no more than 100 μg/day, no more than 90 μg/day, no more than 80 μg/day, no more than 70 μg/day, no more than 60 μg/day, no more than 50 μg/day, no more than 40 μg/day, no more than 30 μg/day, no more than 20 μg/day, no more than 10 μg/day, no more than 5 μg/day, no more than 1 μg/day, no more than 900 ng/day, no more than 800 ng/day, no more than 700 ng/day, no more than 600 ng/day, no more than 500 ng/day, no more than 400 ng/day, no more than 300 ng/day, no more than 200 ng/day, no more than 100 ng/day, no more than 50 ng/day, no more than 40 ng/day, no more than 30 ng/day, no more than 20 ng/day, or no more than 10 ng/day.

798. A method for treating an otolaryngologic condition via the controlled, sustained release of a therapeutic agent, the method comprising:

providing a depot of any one of the clauses herein.

799. A method for treating an otolaryngologic condition via the controlled, sustained release of a therapeutic agent, the method comprising:
positioning a depot of any one of the clauses herein at a treatment site proximate a nasal cavity of a patient; and
delivering the therapeutic agent to the treatment site for a period of time.

800. The method of any one of the clauses herein, further comprising securing the depot at nasal cavity, the frontal sinus, the sphenoid sinus, the ethmoid sinus, the maxillary sinus, the superior nasal concha, the middle nasal concha, the inferior nasal concha, the vestibule, or the nasopharynx.

801. The method of any one of the clauses herein, further comprising securing the depot to a portion of the nasal cavity.

802. The method of any one of the clauses herein, wherein the treatment site comprises the nasal cavity, the frontal sinus, the sphenoid sinus, the ethmoid sinus, the maxillary sinus, the superior nasal concha, the middle nasal concha, the inferior nasal concha, the vestibule, or the nasopharynx.

803. The method of any one of the clauses herein, wherein the period of time is no less than 3 days, no less than 4 days, no less than 5 days, no less than 6 days, no less than 7 days, no less than 8 days, no less than 9 days, no less than 10 days, no less than 11 days, no less than 12 days, no less than 13 days, no less than 2 weeks, no less than 3 weeks, no less than 4 weeks, no less than 5 weeks, no less than 8 weeks, no less than 2 months, no less than 3 months, no less than 4 months, no less than 6 months, no less than 7 months, no less than 8 months, no less than 9 months, no less than 10 months, no less than 12 months, no less than 18 months, no less than 24 months, no less than 30 months, no less than 36 months.

804. A system for treating an otolaryngologic condition via the controlled, sustained release of a therapeutic agent, the system comprising:

the depot of any one of clauses herein; and

a delivery device configured to position the depot at a target site of a patient's nasal cavity.

805. The system of any one of the clauses herein, wherein the treatment site includes the nasal cavity, the frontal sinus, the sphenoid sinus, the ethmoid sinus, the maxillary sinus, the superior nasal concha, the middle nasal concha, the inferior nasal concha, the vestibule, or the nasopharynx.

806. The system of any one of the clauses herein, wherein the delivery device is a syringe.

807. A system for treating an otolaryngologic condition, comprising:

a plurality of depots, each depot comprising a depot of any one of clauses herein; and

a delivery device configured to position the depots at a target site of a patient's nasal cavity.

808. A cover comprising a depot configured to provide for the controlled, sustained release of a therapeutic agent, the depot comprising:
a therapeutic region comprising a therapeutic agent; and
a control region comprising a bioresorbable polymer and a releasing agent mixed with the polymer, wherein the releasing agent is configured to dissolve when the depot is placed in vivo to form diffusion openings in the control region;
wherein the cover is configured to at least partially cover the breast implant and, while implanted, release the therapeutic agent for a period of time.

809. An assembly comprising a depot disposed along an outer portion of a breast implant, the depot configured to provide for the controlled, sustained release of a therapeutic agent, the depot comprising:
a therapeutic region comprising a therapeutic agent; and
a control region comprising a bioresorbable polymer and a releasing agent mixed with the polymer, wherein the releasing agent is configured to dissolve when the depot is placed in vivo to form diffusion openings in the control region;
wherein the breast implant is configured to be implanted in a breast of a patient and, while implanted, release the therapeutic agent for a period of time.

810. A depot configured to cover at least a portion of a breast implant and provide for the controlled, sustained release of a therapeutic agent, the depot comprising:
a therapeutic region comprising a therapeutic agent; and
a control region comprising a bioresorbable polymer and a releasing agent mixed with the polymer, wherein the releasing agent is configured to dissolve when the depot is placed in vivo to form diffusion openings in the control region;
wherein the depot is configured to cover the breast implant and, while implanted, release the therapeutic agent for a period of time.

811. The depot of any one of the clauses herein, wherein the therapeutic agent comprises one or more of: an antimicrobial agent, an anti-inflammatory agent, an anti-scarring agent, and a leukotriene inhibitor.

812. The depot of any one of the clauses herein, wherein the therapeutic agent comprises at least one of montelukast or zafirlukast.

813. The depot of any one of the clauses herein, wherein the therapeutic agent comprises at least one of tranilast.

814. The depot of any one of the clauses herein, wherein the therapeutic agent comprises triamcinolone.

815. The depot of any one of the clauses herein, wherein the therapeutic agent comprises perfenidone.

816. The depot of any one of the clauses herein, wherein the therapeutic agent comprises an anti-adhesion barrier solution.

817. The depot of any one of the clauses herein, wherein the therapeutic agent comprises an anti-bacterial agent.

818. The depot of any one of the clauses herein, wherein the therapeutic region comprises at least 10 mg, at least 20 mg, at least 30 mg, at least 40 50 mg, at least 100 mg, at least 150 mg, at least 200 mg, at least 300 mg, at least 400 mg, at least 500 mg, at least 600 mg, at least 700 mg, at least 800 mg, at least 1 g, at least 1.25 g, at least 1.5 g, at least 1.75 g, at least 2.0 g, at least 2.25 g, at least 2.5 g, at least 2.75 g, at least 3.0 g, at least 3.25 g, at least 3.5 g, at least 3.75 g, at least 4.0 g, at least 4.25 g, at least 4.5 g, at least 4.75 g, or at least 5.0 g.

819. The depot of any one of the clauses herein, wherein the therapeutic region is configured to release the therapeutic agent continuously over the period of time.

820. The depot of any one of the clauses herein, wherein the period of time is no less than 4 days, no less than 5 days, no less than 6 days, no less than 7 days, no less than 8 days, no less than 9 days, no less than 10 days, no less than 11 days, no less than 12 days, no less than 13 days, no less than 2 weeks, no less than 3 weeks, no less than 4 weeks, no less than 5 weeks, no less than 6 weeks, no less than 7 weeks, no less than 8 weeks, no less than 2 months, no less than 3 months, no less than 4 months, no less than 6 months, no less than 7 months, no less than 8 months, no less than 9 months, no less than 10 months, no less than 12 months, no less than 1 year.

821. The depot of any one of the clauses herein, wherein the depot substantially encapsulates the breast implant.

822. The depot of any one of the clauses herein, wherein the depot comprises an aperture configured to receive the breast implant therethrough.

823. The depot of any one of the clauses herein, wherein the depot circumferentially surrounds at least a portion of the breast implant.

824. A method for treating or preventing capsular contracture of a breast implant, the method comprising:

providing a breast implant; and

providing a depot of any one of the clauses herein.

825. A method for treating and/or preventing capsular contracture, the method comprising:
positioning a depot of any one of the clauses herein at a treatment site proximate a breast implant of a patient; and
delivering the therapeutic agent to the treatment site for a period of time that is no less than 3 days.

826. The method of any one of the clauses herein, wherein positioning the depot comprises covering at least a portion of the breast implant with the depot.

827. The method of any one of the clauses herein, wherein positioning the depot comprises coupling the depot to the breast implant and then implanting the breast implant in the patient.

828. The method of any one of the clauses herein, wherein the therapeutic agent comprises at least one of montelukast or zafirlukast.

829. The method of any one of the clauses herein, wherein the therapeutic agent comprises at least one of tranilast.

830. The method of any one of the clauses herein, wherein the therapeutic agent comprises triamcinolone.

831. The method of any one of the clauses herein, wherein the therapeutic agent comprises perfenidone.

832. The method of any one of the clauses herein, wherein the therapeutic agent comprises an anti-adhesion barrier solution.

833. The method of any one of the clauses herein, wherein the therapeutic agent comprises an anti-bacterial agent.

834. The method of any one of the clauses herein, wherein the therapeutic region comprises at least 10 mg, at least 20 mg, at least 30 mg, at least 40 50 mg, at least 100 mg, at least 150 mg, at least 200 mg, at least 300 mg, at least 400 mg, at least 500 mg, at least 600 mg, at least 700 mg, at least 800 mg, at least 1 g, at least 1.25 g, at least 1.5 g, at least 1.75 g, at least 2.0 g, at least 2.25 g, at least 2.5 g, at least 2.75 g, at least 3.0 g, at least 3.25 g, at least 3.5 g, at least 3.75 g, at least 4.0 g, at least 4.25 g, at least 4.5 g, at least 4.75 g, or at least 5.0 g.

835. The method of any one of the clauses herein, wherein the period of time is no less than two weeks, no less than three weeks, no less than four weeks, no less than five weeks, no less than 8 weeks, no less than 2 months, no less than 3 months, no less than 4 months, no less than 6 months, no less than 7 months, no less than 8 months, no less than 9 months, no less than 10 months, no less than 12 months, no less than 1 year.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 depicts the release of therapeutic agent over time from a prior art drug delivery system.

FIG. 2 is an isometric view of a depot configured in accordance with the present technology.

FIG. 3A depicts an example release profile over time of one or more depots of the present technology.

FIG. 3B depicts an example release profile over time of one or more depots of the present technology.

FIG. 4 is an isometric view of a depot in accordance with some embodiments of the present technology.

FIG. 5 is an isometric view of a depot in accordance with some embodiments of the present technology.

FIG. 6 is a cross-sectional view of a depot in accordance with some embodiments of the present technology.

FIG. 7 is a cross-sectional view of a depot in accordance with some embodiments of the present technology.

FIG. 8 is a cross-sectional view of a depot in accordance with some embodiments of the present technology.

FIG. 9A is an isometric view of a depot in accordance with some embodiments of the present technology.

FIG. 9B is a cross-sectional view of the depot shown in FIG. 9A.

FIG. 10 is a cross-sectional view of a depot in accordance with some embodiments of the present technology.

FIG. 11 is a cross-sectional view of a depot in accordance with some embodiments of the present technology.

FIG. 12 is a cross-sectional view of a depot in accordance with some embodiments of the present technology.

FIG. 13 is an isometric view of a depot in accordance with some embodiments of the present technology.

FIGS. 14A-H are depots having different cross-sectional areas and shapes in accordance with the present technology.

FIGS. 15A-15E depict various depot embodiments including a barrier region in accordance with the technology.

FIG. 16 is a perspective view of a depot in accordance with some embodiments of the present technology.

FIG. 17 is cross-sectional view of a depot in accordance with some embodiments of the present technology.

FIG. 18 is cross-sectional view of a depot in accordance with some embodiments of the present technology.

FIG. 19 is cross-sectional view of a depot in accordance with some embodiments of the present technology.

FIG. 20A is a perspective view of a depot in accordance with some embodiments of the present technology.

FIG. 20B is cross-sectional view of the depot shown in FIG. 20A taken along line B-B.

FIG. 20C is cross-sectional view of the depot shown in FIG. 20A taken along line C-C.

FIG. 20D is a perspective view of a depot in accordance with some embodiments of the present technology.

FIG. 21 is a perspective view of a depot in accordance with some embodiments of the present technology.

FIG. 22 is a perspective view of a depot in accordance with some embodiments of the present technology.

FIG. 23 is a perspective view of a depot in accordance with some embodiments of the present technology.

FIG. 24 is a perspective view of a depot in accordance with some embodiments of the present technology.

FIG. 25A is a side cross-sectional view of a depot in accordance with some embodiments of the present technology.

FIG. 25B is a cross-sectional view of the depot shown in FIG. 25A taken along line B-B.

FIG. 26 is a side cross-sectional view of a depot in accordance with some embodiments of the present technology.

FIG. 27 is a side cross-sectional view of a depot in accordance with some embodiments of the present technology.

FIG. 28 is a perspective view of a depot in accordance with some embodiments of the present technology.

FIG. 29 is a side cross-sectional view of a depot in accordance with some embodiments of the present technology.

FIG. 30 is a side cross-sectional view of a depot in accordance with some embodiments of the present technology.

FIG. 31 is a side cross-sectional view of a depot in accordance with some embodiments of the present technology.

FIG. 32A is a side cross-sectional view of a depot in accordance with some embodiments of the present technology.

FIG. 32B is a cross-sectional view of the depot shown in FIG. 32A taken along line B-B.

FIG. 32C is a side cross-sectional view of a depot in accordance with some embodiments of the present technology.

FIG. 32D is a side cross-sectional view of a depot in accordance with some embodiments of the present technology.

FIG. 33A is a side cross-sectional view of a depot in accordance with some embodiments of the present technology.

FIG. 33B depicts example release profiles over time of the depot shown in FIG. 33A.

FIG. 34A is a side cross-sectional view of a depot in accordance with some embodiments of the present technology.

FIG. 34B depicts example release profiles over time of the depot shown in FIG. 34A.

FIG. 35A is a side cross-sectional view of a depot in accordance with some embodiments of the present technology.

FIG. 35B depicts example release profiles over time of the depot shown in FIG. 35A.

FIG. 36A is a perspective view of a depot in accordance with some embodiments of the present technology.

FIG. 36B is a perspective view of a depot in accordance with some embodiments of the present technology.

FIG. 37A is a side view of a depot in a straightened state in accordance with some embodiments of the present technology.

FIG. 37B is a side view of the depot shown in FIG. 37A in a curved state.

FIG. 38A is a side view of a depot in a straightened state in accordance with some embodiments of the present technology.

FIG. 38B is a side view of the depot shown in FIG. 38A in a curved state.

FIG. 39A is a perspective view of a depot in a straightened state in accordance with some embodiments of the present technology.

FIG. 39B is cross-sectional view of the depot shown in FIG. 39A taken along line B-B.

FIG. 39C is a side view of the depot shown in FIG. 39A in a curved state.

FIG. 40 is a side view of a depot deployed at a target site in a body in accordance with some embodiments of the present technology.

FIG. 41 is a side view of a depot deployed at a target site in a body in accordance with some embodiments of the present technology.

FIG. 42 is a side view of a depot in accordance with some embodiments of the present technology.

FIG. 43 is a side view of a depot in accordance with some embodiments of the present technology.

FIGS. 44A and 44B are perspective views of depots in accordance with some embodiments of the present technology.

FIG. 45A-C are perspective, top, and side views, respectively, of a depot in accordance with some embodiments of the present technology.

FIG. 46A is an end view of a depot in a curled state in accordance with some embodiments of the present technology.

FIG. 46B is a side view of the depot shown in FIG. 46A in an uncurled state.

FIG. 47 illustrates a plurality of depots in accordance with some embodiments of the present technology.

FIG. 48A is an end view of a plurality of depots in accordance with some embodiments of the present technology.

FIG. 48B is a side view of the depots shown in FIG. 48A.

FIG. 48C illustrates a method of manufacturing the depots shown in FIGS. 48A and 48B.

FIG. 49 depicts the maximum flexural load of an implant over time from testing performed on implant samples submerged in buffered solution.

FIG. 50 is a schematic representation of core acidification of the prior art.

FIG. 51 is a scanning electron microscope image of a polymer tablet of the prior art after 20 days of degradation.

FIG. 52A is a schematic representation of the degradation of the depots of the present technology.

FIGS. 52B and 52C are scanning electron microscope (“SEM”) images of cross-sections of depots of the present technology at different timepoints during degradation.

FIGS. 53A-C are partially schematic perspective views of a delivery system for subcutaneously delivering a depot to a target site in accordance with some embodiments of the present technology.

FIG. 54 illustrates a depot coupled to an implantable medical device in accordance with embodiments of the present technology.

FIG. 55 is an anatomical cross-sectional illustration of an eye including multiple depots in accordance with embodiments of the present technology.

FIG. 56 illustrates anterior and lateral anatomical views of a nasal cavity and paranasal sinuses.

FIG. 57 is an anatomical cross-sectional illustration of a nasal cavity including multiple depots in accordance with embodiments of the present technology.

FIG. 58 illustrates a breast implant capsular contracture associated with breast implant surgery.

FIG. 59 illustrates a depot coupled to a breast implant in accordance with embodiments of the present technology.

### DETAILED DESCRIPTION

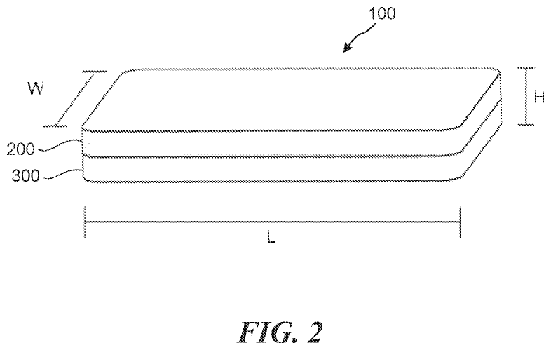

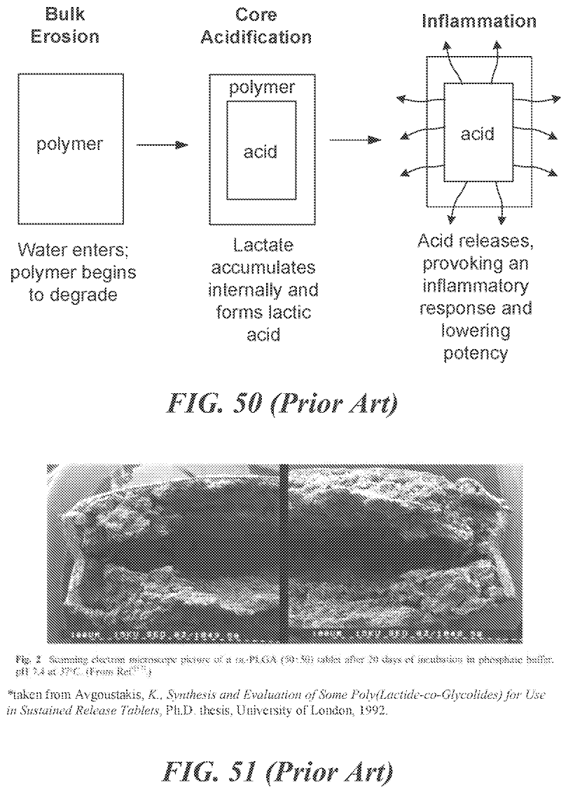

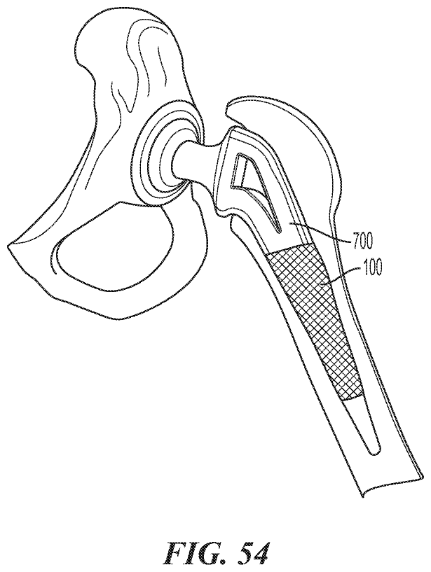

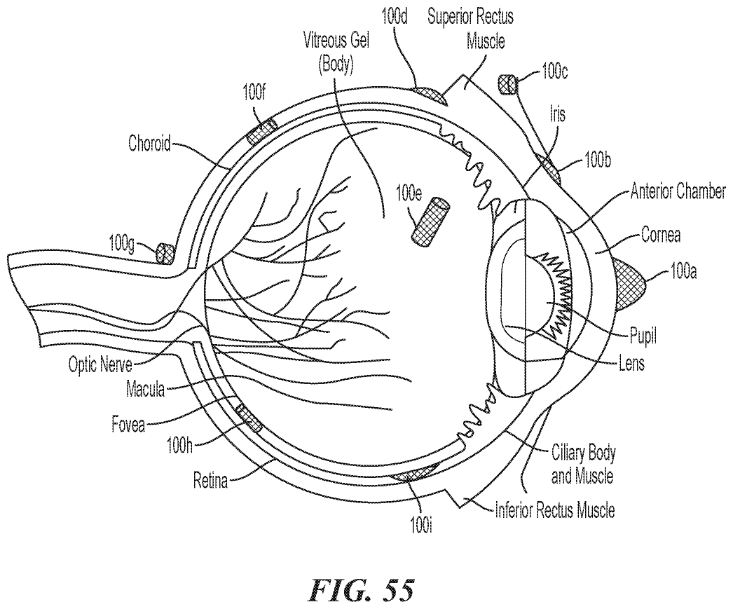

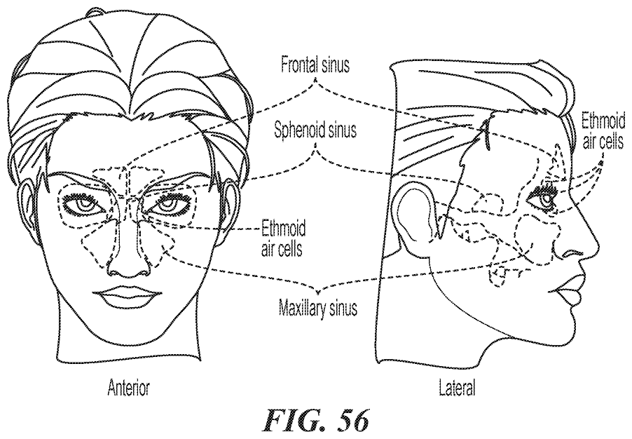

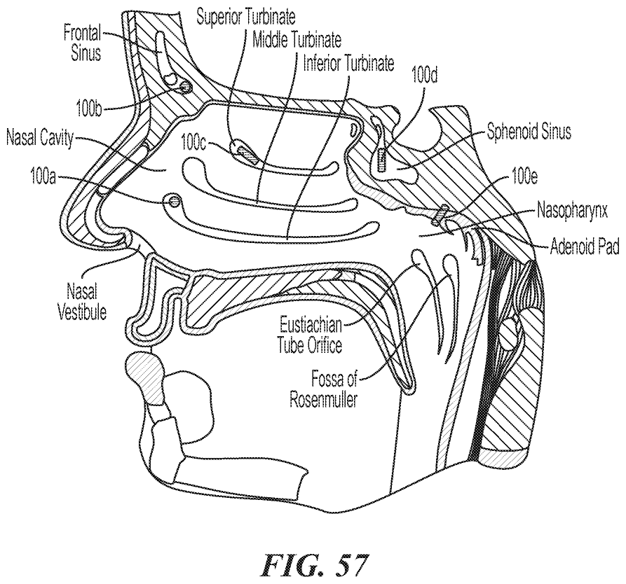

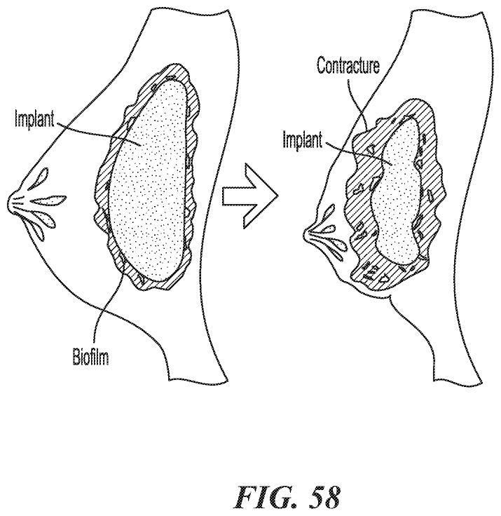

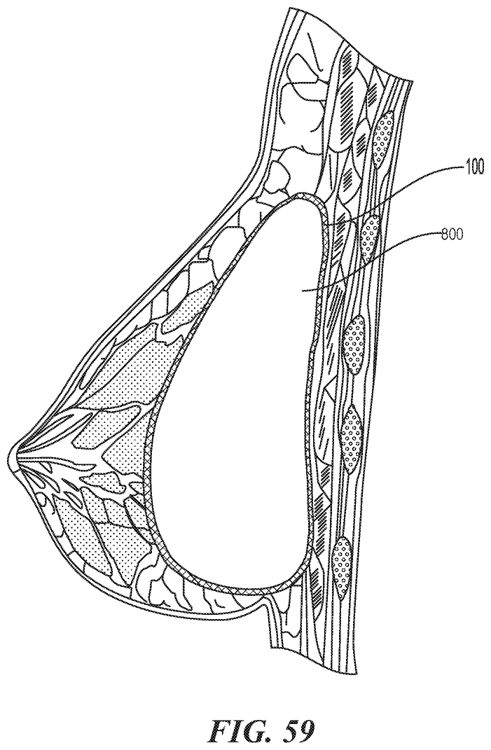

The present technology relates to implantable depots for the sustained, controlled release of therapeutic agents, and associated devices, systems, and methods of use. Examples of the depots of the present technology and associated release kinetics are described below with reference to FIGS. 2-52C and Section I. Use of the depots of the present technology for treating symptoms associated with T2D is described below with reference to Section II. Use of the depots of the present technology for treating symptoms associated with a mental illness is described below with reference to Section III. Use of the depots of the present technology for treating cardiovascular disease is described below with reference to Section IV. Use of the depots of the present technology for treating or preventing symptoms associated with HIV or malaria is described below with reference to Section V. Use of the depots of the present technology for treating or preventing infection associated with implantable medical devices (IMD) is described below with reference to FIG. 54 and Section VI. Use of the depots of the present technology for treating ocular conditions is described below with reference to FIG. 55 and Section VII. Use of the depots of the present technology for treating otolaryngologic conditions are described below with reference to FIGS. 56-57 and Section VIII. Finally, use of the depots of the present technology for treating or prevent capsular contracture or other conditions associated with breast implants is described below with reference to FIGS. 58 and 59 and Section IX.

### I. Example Depots of the Present Technology

Disclosed herein are implantable depots and associated devices, systems, and methods for treating certain conditions via sustained, controlled release of one or more therapeutic agents while the depot is implanted at a treatment site in vivo. As is understood in the art, “release” of the therapeutic agent includes movement of the therapeutic agent away from the depot, as well as the sustained presence of the therapeutic agent at the treatment site following implantation of the depot, regardless of the relative movement of the therapeutic agent with respect to the confines of the depot. Thus, any therapeutic agent that remains substantially stationary relative to its position when first implanted is still considered “released” so long as it provides a therapeutic benefit at the treatment site.

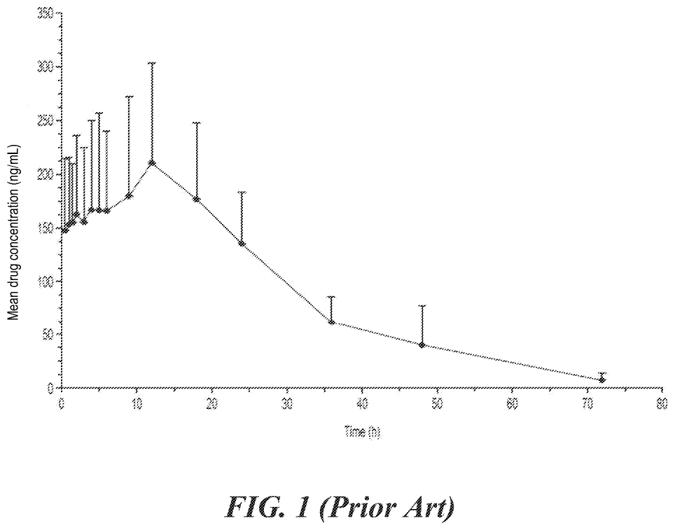

As noted previously, prior art drug delivery systems often suffer from a lack of a true controlled release mechanism in that they typically provide a burst of drug upon contact with surrounding physiologic fluids followed by a residual release of drug. For example, FIG. 1 shows an example prior art biodegradable polymer-based delivery system, in which the drug concentration in plasma peaked within 15 hours of implantation, thereby illustrating a duration of effect that is inadequate.

Disclosed herein are implantable depots and associated devices, systems, and methods, e.g., for treating certain conditions via sustained, controlled release of a therapeutic agent while the depot is implanted at a treatment site in vivo. While implanted in vivo, the depot(s) are configured to release a therapeutic agent to the treatment site in a controlled, prescribed manner for extended periods following implantation.

As used herein, a “depot” comprises a composition configured to administer at least one therapeutic agent to a treatment site in the body of a patient in a controlled, sustained manner. The depot also comprises the therapeutic agent itself. A depot may comprise a physical structure or carrier to configured to perform or enhance one or more functions related to treatment, such as facilitating implantation and/or retention in a treatment site (e.g., tissue at the intracapsular and/or extracapsular space of a knee joint, subcutaneously at a patient's abdomen, within the bladder, etc.), modulating the release profile of the therapeutic agent (e.g., creating a two-phase release profile), increasing release towards a treatment site, reducing release away from a treatment site, or combinations thereof. In some embodiments, a “depot” includes but is not limited to films, sheets, strips, ribbons, capsules, coatings, matrices, wafers, pills, pellets, or other pharmaceutical delivery apparatus or a combination thereof. Moreover, as used herein, “depot” may refer to a single depot, or may refer to multiple depots. As an example, the statement “The depot may be configured to release 2 g of therapeutic agent to a treatment site” describes (a) a single depot that is configured to release 2 g of therapeutic agent to a treatment site, and (b) a plurality of depots that collectively are configured to release 2 g of therapeutic agent to a treatment site.

FIG. 2 is an isometric view of an implantable depot 100 in accordance with several embodiments of the present technology. The depot 100 may be a thin, multi-layered polymer film configured to be implanted at a treatment site comprising a therapeutic region 200 containing a therapeutic agent, and a control region 300 configured to regulate the release of the therapeutic agent from the depot 100 in a controlled and sustained manner. The depot 100 may include a high therapeutic payload of the therapeutic agent, especially as compared to other known films of equal thickness or polymer weight percentage, while exhibiting mechanical properties (e.g., flexural strength) sufficient to withstand storage, handling, implantation, and/or retention in the treatment site. For example, in some embodiments, the depot 100 comprises at least 50% by weight of the therapeutic agent.

The control region 300 may comprise at least one bioresorbable polymer and at least one releasing agent mixed with the polymer, and the therapeutic region 200 may comprise at least one bioresorbable polymer and at least one releasing agent mixed with the polymer and the therapeutic agent. The control region 300 may optionally include a therapeutic agent, or the control region 300 may include no therapeutic agent at all. The therapeutic region 200 may optionally include no releasing agent at all. The releasing agent in the control region 300 may be the same or may be different from the releasing agent in the therapeutic region 200. The bioresorbable polymer in the control region 300 may be the same or may be different from the bioresorbable polymer in the therapeutic region 200. As detailed below, in some embodiments the therapeutic region 200 and/or the control region 300 may have different constituents and/or formulations.

When exposed to a fluid (e.g., physiologic fluid), the releasing agent can have a dissolution rate that is faster than the degradation rate of the bioresorbable polymer. Accordingly, when a fluid contacts the depot 100 (e.g., after implantation of the depot 100 in a treatment site), the releasing agent dissolves within the surrounding polymer of the control region 300 and/or therapeutic region 200 faster than the polymer degrades. As the releasing agent dissolves, the space vacated by the dissolved releasing agent forms diffusion openings (e.g., channels, voids, pores, etc.) in the surrounding polymer region. The formation of diffusion openings may enhance the release of therapeutic agent from the polymer region and into the surrounding physiologic fluid. In some embodiments, the release rate of the therapeutic agent is higher when there are diffusion openings in the polymer region, compared to when there are no diffusion openings in the polymer region.

The concentration and type of releasing agent, among other parameters, can be selected to regulate the release of the therapeutic agent from the therapeutic region 200 and/or through the control region 300 into the surrounding fluid at a controlled dosage rate over a desired period of time. For example, a higher concentration of releasing agent may increase the release rate of the therapeutic agent, while a lower concentration of releasing agent may decrease the release rate of the therapeutic agent. The therapeutic region 200 may comprise a different concentration and/or type of releasing agent than the control region 300, or may comprise the same concentration and/or type of releasing agent.

The position and/or geometry of the control region 300 can be configured to modulate the release profile of the therapeutic agent from the therapeutic region 200. As shown in FIG. 2, at least a portion of the control region 300 may be disposed on or adjacent the therapeutic region 200 such that, when the depot 100 is initially positioned in vivo, the control region 300 is between at least a portion of the therapeutic region 200 and physiologic fluids at the treatment site. For example, the control region 300 can cover all or a portion of one or more surfaces of the therapeutic region 200. When the depot 100 is exposed to physiologic fluids, the therapeutic agent elutes from the exposed surfaces of the therapeutic region 200 and through the control region 300 by way of the diffusion openings created by dissolution of the releasing agent. In general, the therapeutic agent elutes from the exposed surfaces of the therapeutic region 200 at a faster (e.g., greater) rate than through the control region 300. As a result, the control region 300 prolongs the release of the therapeutic agent from the therapeutic region 200 to provide for longer release times and regulates the dosage rate, e.g., to provide the desired effects and avoid complications related to overdosing.

The depot of the present technology is configured to release a therapeutic agent in a highly controlled, predetermined manner that is specifically tailored to the medical condition being treated and the therapeutic agent used. As described in greater detail below in Section II, the release kinetics of the depots may be customized for a particular application by varying one or more aspects of the depot's composition and/or structure, such as the shape and/or size of the depot, therapeutic region 200, and/or control region 300; the exposed surface area of the therapeutic region 200; the type of polymer (in the therapeutic region 200 and/or in the control region 300); the weight percentage of the therapeutic agent, the polymer, and/or the releasing agent (within a particular region or generally throughout the depot 100); and the composition of the therapeutic region 200 and the control region 300.

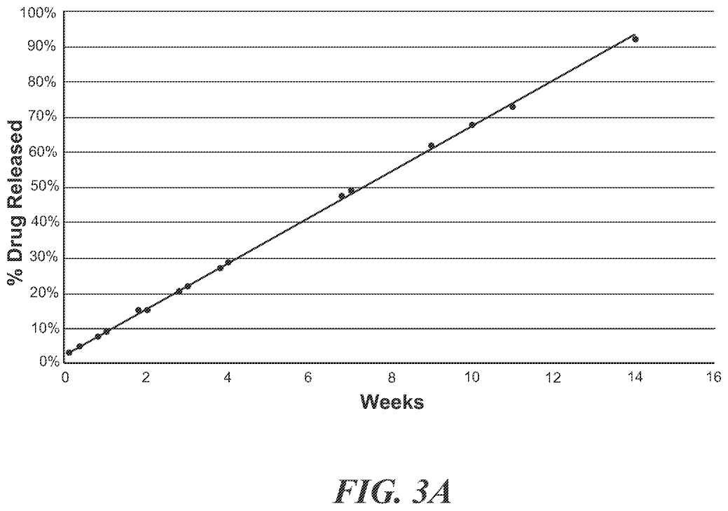

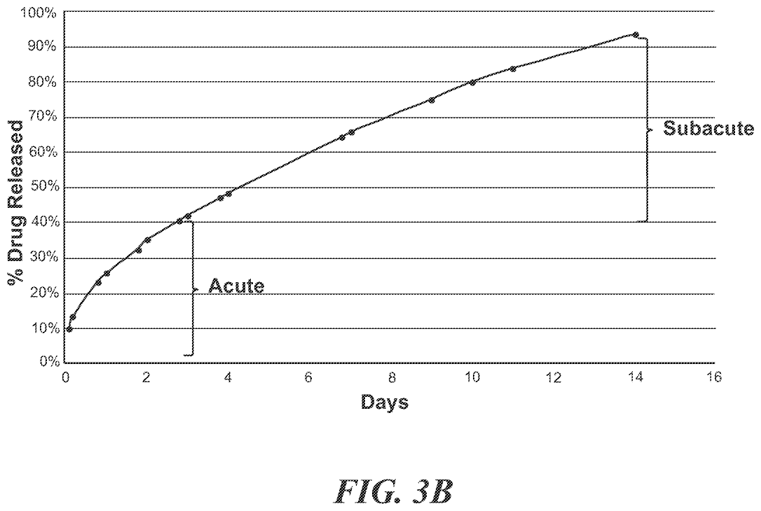

As shown in FIG. 3A, in some embodiments the depot 100 (or a system of depots 100) is configured to release a therapeutic agent at a substantially constant rate. As shown in FIG. 3B, in some embodiments the depot 100 (or a system of depots 100) is configured to release a disproportionately larger volume of a therapeutic agent per day for a first period of time than for a longer second period of time. FIGS. 3A and 3B illustrate two example release profiles, however the depot 100 (or a system of depots 100) can be configured to achieve a wide range of different release profiles depending on the particular clinical needs and underlying condition being addressed. For example, while certain therapeutic agents may be beneficially administered according to a zero-order release profile as shown in FIG. 3A, in other instances in the therapeutic agent may be beneficially administered using a second-order release profile as shown in FIG. 3B. In particular embodiments, the depot 100 may be configured to release the therapeutic agent at a constant rate for a first period of time and at a non-constant rate for a second period of time (which may occur before or after the first period of time). In addition to varying the release profile (e.g., zero order, first order, second order, etc.), the overall release time can be precisely controlled by adjusting the structure, composition, and/or the process by which the depot is manufactured, as described in more detail below. For example, as shown in FIG. 3A, more than 90% of the therapeutic agent may be released after 14 weeks, while in FIG. 3B, more than 90% of the therapeutic agent is released after 14 days.

In some embodiments, the depot 100 is configured to release no more than 20%, no more than 25%, no more than 30%, no more than 35%, no more than 40%, no more than 45%, no more than 50%, no more than 55%, no more than 60%, no more than 65%, or no more than 70% of the therapeutic agent in the first day, 2 days, 3 days, 4 days, 5 days, 6 days, 8 days, 9 days, 10 days, 11 days, 12 days, or 13 days, or in the first week, 2 weeks, 3 weeks, 4 weeks, 5 weeks, 6 weeks, 7 weeks, 8 weeks, 9 weeks, 10 weeks, 11 weeks, 12 weeks, 13 weeks, or 14 weeks, 15 weeks, 16 weeks, 17 weeks, 18 weeks, 19 weeks, 20 weeks, 21 weeks, 22 weeks, 23 weeks, or 24 weeks of the duration of release, and wherein at least 75%, at least 80%, at least 85%, at least 90%, at least 95%, or 100% of the remaining therapeutic agent is released in the remaining days of the duration of release. The intended duration of release may be at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, or at least 24 weeks.

In some embodiments, the depot 100 is configured to release at least 70%, at least 75%, at least 80%, at least 85%, at least 90%, at least 95%, at least 96%, at least 97%, at least 98%, at least 99%, or 100% of the therapeutic agent in the depot 100 within the intended duration of treatment. The intended duration of treatment may be at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 40 days, at least 50 days, at least 60 days, at least 70 days, at least 90 days, at least 100 days, at least 200 days, at least 300 days, or at least 365 days.

In some embodiments, the depot 100 can be configured to release select amounts of therapeutic agent over defined time periods (e.g., each day, each week, etc.). The particular amounts of therapeutic agent delivered can be selected depending on the particular therapeutic agent. In some embodiments, the depot 100 is configured to release a sufficient amount of therapeutic agent to provide therapeutic efficacy while also not releasing an excessive amount of therapeutic agent that would result in toxicity or other deleterious effects. For example, in the case of bupivacaine hydrochloride as the therapeutic agent, the depot 100 can be configured to release from about 50 mg/day to about 600 mg/day, 100 mg/day to about 500 mg/day, or from about 100 mg/day to about 400 mg/day, or from about 100 mg/day to about 300 mg/day of bupivacaine hydrochloride to the treatment site. The particular dosage ranges, upper limits, and lower limits, can be selected depending on the particular therapeutic agent(s) to achieve the desired effects. In general, the release rate can be selected to deliver the desired dosage to provide the extent of desired therapeutic effect, control toxicity, and deliver the therapeutic agent for a sufficient period of time

In some embodiments, the depot 100 is configured to release the therapeutic agent at a treatment site in vivo and/or in the presence of one or more fluids for no less than 1 day, no less than 2 days, no less than 3 days, no less than 4 days, no less than 5 days, no less than 6 days, no less than 7 days, no less than 8 days, no less than 9 days, no less than 10 days, no less than 11 days, no less than 12 days, no less than 13 days, no less than 14 days, no less than 15 days, no less than 16 days, no less than 17 days, no less than 18 days, no less than 19 days, no less than 20 days, no less than 21 days, no less than 22 days, no less than 23 days, no less than 24 days, no less than 25 days, no less than 26 days, no less than 27 days, no less than 28 days, no less than 29 days, no less than 30 days, no less than 40 days, no less than 50 days, no less than 60 days, no less than 70 days, no less than 90 days, no less than 100 days, no less than 200 days, no less than 300 days, or no less than 365 days.

The release kinetics of the depots of the present technology may be tuned for a particular application by varying one or more aspects of the depot's structure, such as the exposed surface area of the therapeutic region 200, the porosity of the control region 300 during and after the releasing agent dissolves, the concentration of the therapeutic agent in the therapeutic region, the post-manufacturing properties of the polymer, the structural integrity of the depots to avoid a sudden release of the therapeutic agent, the relative thicknesses of the therapeutic region 200 compared to the control region 300, and other properties of the depots. Several embodiments of depots of the present technology combine one or more of these properties in a manner that produces exceptional sustained, controlled release profiles in animal studies that significantly outperform existing injectable or implantable systems, while also overcoming the shortcomings of disclosed prophetic devices. This enables depots of the present technology to at least reduce, if not replace, other existing treatment systems.

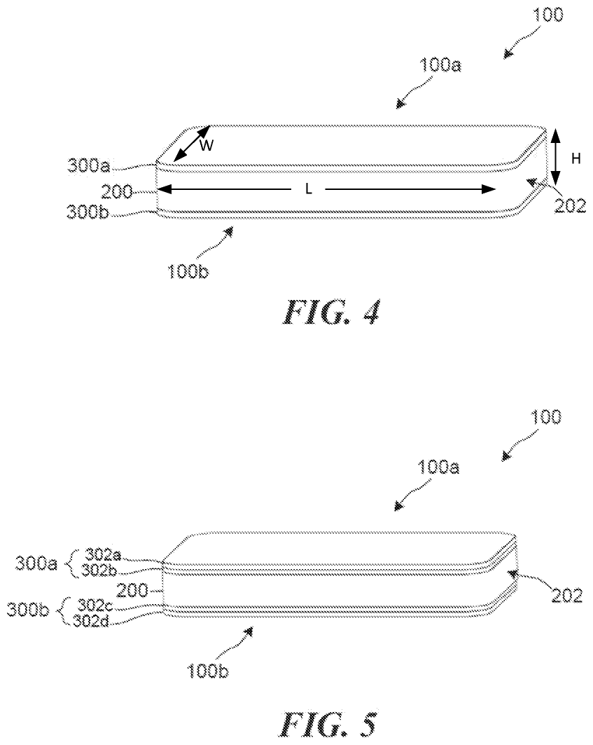

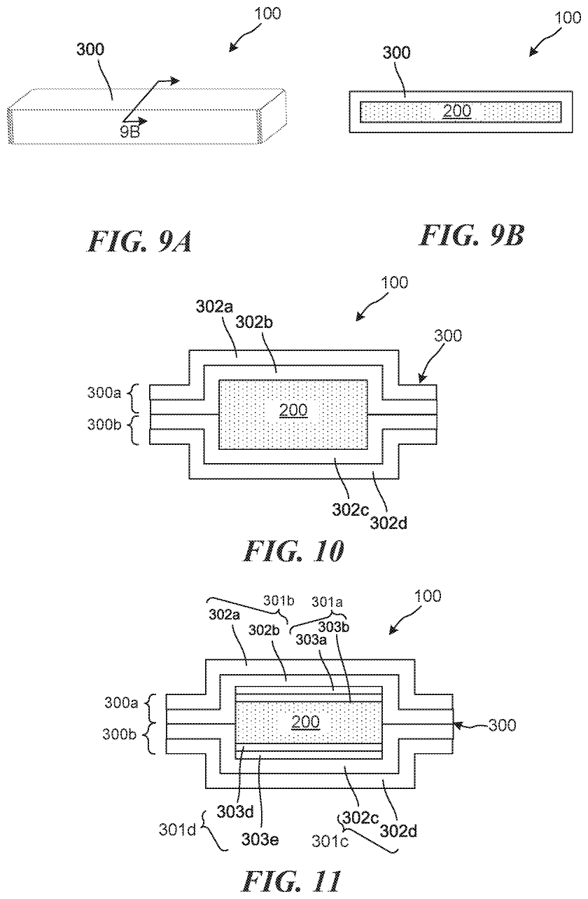

For example, the release profile can be tuned by, at least in part, controlling the amount of exposed surface area of the therapeutic region 200 because depots having a therapeutic region 200 covered only partially by a control region 300 (see, for example, FIGS. 2, 4-8, 13, 15-27, and 32-41) will generally release a higher proportion of the total payload over a shorter period of time as compared to embodiments where the therapeutic region 200 is completely encapsulated by the control region 300 (see, for example, FIGS. 9A-12). More specifically, depot designs having a therapeutic region 200 with exposed surfaces can be configured to release the therapeutic agent at a high, substantially linear rate for a first period of time and then at a lower, substantially linear rate for a second period of time. Alternatively, depot designs having a therapeutic region 200 with surfaces that are substantially covered by one or more control regions 300 may achieve a zero-order release such that the release of the payload of therapeutic agent is at substantially the same rate. Various examples of different depot configurations are shown and described with respect to FIGS. 4-48B. Features of any one of these example depot configurations may be combined with any other depot configuration disclosed herein.

As shown in FIG. 4, in some embodiments the depot 100 may comprise a multi-layer polymer film having a therapeutic region 200 and first and second control regions 300a, 300b positioned at opposite surfaces 100a, 100b of the therapeutic region 200. The depot 100 may be in the form of a flexible, rectangular strip having a length L, a width W, and a height H (or thickness). In some embodiments, the depot 100 has (a) a length L of from about 5-40 mm, about 10-30 mm, about 15-20 mm, about 20-35 mm, about 20-30 mm, about 20-25 mm, about 26-30 mm, about 5 mm, about 10 mm, about 11 mm, about 12 mm, about 13 mm, about 14 mm, about 15 mm, about 16 mm, about 17 mm, about 18 mm, about 19 mm about 20 mm, about 21 mm, about 22 mm, about 23 mm, about 24 mm, about 25 mm, about 26 mm, about 27 mm, about 28 mm, about 29 mm, about 30 mm, about 10-15 mm, about 12-16 mm, about 15-20 mm, about 21-23 mm, about 22-24 mm, about 23-25 mm, about 24-26 mm, about 25-27 mm, about 26-28 mm, about 27-29 mm, or about 28-30 mm, (b) a width W of from about 5-40 mm, about 10-30 mm, about 15-20 mm, about 20-35 mm, about 20-30 mm, about 20-25 mm, about 26-30 mm, about 5 mm, about 10 mm, about 11 mm, about 12 mm, about 13 mm, about 14 mm, about 15 mm, about 16 mm, about 17 mm, about 18 mm, about 19 mm about 20 mm, about 21 mm, about 22 mm, about 23 mm, about 24 mm, about 25 mm, about 26 mm, about 27 mm, about 28 mm, about 29 mm, about 30 mm, about 10-15 mm, about 12-16 mm, about 15-20 mm, about 21-23 mm, about 22-24 mm, about 23-25 mm, about 24-26 mm, about 25-27 mm, about 26-28 mm, about 27-29 mm, or about 28-30 mm (c) a height H of from about 0.4 mm to about 4 mm, about 1 mm to about 3 mm, about 1 mm to about 2 mm, at least 0.4 mm, at least 0.5 mm, at least 0.6 mm, at least 0.7 mm, at least 0.8 mm, at least 0.9 mm, at least 1 mm, at least 1.2 mm, at least 1.4 mm, at least 1.5 mm, at least 1.6 mm, at least 1.7 mm, at least 1.8 mm, at least 2 mm, at least about 3 mm, no more than 0.5 mm, no more than 0.6 mm, no more than 0.7 mm, no more than 0.8 mm, no more than 0.9 mm, etc.). In some embodiments, the depot 100 may have other shapes and/or dimensions, such as those detailed below

Additionally, some embodiments of the depot shown in FIG. 4 are configured such that a thickness of the control regions 300a and 300b, either individually or collectively, is less than or equal to 1/10 of a thickness of the therapeutic region 200. The thickness of the control regions 300a and 300b, either individually or collectively, can further be no more than 1/12.5, 1/15, 1/17.5, 1/20, 1/22.5, 1/25, 1/30, 1/40, 1/50, 1/75, or 1/100 of the thickness of the therapeutic region 200. In those embodiments with multiple sub-control regions, one or more of the sub-control regions may individually be less than or equal to 1/10, 1/12.5, 1/15, 1/17.5, 1/20, 1/22.5, 1/25, 1/27.5, 1/30, 1/32.5, 1/35, 1/37.5, 1/40, 1/42.5, 1/45, 1/47.5, 1/50, 1/55, 1/60, 1/65, 1/70, 1/75, 1/80, 1/85, 1/90, 1/95, or 1/100 of a thickness of the therapeutic region. In those embodiments where the control region comprises a single control region, the control region may have a thickness that is less than or equal to 1/10, 1/12.5, 1/15, 1/17.5, 1/20, 1/22.5, 1/25, 1/27.5, 1/30, 1/32.5, 1/35, 1/37.5, 1/40, 1/42.5, 1/45, 1/47.5, 1/50, 1/55, 1/60, 1/65, 1/70, 1/75, 1/80, 1/85, 1/90, 1/95, or 1/100 of a thickness of the therapeutic region. In those embodiments with multiple sub-control regions, one or more of the sub-control regions may individually be less than or equal to 1/10, 1/12.5, 1/15, 1/17.5, 1/20, 1/22.5, 1/25, 1/27.5, 1/30, 1/32.5, 1/35, 1/37.5, 1/40, 1/42.5, 1/45, 1/47.5, 1/50, 1/55, 1/60, 1/65, 1/70, 1/75, 1/80, 1/85, 1/90, 1/95, or 1/100 of a thickness of the depot. In those embodiments where the control region comprises a single control region, the control region may have a thickness that is less than or equal to 1/10, 1/12.5, 1/15, 1/17.5, 1/20, 1/22.5, 1/25, 1/27.5, 1/30, 1/32.5, 1/35, 1/37.5, 1/40, 1/42.5, 1/45, 1/47.5, 1/50, 1/55, 1/60, 1/65, 1/70, 1/75, 1/80, 1/85, 1/90, 1/95, or 1/100 of a thickness of the depot.

The control regions 300a, 300b may only cover a portion of the therapeutic region 200 such that a portion of each of the lateral surfaces (e.g., sidewall) of the therapeutic region 200 is exposed to physiologic fluids immediately upon implantation of the depot 100 in vivo. When the depot 100 is exposed to physiologic fluids (or any similar fluid in an in vitro setting), the therapeutic agent will elute from the exposed surfaces 202 (in addition to through the control regions 300a, 300b), such that the therapeutic agent is released faster than if the therapeutic region 200 had no exposed regions. As such, the surface area of the exposed surfaces 202 may be tailored to provide an initial, controlled burst, followed by a tapering release (for example, similar to that shown in FIG. 3A). The initial, more aggressive release of the therapeutic agent is slowed in part by the control regions 300a, 300b that initially reduce the surface area of the therapeutic region 200 exposed to the fluids. Unlike the depots 100 of the present technology, many conventional drug-eluting technologies provide an initial, uncontrolled burst release of drug when exposed to physiologic fluids. Several embodiments of depots of the present technology not only enable enough therapeutic agent to be implanted for several days', weeks', or months' worth of dosage to achieve a sustained, durable, in vivo pharmacological treatment, but they also release the therapeutic agent as prescribed and thereby prevent a substantial portion of the entire payload being released in an uncontrolled manner that could potentially result in complications to the patient and/or reduce the remaining payload such that there is not enough therapeutic agent remaining in the depot to deliver a therapeutic amount for the remaining duration of release.

In some embodiments, the depot 100 shown in FIG. 4 is configured such that about 20% to about 50% of the therapeutic agent is released in the first about 3 days to about 5 days of the 14 days, and wherein at least 80% of the remaining therapeutic agent is released in the last about 9 days to about 11 days of the 14 days. In some embodiments, the depot 100 shown in FIG. 4 is configured to release about 100 mg to about 500 mg of therapeutic agent to the treatment site per day, and in some cases no more than 400 mg or no more than 300 mg of therapeutic agent per day within the first 3 days of implantation and no more than 200 mg per day in the remaining days.

Several embodiments of the depot 100 shown in FIG. 4 are also configured to maintain their structural integrity even after a substantial portion of the releasing agent has eluted from the depot 100. As the releasing agent(s) dissolves and therapeutic agent(s) elutes, the functional mechanical aspects of the depot 100 may change over time. Such mechanical aspects include structural integrity, flexural strength, tensile strength, or other mechanical characteristics of the depot. If a depot 100 experiences too much degradation too fast, it may fail mechanically and release an undesirable burst of therapeutic agent into the body. Several embodiments of depots 100 shown in FIG. 4 are loaded with enough therapeutic agent to deliver 100 mg to 500 mg of the therapeutic agent per day while still being able to maintain its structural integrity such that depot remains largely intact up to at least 14 days after implantation. A depot can be sufficiently intact, for example, if it does not fracture into multiple component pieces with two or more of the resulting pieces being at least 5% of the previous size of the depot. Alternatively, or additionally, a depot can be considered to be sufficiently intact if the release rate of the therapeutic agent does not increase by more than a factor of three as compared to the release rate of therapeutic agent in a control depot submerged in a buffered solution.

The therapeutic agent can be at least 50%-95% by weight of the total weight of the depot 100 before implantation, or 55%-85% by weight of the total weight of the depot 100 before implantation, or 60%-75% by weight of the total weight of the depot 100 before implantation. Likewise, the polymer may be no more than 5%-50% by weight of the total weight of the depot 100 before implantation, or 10%-50% by weight of the total weight of the depot 100 before implantation, or 15%-45% by weight of the total weight of the depot 100 before implantation, or 20%-40% by weight of the total weight of the depot 100 before implantation, or no more than 25%, no more than 30%, no more than 35%, or no more than 40%. The ratio of the mass of the therapeutic agent in the depot 100 to the mass of the polymer in the depot 100 can be at least 16:1, 15:1, 14:1, 13:1, 12:1, 11:1, 10:1, 9:1, 8:1, 7:1, 6:1, 5:1, 4:1, 3:1, or 2:1.

In some embodiments, one or more control regions 300 of the depot 100 may comprise two or more sub-control regions. For example, as shown in FIG. 5, the depot 100 may have a first control region 300a and a second control region 300b, each of which comprises first and second sub-control regions 302a, 302b and 302c, 302d, respectively. The first and second control regions 300a, 300b and/or one, some or all of the sub-control regions 302a-302d may have the same or different amounts of releasing agent, the same or different concentrations of releasing agent, the same or different releasing agents, the same or different amounts of polymer, the same or different polymers, the same or different polymer to releasing agent ratios, and/or the same or different thicknesses. In some embodiments, the concentration of the releasing agent in the individual outer control sub-regions 302a, 302d is less than the concentration of the releasing agent in the individual inner control sub-regions 302b, 302c such that the outer portion of the collective control region will elute the therapeutic agent more slowly than the inner portion of the collective control region. In some embodiments, the concentration of the releasing agent in the individual outer control sub-regions 302a, 302d is greater than the concentration of the releasing agent in the individual inner control sub-regions 302b, 302c. In those embodiments where the control region includes more than two sub-regions, the concentration of releasing agent per sub-region or layer may increase, decrease, or remain constant as the sub-control regions are farther away from the therapeutic region 200.

In certain embodiments, the outer control sub-regions include at least 5% by weight of the releasing agent, at least 10% by weight of the releasing agent, at least 15% by weight of the releasing agent, at least 20% by weight of the releasing agent, at least 25% by weight of the releasing agent, at least 30% by weight of the releasing agent, at least 35% by weight of the releasing agent, at least 40% by weight of the releasing agent, at least 45% by weight of the releasing agent, or at least 50% by weight of the releasing agent. In some embodiments, the inner control sub-regions include at least 5% by weight of the releasing agent, at least 10% by weight of the releasing agent, at least 15% by weight of the releasing agent, at least 20% by weight of the releasing agent, at least 25% by weight of the releasing agent, at least 30% by weight of the releasing agent, at least 35% by weight of the releasing agent, at least 40% by weight of the releasing agent, at least 45% by weight of the releasing agent, or at least 50% by weight of the releasing agent. In some embodiments, the outer control sub-regions may include a first amount of the releasing agent and the inner control sub-regions may include a second amount of the releasing agent, where the second amount is at least 200%, at least 300%, at least 400%, or at least 500% greater than the first amount.

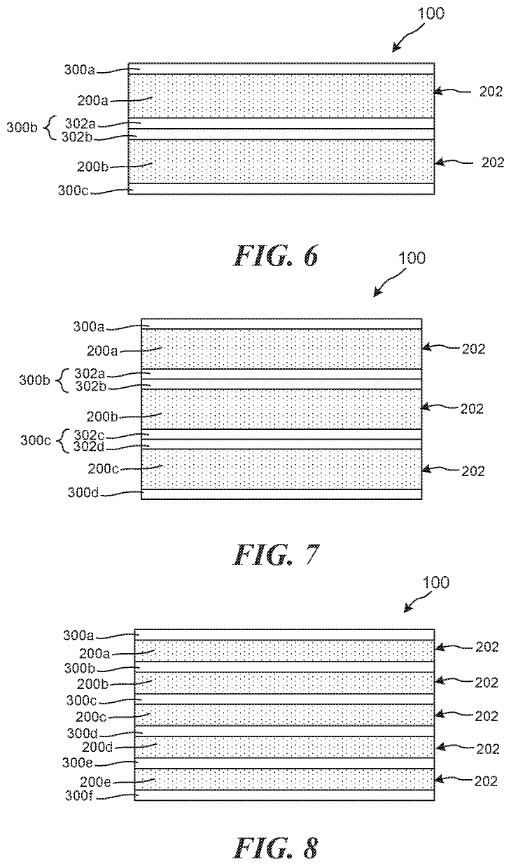

FIGS. 6-8 show depot embodiments having a plurality of alternating therapeutic regions 200 and control regions 300 in accordance with the present technology. The depot 100 may have two or more control regions 300 and/or sub-regions 302 (e.g., 2, 3, 4, 5, 6, 7, 8, 9, 10, 20, etc.), and the depot 100 may have one or more therapeutic regions 200 and/or sub-regions 202 (e.g., 1, 2, 3, 4, 5, 6, 7, 10, 15, 20, etc.) surrounded by at least one control region 300 and/or sub-region 302. In some embodiments, each of the therapeutic regions 200 may comprise a single layer and/or each of the control regions 300 may comprise a single layer. In some embodiments, one, some, or all of the therapeutic regions 200 may comprise multiple layers and/or one, some, or all of the control regions 300 may comprise multiple layers. In some embodiments, for example as shown in FIGS. 6 and 7, two or more sub-regions 302a-b (FIG. 6) and 302a-b and 302c-d (FIG. 7) may be adjacent to each other between sub-regions 202 of the therapeutic region 200. Moreover, one or more of the individual control regions 300 and/or one or more of the therapeutic regions 200 may have the same or different amounts and/or types of releasing agent, and one or more of the therapeutic regions may have the same or different amounts and/or types of therapeutic agent.

The embodiments shown in FIGS. 6-8 may be beneficial where the therapeutic region comprises a large payload of the therapeutic agent (e.g., equivalent to many days, weeks or months of dosage). These embodiments may be beneficial because, with such a large payload, should the therapeutic region 200 be exposed to the body abruptly, the entire payload may be released prematurely, subjecting the patient to an abnormally and undesirably high dose of the therapeutic agent. For example, if the integrity of the control region 300 were compromised, the patient may be exposed in vivo to the therapeutic agent at a higher rate than intended, potentially resulting in a clinical complication. In the event that a control region 300 is compromised, it is desirable for the patient to be subjected only to a fraction of the total payload, whereby the fraction to which the patient is exposed if prematurely released would be within safety margins for the particular therapeutic agent. The structural integrity of the control regions 300, as well as that of the therapeutic region(s) 200, is an important property for depots with large masses of therapeutic agents that are to be delivered over a long period of time.

To address this concern, in some embodiments of the present technology, the depot 100 may comprise multiple therapeutic regions 200 separated by one or more control regions 300 (for example, as shown in FIGS. 6-8). Such a configuration allows the therapeutic agent in each therapeutic region 200 (which carries a fraction of the total payload), to be individually sequestered. In the event a particular control region is compromised, only the fractional payload corresponding to the therapeutic region associated with the compromised control region would prematurely release. For example, in some of the foregoing embodiments, the total payload of the depot 100 may be at least 100 mg, at least 150 mg, at least 200 mg, at least 300 mg, at least 400 mg, at least 500 mg, at least 600 mg, at least 700 mg, at least 800 mg, at least 900 mg, or at least 1000 mg of therapeutic agent. Likewise, in some embodiments the fractional payload of each therapeutic region or sub-region may be up to 1%, up to 5%, up to 10%, up to 15%, up to 20%, up to 25%, up to 30%, up to 40%, up to 50%, up to 60%, up to 70%, up to 80%, up to 90%, or up to 100% of the total payload contained within the depot 100. As a result, if any single sub-region 202 of the therapeutic region 200 is compromised, it can release only a proportionate fraction of the total payload of the depot.

In some embodiments, each of the therapeutic regions and each of the control regions is a micro-thin layer, i.e., having a layer thickness that is less than 1 mm. In some embodiments, the depot comprises from about 2 to about 100 therapeutic regions, or from about 2 to about 50 therapeutic regions, or from about 2 to about 10 therapeutic regions.

FIGS. 9A-11 show some aspects of the present technology in which the depots 100 may have one or more therapeutic regions 200 completely enclosed or surrounded by one or more control regions 300. In contrast to the previously described embodiments, at least one therapeutic region of such fully-enclosed embodiments does not have any exposed surface area. For example, as shown in FIGS. 9A and 9B, in some embodiments the depot 100 may comprise a therapeutic region 200 surrounded or fully-enclosed by a control region 300 such that no portion of the therapeutic region 200 is exposed through the control region 300. As a result, the control region 300 substantially prevents contact between the therapeutic agent and physiologic fluids, thereby preventing an uncontrolled, burst release of the therapeutic agent when implanted. Over time, the releasing agent imbedded in the polymer of the control region 300 contacts physiologic fluids and dissolves, thereby forming diffusion openings in the control region. The combination of the restriction imposed by the control region and the diffusion openings formed by dissolution of the releasing agent enables a controlled release of the therapeutic agent from the depot over the course of several days, weeks, or months. Although the depot 100 is shown as a rectangular, thin film in FIGS. 9A and 9B, in other embodiments the depot 100 may have other shapes, sizes, or forms.

FIG. 10 illustrates a depot 100 having a therapeutic region 200 fully-enclosed by a control region 300 having a first control region 300a and a second control region 300b. As depicted in FIG. 10, in some embodiments the therapeutic region 200 may be sandwiched between the first control region 300a and the second control region 300b, and the first and second control regions 300a-b may be bonded via heat compression around the therapeutic region 200 to enclose the therapeutic region 200 therebetween. In certain embodiments, a bioresorbable polymer may be wrapped around the entire depot and sealed on the top or bottom surface creating a control region structure similar to that depicted in FIG. 9A. The outer portion of the first and second control regions 300a-b may be incorporated as the final wrapped layer to seal the edges. Additionally, the first and second control regions 300a-b can be integrally formed with each other using dip coating and/or spray coating techniques, such as dipping the therapeutic region 200 in a solution of the control region material or spraying a solution of control region material onto the surfaces of the therapeutic region 200.

In FIG. 10, the first control region 300a can have first and second sub-regions 302a-b, and the second control region 300b can have first and second sub-regions 302c-d. The first control region 300a can define a top control region member, and the first and second sub-regions 302a-b can comprise a first top control layer and a second top control layer, respectively. The second control region 300b can define a bottom control region member, and the first and second sub-regions 302c-d can comprise a first bottom control layer and a second bottom control layer, respectively. The first and second top/bottom control layers can be any variation of the first and second control sub-regions discussed above with reference to FIG. 5. In addition, the first top control layer of the top control region member may have the same or different properties (e.g., thickness, polymer, releasing agent, concentration of releasing agent, total amount of releasing agent, polymer to releasing agent ratio, etc.) as the first bottom control layer of the bottom control region member. Similarly, the second top control layer of the top control region member may have the same or different properties as the second bottom control layer of the bottom control region member. Variations in the loading and construction of the layers may be designed into the depot 100 to achieve a release profile or kinetics that suits the objectives of the intended therapy. In other embodiments, the first control region 300a and/or the second control region 300b has a single layer.

FIG. 11 shows some embodiments in which the depot 100 may have a therapeutic region 200 fully-enclosed by a control region 300 having different sub-region configurations. The depot 100 of FIG. 11 includes a first control region 300a and a second control region 300b that together fully enclose the therapeutic region 200. In contrast to the depot 100 shown in FIG. 10, the first control region 300a has an outer top control region 301a with first and second top sub-control regions 302a and 302b, respectively, and an inner top control region 301b with first and second top layers 303a and 303b. The first and second top layers 303a-b are over only the top surface of the therapeutic region 200, while the first and second top sub-control regions 302a-b cover a portion of the lateral surfaces of the therapeutic region 200 and the inner top control region 301b. The second control region 300b has an outer bottom control region 301c with first and second bottom sub-control regions 302c and 302d, respectively, and an inner bottom control region 301d with first and second bottom layers 303d and 303e, respectively. As such, when the depot 100 is positioned at the treatment site in vivo, the outer top and bottom control regions 301a and 301c are between: (a) the therapeutic region 200 and the inner top and bottom control regions 301b and 301d, respectively, and (b) physiologic fluids at the treatment site. In certain embodiments, such as that shown in FIG. 11, one or more of the outer top/bottom control regions 301a/301c may comprise one or more control sub-regions, and one or more inner top/bottom control regions 301b/301d may include one or more control sub-regions.

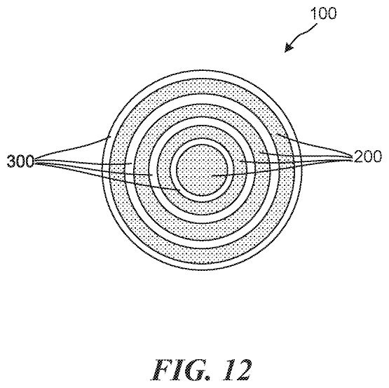

FIG. 12 shows a cross-section of a spherical depot 100 in accordance with several embodiments of the present technology having a plurality of alternating therapeutic regions 200 and control regions 300 in accordance with the present technology. The depot 100 may have two or more control regions 300 (e.g., 2, 3, 4, 5, 6, 7, 8, 9, 10, 20, etc.), and the depot may have one or more therapeutic regions 200 (e.g., 1, 2, 3, 4, 5, 6, 7, 10, 15, 20, etc.) surrounded by at least one control region 300. In some embodiments, each of the therapeutic regions 200 may comprise a single layer and/or each of the control regions 300 may comprise a single layer. In some embodiments, one, some, or all of the therapeutic regions 200 may comprise multiple layers and/or one, some, or all of the control regions 300 may comprise multiple layers. Moreover, one or more of the individual control regions 200 and/or one or more of the therapeutic regions 300 may have the same or different amounts and/or types of releasing agent, and one or more of the therapeutic regions 200 may have the same or different amounts and/or types of therapeutic agent.

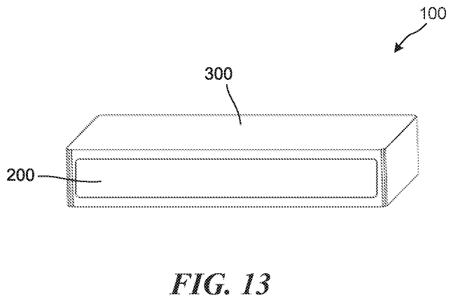

FIG. 13 shows a depot 100 in accordance with several embodiments of the present technology having a therapeutic region 200 enclosed on the top and bottom surfaces as well as two of the four lateral surfaces by a control region 300. This configuration is expected to release the therapeutic agent more slowly, at least initially, compared to a depot with the same dimensions and fully exposed lateral surfaces (see, e.g., the depot 100 shown in FIG. 4).

The release kinetics of the depots of the present technology may also be tuned for a particular application by varying the shape and size of the depot 100. Depending on the therapeutic dosage needs, anatomical targets, etc., the depot 100 can be different sizes, shapes, and forms for implantation and/or injection in the body by a clinical practitioner. The shape, size, and form of the depot 100 should be selected to allow for ease in positioning the depot at the target tissue site, and to reduce the likelihood of, or altogether prevent, the depot from moving after implantation or injection. In some embodiments, the depot is a flexible solid that is structurally capable of being handled by a clinician during the normal course of a surgery without breaking into multiple pieces and/or losing its general shape. Additionally, the depot may be configured to be placed within the body at a treatment site and release the therapeutic agent in vivo for up to 3 days or longer without breaking into multiple pieces.

Some of the form factors producible from the depot 100 or to be used adjunctive to the depot for implantation and fixation into the body include: strips, ribbons, hooks, rods, tubes, patches, corkscrew-formed ribbons, partial or full rings, nails, screws, tacks, rivets, threads, tapes, woven forms, t-shaped anchors, staples, discs, pillows, balloons, braids, tapered forms, wedge forms, chisel forms, castellated forms, stent structures, suture buttresses, coil springs, sponges, capsules, coatings, matrices, wafers, sheets, strips, ribbons, pills, and pellets.

The depot 100 may also be processed into a component of the form factors mentioned in the previous paragraph. For example, the depot could be rolled and incorporated into tubes, screws, tacks, or the like. In the case of woven embodiments, the depot may be incorporated into a multi-layer woven film/braid/mesh wherein some of the filaments used are not the inventive device. In one example, the depot is interwoven with Dacron, polyethylene or the like. For the sake of clarity, any form factor corresponding to the depot of the present technology, including those where only a portion or fragment of the form factor incorporates the depot, may be referred to herein as a “depot.”

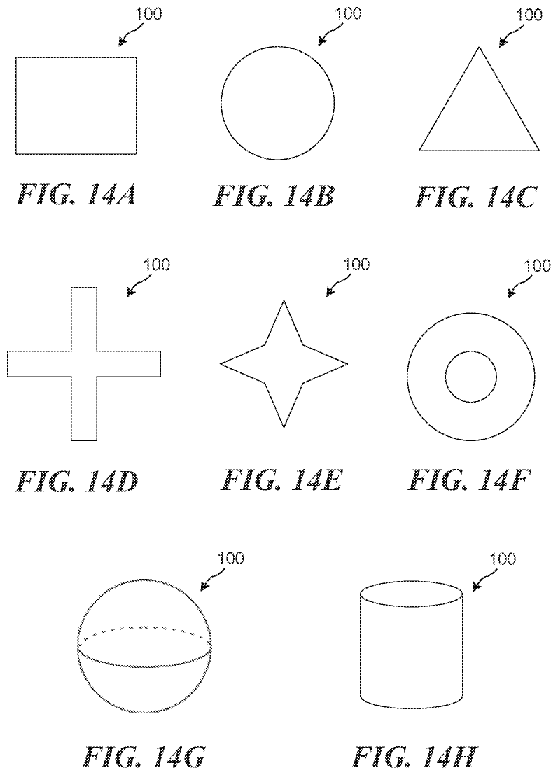

As shown in the cross-sectional views of FIGS. 14A-14H, in various embodiments, the depot 100 can be shaped like a sphere, a cylinder such as a rod or fiber, a flat surface such as a disc, film, ribbon, strip or sheet, a paste, a slab, microparticles, nanoparticles, pellets, mesh or the like. FIG. 14A shows a rectilinear depot 100. FIG. 14B shows a circular depot 100. FIG. shows a triangular depot 100. FIG. 14D show cross-like depot 100, FIG. 14E shows a star-like depot 100, and FIG. 14F shows a toroidal depot 100. FIG. 14G shows a spheroid depot 100, and FIG. 14H shows a cylindrical depot 100. The shape of the depot 100 can be selected according to the anatomy to fit within a given space and provide the desired fixation and flexibility properties. This is because the fit, fixation and flexibility of the depot may enhance the ease of implanting the depot, ensure delivery of the therapeutic agent to the target site, and prolong the durability of the implant in dynamic implant sites.

In some embodiments, the depot 100 may be configured to release the therapeutic agent in an omnidirectional manner. In other embodiments, the depot may include one or more barrier regions 400 covering one or more portions of the therapeutic region 200 and/or control region 300, such that release of the therapeutic agent is limited to certain directions. The barrier region 400 may provide structural support for the depot. The barrier region 400 may comprise a low porosity, high density of bioresorbable polymer configured to provide a directional release capability to the depot. In this configuration, the substantial impermeability of this low porosity, high density polymer structure in the barrier region 400 blocks or impedes the passage of agents released from the therapeutic region 200. Accordingly, the agents released from the therapeutic region 200 take a path of less resistance through the control region 300 opposite from the barrier region 400, particularly following the creation of diffusion openings in the control region 300.

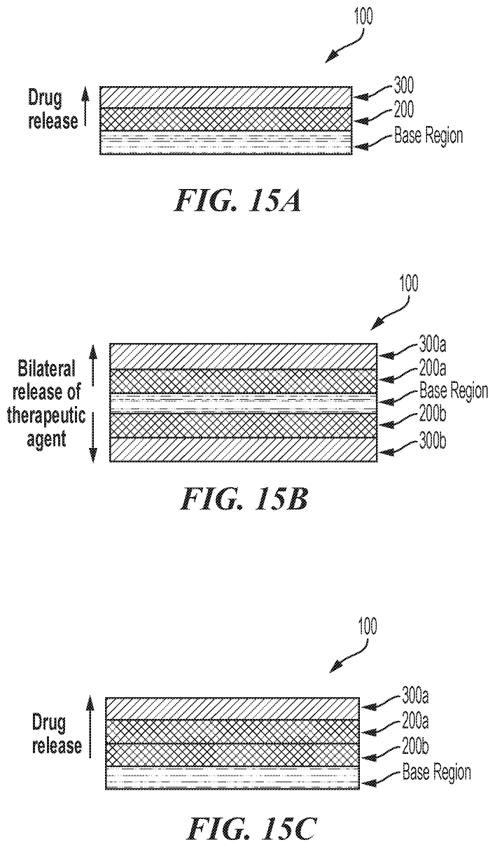

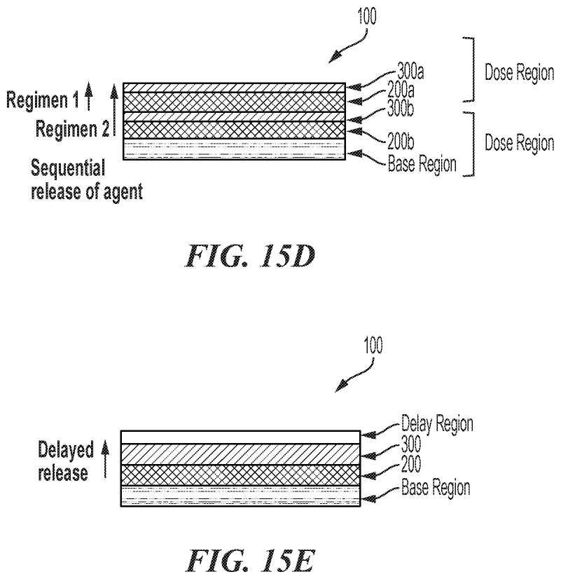

An example a depot 100 of the present technology having a barrier region 400 is shown in FIG. 15A. The barrier region 400 may comprise a low porosity, high density of bioresorbable polymer configured to provide a directional release capability to the multi-region depot. In this configuration, the low porosity, high density polymer structure in the barrier region 400 blocks or impedes passage of agents release from the therapeutic region 200. Accordingly, the agents released from the therapeutic region 200 take a path of lesser resistance through the control region opposite from the barrier region 400, particularly following the creation of channels in the control region. In an additional embodiment, the porosity of other regions of the multi-region depot can be varied to facilitate the release of therapeutic agent. For example, in this embodiment, the barrier region 400, the therapeutic region 200, and the control region 300 of the multi-region depot depicted in FIG. 15A may have different porosities ranging from low porosity in the barrier region 400 to higher porosities in the therapeutic agent and control regions to facilitate the release of therapeutic agent from the multi-region depot. In additional embodiments, the porosities of the edges of the multi-region depot, or within portions of any of the individual regions, can be varied to properly regulate or manipulate the release of therapeutic agent.

In the embodiment depicted in FIG. 15B, the multi-region depot provides for a bilateral or bidirectional release of therapeutic agent. This bidirectional release capability is accomplished through symmetric regioning about a high-density barrier region 400, wherein, as described above, the therapeutic agent releases along a path of less resistance, thereby releasing away from the high density barrier region 400. More specifically, disposed on one side of the barrier region 400 is a control region 300a and a therapeutic region 200a and, disposed on the other side of the barrier region 400, is a control region 300b and a therapeutic region 200b that are substantially similar to the pair on the other side. These pairs on either side of the barrier region 400 are configured to produce substantially equivalent, bidirectional release of therapeutic agent. In an alternate embodiment, a bidirectional release that is not equivalent (i.e., the therapeutic agent and/or rate of release in each direction is not the same) may be accomplished by asymmetric regioning, whereby the control region and therapeutic region pairs on either side of the barrier region 400 are substantially different.

In additional embodiments, it may be desirable for the multi-region depot to release multiple therapeutic agents. This capability can be particularly useful when multimodal pharmacological therapy is indicated. In the embodiment shown in FIG. 15C, the multi-region depot comprises a topmost or outermost control region 300a, a first therapeutic region 200a adjacent to the control region, a second therapeutic region 200b adjacent to the first therapeutic region 200a, and a barrier region 400 adjacent to the second therapeutic region 200b. In this embodiment, the first therapeutic region 200a and the second therapeutic region 200b comprise a first therapeutic agent and a second therapeutic agent, respectively. In certain embodiments, the first and second therapeutic agents are different. In one embodiment, the multi-region depot is configured to release the first and second therapeutic agents in sequence, simultaneously, or in an overlapping fashion to yield a complementary or synergistic benefit. In this configuration, the presence and function of the control region 300a may also ensure consistent and, if desired, substantially even release of multiple therapeutic agents residing beneath. Since many conventional drug delivery devices can fail to provide an even release of multiple drugs with different molecular weights, solubility, etc., the role of the control region in achieving a substantially even release of different therapeutic agents can be a significant advantage.

In some embodiments, the first therapeutic agent and second therapeutic agent are the same therapeutic agent but are present in the first and second therapeutic regions, respectively, in different relative concentrations to represent different dosages to be administered. In some embodiments, the first and second therapeutic agents of the first and second therapeutic regions, respectively, may have no clinical association or relationship whatsoever. For example, in some embodiments, it may be clinically desirable to administer in the vicinity of the surgical site both a first therapeutic agent for several days or weeks following the surgery and a second therapeutic agent for several weeks or months following the surgery. In this embodiment, the first therapeutic region 200a may comprise a therapeutically effective dose of the first therapeutic agent and the second therapeutic region 200b may comprise a therapeutically effective dose of the second therapeutic agent.

In some embodiments, as shown in FIG. 15D, the depot 100 comprises a first dosage region and a second dosage region, wherein the first and second dosage regions correspond to first and second dosage regimens. More specifically, each dosage region comprises a control region and therapeutic region pair, wherein each pair is configured for controlled release of a therapeutic agent from the therapeutic region 200a, 200b in accordance with a predetermined dosage regimen. In this exemplary embodiment, the first dosage region, and the control region and therapeutic region pair therein, would be sized, dimensioned, and configured such that the multi-region depot releases the first therapeutic agent in a manner that is consistent with the prescribed first dosage regimen. Similarly, the second dosage region, and the control region and therapeutic region pair therein, would be sized, dimensioned and configured such that the multi-region depot releases the second therapeutic agent in a manner that is consistent with the prescribed second dosage regimen. In another embodiment, the first and second dosage regions may correspond to dosage regimens utilizing different therapeutic agents. In one embodiment, the multi-region depot 100 is configured to administer the first and second dosage regimens in sequence, simultaneously, or in an overlapping fashion to yield a complementary or synergistic benefit. In an alternate embodiment of this scenario, the first and second dosage regimens, respectively, may have no clinical association or relationship whatsoever.

Certain embodiments of the present invention utilize delayed release agents. As illustrated in FIG. 15E, the depot 100 may include a barrier region 400 as the outermost (i.e., topmost) region to the multi-region depot and adjacent to a control region 300 comprising a releasing agent. The barrier region 400 presents a barrier to physiologic fluids from reaching and dissolving the releasing agent within the control region. In one embodiment, the barrier region 400 may comprise a delayed release agent mixed with a bioresorbable polymer, but without a releasing agent. Delayed release agents are different from the releasing agents used in the multi-region depot of the invention. Delayed release agents dissolve in physiological fluids more slowly than do releasing agents and thus provide the possibility for release of a therapeutic agent a defined amount of time following implantation of the multi-region depot. In embodiments where a delayed release agent is not present in the barrier region 400, it may take more time for the physiological fluids to traverse the barrier region 400 and contact the releasing agent. Only when the physiological fluids make contact with the control region will the releasing agent begin to dissolve, thus allowing the controlled release of the therapeutic agent. Delayed release agents may be advantageously used in the therapeutic methods of the invention wherein the therapeutic agent is not immediately required. For example, a nerve blocking agent may be injected prior to a surgical procedure, numbing the entire area around a surgical site. The controlled release of a local anesthetic is not required in such a surgery until the nerve block wears off.

Suitable delayed release agents for use in the present invention are pharmaceutically acceptable hydrophobic molecules such as fatty acid esters. Such esters include, but are not limited to, esters of myristoleic acid, sapienic acid, vaccenic acid, stearic acid, arachidic acid, palmitic acid, erucic acid, oleic acid, arachidonic acid, linoleic acid, linoelaidic acid, eicosapentaenoic acid, docosahexaenoic acid. Preferred esters include stearic acid methyl ester, oleic acid ethyl ester, and oleic acid methyl ester. Other suitable delayed release agents include tocopherol and esters of tocopherol, such as tocopheryl nicotinate and tocopheryl linolate.

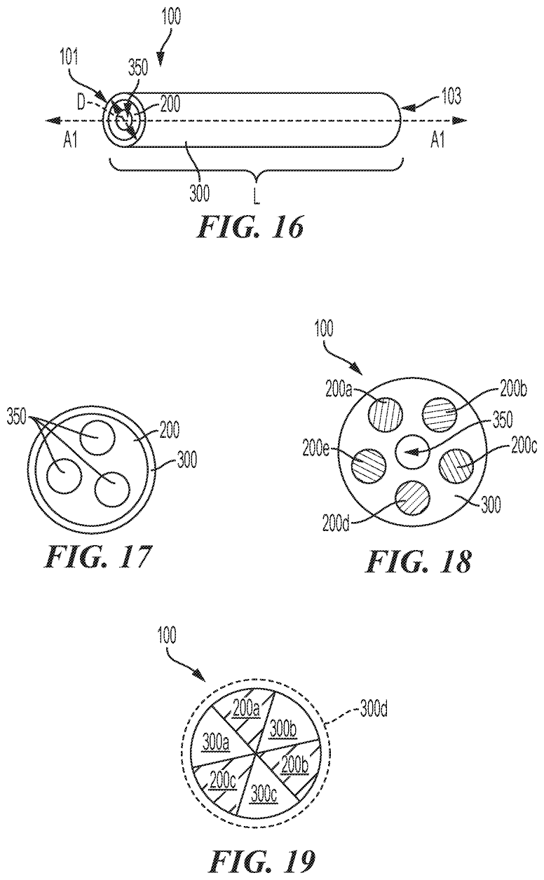

FIGS. 16-31 illustrate various examples of depots 100 having an elongated form. As depicted in FIG. 16, an “elongated depot” or an “elongated form” as used herein refers to a depot configuration in which the depot 100 has a length L between its ends along a first axis A1 (e.g., a longitudinal axis) that is at least 1.1, 1.2, 1.3, 1.4, 1.5, 1.6, 1.7, 1.8, 1.9, 2, 3, 4, 5, 6, 7, 8, 9, 10, 20, or 30 times greater than a maximum dimension D of a cross-sectional slice of the depot 100 within a plane orthogonal to the first axis A1. The elongated depots 100 described herein may include a therapeutic region 200 containing a therapeutic agent (such as any of the therapeutic agents described herein) and a control region 300 at least partially surrounding the therapeutic region 200 to control release of the therapeutic agent from the depot 100. The therapeutic region 200 may optionally include a bioresorbable polymer (such as any of the polymers described herein) and/or a releasing agent (such as any of the releasing agents described herein). The control region 300 may include a bioresorbable polymer (such as any of the polymers described herein) mixed with a releasing agent (such as any of the releasing agents described herein), but does not include any therapeutic agent at least prior to implantation. In some embodiments, the control region 300 may include some therapeutic agent prior to implantation, for example having a lower concentration of therapeutic agent than the therapeutic region 200. As discussed in greater detail below, the thickness of the control region 300, the concentration of releasing agent in the control region 300, the amount of exposed (uncovered) surface area of the therapeutic region 200, the shape and size of the depot 100, and other suitable parameters may be varied to achieve a desired release profile for the sustained, controlled release of the therapeutic agent from the depot 100.

In the embodiments shown in FIGS. 16-31, the elongated depot 100 has a cylindrical, columnar, and/or rod-like shape such that the cross-sectional shape is a circle and the cross-sectional dimension D is the diameter of the circle. In some embodiments, however, the elongated depot 100 may have another elongated configuration and/or cross-sectional shape along all or a portion of its length L. For example, the depot 100 may be in the form of a ribbon-like strip and thus have a square or rectangular cross-sectional shape. In other embodiments, the elongated depot 100 may have a circular, triangular, rhomboid, or other polygonal or non-polygonal cross-sectional shape based on the desired application. The elongated depot 100 may be a solid or semi-solid formulation with sufficient column strength to be pushed or pulled from a delivery device and sufficient durability and/or structural integrity to maintain its shape while the therapeutic agent is released into the surrounding anatomy for the desired duration of release.

A length L of the elongated depot 100 can be about 2 mm to about 300 mm, about 10 mm to about 200 mm, or about 10 mm to about 100 mm. In some embodiments, the maximum cross-sectional dimension D of the depot 100 can be between about 0.01 mm to about 5 mm, between about 0.1 mm to about 3 mm, or between about 0.5 mm to about 2 mm. The elongated form may be particularly well suited for injection or insertion to a subcutaneous, intramuscular, or other location through a needle or other suitable delivery device. Additionally or alternatively, the elongated depots 100 may be implanted using other techniques, for example surgical implantation through an open incision, a minimally invasive procedure (e.g. laparoscopic surgery), or any other suitable technique based on the application.

FIG. 16 illustrates an example of an elongated, generally cylindrical depot 100 comprising tubular, concentric therapeutic and control regions 200 and 300. The therapeutic region 200 comprises a tubular sidewall having an outer surface covered by the control region 300 and an exposed inner surface defining a lumen 350 that extends through the length L of the depot 100. The lumen 350 can be devoid of any material such that when the depot 100 is exposed to physiological fluid in vivo, the inner surface of the therapeutic region 200 is in direct contact with the fluid, thereby enhancing release of the therapeutic agent (relative to an elongated depot without a lumen through the therapeutic region). As shown in FIG. 16, the end surfaces of the therapeutic region 200 at the longitudinal ends 101, 103 of the depot 100 may also remain exposed/uncovered by the control region 300 (only one end surface is visible in FIG. 16). In some embodiments, the elongated depot 100 may include multiple, layered control regions 300 having the same composition or different compositions and/or the same thickness or different thicknesses. In these and other embodiments, the control region 300 may extend over one or both end surfaces of the therapeutic region 200. In particular embodiments, the lumen 350 extends through only a portion of the length L of the depot 100 and/or the tubular therapeutic region 200 is not concentric with the control region 300.

In some embodiments, the elongated depot 100 may include multiple lumens (e.g., two, three, four, five, six, etc.) extending through all or a portion of the length of the depot 100 and/or the length of the therapeutic region 200. For example, FIG. 17 is an end view of an elongated depot 100 having an inner therapeutic region 200 and an outer core region 300 covering an outer surface of the therapeutic region 200 along its length. In this particular example, the depot 100 includes three lumens 350 extending through the length of the therapeutic region 200. In the illustrated embodiment, each of the lumens 350 has a substantially circular cross-section and similar dimensions. In other embodiments, the lumens 350 may have other cross-sectional shapes, and/or the dimensions of each lumen 350 may vary from one another. In some embodiments, the elongated depot 100 may include multiple, layered control regions 300 having the same composition or different compositions and/or the same thickness or different thicknesses. In these and other embodiments, the control region 300 may extend over one or both end surfaces of the therapeutic region 200.

As shown in the end view of FIG. 18, the depot 100 can include a plurality of separate therapeutic regions 200 (labeled 200a-200e) extending longitudinally along the length of the depot 100. Although the depot 100 is shown having five therapeutic regions 200, in other embodiments the depot 100 may have more or fewer therapeutic regions 200 (e.g., two, three, four, six, seven, eight, etc.). The therapeutic regions 200 may be separated from one another by the control region 300. In the illustrated example, a central lumen 350 extends through the length of the control region 300, and the therapeutic regions 200 are distributed around the central lumen 350. In other embodiments, the elongated depot 100 may not include a lumen extending through any of its regions and may be solid across its cross-sectional dimension.

The therapeutic regions 200a-200e may have the same or different compositions, shapes, and/or dimensions. For example, the therapeutic regions 200a-200e may contain the same or different therapeutic agents, the same or different amount of therapeutic agent, the same or different polymers, and/or the same or different concentrations of releasing agents, depending on the desired release profile of each of the therapeutic regions 200a-200e. In the illustrated embodiment, each of the elongated therapeutic regions 200 has a substantially circular cross-section and similar dimensions. In other embodiments, the elongated therapeutic regions 200 may have other cross-sectional shapes and/or dimensions. In some embodiments, the elongated depot 100 may include one or more additional control regions 300 layered on top of the inner control region 300 surrounding the therapeutic regions 200a-200e. having the same composition or different compositions and/or the same thickness or different thicknesses. In these and other embodiments, the control region 300 may extend over one or both end surfaces of the therapeutic region 200.

FIG. 19 illustrates another embodiment of an elongated depot 100 in which the cross-sectional area is composed of three elongated therapeutic regions 200a-200c separated radially from one another by three elongated control regions 300. In the illustrated embodiment, each of the separate regions intersects at a center in a pie-shaped configuration, however the constituent control regions 300a-300c and therapeutic regions 200a-200c can take any shape and form in different embodiments. Optionally, the depot 100 may include an additional control region 300d covering an outer surface of the more inner therapeutic regions 300a-300c and control regions 300a-300c to provide another layer of controlled release. In some embodiments, the elongated depot 100 may include multiple, layered control regions 300 having the same composition or different compositions and/or the same thickness or different thicknesses. In these and other embodiments, the control region 300 may extend over one or both end surfaces of the therapeutic region 200.

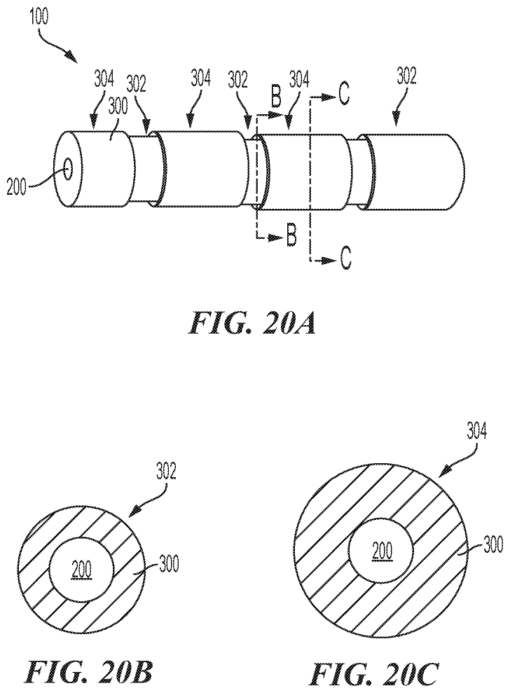

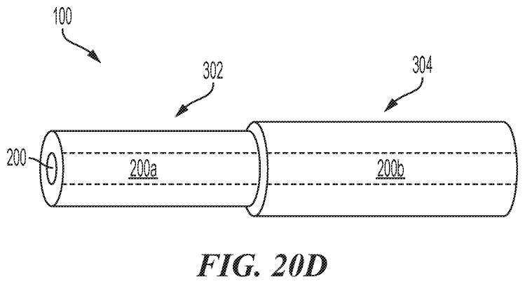

In certain instances, it may be beneficial to provide an elongated depot 100 having an inner therapeutic region 200 and an outer control region 300 of variable thickness and/or non-uniform coverage. Several examples of such depots 100 are shown FIGS. 20A-24. As depicted in FIGS. 20A-20C, the depot 100 can include an elongated therapeutic region 200 having a substantially uniform cross-sectional profile. The outer control region 300 radially surrounds the therapeutic region 200 along the length of the depot 100 and has a thickness that varies along the length of the depot 100. As shown in FIG. 20A, the control region 300 may have alternating first and second portions 305, 307 along its length. The first portions 302 can have a first thickness and the second portions 304 can have a second thickness greater than the first thickness. As such, the first portions 302 form annular grooves within the control region 300 at the outer surface of the depot 100. When implanted, the thinner first portions 302 may release the therapeutic agent more quickly than the thicker second portions 304, as the therapeutic agent has less control region to travel through before leaving the depot 100. By separately providing for faster-releasing portions and slower-releasing portions of the depot 100, the overall release rate of therapeutic agent from the therapeutic region 200 to a treatment site can be precisely tailored to a desired application. In addition to controlling the overall release rate, the release of therapeutic agent(s) can be spatially controlled, for example by directing a first therapeutic agent towards a first portion of the treatment site and a second therapeutic agent towards a second portion of the treatment site.

As shown in FIG. 20D, in some embodiments the elongated therapeutic region 200 may have different therapeutic agents disposed at different sections 200a, 200b along the length of the therapeutic region 200, where each section having a different therapeutic agent is axially aligned with a corresponding section of the control region 300 that has a thickness that is specific to the desired release profile of the underlying therapeutic agent. For example, in some applications it may be beneficial to release a first therapeutic agent at a faster rate and shorter duration and a second therapeutic agent at a slower rate for a longer duration. In such instances, the elongated therapeutic region 200 may have a first section 200a containing the first therapeutic agent (and optionally a polymer and/or releasing agent) and a second section 200b adjacent the first section 200a along the length of the therapeutic region 200 that has a second therapeutic agent (and optionally a polymer and/or releasing agent). The first section 302 of the control region 300 surrounding the first section 200a may have a thickness that is less than a thickness of the second section 304 of the control region 300 surrounding the second section 200b. As such, the first therapeutic agent contained in the first section 200a may release at a faster rate than the second therapeutic agent contained in the second section 200b. In some embodiments, a depot 100 can be configured to deliver two, three, four, five, or more different therapeutic agents, any or all of which can have different rates and times of release from the depot 100.

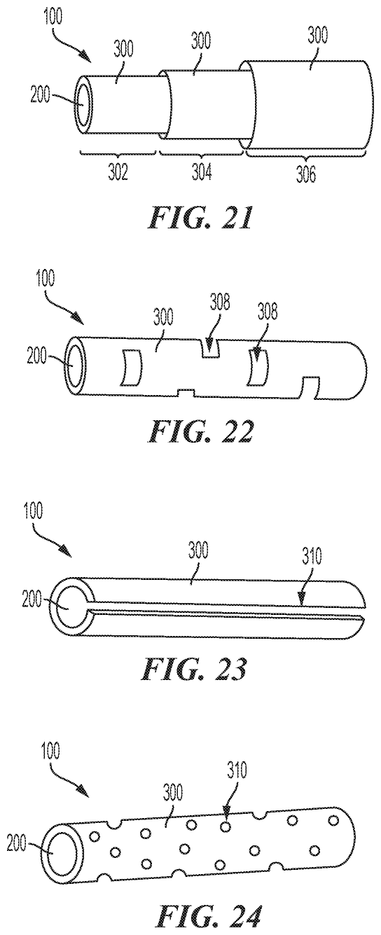

FIG. 21 illustrates another embodiment of an elongated depot 100 comprising an inner therapeutic region 200 radially surrounded by an outer control region 300. In the illustrated embodiment, the control region 300 includes three discrete sections 302, 304, 306 having increasing thickness. Although these increases in thickness are shown as step-changes between discrete sections, in other embodiments there may be a gradual taper or change in thickness of the control region 300 over the length of the depot 100. In some embodiments, the number of discrete sections may be varied as desired (e.g., two, four, five, six, seven, eight, nine, ten, or more discrete sections), and each discrete section may have an increased or decreased thickness and/or length relative to adjacent discrete sections. Each discrete section may be positioned around a corresponding section of the therapeutic region 200, and each section of the therapeutic region may include the same therapeutic agent, or may include different therapeutic agents as described with respect to FIG. 20D.

FIGS. 22-26 depict examples of elongated depots 100 comprising an inner therapeutic region 200 radially surrounded by an outer control region 300, where the outer control region 300 has one or more windows or openings extending through the entire thickness of the control region 300 to expose the underlying therapeutic region 200 through the opening(s). The openings can be notched into or laser cut from the control region 300, or the therapeutic region 200 can be masked while the control region 300 is applied (e.g., via spray- or dip-coating) to achieve the desired openings. The opening(s) provide a more rapid release route for the therapeutic agent to operate in concert with the more gradual release of therapeutic agent through the covered portions of the therapeutic region. The geometry of the opening(s) may be varied as desired, and can include squares, rectangles, circles, ellipses, slits, polygonal shapes, linear shapes, non-linear shapes, or combinations thereof.

As shown in FIG. 22, in some embodiments the openings may comprise a plurality of windows 308, some or all of which may extend around all or a portion of the circumference of the depot 100 and may be spaced apart along the length of the depot 100. FIG. 23 illustrates another embodiment of an elongated depot 100 in which the control region 300 is provided with a single elongated slit or opening 310. The opening 310 extends along the entire length of the control region 300 and/or depot 100 such that the control region 300 has a C-shape in cross-section. In the illustrated embodiment, the opening 310 extends substantially straight along a path parallel to the long axis of the depot 100, however in other embodiments the opening 310 may be curved, wind helically around the depot 100, or take any other suitable shape. The depot 100 shown in FIG. 24 is similar to that of FIGS. 22 and 23 except that the openings 350 are a plurality of circular holes or apertures extending through the thickness of the control region 300.

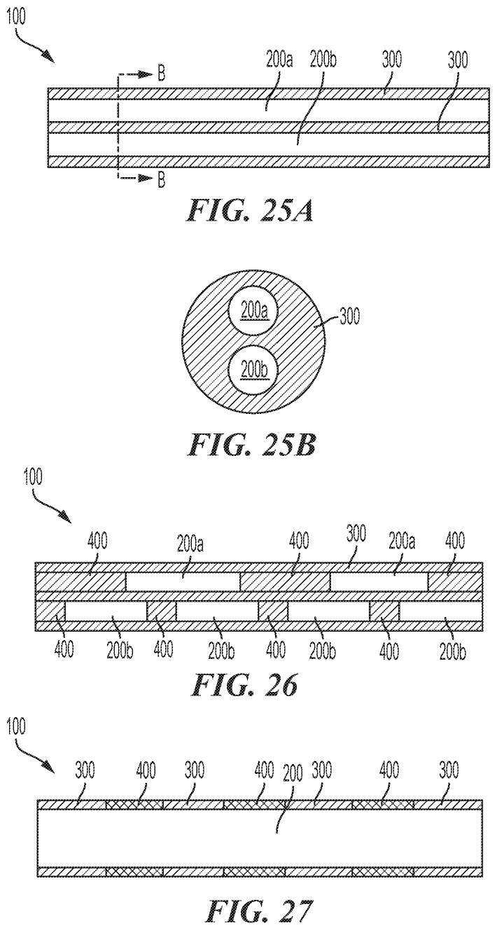

FIG. 25A and 25B are side and end cross-sectional views, respectively, of an elongated depot 100 comprising first and second elongated therapeutic regions 200a and 200b extending longitudinally within a surrounding control region 300. In the depicted embodiment, the central longitudinal axes of first and second therapeutic regions 200a and 200b are offset from each other and from the central longitudinal axis of the control region 300. In some embodiments, the first therapeutic region 200a can be configured to release the therapeutic agent more quickly than the second therapeutic region 200b, for example by varying the releasing agent concentration (if present), the therapeutic agent concentration, the polymer composition (if present), or other properties of the respective therapeutic regions 200a and 200b. The first and second therapeutic regions 200a and 200b can contain the same or different therapeutic agents.

The depot 100 shown in FIG. 26 is similar to that of FIG. 25A except that each therapeutic region 200a is interspersed along its length by barrier regions 400. As noted previously, certain embodiments of the depots 100 described herein employ barrier regions that present a barrier to physiologic fluids. In one embodiment, one or more of the barrier regions 400 may comprise a bioresorable polymer without any releasing agent. In another embodiment, one or more of the barrier regions 400 can include a delayed release agent mixed with a bioresorbable polymer, but without a releasing agent.

As depicted in FIG. 26, the first therapeutic region 400a is interspersed with three barrier regions 400 of a first length, while the second therapeutic region 200b is interspersed with four delayed release regions 400 having a shorter length. The relative lengths, number, composition, and spacing of the barrier regions 400 can be selected to achieve the desired release profiles. In operation, an exposed portion of the first or second therapeutic regions 200a or 200b may release therapeutic agent relatively quickly. However, once the therapeutic region 200a or 200b has been eroded and the exposed face of the depot 100 is a barrier region 400, the release of therapeutic agent from that particular therapeutic region may drop significantly. Accordingly, the use of such barrier regions 400 can allow for highly controlled release, with multiple periods of relatively steady release of therapeutic agent punctuated by periods in which little or no therapeutic agent is released due to the presence of the barrier regions 400.

FIG. 27 illustrates a depot 100 in which the inner therapeutic region 200 is continuous along the length of the depot 100, while the control region 300 is punctuated by barrier regions 400. The incorporation of these barrier regions 400 reduces the exposed surface area of the control region 300 and thereby decreases the rate of release of therapeutic agent from the depot 100.

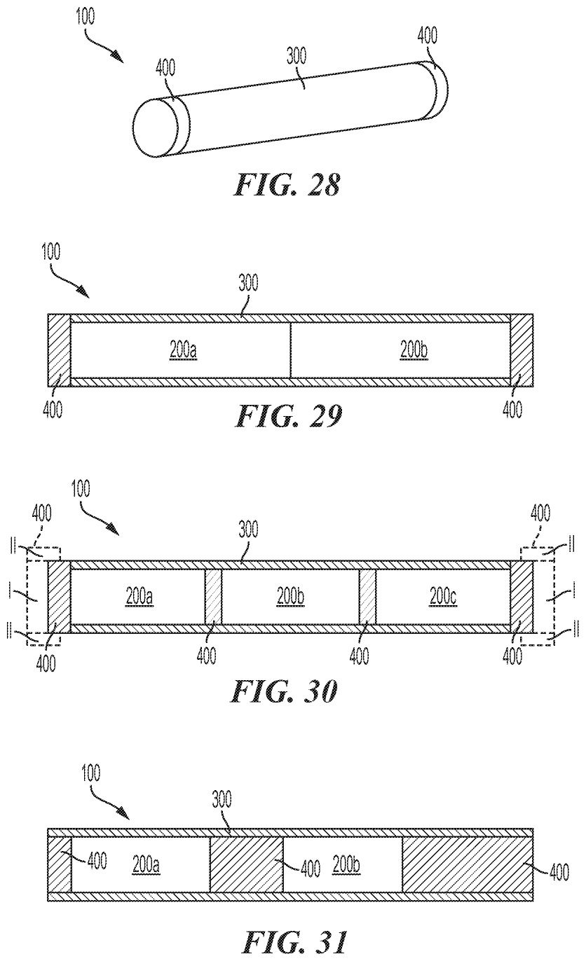

In the embodiments shown in FIG. 28-31, the elongated, columnar depot 100 includes first and second end caps formed of barrier regions 400. This configuration can eliminate the exposed surface at the ends of the columnar structure, thereby reducing the rate of release of therapeutic agent from the therapeutic region 200. As seen in FIGS. 28 and 29, the end caps formed of barrier regions 400 can have a diameter or cross-sectional transverse dimension substantially similar to that of the control region 300, such that the outer surface of the control region 300 is coplanar with a radially outermost surface of the barrier regions 400 forming the end caps.

In the embodiment shown in FIG. 29, the depot 100 includes first and second therapeutic regions 200a and 200b that are coaxially aligned and directly adjacent to one another (e.g., arranged in an end-to-end fashion along their longitudinal axes), while in FIGS. 30 and 31 the adjacent therapeutic regions 200a-200c are separated from one another by intervening barrier regions 400. FIG. 30 additionally shows optional end caps 400 that extend further radially, for example as shown in Section I, the end caps formed by barrier regions 400 can have the same diameter or transverse dimension as the control region 300, or alternatively as shown in Section II, the barrier regions 400 forming the end caps can project radially beyond the control region 300. In some embodiments, as best seen in FIG. 31, the thickness of the barrier regions 400 can vary across the depot 100 in order to achieve the desired release profile.

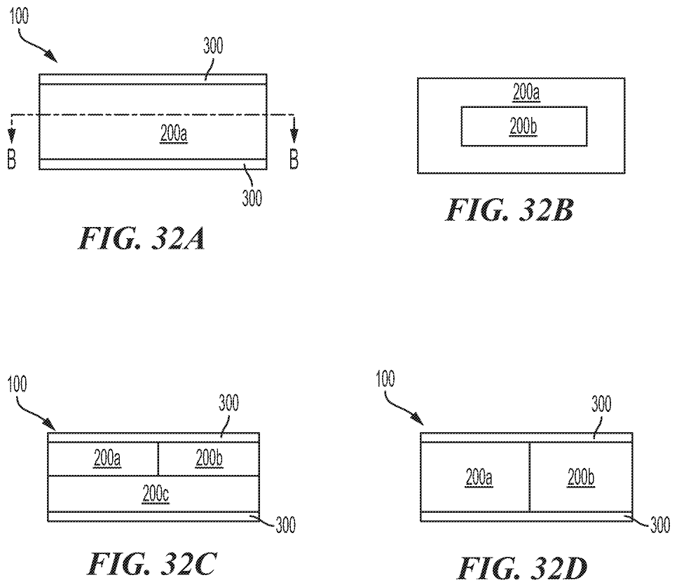

FIGS. 32A-35B illustrate various configurations of a depot 100 containing one or more therapeutic regions 200 that are at least partially surrounded by one or more control regions 300 and/or one or more barrier regions 400, with a form factor configured to provide the desired release profile. As noted previously, different therapeutic regions 200 can vary from one another in the composition of therapeutic agent(s) contained therein, the concentration of therapeutic agent(s) contained therein, polymer composition, or any other parameter that can vary the release profile. Similarly, in some embodiments the depot 100 may include multiple, layered control regions 300 and/or barrier regions 400 having the same composition or different compositions and/or the same thickness or different thicknesses. These depots 100 that include a plurality of different therapeutic regions 200, a plurality of different control regions 300, and/or a plurality of different barrier regions 400 can allow for controlled release of a single therapeutic agent or multiple different therapeutic agents according to a desired release profile. For example, in some applications it may be beneficial to release a first therapeutic agent at a faster rate and shorter duration and a second therapeutic agent at a slower rate for a longer duration. As described in more detail below, by varying the configuration and composition of the depots 100, the release profile of therapeutic agent(s) can be sequential (in the case of multiple therapeutic agents), delayed, zero-order, or otherwise.

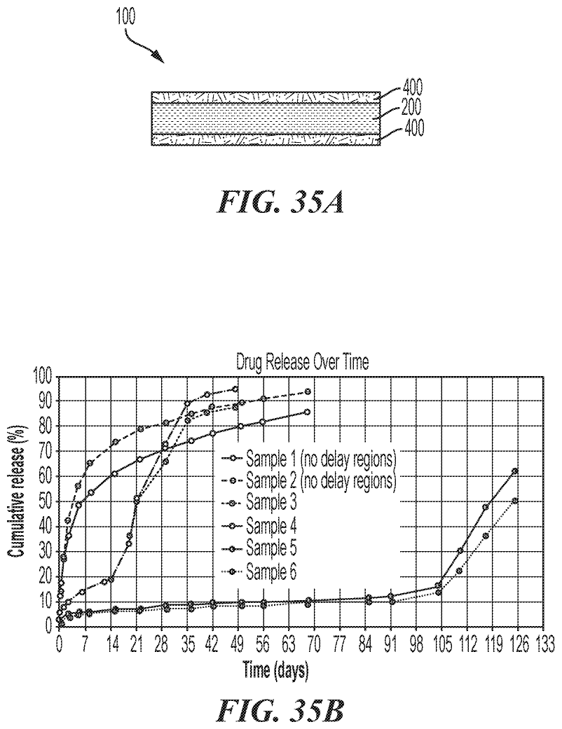

In some embodiments, a plurality of depots can be provided together (for example as a kit, an assembly, pre-loaded into a delivery device such as a syringe, etc.). In some embodiments, the depots can have a variety of different release profiles. For example, a system can include a plurality of depots selected from at least two of the following groups: (1) depots configured to provide for a substantially immediate burst release of therapeutic agent, (2) depots configured to provide for a substantially first-order release of therapeutic agent, (3) depots configured to provide for a substantially zero-order release of therapeutic agent, and (4) depots configured to exhibit delayed release of therapeutic agents (as discussed below with respect to FIGS. 35A-35B).

FIG. 32A shows a side view of a depot 100, and FIG. 32B shows a cross-sectional view taken along line B-B in FIG. 32A. As seen in FIGS. 32A-32B, in some embodiments the first therapeutic region 200a can envelop or at least partially or completely surround the second therapeutic region 200b. As a result, the first therapeutic region 200a will release its therapeutic agent(s) first, and release of therapeutic agent(s) from the second therapeutic region 200b will be relatively delayed. In some embodiments, the first therapeutic region 200a completely encapsulates the second therapeutic region 200b, such that no surfaces of the second therapeutic region 200b are directly exposed to physiologic fluids upon implantation in a patient's body. In other embodiments, the second therapeutic region 200b can be exposed along at least one face, thereby allowing more immediate release of therapeutic agent from the second therapeutic region 200b. In the illustrated embodiment, the first and second therapeutic regions 200a and 200b are arranged concentrically around the long axis of the depot 100, however in other embodiments the second therapeutic region 200b may be off-center, such that the first therapeutic region 200a is thicker along one side of the second therapeutic region 200b than along another side.

In the embodiment shown in FIG. 32C, first and second therapeutic regions 200a and 200b are arranged in an end-to-end fashion (e.g., in direct contact with one another), while a parallel third therapeutic region 200c extends along the length of the depot 100 and contacts both the first and second therapeutic regions 200a and 200b. FIG. 32D illustrates another embodiment in which first and second therapeutic regions 200a and 200b are arranged end-to-end and aligned along the length of the depot 100. These embodiments may be used to achieve directional release of therapeutic agents, e.g., the therapeutic agent of the first therapeutic region 200a is primarily released from a first end of the depot 100, and the therapeutic agent of the second therapeutic region 200b is primarily released from a second, opposite end of the depot 100, while the therapeutic agent of the third therapeutic region 200c releases from both ends of the depot 100.

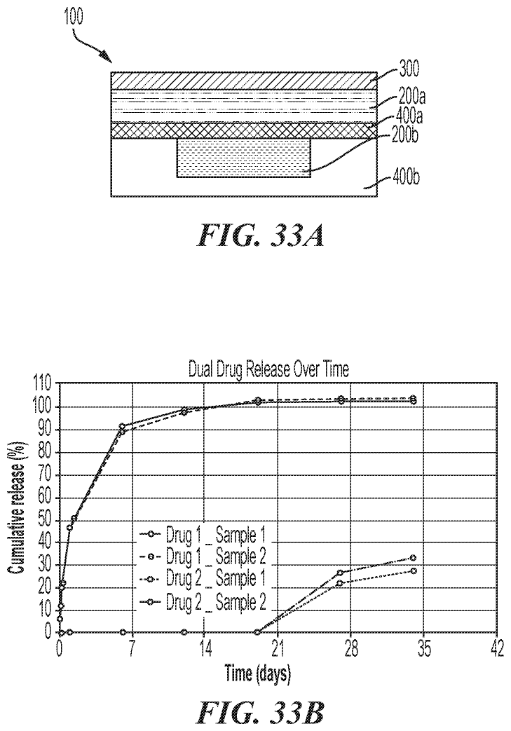

FIG. 33A illustrates a depot 100 configured to release therapeutic agent(s) from first and second therapeutic regions 200a and 200b in a sequential manner. As seen in FIG. 33A, the first therapeutic region 200a is partially covered by an overlying control region 300. The first therapeutic region 200a in turn overlies a first barrier region 400a. In the illustrated embodiment, the first therapeutic region 200a, the control region 300, and the first barrier region 400a each extend the entire length of the depot 100 and are each exposed along the side surfaces of the depot 100, however in other embodiments side surfaces may be covered completely or partially by a control region 300 and/or a barrier region 400. Beneath the first barrier region 400a is the second therapeutic region 200b, which may contain the same or different polymer composition and/or therapeutic agent as the first therapeutic region 200a. The second therapeutic region 200b is surrounded laterally by a second barrier region 400b, which also extends beneath the second therapeutic region 200b. As a result, the second therapeutic region 200b has at least one surface in contact with the first barrier region 400a and one or more remaining surfaces in contact with the second barrier region 400b, such that the second therapeutic region 200b is completely encapsulated by the first and second barrier regions 400a, 400b. In some embodiments, one or both of the barrier regions 400a and 400b can be substituted for control regions having a lower concentration of release agent than the control region 300.

As noted previously, barrier regions may present a barrier to physiologic fluids, for example by comprising a bioresorbable polymer without any releasing agent, or a delayed release agent mixed with a bioresorbable polymer, but without a releasing agent. The first barrier region 400a and the second barrier region 400b may differ from one another in composition, thickness, or any other parameters affecting dissolution of the barrier regions 400a and 400b. In some embodiments, the second barrier region 400b can be configured to dissolve more slowly than the first barrier region 400a, such that, after the first barrier region 400a has partially or completely dissolved, the second barrier region 400b remains intact and continues to block or delay passage of physiologic fluids therethrough.

In operation, the first barrier region 400a dissolves more slowly than either the control region 300 or the first and second therapeutic regions 200a and 200b, and therefore presents a barrier to physiological fluids passing through the first barrier region 400a. As a result, when the depot 100 is first placed into contact with physiologic fluids, the release agent of the control region 300 may begin to dissolve, thereby creating diffusion openings for the therapeutic agent(s) in the first therapeutic region 200a to escape therethrough. The therapeutic agent(s) in the first therapeutic region 200a may also escape directly through the exposed surfaces of the first therapeutic region 200a. However, at least in the initial period following implantation, the first barrier region 400a may stop or slow the passage of physiologic fluids through the barrier region 400a and to the underlying second therapeutic region 200b, such that the therapeutic agent(s) within the second therapeutic region 200b exhibits minimal or no release in the initial period. After a first period of time, the control region 300, first therapeutic region 200a and/or the first barrier region 400a may be partially or completely dissolved, thereby allowing at least some physiologic fluid to pass therethrough and come into contact with the second therapeutic region 200b. At this point, therapeutic agent(s) contained within the second therapeutic region 200b may begin to be released from the depot 100, for example by passing through openings formed in the first or second barrier regions 400a and 400b. Accordingly, the depot 100 can be configured such that all or substantially all (e.g., more than 80%, more than 90%) of the therapeutic agent(s) from the first therapeutic region 200a are released from the depot 100 before the therapeutic agent(s) from the second therapeutic region 200b are released in any substantial quantity (e.g., more than 1%, more than 5%, more than 10% of the therapeutic agent(s) contained within the second therapeutic region 200b). In some embodiments, the therapeutic agent(s) from the second therapeutic region 200b are not released in any substantial quantity until at least 12 hours, at least 18 hours, at least 24 hours, at least 36 hours, at least 48 hours, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks after implantation of the depot 100 and/or after release of substantially all of the therapeutic agent(s) from the first therapeutic region 200a.

In one example, the control region 300 is a PLGA film with a releasing agent, the first therapeutic region 200a is a PLGA film loaded with a first therapeutic agent (e.g., bupivacaine), the first barrier region 400a is a PLGA film with no releasing agent, the second therapeutic region 200b is a PLCL film loaded with a second therapeutic agent (e.g., 5-fluorouracil), and the second barrier region 400b is a PLCL film with no releasing agent. As will be understood, the particular polymers, therapeutic agents, releasing agents, concentrations thereof, and dimensions can be selected to achieve the desired release profiles of the first and second therapeutic agents and to achieve the desired total erosion of the depot 100 after a predetermined period of time.

Examples of the release profile from the depot 100 of FIG. 33A are illustrated in FIG. 33B. In this example, Samples 1 and 2 were each prepared with a configuration as shown in FIG. 33A with a thickness of approximately 1.8 mm and a length and width of approximately 20 mm. The control region 300 includes PLGA with polysorbate 20, commercially known as Tween 20™ as a releasing agent, with the ratio of Tween to polymer of 5:10. The first therapeutic region 200a includes a PLGA polymer with Tween 20 and bupivacaine HCl, with the ratio of tween to polymer to bupivacaine of 1:10:20. The first barrier region 400a includes a PLGA film with no releasing agent or therapeutic agent, and the second barrier region 400b includes a PLCL film with no releasing agent or therapeutic agent. The second therapeutic region 200b includes a PLCL polymer with 5-FU and no releasing agent, with a polymer to 5-FU ratio of 1:1.

Referring to FIG. 33B, the “Drug 1” lines illustrate release of a first therapeutic agent from the first therapeutic region 200a. The “Drug 2” lines illustrate release of a second therapeutic agent from the second therapeutic region 200b, which is not released in any substantial amount until a first period has passed (approximately 19 days in the embodiment of FIG. 33B), after which the second therapeutic agent begins to release from the depot 100. The result is a sequential release in which the first therapeutic agent is substantially completely released (e.g., more than 80%, more than 90%, more than 95%, more than 99% of the first therapeutic agent is released from the depot 100) before the second therapeutic agent begins to be released in any significant amount (e.g., more than 1%, more than 5%, or more than 10% of the second therapeutic agent is released from the depot 100).

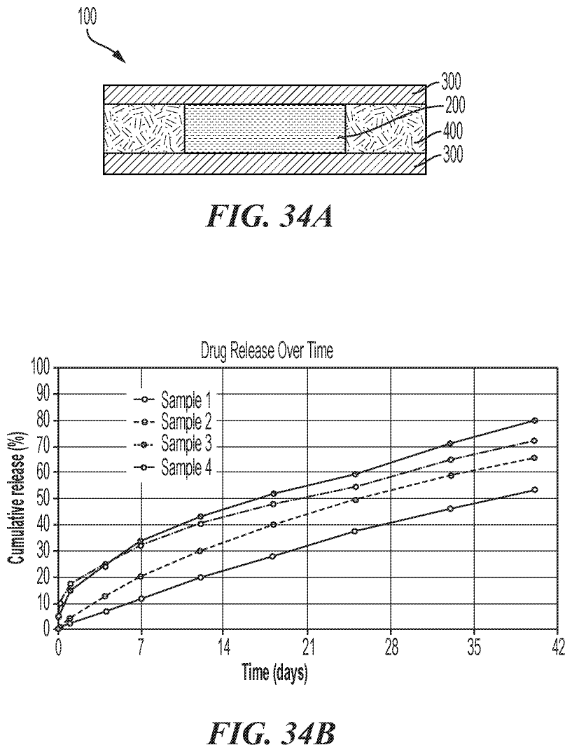

FIG. 34A illustrates a depot 100 configured to release a therapeutic agent from a therapeutic region 200 in accordance with a substantially zero-order release profile. In the illustrated embodiment, the depot 100 includes a therapeutic region 200 that is laterally surrounded by one or more barrier regions 400. In some embodiments, the therapeutic region 200 and the barrier 400 can have a substantially similar thickness such that upper and lower surfaces of the therapeutic region and the barrier region 400 are substantially coplanar. First and second control regions 300 can be disposed over upper and lower surfaces of both the therapeutic region 200 and the barrier region 400, such that the therapeutic region 200 is completely encapsulated by the first and second control regions 300 and the barrier region 400.

When the depot 100 is placed in contact with physiological fluids (e.g., when implanted at a treatment site in vivo), the release agent in the control regions 300 will begin to dissolve to form diffusion openings therein, after which therapeutic agent(s) contained within the therapeutic region 200 may begin to pass through to be released from the depot 100. By virtue of the laterally disposed barrier regions 400, little or no therapeutic agent may pass from the therapeutic region 200 through the barrier regions 400 for at least a period of time (e.g., at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks). As a result, substantially linear release of therapeutic agent can be achieved by controlling the dimensions and composition of the control regions 300 and the therapeutic region 200. As used herein, “substantially linear” includes a release profile in which the rate of release over the specified time period does not vary by more than 5%, or more than 10% from the average release rate over the time period. The substantially linear release profile can be maintained over a desired period of time, e.g., over at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks.

In one example, the control region 300 can be a PLCL or PLGA film containing a releasing agent, the therapeutic region can be a PLCL film loaded with a therapeutic agent (e.g., bupivacaine; 5-fluorouracil, etc.), and the barrier region 400 can be a PLCL film with no releasing agent. As will be understood, the particular polymers, therapeutic agents, releasing agents, concentrations thereof, and dimensions can be selected to achieve the desired release profiles of the therapeutic agent(s) and to achieve the desired total erosion of the depot 100 after a predetermined period of time (e.g., approximately 40 days).

Examples of the release profile from the depot 100 of FIG. 34A are illustrated in FIG. 34B, with four samples with varying polymer configurations illustrated. In this example, Samples 1-4 were each prepared with a configuration as shown in FIG. 34A with a thickness of approximately 0.8 mm and a length and width of approximately 20 mm. Samples 1 and 2 were prepared using the same configuration, in which the control region 300 includes a PLCL polymer and Tween as a releasing agent with a Tween to polymer ratio of 1:2. The therapeutic region 200 includes a PLCL polymer with 5-FU and no releasing agent, with a polymer to 5-FU ratio of 1:1, and the barrier region 400 includes a PLCL polymer with no releasing agent. Samples 3 and 4 were prepared using the same configuration, in which the control region 300 includes a PLGA polymer and Tween as a releasing agent with a Tween to polymer ratio of 1:2. The therapeutic region 200 includes a PLCL polymer with 5-FU and no releasing agent, with a polymer to 5-FU ratio of 1:1, and the barrier region 400 includes a PLGA polymer with no releasing agent.

As seen in FIG. 34B, by varying the polymer configurations (e.g., composition, release agent, thickness, etc.), the zero-order release profile can be tuned to release at different rates. In some embodiments, there is an initially higher rate of release for a first short period (e.g., approximately 1 day in the illustrated examples), followed by a substantially linear release for the remaining period of time.

FIG. 35A illustrates a depot 100 configured to release a therapeutic agent from a therapeutic region 200 in accordance with a delayed release profile, in which little or none of the therapeutic agent(s) are released in a first period (e.g., less than 10%, less than 20% of the therapeutic agent(s) are released), followed by a rapid increase in release rate during a second period in which the therapeutic agent is released from the depot 100. In the illustrated embodiment, the depot 100 includes a therapeutic region 200 that is at least partially surrounded on opposing sides (e.g., over top and bottom surfaces) by barrier regions 400. In some embodiments, the therapeutic region 200 and the barrier region 400 can have a substantially similar length and width such that the therapeutic region 200 is exposed at one or more side surfaces of the depot 100.

When the depot 100 is placed in contact with physiological fluids (e.g., when implanted at a treatment site in vivo), the therapeutic agent(s) contained within the therapeutic region 200 will pass from the therapeutic region 200 into the surrounding environment through the exposed side surface(s) of the therapeutic region 200. In some embodiments, little or none of the therapeutic agent passes through the barrier regions 400 during an initial period. During this period, a relatively small portion of the therapeutic agent may be released through the exposed side surfaces (e.g., less than 20%, less than 15%, less than 10%, or less than 5% of the therapeutic agent may be released). After the first time period, the barrier regions 400 may begin to degrade, after which the therapeutic agent begins to be released through openings formed in the barrier regions 400. As a result, the depot 100 achieves a delayed release in which little or none of the therapeutic agent is released over a first time period (e.g., more than 1 week, more than 2 weeks, more than 3 weeks, more than 4 weeks, more than 5 weeks, more than 6 weeks, more than 7 weeks, more than 8 weeks, more than 9 weeks, more than 10 weeks), after which the therapeutic agent is released from the depot 100 at an increased rate. In some embodiments, the exposed side surfaces of the therapeutic region 200 can be partially or completely covered by one or more control regions 300 and/or by one or more barrier regions 400, which can further delay release of the therapeutic agent from the therapeutic region 200.

In one example, the therapeutic region 200 can be a PLCL film loaded with a therapeutic agent (e.g., bupivacaine; 5-fluorouracil, etc.), and the barrier regions 400 can be PLGA film with no release agent or PLCL film with no release agent. As will be understood, the particular polymers, therapeutic agents, concentrations thereof, and dimensions can be selected to achieve the desired release profiles of the therapeutic agent and to achieve the desired total erosion of the depot 100 after a predetermined period of time.

Examples of the release profile from the depot 100 of FIG. 35A are illustrated in FIG. 35B. Samples 1 and 2 illustrate a release profile for a bare therapeutic region with no surrounding barrier regions. In samples 1 and 2, release of the therapeutic agent commences immediately after exposure to fluid. Samples 3-6 were each prepared with a configuration as shown in FIG. 35A. Samples 3 and 4 were prepared using the same configuration, in which the control region 300 includes a PLCL polymer and Tween as a releasing agent with a Tween to polymer ratio of 1:2. The therapeutic region 200 includes a PLCL polymer with 5-FU and no releasing agent, with a polymer to 5-FU ratio of 1:1, and the barrier region 400 includes a PLCL polymer with no releasing agent.

Samples 3-6 illustrate different examples of release profiles for the depot 100 of FIG. 35A with varying polymer configurations illustrated. In samples 3 and 4, the barrier regions 400 are made of a PLGA polymer, while in samples 5 and 6, the barrier regions 400 are made of a PLCL polymer. In samples 3 and 4, release of the therapeutic agent is delayed for approximately 2 weeks (e.g., less than 20%, less than 15%, less than 10%, or less than 5% of the therapeutic agent is released from the depot 100), after which the therapeutic agent is released from the depot 100 at an increased rate (e.g., at least 2 times, at least 3 times, at least 4 times, at least 5 times, or at least 10 times of the initial release rate). In samples 5 and 6, release of the therapeutic agent delayed for approximately 15 weeks (e.g., less than 20%, less than 15%, less than 10%, or less than 5% of the therapeutic agent is released from the depot 100), after which the therapeutic agent is released at an increased rate (e.g., at least 2 times, at least 3 times, at least 4 times, at least 5 times, or at least 10 times of the initial release rate). The barrier regions 400 in samples 3 and 4 are configured to degrade more quickly than the barrier regions 400 in samples 5 and 6, because PLGA degrades more quickly than PLCL. As a result, the delay period in samples 3 and 4 is shorter than the delay period in samples 5 and 6. In various embodiments, the degradation rate of the barrier regions 400 can be tuned by varying dimensions, selecting different polymers, or making any other suitable modifications to the barrier regions 400. By varying the polymer configurations (e.g., composition, thickness, etc.), the delayed release profile can be tuned to have different delay periods (e.g., an initial period during which little or none of the therapeutic agent is released) and to release the therapeutic agent at different rates following the delay period.

In some embodiments, it can be beneficial to provide a plurality of pre-formed openings or apertures extending through the depot 100, either in a regular or irregular pattern. Such openings can provide additional pathways for a therapeutic agent to pass from the therapeutic region to the treatment site, and as such can be controlled to vary the desired release profile. For example, in some embodiments the openings or apertures permit at least some of the therapeutic agent to be released directly from the therapeutic region 200 to the surrounding area, without passing through any overlying control region 300. These pre-formed openings or apertures may differ from diffusion openings formed by dissolution of releasing agent in that the openings or apertures are formed in the depot 100 prior to implantation in the patient's body. The openings or apertures may be used in combination with diffusion openings formed by dissolution of releasing agent to modulate the release profile of therapeutic agent. For example, a depot 100 having openings or apertures may release therapeutic agent at a higher rate than a depot 100 without openings or apertures.

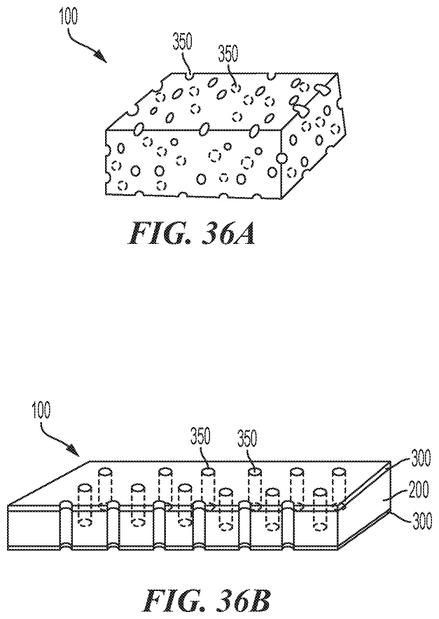

FIG. 36A illustrates a depot 100 with a sponge-like configuration in which a plurality of irregular openings 350 are formed through the depot 100. In some embodiments, such a depot 100 may be formed by introducing air or otherwise agitating the polymer composition during formation of the depot 100 and while encouraging the solvent to evaporate, resulting in a porous depot 100 with a plurality of openings therein. Such a depot 100 can be substantially uniform in its composition or can include an outer control region and an inner therapeutic region, one or both of which are permeated by some or all of the openings formed in the depot 100.

FIG. 36B illustrates a depot 100 in which a plurality of openings 350 extend through a thickness of the depot 100. In the illustrated embodiment, the openings 350 are substantially cylindrical and pass through upper and lower control regions 300 as well as an inner therapeutic region 200 along substantially parallel trajectories. In other embodiments, the openings 350 can assume other cross-sectional shapes, extend along other axes, and/or vary among one another in orientation, size, shape, etc.

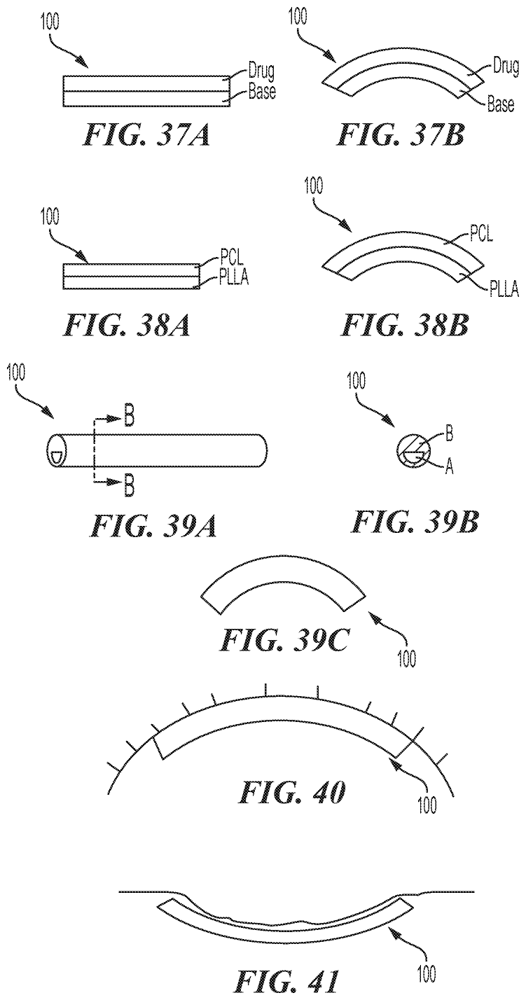

In some instances, it can be useful to provide a depot that has a curved, bent, or rounded configuration. For example, such curved depots can beneficially provide adequate contact with a curved surface area of a treatment site, such as the interior of a bladder, an abdominal wall, a surface of a tumor, or any other suitable treatment site. In some embodiments, the depot can have a substantially straight configuration prior to being deployed in vivo and the curved configuration can be achieved after the depot 100 is deployed in vivo in the presence of physiological fluids, while in other embodiments the depot 100 can have maintain the curved configuration both prior to and after being deployed in vivo. FIGS. 37A-40 illustrate various examples of depots 100 having curved configurations. With reference to FIGS. 37A-B, the depot 100 can have an actuating region 320 that is less elastic than a therapeutic region 200. For example, the actuating region 320 can have a different composition, different dimensions, and/or can be manufactured according to different processes than the therapeutic region 200. By stretching the depot 100 beyond the elastic hysteresis point of the less elastic actuating region 320, the depot 100 can transition from the substantially straightened configuration (shown in FIG. 37A) to the curved configuration (shown in FIG. 37B), in which the less elastic actuating region 320 pulls the depot 100 into the curved shape. In some embodiments, this stretching can occur after implantation, while in other instances the stretching is performed during manufacturing or by a surgeon before implantation. In some embodiments, this transition involves plastic deformation of the depot 100, such that the depot 100 maintains the curved shape even after the stretching force has been removed.

A similar result can be achieved by varying the polymer compositions of different layers or regions as in FIGS. 38A-38B. For example a first region 322 may have a polymer composition that is more hydrophilic than a second region 324, and accordingly the first region 322 may absorb more water or other fluids when implanted in vivo than the second region 324. In various embodiments, either or both of the first and second regions 322, 324 can carry a therapeutic agent. In the embodiment illustrated in FIGS. 38A-38B, the second region 324 is made of poly(L-lactic acid) (PLLA) and the first region 322 is made of polycaprolactone (PCL). In the presence of water, the PCL will experience a higher water uptake than the PLLA when placed in the presence of fluids. As a result, the PCL expands to a greater degree than the PLLA, resulting in a transition from the straightened state (shown in FIG. 38A) to the curved state (shown in FIG. 38B). In this embodiment, the depot 100 may advantageously retain the straightened state until it is deployed in vivo at the treatment site, at which point the depot 100 will begin to absorb water, resulting in a transition to the curved state.

FIGS. 39A-39C illustrate another mechanism for achieving a curved depot. As shown in FIGS. 39A and 39B, the depot 100 may include an outer region B and an axially offset inner region A. The inner region A can have a different composition (e.g., different polymer, the presence of therapeutic agent, etc.) compared to the outer region B. Because the inner region A if offset from the axial centerline of the depot 100, a difference in elasticity or expansion between the inner region A and the outer region B can result in curvature of the depot 100. In one example, the inner region A may include PLLA and the outer region B may include PCL, such that when exposed to water, outer region B expands more than the inner region A, resulting in a curved state.

As noted previously, a curved depot 100 may advantageously be deployed against a curved treatment site, for example in apposition with a concavely curved tissue surface (e.g., the interior of the bladder) as shown in FIG. 40, or in apposition with a convexly curved tissue surface (e.g., over a surface of a protruding tumor) as shown in FIG. 41. In other embodiments, the depot 100 may be configured to have a more complex curvature, for example at least one concave region and at least one convex region, or having different regions with different degrees of curvature. Such complex curvature can be tailored to achieve tissue apposition at a desired treatment site, and can improve delivery of therapeutic agent to the treatment site.

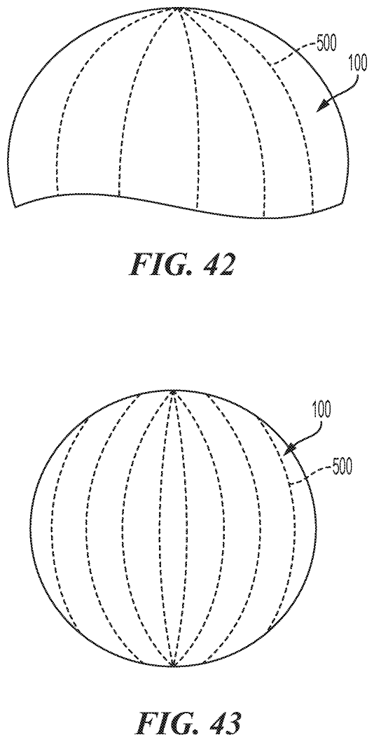

As shown in FIGS. 42 and 43, in some embodiments a treatment device can include an anchoring member 500 and a depot 100 carried on a surface of the anchoring member 500. The anchoring member 500 may be a generally hemispherical (as in FIG. 42), spherical (as in FIG. 43), or other suitable structure configured to expand from a low-profile state to a deployed state in apposition with a treatment site. The anchoring member 500 is configured to provide structural support to the treatment device, engage the adjacent anatomy (e.g., a bladder, etc.) to secure the treatment device to a selected treatment site.

In some embodiments, the depot 100 is bonded or otherwise adhered to the surface of the anchoring member 500. In other embodiments, the treatment device may include a depot 100 without an anchoring member 500. The depot 100 may comprise a biocompatible carrier loaded with one or more therapeutic agents and configured for a controlled, sustained release of the therapeutic agent(s) following in vivo placement of the depot. In some embodiments, the depot may be a thin, multilayer film loaded with a therapeutic agent, wherein, as described herein, the depot 100 is configured to release the therapeutic agent(s) at the treatment site.

In some embodiments the structure forming the anchoring member 500 may be a mesh structure. As used herein, “mesh” or “mesh structure” refers to any material (or combination of materials) having one or more openings extending therethrough. For example, in some embodiments, the anchoring member 500 comprises a plurality of filaments (e.g., wires, threads, sutures, fibers, etc.) that have been braided or woven into a tubular shape and heat set. In some embodiments, the mesh structure may be a stent formed of a laser-cut tube or laser-cut sheet, or the mesh structure may be a stent formed via thin film deposition. The anchoring member 500 may be in the form of a flat wire coil attached to a single longitudinal strut, a slotted tube, a helical band that extends circumferentially and longitudinally along the length of the anchoring member, a modular ring, a coil, a basket, a plurality of rings attached by one or more longitudinal struts, a braided tube surrounding a stent, a stent surrounding a braided tube, and/or any suitable configuration or embodiment disclosed herein.

In some embodiments, the anchoring member 500 may be formed of a superelastic material (e.g., nickel-titanium alloys, etc.) or other resilient materials such as stainless steel, cobalt-chromium alloys, etc. configured to self-expand when released from a delivery catheter. For example, the anchoring member may self-expand when pushed through the distal opening of the catheter, or by the delivery catheter being pulled proximally of the anchoring member. In some embodiments the anchoring member 500 may self-expand upon release from other constraining mechanisms (e.g., removable filaments, etc.). In some embodiments, the anchoring member 500 may be expanded manually (e.g., via balloon expansion, a push wire, a pull wire, etc.).

In some embodiments, the anchoring member 500 includes gold, magnesium, iridium, chromium, stainless steel, zinc, titanium, tantalum, and/or alloys of any of the foregoing metals or including any combination of the foregoing metals. In some embodiments, the anchoring member 500 may include collagen or other suitable bioresorbable or biodegradeable materials such as PLA, PLG, PLGA etc. In certain embodiments, the metal comprising the mesh structure may be highly polished and/or surface treated to further improve its hemocompatibility. The anchoring member 500 may be constructed solely from metallic materials without the inclusion of any polymer materials, or may include a combination of polymer and metallic materials. For example, in some embodiments the anchoring member 500 may include silicone, polyurethane, polyethylene, polyesters, polyorthoesters, polyanhyrides, and other suitable polymers. This polymer may form a complete sphere or hemisphere to block passage of tumor or drug though the anchoring member 500, or it may have microscopic pores to allow passage of drug but not tumor cells, or it may have small or large openings. In addition, all or a portion of the anchoring member may include a radiopaque coating to improve visualization of the device during delivery, and/or the anchoring member 500 may include one or more radiopaque markers.

In some embodiments, the anchoring member 500 may have other suitable shapes, sizes, and configurations. To improve fixation, in some embodiments the anchoring member 500 may have one or more protrusions extending radially outwardly from the mesh structure along all or a portion of its length, the one or more protrusions being configured to engage with tissue at the treatment site. For example, the anchoring member 500 may include one or more barbs, hooks, ribs, tines, and/or other suitable traumatic or atraumatic fixation members.

As previously mentioned, the depot 100 may be bonded or otherwise adhered to an outer surface of the anchoring member 500. For example, the depot 100 may be bonded to the anchoring member 500 by adhesive bonding, such as cyanoacrylate or UV curing medical grade adhesive, chemical or solvent bonding, and/or thermal bonding, and other suitable means. The depot 100 may also be sewn or riveted to the anchoring member 500. In some embodiments, the depot 100 may be woven into the anchoring member 500 at one or more sections of the anchoring member 500. In some embodiments, the anchoring member 500 may be dip coated in a solution comprising the material elements of the depot 100, and/or the anchoring member 500 may be spray coated with the depot 100. Sections of the anchoring member 500 may be selectively masked such that only certain portions of the anchoring member 500 may be coated with the depot 100. In some embodiments, the anchoring member 500 may be originally in the form of a sheet, and the sheet may be embedded into the depot 100 (for example, with the depot 100 as a multilayer film construction.) The resulting sheet structure (i.e., the anchoring member 500 embedded within the depot 100) may be rolled into a tubular structure (with or without the adjacent ends attached) for delivery into the body. In some embodiments, the depot may be coated with a bioresorbable adhesive derived from polyethylene glycol (PEG or PEO), for example, or from other hydrogels. The PEG or hydrogel may also be integral to the depot 100 via mixing in solution with the depot materials and not a separate coating.

The depot 100 may be disposed along all or a portion of the surface of the anchoring member 500, all or a portion of the circumference of the mesh structure, and/or cover or span all or some of the openings in the mesh structure depending on the local anatomy of the treatment site. For example, the volume, shape, and coverage of the tumor may vary patient-to-patient. In some cases, it may be desirable to use a treatment device having a depot 100 extending around the entire outer surface and/or inner surface of the anchoring member 500. In other cases, it may be desirable to use a treatment device having a depot 100 extending around less than the entire outer surface and/or inner surface of the anchoring member 500 to reduce exposure of potentially healthy tissue to the chemotherapeutic agents.

In some cases, the depot 100 may be elastically expandable, such that the depot 100 expands with the anchoring member 500 as it is deployed. The depot 100 may also be less elastic but can be folded for delivery in a compact form. Alternatively, the depot 100 could be configured to change shape as it is expanded. For example, a tubular depot could have a pattern of overlapping longitudinal slots, so that it expands into a diamond-shaped pattern as it is expanded. The expanded pattern of the depot 100 may align with the pattern of the anchoring member 500, or it may be totally independent of the anchoring member 500. This approach may enable the highest volume of therapeutic agent to be delivered in the most compact delivery format, while still enabling expansion on delivery and flexion, compression and expansion while positioned at the treatment site.

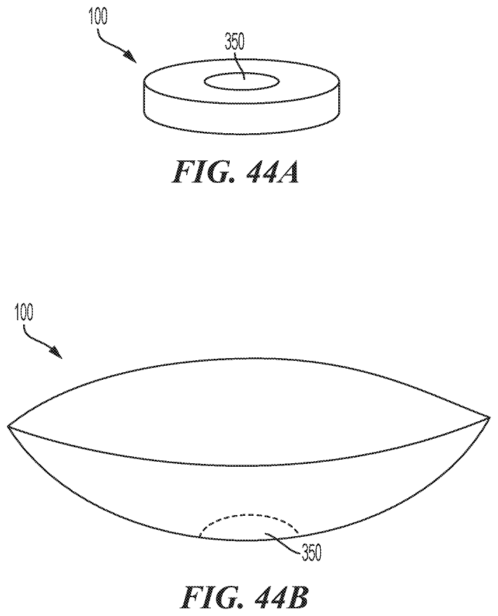

In certain cases, it can be useful to provide a depot 100 with a larger opening or lumen 350 therethrough. For example, a depot 100 deployed in a bladder may benefit from a relatively large opening that allows urine to pass therethrough. Such an opening can reduce the risk of the depot 100 interfering with normal physiological function. FIGS. 44A and 44B illustrate two different embodiments of such depots 100. As seen in FIG. 44A, the depot 100 can be substantially annular or ring-like structure with a central opening 350. For example, the central opening 350 can have a greatest transverse dimension that is more than 10%, more than 20%, more than 30%, more than 40%, or more than 50% of the length of a maximum transverse dimension and the annular depot 100. In the embodiment shown in FIG. 44B, the depot 100 can be a curved (e.g., semi-spherical or semi-ellipsoid) structure with a central opening 350 configured to allow fluid to pass therethrough. Although single openings 350 are illustrated in these embodiments, in other embodiments there may be two or more openings 350 configured to facilitate normal physiological function when the depot 100 is implanted at a treatment site.

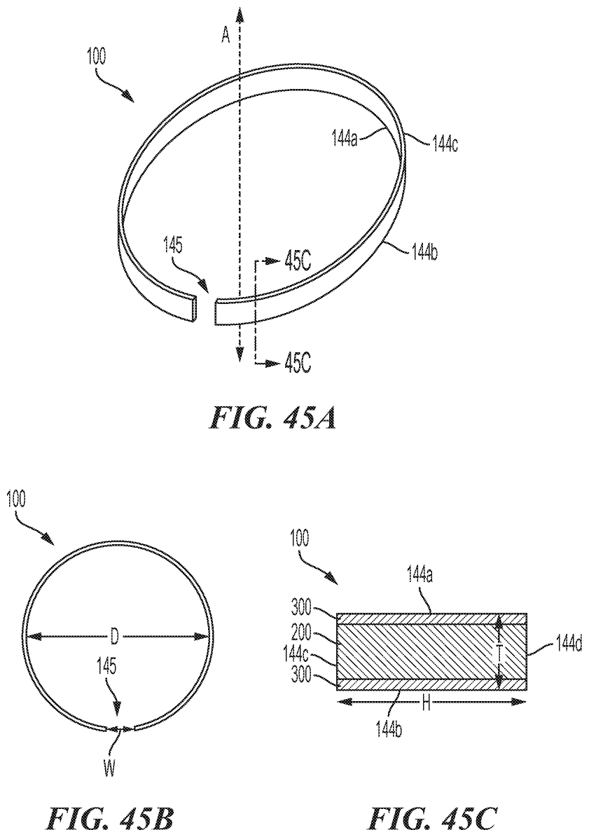

FIGS. 45A-C illustrate perspective, top, and cross-sectional views, respectively, of a depot 100 having an annular semi-annular shape. As illustrated, the depot 100 is an elongated strip, ribbon, or band that curls about an axis A. The depot 100 in the form of an elongated strip has an inwardly facing lateral surface 144a and an outwardly facing lateral surface 144b each having a width W. First and side second surfaces 144c and 144d can extend between the lateral surfaces 144a and 144b, defining a thickness T, such that the depot has a substantially rectangular cross-section as seen in FIG. 45C. In some embodiments, the band can have a thickness T of between about 0.1 mm and about 10 mm, or between about 0.5 mm and about 5 mm, or about 2 mm. In some embodiments, the depot 100 can have a height H of between about 0.1 mm and about 10 mm, or between about 0.5 mm and about 5 mm, or about 1 mm. The depot 100 can be curled about the axis A such that first and seconds ends are adjacent to one another, while leaving a gap 145 therebetween. In this curled configuration, the depot 100 is characterized by an inner diameter D. In some embodiments, for example for use in a bladder, the diameter D can be between about 2 cm and about 20 cm, for example between about 2 cm and about 10 cm, or between about 4 cm and about 8 cm, or approximately 6 cm. In some embodiments, the depot 100 can have a length of between about 20 cm and about 100 cm, for example between about 30 cm and about 50 cm, or approximately 38 cm.

In some embodiments, the ends can be joined together, creating a closed annular shape. As seen in FIG. 45C, in some embodiments the depot 100 includes a control region 300 disposed on the inwardly facing lateral surface 144a and another control region 300b disposed on the outwardly facing lateral surface 144b. In some embodiments, a therapeutic region 200 disposed between the two control regions 200 can be partially or completely exposed along the side surface 144c. Optionally, the therapeutic region 200 can also be partially or completely exposed along an opposing side surface 144d disposed opposite the first side surface 144c.

In some embodiments, the depot 100 of FIGS. 45A-45C can be delivered to the treatment site in a compressed configuration, either straightened longitudinally, or curled tightly about a central axis, or other compressed state. When delivered, the depot 100 can expand into the annular or semi-annular configuration as shown in FIG. 45A. In some embodiments, the depot 100 can be positioned such that the outwardly facing lateral surface 144b is in apposition with tissue along at least a portion of its length.

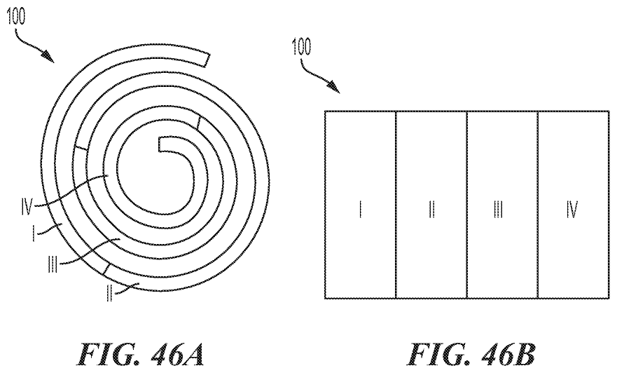

FIG. 46A shows an end view of a depot 100 in a spirally curled state and FIG. 46B shows a side view of the depot 100 in an uncurled state. The depot 100 includes a plurality of segments I-IV having different structural and mechanical properties that cause the depot 100 to assume the spirally curled configuration shown in FIG. 46A when placed in the presence of physiological fluids in vivo at a treatment site. For example, the different segments I-IV can vary in polymer composition, therapeutic agent, concentration of therapeutic agent, concentration of release agent, or any other parameter that affects the mechanical and structural properties of the depot 100, resulting in a spirally wound depot 100 as seen in FIG. 46A. In some embodiments, the spiral winding can facilitate placement of the depot 100 at a treatment site, and/or improve attachment to anatomical tissue at the treatment site.

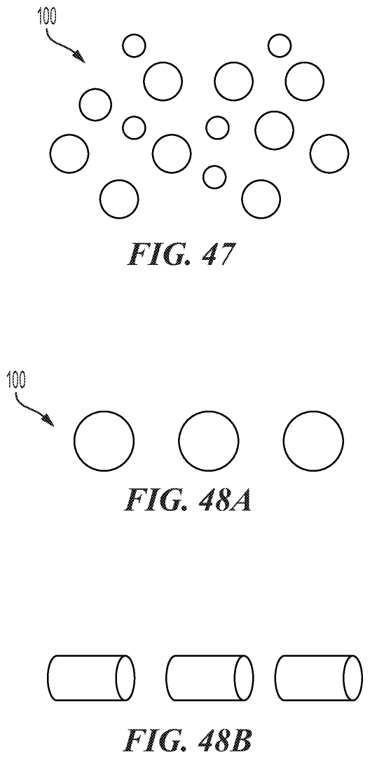

FIG. 47 illustrates a plurality of depots 100 in the form of microbeads, microspheres or particles. In various embodiments, each microbead can include a therapeutic region at its core and one or more control regions partially, substantially, or completely surrounding the therapeutic region. In some embodiments, the microbead may include multiple, layered control regions and/or therapeutic regions having the same composition or different compositions and/or the same thickness or different thicknesses. The release profile of any particular microbead is determined by its size, composition, and the thickness of the control region and therapeutic region. In some embodiments, a plurality of microbeads are provided having varying dimensions, varying shapes (e.g. spherical, ellipsoid, etc.), varying polymer compositions, varying concentration of therapeutic agent in the therapeutic region, varying concentration of releasing agent in the control region, or variation of any other parameters that affect the release profile. As a result, the composite release profile of the plurality of microbeads can be finely tuned to achieve the desired cumulative release of therapeutic agent to the treatment site. In various embodiments, some or all of the microbeads can have a diameter or largest cross-sectional dimension of between about 0.01 to about 5 mm, or between about 0.1 mm to about 1.0 mm. In some embodiments, some or all of the microbeads can have a diameter or largest cross-sectional dimension that is less than about 5 mm, less than about 2 mm, less than about 1.0 mm, less than about 0.9 mm, less than about 0.8 mm, less than about 0.7 mm, less than about 0.6 mm, less than about 0.5 mm, less than about 0.4 mm, less than about 0.3 mm, less than about 0.2 mm, or less than about 0.1 mm.

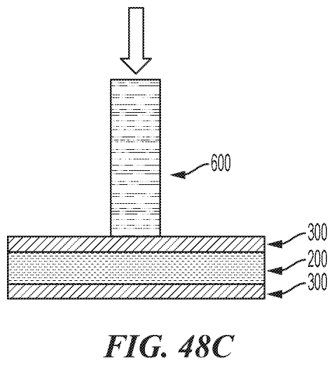

FIGS. 48A and 48B illustrate end and side views, respectively, of a plurality of depots 100 in the form of pellets. In the illustrated embodiment, the pellets are substantially cylindrical, however the particular shape and dimensions of the pellets may vary to achieve the desired release kinetics and form factor. For example, the pellets can have rounded ends (e.g., ellipsoid), and/or can have a cross-sectional shape that is circular, elliptical, square, rectangular, regular polygonal, irregular polygonal, or any other suitable shape. In some embodiments, each pellet can include an inner therapeutic region at least partially surrounded by an outer control region. In some embodiments, the pellet may include multiple, layered control regions and/or therapeutic regions having the same composition or different compositions and/or the same thickness or different thicknesses. As with the microbeads shown in FIG. 47, individual pellets of the plurality can vary from one another in one or more of shape, polymer composition, concentration of therapeutic agent in the therapeutic region, concentration of the releasing agent in the control region, thickness of the control region, thickness of the therapeutic region, and any other parameter that affect the release profile. As a result, the composite release profile of the plurality of pellets can be finely tuned to achieve the desired cumulative release of therapeutic agent to the treatment site.

In various embodiments, the depot can be different sizes, for example, the depot may be a length of from about 0.4 mm to 100 mm and have a diameter or thickness of from about 0.01 to about 5 mm. In various embodiments, the depot may have a layer thickness of from about 0.005 to 5.0 mm, such as, for example, from 0.05 to 2.0 mm. In some embodiments, the shape may be a rectangular or square sheet having a ratio of width to thickness in the range of 20 or greater, 25 or greater, 30 or greater, 35 or greater, 40 or greater, 45 or greater, or 50 or greater.

In some embodiments, a thickness of the control region (a single sub-control region or all sub-control regions combined) is less than or equal to 1/10, 1/12.5, 1/15, 1/17.5, 1/20, 1/22.5, 1/25, 1/27.5, 1/30, 1/32.5, 1/35, 1/37.5, 1/40, 1/42.5, 1/45, 1/47.5, 1/50, 1/55, 1/60, 1/65, 1/70, 1/75, 1/80, 1/85, 1/90, 1/95, or 1/100 of a thickness of the therapeutic region. In those embodiments with multiple sub-control regions, one or more of the sub-control regions may individually be less than or equal to 1/10, 1/12.5, 1/15, 1/17.5, 1/20, 1/22.5, 1/25, 1/27.5, 1/30, 1/32.5, 1/35, 1/37.5, 1/40, 1/42.5, 1/45, 1/47.5, 1/50, 1/55, 1/60, 1/65, 1/70, 1/75, 1/80, 1/85, 1/90, 1/95, or 1/100 of a thickness of the therapeutic region. In those embodiments where the control region comprises a single control region, the control region may have a thickness that is less than or equal to 1/10, 1/12.5, 1/15, 1/17.5, 1/20, 1/22.5, 1/25, 1/27.5, 1/30, 1/32.5, 1/35, 1/37.5, 1/40, 1/42.5, 1/45, 1/47.5, 1/50, 1/55, 1/60, 1/65, 1/70, 1/75, 1/80, 1/85, 1/90, 1/95, or 1/100 of a thickness of the therapeutic region. In those embodiments with multiple sub-control regions, one or more of the sub-control regions may individually be less than or equal to 1/10, 1/12.5, 1/15, 1/17.5, 1/20, 1/22.5, 1/25, 1/27.5, 1/30, 1/32.5, 1/35, 1/37.5, 1/40, 1/42.5, 1/45, 1/47.5, 1/50, 1/55, 1/60, 1/65, 1/70, 1/75, 1/80, 1/85, 1/90, 1/95, or 1/100 of a thickness of the depot. In those embodiments where the control region comprises a single control region, the control region may have a thickness that is less than or equal to 1/10, 1/12.5, 1/15, 1/17.5, 1/20, 1/22.5, 1/25, 1/27.5, 1/30, 1/32.5, 1/35, 1/37.5, 1/40, 1/42.5, 1/45, 1/47.5, 1/50, 1/55, 1/60, 1/65, 1/70, 1/75, 1/80, 1/85, 1/90, 1/95, or 1/100 of a thickness of the depot.

In some embodiments, the depot 100 has a width and a thickness, and a ratio of the width to the thickness is 21 or greater. In some embodiments, the ratio is 22 or greater, 23 or greater, 24 or greater, 25 or greater, 26 or greater, 27 or greater, 28 or greater, 29 or greater, 30 or greater, 35 or greater, 40 or greater, 45 or greater, or 50 or greater.

In some embodiments, the depot 100 has a surface area and a volume, and a ratio of the surface area to volume is at least 1, at least 1.5, at least 2, at least 2.5, or at least 3.

A. Flexural Load and Mechanical Integrity

In any of the foregoing embodiments shown and described above with respect to FIGS. 2-48C, dissolution of the releasing agent(s) and elution of the therapeutic agent(s) can change functional mechanical aspects of the depot 100 over time. Such mechanical aspects include structural integrity, flexural strength, tensile strength, or other mechanical characteristics of the depot 100. In some instances, undesirable degradation of the depot 100, such as premature degradation, can cause mechanical failure of the depot 100 and a corresponding undesirable burst release of therapeutic agent into the body. Accordingly, it can be beneficial for the depot 100 to maintain sufficient flexural strength and/or mechanical integrity in vivo for at least a predetermined period of time or until a predetermined proportion of therapeutic agent has been released from the depot 100. The depot 100 can be considered to maintain its structural integrity if the depot 100 remains largely intact with only partial or gradual reduction due to elution of therapeutic agent or dissolution of the control layers or releasing agent. The depot 100 can be considered to lose its structural integrity if it separates (e.g., fractures) into multiple component pieces, for example, with two or more of the resulting pieces being at least 5% of the previous size of the depot 100. Alternatively, or additionally, the depot 100 can be considered to lose its structural integrity if the release rate of the therapeutic agent increases by more than a factor of three as compared to the release rate of therapeutic agent in a control depot submerged in a buffered solution.

In some embodiments, the depot 100 is configured to maintain its structural integrity in vivo for at least a predetermined length of time. For example, the depot 100 can be configured to maintain its structural integrity in vivo for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, or at least 30 days, at least 40 days, at least 50 days, at least 60 days, at least 70 days, at least 90 days, at least 100 days, at least 200 days, at least 300 days, or at least 365 days.

In some embodiments, the depot 100 is configured to maintain its structural integrity in vivo until at least a predetermined proportion of therapeutic agent payload has been released from the depot. For example, the depot 100 can be configured to maintain its structural integrity in vivo until at least 5% by weight of the original payload has been released, at least 10% by weight of the original payload has been released, at least 15% by weight of the original payload has been released, at least 20% by weight of the original payload has been released, at least 25% by weight of the original payload has been released, at least 30% by weight of the original payload has been released, at least 35% by weight of the original payload has been released, at least 40% by weight of the original payload has been released, at least 45% by weight of the original payload has been released, at least 50% by weight of the original payload has been released, at least 55% by weight of the original payload has been released, at least 60% by weight of the original payload has been released, at least 65% by weight of the original payload has been released, at least 70% by weight of the original payload has been released, at least 75% by weight of the original payload has been released, at least 80% by weight of the original payload has been released, at least 85% by weight of the original payload has been released, at least 90% by weight of the original payload has been released, or until at least 95% by weight of the original payload has been released.

One aspect of the structural integrity of the depot 100 when it is in vivo can be quantified using a bend test, such as a three-point bend test that measures flexural properties including the flexural strength and/or maximum flexural stress sustained by a specimen before breaking. Such a bend test may represent (e.g., simulate) the forces that the depot 100 will encounter in vivo, for example in an anatomical joint. In one example, a depot can be subjected to a three-point bend test based on ASTM-D790-17, “Standard Test Methods for Flexural Properties of Unreinforced and Reinforced Plastics and Electrical Insulating Materials.” The text of this standard is hereby incorporated by reference in its entirety. The depot 100 may be suspended in a medium configured to simulate in vivo conditions, for example a phosphate buffered saline (PBS) at approximately 37° C. The bend test may be performed after different time periods of submersion in the medium to evaluate changes in the flexural strength of the depot 100 over time in simulated in vivo conditions.

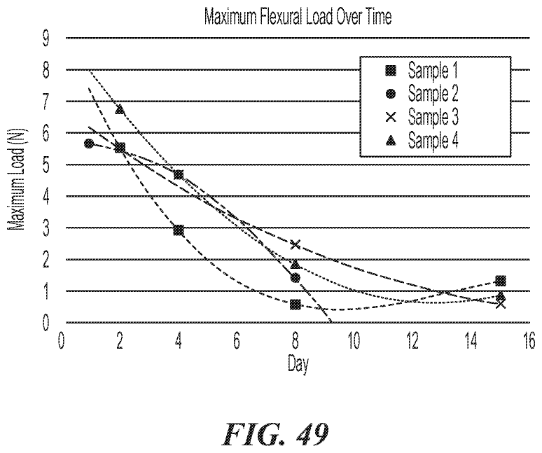

Table 1 shows the maximum flexural load sustained by four different samples of the depot 100 at different time periods following submersion in the medium as measured using a three-point bend test with maximum deflection set at 2.13 mm. The values in Table 1 reflect measurements made from two instances of each of the listed samples. FIG. 49 is a graph illustrating these values plotted graphically and fitted with trendlines. In each of these four samples, the depot 100 includes a therapeutic region 200 surrounded by upper and lower control regions 300a-b as shown and described above with reference to FIG. 4 or 5. The therapeutic region 200 has exposed lateral surfaces 202 between the first and second control regions 300a-b. The depots 100 each have lateral dimensions of approximately 2.5 cm by 1.5 cm, with a thickness of approximately 1 mm.

Sample 1 is a depot having a therapeutic region with a ratio by weight of releasing agent to polymer to therapeutic agent of 0.5:10:20. The polymer in this sample is P(DL)GACL with a PDLLA:PGA:PCL ratio of 6:3:1, the releasing agent is Tween 20, and the therapeutic agent is bupivacaine hydrochloride. In this sample, the depot includes a first control region 300a comprising a single control layer over the upper surface of the therapeutic region 200 and a second control region 300b comprising single control layer over the lower surface of the therapeutic region 200, as shown and described above with reference to FIG. 4. Each control region 300a-b individually has a ratio of releasing agent to polymer of 5:10.

Sample 2 is a depot having a therapeutic region 200 with a ratio by weight of releasing agent to polymer to therapeutic agent of 1:10:20. The polymer in this sample is PLGA with a PLA:PGA ratio of 1:1, the releasing agent is Tween 20, and the therapeutic agent is bupivacaine hydrochloride. Similar to Sample 1, the depot of Sample 2 includes a control region 300 comprising a first control region 300a with a single control layer over the upper surface of the therapeutic region 200 and a second control region 300b comprising a single control layer over the lower surface of the therapeutic region 200, as shown and described above with reference to FIG. 4. Each control region 300a-b individually has a ratio of releasing agent to polymer of 5:10.

Sample 3 is a depot having therapeutic region 200 with a ratio by weight of releasing agent to polymer to therapeutic agent of 5:10:20. The polymer in this sample is P(DL)GACL with a PDLLA:PGA:PCL ratio of 6:3:1, the releasing agent is Tween 20, and the therapeutic agent is bupivacaine hydrochloride. In this sample, the depot includes a control region 300 comprising a first control region 300a with two sub-control regions 302a-b over the upper surface of the therapeutic region 200, and a second control region 300b with two sub-control regions 302c-d, as shown and described above with reference to FIG. 5. Each of the inner sub-control regions 302b and 302c contacts the surface of the therapeutic region 200 and has a ratio of releasing agent to polymer of 5:10, and each of the outer sub-control regions 302a and 302d has a ratio of releasing agent to polymer of 1:10. The depot of Sample 3, therefore, includes a total of four sub-control regions.

Sample 4 is a depot having a therapeutic region 200 with a ratio by weight of releasing agent to polymer to therapeutic agent of 5:10:20. The polymer in this sample is PLGA with a PLA:PGA ratio of 1:1, the releasing agent is Tween 20, and the therapeutic agent is bupivacaine hydrochloride. As with Sample 3, the depot of Sample 4 includes a control region 300 having first and second control region 300a-b that each have two sub-control regions 302a-b and 302c-d, respectively, as shown and described with respect to FIG. 5. The depot of Sample 4 according also has a total of four sub-control regions 302a-d, two over the upper surface of the therapeutic region 200 and two over the lower surface of the therapeutic region 200. The inner of the sub-control regions 302b and 302c has a ratio of releasing agent to polymer of 5:10, and the outer of the sub-control regions 302a and 302d has a ratio of releasing agent to polymer of 1:10.

TABLE 1

| Depot Sample | Day 0 | Day 1 | Day 3 | Day 7 | Day 14 | Day 28 |
|---|---|---|---|---|---|---|
| Sample 1: | No break | 5.553N | 2.903N | 0.569N | 1.263N | Not |
| P(DL)GACL 6:3:1 | | 1.25 lbf | 0.0653 lbf | 0.134 lbf | 0.284 lbf | tested |
| 2 control layers | | | | | | |
| Sample 2: | 5.623N | 5.447N | 4.623N | 1.386N | Not | Not |
| PLGA 1:1 | 1.264 lbf | 1.22 lbf | 1.04 lbf | 0.312 lbf | tested | tested |
| 2 control layers | | | | | | |
| Sample 3: | No | 5.474N | Not | 2.430N | 0.605N | Sample |
| P(DL)GACL 6:3:1 | break | 1.23 lbf | tested | 0.546 lbf | 0.136 lbf | degraded |
| 4 control layers | | | | | | |
| Sample 4: | No | 6.763N | Not | 1.816N | 0.869N | Sample |
| PLGA 1:1 | break | 1.52 lbf | tested | 0.408 lbf | 0.195 lbf | degraded |
| 4 control layers | | | | | | |

As shown in Table 1, all samples were intact and maintained sufficient structural integrity after 14 days of being suspended in the medium to withstand a bending force before fracturing. Although the maximum load tolerated by each sample decreased over time, the flexural strength of these samples at 14 days was sufficient to maintain the structural integrity desired for implantation in an active joint, such as the knee or shoulder. As shown above, for two of the samples tested at 28 days, the samples had degraded such that the test could not be performed because the sample was no longer structurally intact. In such instances, it may be desirable to configure the depots such that all or substantially all the therapeutic agent payload has been released from the depot prior to its degradation and loss of structural integrity.

In this series of experiments summarized in Table 1, the sample depots are generally flexible at Day 0 before submersion in PBS. Following submersion, the flexural strength of the depots decreased such that the depots became more brittle with time. Yet, at 7-14 days, the depots were still sufficiently functionally intact. Without being bound by theory, it is believed that after the therapeutic agent has eluted, the depots gradually become an empty polymer matrix. For example, after 14-28 days in the solution, the depots may weigh only approximately 30% of their starting weight before submersion in the PBS. At this lower weight and in the porous state, the depots may be more brittle, with lower flexural strength and less resistance to bending loads.

As noted above, it can be advantageous for the depots 100 to maintain their structural integrity and flexural strength even while they gradually degrade as the therapeutic agent payload releases into the body. In some embodiments, the depot 100 can be configured such that, in in vitro testing utilizing a three-point bend test, the flexural strength of the depot 100 decreases by no more than 95%, no more than 90%, no more than 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, or no more than 5% after being submerged in PBS for a predetermined period of time. In various embodiments, the predetermined period of time that the depot 100 is submerged in PBS before being subjected to the three-point bend test is 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, after 21 days, after 22 days, 23 days, 24 days, 25 days, 26 days, 27 days, 28 days, after 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more. In at least some embodiments, the change in flexural strength of the depot 100 can be measured between day 0 (e.g., before submersion in the PBS) and a subsequent time after some period of submersion in PBS. In other embodiments, the change in flexural strength of the depot 100 can be measured between day 1 (e.g., after 24 hours of submersion in PBS) and a subsequent time following longer submersion in PBS.

In some embodiments, the depot 100 can be configured such that, in in vitro testing utilizing a three-point bend test, the flexural strength of the depot 100 decreases by no more than 95%, no more than 90%, no more than 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, or no more than 5% over the time period in which a predetermined percentage of the initial therapeutic agent payload is released while the depot 100 is submerged in PBS. In various embodiments, the predetermined percentage of payload released when the depot 100 is submerged in PBS before being subjected to the three-point bend test is about 5%, about 10%, about 15%, about 20%, about 25%, about 30%, about 35%, about 40%, about 45%, about 50%, about 55%, about 60%, about 65%, about 70%, about 75%, about 80%, about t 85%, about 90%, or about 95%. As noted above, in at least some embodiments, the change in flexural strength of the depot 100 can be measured between day 0 (prior to submersion in PBS) or day 1 (after 24 hours of submersion in PBS) and a subsequent following longer submersion in PBS.

In some embodiments, the depot 100 has (a) lateral dimensions of about 1.0-3.0 cm, (b) a thickness of about 0.5-2.5mm, and (c) a payload of therapeutic agent sufficient to release about 100 mg to about 500 mg of therapeutic agent per day for up to 7, 8, 9, 10, 11, 12, 13, 14, 15, 16, 17, 18, 19, or 20 days, and the depot 100 is configured to remain sufficiently mechanically intact to provide sustained, controlled release of therapeutic agent for at least 3 days, at least 4 days, at least 5 days, at least 6 days, at least 7 days, at least 8 days, at least 9 days, at least 10 days, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 22 weeks, at least 24 weeks, or more. Such embodiments of the depot 100 can comprise the therapeutic region 200 with a therapeutic agent and the control region 300. The control region 300 can have first and second control regions 300a-b, such as those shown and described above with reference to FIGS. 4-13, and the control region 300 comprises a bioresorbable polymer and a releasing agent mixed with the bioresorbable polymer. The releasing agent is configured to dissolve when the depot 100 is placed in vivo to form diffusion openings in the control region 300. The depot 100 is further configured such that, following submersion of the depot 100 in a buffer solution for seven days, the flexural strength of the depot 100 decreases by no more than 75%, or by no more than 70%, or by no more than 65%, or by no more than 60%, or by no more than 55%, or by no more than 50%, or by no more than 45%

In some embodiments, the depot 100 has (a) lateral dimensions of about 1.0-3.0 cm, (b) a thickness of about 0.5-2.5mm, and (c) a payload of therapeutic agent sufficient to release about 100 mg to about 500 mg of therapeutic agent per day for up to 7, 8, 9, 10, 11, 12, 13, 14, 15, 16, 17, 18, 19, or 20 days, and the depot 100 is configured to remain sufficiently mechanically intact to provide sustained, controlled release of therapeutic agent for at least 7 days. Such embodiments of the depot 100 can comprise the therapeutic region 200 with a therapeutic agent and the control region 300. The control region 300 can have first and second control regions 300a-b, such as those shown and described above with reference to FIGS. 4-13, and the control region 300 comprises a bioresorbable polymer and a releasing agent mixed with the bioresorbable polymer. The releasing agent is configured to dissolve when the depot 100 is placed in vivo to form diffusion openings in the control region 300. The depot is further configured such that, following submersion of the depot in buffer solution until approximately 75% of the therapeutic agent by weight has been released, the flexural strength of the depot decreases by no more than 75%, or by no more than 70%, or by no more than 65%, or by no more than 60%, or by no more than 55%, or by no more than 50%, or by no more than 45%.

B. Therapeutic Region

The total payload and release kinetics of the depots 100 of the present technology may be tuned for a particular application by varying the composition of the therapeutic region 200. In many embodiments, the therapeutic region 200 may include a high therapeutic payload of a therapeutic agent, especially as compared to other known polymer devices of equal thickness or polymer weight percentage. For example, the depots 100 of the present technology may comprise at least 15% by weight of the therapeutic agent, at least 20% by weight of the therapeutic agent, at least at least 25% by weight of the therapeutic agent, at least 30% by weight of the therapeutic agent, at least 35% by weight of the therapeutic agent, at least 40% by weight of the therapeutic agent, at least 45% by weight of the therapeutic agent, at least 50% by weight of the therapeutic agent, at least 55% by weight of the therapeutic agent, at least 60% by weight of the therapeutic agent, at least 65% by weight of the therapeutic agent, at least 70% by weight of the therapeutic agent, at least 75% by weight of the therapeutic agent, at least 80% by weight of the therapeutic agent, at least 85% by weight of the therapeutic agent, at least 90% by weight of the therapeutic agent, at least 95% by weight of the therapeutic agent, or 100% by weight of the therapeutic agent.

The therapeutic agent may be any of the therapeutic agents disclosed herein, for example in Section C (“Therapeutic Agents”) below.

In various embodiments of the depots 100 disclosed herein, the therapeutic region 200 may take several different forms. In some embodiments (for example, FIG. 4), the therapeutic region 200 may comprise a single layer comprised of a therapeutic agent, a therapeutic agent mixed with a bioresorbable polymer, or a therapeutic agent mixed with a bioresorbable polymer and a releasing agent. In some embodiments, the therapeutic region 200 itself may comprise a structure having multiple layers or sub-regions of therapeutic agent (and/or bioresorbable polymer and/or releasing agent). Some or all layers or sub-regions of such a multiple layer therapeutic region 200 may be directly adjacent (i.e., in contact with) one another (laterally or axially), and/or some or all layers or sub-regions may be spaced apart with one or more other regions therebetween (such as control region(s) 300 and/or barrier region(s))). In some embodiments, 2, 3, 4, 5, 6, 7, 8, 9, 10 or more therapeutic sub-regions or layers may be grouped together and spaced apart from another therapeutic region or group of therapeutic sub-regions or layers (having the same or different numbers of layers as the other group) with one or more other regions therebetween (such as control region(s) 300 and/or barrier region(s))) (see, for example, FIG. 5, FIG. 6, etc.).

In any of the depot embodiments disclosed herein, the ratio of the mass of the therapeutic agent in the depot to the mass of polymer in the depot is at least 3:1, 3.5:1, 4:1, 4.5:1, 5:1, 5.5:1, 6:1, 6.5:1, 7:1, 8:1, 9:1, 10:1, 11:1, 12:1, 13:1, 14:1, 15:1, or 16:1.

In any of the depot embodiments disclosed herein, the ratio of the mass of the polymer in the therapeutic region 200 to the mass of therapeutic agent in the therapeutic region 200 is at least 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, or 1:10.

In any of the embodiments disclosed herein, the weight ratio of releasing agent to polymer in the therapeutic region 200 may be 1:1, 1:1.5, 1:2, 1:3, 1:4, 1:5, 1:6, 1:7, 1:8, 1:9, 1:10, 1:11, 1:12, 1:13, 1:14, 1:15, or 1:16.

In some embodiments, the ratio of releasing agent to polymer to therapeutic agent in the therapeutic region 200 is of from about 0.1:10:20 to about 2:10:20, about 0.1:10:20 to about 1:10:20, about 0.1:10:20 to about 0.5:10:20, about 0.5:10:20 to about 0.1:10:20, or about 0.5:10:20 to about 1:10:20.

In any of the embodiments disclosed herein having a single therapeutic region 200, the therapeutic region 200 may have a thickness of from about 5 μm to 100 μm, 5 μm to 50 μm, 5 μm to 25 μm, 5 μm to 10 μm, 5 μm to 7 μm, 7 μm to 9 μm, 10 μm to 80 μm, 10 μm to 70 μm, 10 μm to 60 μm, 20 μm to 60 μm, 15 μm to 50 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, 100 μm to 2 mm, 100 μm to 1.5 mm, 100 μm to 1 mm, 100 μm to 200 μm, 200 μm to 300 μm, 300 μm to 400 μm, 400 μm to 500 μm, 500 μm to 600 μm, 600 μm to 700 μm, 700 μm to 800 μm, 800 μm to 900 μm, 900 μm to 1 mm, 1 mm to 1.5 mm, 200 μm to 600 μm, 400 μm to 1 mm, 500 μm to 1.1 mm, 800 μm to 1.1 mm, about 200 μm, about 300 μm, about 400 μm, about 500 μm, about 600 μm, about 700 μm, about 800 μm, about 900 μm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, or about 2 mm.

In those embodiments having multiple therapeutic regions and/or sub-regions, the individual sub-regions or combinations of some or all sub-regions may have a thickness of from about 5 μm to 100 μm, 5 μm to 50 μm, 5 μm to 25 μm, 5 μm to 10 μm, 5 μm to 7 μm, 7 μm to 9 μm, 10 μm to 80 μm, 10 μm to 70 μm, 10 μm to 60 μm, 20 μm to 60 μm, 15 μm to 50 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, 100 μm to 2 mm, 100 μm to 1.5 mm, 100 μm to 1 mm, 100 μm to 200 μm, 200 μm to 300 μm, 300 μm to 400 μm, 400 μm to 500 μm, 500 μm to 600 μm, 600 μm to 700 μm, 700 μm to 800 μm, 800 μm to 900 μm, 900 μm to 1 mm, 1 mm to 1.5 mm, 200 μm to 600 μm, 400 μm to 1 mm, 500 μm to 1.1 mm, 800 μm to 1.1 mm, about 200 μm, about 300 μm, about 400 μm, about 500 μm, about 600 μm, about 700 μm, about 800 μm, about 900 μm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, or about 2 mm.

The therapeutic regions 200 of the present technology may comprise at least 15% by weight of the therapeutic agent, at least 20% by weight of the therapeutic agent, at least at least 25% by weight of the therapeutic agent, at least 30% by weight of the therapeutic agent, at least 35% by weight of the therapeutic agent, at least 40% by weight of the therapeutic agent, at least 45% by weight of the therapeutic agent, at least 50% by weight of the therapeutic agent, at least 55% by weight of the therapeutic agent, at least 60% by weight of the therapeutic agent, at least 65% by weight of the therapeutic agent, at least 70% by weight of the therapeutic agent, at least 75% by weight of the therapeutic agent, at least 80% by weight of the therapeutic agent, at least 85% by weight of the therapeutic agent, at least 90% by weight of the therapeutic agent, at least 95% by weight of the therapeutic agent, or 100% by weight of the therapeutic agent.

In any of the embodiments disclosed herein, the therapeutic region 200 may include of from about 0.1%-10% by weight of the releasing agent, about 0.1%-6% by weight of the releasing agent, 0.2%-10% by weight of the releasing agent, about 0.3%-6% by weight of the releasing agent, about 0.1%-1% by weight of the releasing agent, about 0.1%-0.5% by weight of the releasing agent, 1%-2% by weight of the releasing agent, about 1%-3% by weight of the releasing agent, or about 2%-6% by weight of the releasing agent. In those embodiments having multiple therapeutic regions or sub-regions, one or more of the therapeutic regions or sub-therapeutic regions may individually include of from about 0.1%-10% by weight of the releasing agent, about 0.1%-6% by weight of the releasing agent, 0.2%-10% by weight of the releasing agent, about 0.3%-6% by weight of the releasing agent, about 0.1%-1% by weight of the releasing agent, about 0.1%-0.5% by weight of the releasing agent, 1%-2% by weight of the releasing agent, about 1%-3% by weight of the releasing agent, or about 2%-6% by weight of the releasing agent. The therapeutic region 200 may not include any releasing agent. In those embodiments having multiple therapeutic regions and/or sub-regions, one, some, or all of the individual therapeutic regions and/or sub-regions may not include any releasing agent.

In any of the embodiments disclosed herein, the therapeutic region 200 may include no more than 5% by weight of the polymer, no more than 10% by weight of the polymer, no more than 15% by weight of the polymer, no more than 20% by weight of the polymer, no more than 25% by weight of the polymer, no more than 30% by weight of the polymer, no more than 35% by weight of the polymer, no more than 40% by weight of the polymer, no more than 45% by weight of the polymer, or no more than 50% by weight of the polymer. In those embodiments having multiple therapeutic regions or sub-regions, one or more of the therapeutic regions or sub-therapeutic regions may individually include no more than 5% by weight of the polymer, no more than 10% by weight of the polymer, no more than 15% by weight of the polymer, no more than 20% by weight of the polymer, no more than 25% by weight of the polymer, no more than 30% by weight of the polymer, no more than 35% by weight of the polymer, no more than 40% by weight of the polymer, no more than 45% by weight of the polymer, or no more than 50% by weight of the polymer. In some embodiments, the therapeutic region 200 may not include any polymer.

In those embodiments disclosed herein where the therapeutic region 200 includes multiple therapeutic regions or sub-regions, some or all of the therapeutic regions or sub-therapeutic regions may have the same or different amounts of releasing agent, the same or different concentrations of releasing agent, the same or different releasing agents, the same or different amounts of polymer, the same or different polymers, the same or different polymer to releasing agent ratios, the same or different amounts of therapeutic agents, the same or different types of therapeutic agents, and/or the same or different thicknesses. Moreover, a single therapeutic region or sub-region may comprise a single type of polymer or multiple types of polymers, a single type of releasing agent or multiple types of releasing agents, and/or a single type of therapeutic agent or multiple types of therapeutic agents. In those embodiments having multiple therapeutic regions and/or sub-regions, one, some, or all of the individual therapeutic regions and/or sub-regions may not include any polymer.

In some embodiments the therapeutic region 200 (or one or more therapeutic sub-regions) comprises the therapeutic agent as an essentially pure compound or formulated with a pharmaceutically acceptable carrier such as diluents, adjuvants, excipients or vehicles known to one skilled in the art

C. Control Region

The composition of the control region 300 may also be varied. For example, in many embodiments, the control region 300 does not include any therapeutic agent at least prior to implantation of the depot at the treatment site. In some embodiments, the control region 300 may include a therapeutic agent which may be the same as or different than the therapeutic agent in the therapeutic region 200.

Within the control region 300, the amount of releasing agent may be varied to achieve a faster or slower release of the therapeutic agent. In those embodiments where both the therapeutic region 200 and control region 300 include a releasing agent, the type of releasing agent within the therapeutic region 200 may be the same or different as the releasing agent in the control region 300. In some embodiments, a concentration of a first releasing agent within the control region is the greater than a concentration of a second releasing agent (the same or different as the first releasing agent) within the therapeutic region. In some embodiments, a concentration of the releasing agent within the control region is less than a concentration of the releasing agent within the therapeutic region. In some embodiments, a concentration of the releasing agent within the control region 300 is the same as a concentration of the releasing agent within the therapeutic region 200.

In various embodiments of the depots disclosed herein, the control region 300 may take several different forms. In some embodiments (for example, FIG. 4), the control region 300 may comprise a single layer on either side of the therapeutic region 200 comprised of a bioresorbable polymer mixed with a releasing agent. In some embodiments, the control region 300 itself may comprise a structure having multiple layers or sub-regions of bioresorbable polymer and releasing agent. Some or all layers or sub-regions of such a multiple layer control region 300 may be directly adjacent (i.e., in contact with) one another (laterally or axially), and/or some or all layers or sub-regions may be spaced apart with one or more other regions therebetween (such as therapeutic region(s) 200 and/or barrier region(s))). In some embodiments, 2, 3, 4, 5, 6, 7, 8, 9, 10 or more control sub-regions or layers may be grouped together and spaced apart from another control region or group of control sub-regions or layers (having the same or different numbers of layers as the other group) with one or more other regions therebetween (such as therapeutic region(s) 200 and/or barrier region(s))) (see, for example, FIG. 5, FIG. 6, etc.).

Without being bound by theory, it is believed that such a multilayer configuration improves the control region's ability to control the release of the therapeutic agent as compared to a single layer control region, even if the multilayer configuration has the same or lower thickness as the single layer control region. The channels left by dissolution of the releasing agent in both microlayers and/or sub-regions of the control region create a path for a released therapeutic agent to travel that is longer and, potentially, more cumbersome to traverse as compared to the more direct path created by the channels in the single layer control region. The control region(s) and/or sub-regions thereby regulate the therapeutic agent release rate by allowing a releasing agent to form independent non-contiguous channels through one or more control regions and/or sub-regions. In those embodiments having multiple control layers or sub-regions, some or all of the control layers or sub-regions may be heat compressed together. The one or more control regions, heat-compressed first or not, may be heat compressed together with the therapeutic region 200. Having a control region 300 with multiple layers may provide a more linear, controlled release of the therapeutic agent over time (beyond the first day of implantation). In addition, layering of the control region 300 may also contribute to a more flexible, structurally competent depot (as compared to a depot having a therapeutic region comprised of pure therapeutic agent). Such durability is beneficial for the clinician when handling/manipulating the depot 100 before and while positioning the depot 100 at a treatment site.

In any of the embodiments disclosed herein having a single control region 300, the thickness of the control region 300 may be of from about 5 μm to 100 μm, 5 μm to 50 μm, 5 μm to 25 μm, 5 μm to 10 μm, 5 μm to 7 μm, 7 μm to 9 μm, 10 μm to 80 μm, 10 μm to 70 μm, 10 μm to 60 μm, 20 μm to 60 μm, 15 μm to 50 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, or about 100 μm. In those embodiments having multiple control regions and/or sub-regions, the individual sub-regions or combinations of some or all sub-regions may have a thickness of from about 5 μm to 100 μm, 5 μm to 50 μm, 5 μm to 25 μm, 5 μm to 10 μm, 5 μm to 7 μm, 7 μm to 9 μm, 10 μm to 80 μm, 10 μm to 70 μm, 10 μm to 60 μm, 20 μm to 60 μm, 15 μm to 50 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, or about 100 μm.

In any of the embodiments disclosed herein, the weight ratio of releasing agent to polymer in the control region 300 may be 2:1, 1.5:1, 1:1, 1:1.5, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, or 1:25.

In any of the embodiments disclosed herein, the control region 300 may include at least 5% by weight of the releasing agent, at least 10% by weight of the releasing agent, at least 15% by weight of the releasing agent, at least 20% by weight of the releasing agent, at least 25% by weight of the releasing agent, at least 30% by weight of the releasing agent, at least 35% by weight of the releasing agent, at least 40% by weight of the releasing agent, at least 45% by weight of the releasing agent, or at least 50% by weight of the releasing agent. In those embodiments having multiple control regions or sub-regions, one or more of the control regions or sub-control regions may individually include at least 5% by weight of the releasing agent, at least 10% by weight of the releasing agent, at least 15% by weight of the releasing agent, at least 20% by weight of the releasing agent, at least 25% by weight of the releasing agent, at least 30% by weight of the releasing agent, at least 35% by weight of the releasing agent, at least 40% by weight of the releasing agent, at least 45% by weight of the releasing agent, or at least 50% by weight of the releasing agent.

In any of the embodiments disclosed herein, the control region 300 may include at least 5% by weight of the polymer, at least 10% by weight of the polymer, at least 15% by weight of the polymer, at least 20% by weight of the polymer, at least 25% by weight of the polymer, at least 30% by weight of the polymer, at least 35% by weight of the polymer, at least 40% by weight of the polymer, at least 45% by weight of the polymer, at least 50% by weight of the polymer, at least 55% by weight of the polymer, at least 60% by weight of the polymer, at least 65% by weight of the polymer, at least 70% by weight of the polymer, at least 75% by weight of the polymer, at least 80% by weight of the polymer, at least 85% by weight of the polymer, at least 90% by weight of the polymer, at least 95% by weight of the polymer, or 100% by weight of the polymer. In those embodiments having multiple control regions or sub-regions, one or more of the control regions or sub-control regions may individually include at least 5% by weight of the polymer, at least 10% by weight of the polymer, at least 15% by weight of the polymer, at least 20% by weight of the polymer, at least 25% by weight of the polymer, at least 30% by weight of the polymer, at least 35% by weight of the polymer, at least 40% by weight of the polymer, at least 45% by weight of the polymer, at least 50% by weight of the polymer, at least 55% by weight of the polymer, at least 60% by weight of the polymer, at least 65% by weight of the polymer, at least 70% by weight of the polymer, at least 75% by weight of the polymer, at least 80% by weight of the polymer, at least 85% by weight of the polymer, at least 90% by weight of the polymer, at least 95% by weight of the polymer, or 100% by weight of the polymer.

In those embodiments disclosed herein where the control region 300 includes multiple control regions or sub-regions, some or all of the control regions or sub-control regions may have the same or different amounts of releasing agent, the same or different concentrations of releasing agent, the same or different releasing agents, the same or different amounts of polymer, the same or different polymers, the same or different polymer to releasing agent ratios, and/or the same or different thicknesses. A single control region or sub-region may comprise a single type of polymer or multiple types of polymers and/or a single type of releasing agent or multiple types of releasing agents.

D. Therapeutic Agents

The therapeutic agent carried by the depots 100 of the present technology may be any biologically active substance (or combination of substances) that provides a therapeutic effect in a patient in need thereof. As used herein, “therapeutic agent” or “drug” may refer to a single therapeutic agent, or may refer to a combination of therapeutic agents. In some embodiments, the therapeutic agent may include only a single therapeutic agent, and in some embodiments, the therapeutic agent may include two or more therapeutic agents for simultaneous or sequential release.

In several embodiments, the therapeutic agent includes an analgesic agent. The term “analgesic agent” or “analgesic” includes one or more local or systemic anesthetic agents that are administered to reduce, prevent, alleviate or remove pain entirely. The analgesic agent may comprise a systemic and/or local anesthetic, narcotics, and/or anti-inflammatory agents. The analgesic agent may comprise the pharmacologically active drug or a pharmaceutically acceptable salt thereof. Suitable local anesthetics include, but are not limited to, bupivacaine, ropivacaine, mepivacaine, etidocaine, levobupivacaine, trimecaine, carticaine, articaine, lidocaine, prilocaine, benzocaine, procaine, tetracaine, chloroprocaine, and combinations thereof. Preferred local anesthetics include bupivacaine, lidocaine, and ropivacaine. Typically, local anesthetics produce anesthesia by inhibiting excitation of nerve endings or by blocking conduction in peripheral nerves. Such inhibition is achieved by anesthetics reversibly binding to and inactivating sodium channels. Sodium influx through these channels is necessary for the depolarization of nerve cell membranes and subsequent propagation of impulses along the course of the nerve. When a nerve loses depolarization and capacity to propagate an impulse, the individual loses sensation in the area supplied by the nerve. Any chemical compound possessing such anesthetic properties is suitable for use in the present technology.

In some embodiments, the therapeutic agent includes narcotics, for example, cocaine, and anti-inflammatory agents. Examples of appropriate anti-inflammatory agents include steroids, such as prednisone, betamethasone, cortisone, dexamethasone, hydrocortisone, and methylprednisolone. Other appropriate anti-inflammatory agents include non-steroidal anti-inflammatory drugs (NSAIDs), such as aspirin, Ibuprofen, naproxen sodium, diclofenac, diclofenac-misoprostol, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, mefenamic acid, and other COX-2 inhibitors, and combinations thereof.

In some embodiments, the therapeutic agent comprises an antibiotic, an antimicrobial or antifungal agent or combinations thereof. For example, suitable antibiotics and antimicrobials include, but are not limited to, amoxicillin, amoxicillin/clavulanate, cephalexin, ciprofloxacin, clindamycin, metronidazole, azithromycin, levofloxacin, sulfamethoxazole/trimethoprim, tetracycline(s), minocycline, tigecycline, doxycycline, rifampin, triclosan, chlorhexidine, penicillin(s), aminoglycides, quinolones, fluoroquinolones, vancomycin, gentamycin, cephalosporin(s), carbapenems, imipenem, ertapenem, antimicrobial peptides, cecropin-mellitin,

WO 2020/210764 PCT/US2020/027852 magainin, dermaseptin, cathelicidin, α-defensins, and α-protegrins. Antifungal agents include, but are not limited to, ketoconazole, clortrimazole, miconazole, econazole, intraconazole, fluconazole, bifoconazole, terconazole, butaconazole, tioconazole, oxiconazole, sulconazole, saperconazole, voriconazole, terbinafine, amorolfine, naftifine, griseofulvin, haloprogin, butenafine, tolnaftate, nystatin, cyclohexamide, ciclopirox, flucytosine, terbinafine, and amphotericin B.

In several embodiments, the therapeutic agent may be an adrenocorticostatic, a β-adrenolytic, an androgen or antiandrogen, an antianemic, a antiparasitic, an anabolic, an anesthetic or analgesic, an analeptic, an antiallergic, an antiarrhythmic, an anti-arteriosclerotic, an antibiotic, an antidiabetic, an antifibrinolytic, an anticonvulsive, an angiogenesis inhibitor, an anticholinergic, an enzyme, a coenzyme or a corresponding inhibitor, an antihistaminic, an antihypertensive, an antihypotensive, an anticoagulant, an antimycotic, an antiseptic, an anti-infective, an antihemorrhagic, a β-receptor antagonist, a calcium channel antagonist, an antimyasthenic, an antiphlogistic, an antipyretic, an antirheumatic, a cardiotonic, a chemotherapeutic, a coronary dilator, a cytostatic, a glucocorticoid, a hemostatic, an immunoglobulin or its fragment, a chemokine, a cytokine, a mitogen, a cell differentiation factor, a cytotoxic agent, a hormone, an immunosuppressant, an immunostimulant, a morphine antagonist, an muscle relaxant, a narcotic, a vector, a peptide, a (para)sympathicomimetic, a (para)sympatholytic, a protein, a cell, a selective estrogen receptor modulator (SERM), a sedating agent, an antispasmodic, a substance that inhibits the resorption of bone, a vasoconstrictor or vasodilator, a virustatic or a wound-healing agent.

In various embodiments, the therapeutic agent comprises a drug used in the treatment of cancer or a pharmaceutically acceptable salt thereof. Such chemotherapeutic agents include antibodies, alkylating agents, angiogenesis inhibitors, antimetabolites, DNA cleavers, DNA crosslinkers, DNA intercalators, DNA minor groove binders, enediynes, heat shock protein 90 inhibitors, histone deacetylase inhibitors, immunomodulators, microtubule stabilizers, nucleoside (purine or pyrimidine) analogs, nuclear export inhibitors, proteasome inhibitors, topoisomerase (I or II) inhibitors, tyrosine kinase inhibitors, and serine/threonine kinase inhibitors. Specific therapeutic agents include, but are not limited to, adalimumab, ansamitocin P3, auristatin, bendamustine, bevacizumab, bicalutamide, bleomycin, bortezomib, busulfan, callistatin A, camptothecin, capecitabine, carboplatin, carmustine, cetuximab, cisplatin, cladribin, cytarabin, cryptophycins, dacarbazine, dasatinib, daunorubicin, docetaxel, doxorubicin, duocarmycin, dynemycin A, epothilones, etoposide, floxuridine, fludarabine, 5 -fluorouracil, gefitinib, gemcitabine, ipilimumab, hydroxyurea, imatinib, infliximab, interferons, interleukins, beta-lapachone, lenalidomide, irinotecan, maytansine, mechlorethamine, melphalan, 6-mercaptopurine, methotrexate, mitomycin C, nilotinib, oxaliplatin, paclitaxel, procarbazine, sub eroylanilide hydroxamic acid (SAHA), 6-thioguanidine, thiotepa, teniposide, topotecan, trastuzumab, trichostatin A, vinblastine, vincristine, vindesine, and tamoxifen.

In some embodiments, the therapeutic agent comprises a botulinum toxin (or neurotoxin) drug used in the treatment of various neuromuscular and/or neuroglandular disorders and neuropathies associated with pain. The botulinum toxin (or neurotoxin) may comprise the pharmacologically active drug or a pharmaceutically acceptable salt thereof. The botulinum toxin (or neurotoxin) as described and used herein may be selected from a variety of strains of Clostridium botulinum and may comprise the pharmacologically active drug or a pharmaceutically acceptable salt thereof. In one embodiment, the botulinum toxin is selected from the group consisting of botulinum toxin types A, B, C, D, E, F and G. In a preferred embodiment, the botulinum toxin is botulinum toxin type A. Commercially available botulinum toxin, BOTOX® (Allergan, Inc., Irvine, Calif.), consists of a freeze-dried, purified botulinum toxin type A complex, albumin and sodium chloride packaged in sterile, vacuum-dried form.

The paralytic effect of botulinum toxin is the most common benefit of commercial therapeutics, where muscles are relaxed in order to treat muscle dystonias, wrinkles and the like. However, it has been shown that in addition to its anti-cholinergic effects on muscle and smooth muscle, the neurotoxin can have therapeutic effects on other non-muscular cell types, and on inflammation itself. For example, it has been shown that cholinergic goblet cells, which produce mucus throughout the airway system, react to and can be shut down by introduction of botulinum toxin. Research also shows that botulinum toxin has direct ant-inflammatory capabilities. All of these therapeutic effects, muscle, smooth muscle, goblet cell and anti-inflammatory affects, may be derived from delivery of the toxin from the inventive devices.

A pharmaceutically acceptable salt refers to those salts that retain the biological effectiveness and properties of neutral therapeutic agents and that are not otherwise unacceptable for pharmaceutical use. Pharmaceutically acceptable salts include salts of acidic or basic groups, which groups may be present in the therapeutic agents. The therapeutic agents used in the present technology that are basic in nature are capable of forming a wide variety of salts with various inorganic and organic acids. Pharmaceutically acceptable acid addition salts of basic therapeutic agents used in the present technology are those that form non-toxic acid addition salts, i.e., salts comprising pharmacologically acceptable anions, such as the hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The therapeutic agents of the present technology that include an amino moiety may form pharmaceutically acceptable salts with various amino acids, in addition to the acids mentioned above. Suitable base salts are formed from bases which form non-toxic salts and examples are the aluminum, calcium, lithium, magnesium, potassium, sodium, zinc and diethanolamine salts.

A pharmaceutically acceptable salt may involve the inclusion of another molecule such as water or another biologically compatible solvent (a solvate), an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

The therapeutic agent or pharmaceutically acceptable salt thereof may be an essentially pure compound or be formulated with a pharmaceutically acceptable carrier such as diluents, adjuvants, excipients or vehicles known to one skilled in the art. The carrier(s) must be “acceptable” in the sense of being compatible with the other ingredients of the formulations and not deleterious to the recipient thereof. For example, diluents include lactose, dextrose, sucrose, mannitol, sorbitol, cellulose, glycine and the like. For examples of other pharmaceutically acceptable carriers, see Remington: THE SCIENCE AND PRACTICE OF PHARMACY (21st Edition, University of the Sciences in Philadelphia, 2005).

The therapeutic agent or pharmaceutically acceptable salt form may be jet milled or otherwise passed through a sieve to form consistent particle sizes further enabling the regulated and controlled release of the therapeutic agent. This process may be particularly helpful for highly insoluble therapeutic agents.

An important criterion for determining the amount of therapeutic agent needed for the treatment of a particular medical condition is the release rate of the drug from the depot of the present technology. The release rate is controlled by a variety of factors, including, but not limited to, the rate that the releasing agent dissolves in vivo into the surrounding fluid, the in vivo degradation rate of the bioresorbable polymer or copolymer utilized. For example, the rate of release may be controlled by the use of multiple control regions between the therapeutic region and the physiological fluid. See, for example, FIGS. 6-8.

Suitable dosage ranges utilizing the depot of the present technology are dependent on the potency of the particular therapeutic agent, but are generally about 0.001 mg to about 500 mg of drug per kilogram body weight, for example, from about 0.1 mg to about 200 mg of drug per kilogram body weight, and about 1 to about 100 mg/kg-body wt. per day. Dosage ranges may be readily determined by methods known to one skilled in the art.

In some aspects of the technology, the therapeutic region 200 may include multiple layers. In such embodiments, the multiple layers may improve efficient loading of therapeutic agents. For example, multilayering may be a direct and effective way of loading substantial amounts of therapeutic agent. It can often be challenging to load a large amount of therapeutic agent in a single film layer, even by increasing the drug to polymer ratio or increasing the thickness of the layer. Even when the thickness of the therapeutic region can be theoretically increased to load more drug, consistent fabrication of a thick therapeutic region via casting could prove to be a challenge. In contrast, the stacking and bonding of thin films or sheets, each with a predetermined load of therapeutic agent, may present as a more reliable casting alternative. Data from an example of loading an analgesic (i.e., ropivacaine) is provided in Table 2.

TABLE 2

| | Drug load (ug) | Thickness (mm) |
|---|---|---|
| Single layer | 212.66 | 0.019 |
| Five layers | 1120.83 | 0.046 |
| Multiple | 5.27 | 2.42 |

As but one example, a single layer loaded with ropivacaine and having a thickness of 0.019 mm was produced. A 5-layer film sample, where each layer was loaded with ropivacaine, having a thickness of 0.046 mm was also produced. Even though the thickness of the 5-layer film sample was only 2.42 times the thickness of the single layer, the load of therapeutic agent in the 5-layer sample was 5.27 times that of the single layer sample. Accordingly, the multilayering approach enabled a substantially higher density of therapeutic agent.

As described above, heat compression bonding of multiple layers enables an effective reduction in film thickness and an increased density of therapeutic agent loading. In the example illustrated in Table 2, the multilayer structure enabled a 124% increase in the density of the therapeutic agent. In other embodiments, the increase in density of the therapeutic agent enabled by a multilayer structure of the therapeutic region may be approximately 50%, 75%, 100%, 125%, 150% or 200%.

E. Polymers

The depots 100 of the present technology are comprised of bioresorbable polymers. In some embodiments, both the therapeutic region 200 and the control region 300 comprise a polymer (or mix of polymers), which can be the same or different polymer (or mix of polymers) in the same or different amount, concentration, and/or weight percentage. In some embodiments, the control region 300 comprises a polymer and the therapeutic region 200 does not include a polymer. In some embodiments, the therapeutic region 200 comprises a polymer and the control region 300 does not include a polymer. At least as used in this section, “the polymer” applies to a polymer that may be used in the therapeutic region 200 and/or in the control region 300.

The bioresorbable polymers used in the present technology preferably have a predetermined degradation rate. The terms “bioresorbable,” or “bioabsorbable,” mean that a polymer will be absorbed within the patient's body, for example, by a cell or tissue. These polymers are “biodegradable” in that all or parts the polymeric film will degrade over time by the action of enzymes, by hydrolytic action and/or by other similar mechanisms in the patient's body. In various embodiments, the bioresorbable polymer film can break down or degrade within the body to non-toxic components while a therapeutic agent is being released. Polymers used as base components of the depots of the present technology may break down or degrade after the therapeutic agent is fully released. The bioresorbable polymers are also “bioerodible,” in that they will erode or degrade over time due, at least in part, to contact with substances found in the surrounding tissue, fluids or by cellular action.

Criteria for the selection of the bioresorbable polymer suitable for use in the present technology include: 1) in vivo safety and biocompatibility; 2) therapeutic agent loading capacity; 3) therapeutic agent releasing capability; 4) degradation profile; 5) potential for inflammatory response; and 6) mechanical properties, which may relate to form factor and manufacturability. As such, selection of the bioresorbable polymer may depend on the clinical objectives of a particular therapy and may involve trading off between competing objectives. For example, PGA (polyglycolide) is known to have a relatively fast degradation rate, but it is also fairly brittle. Conversely, polycaprolactone (PCL) has a relatively slow degradation rate and is quite elastic. Copolymerization provides some versatility if it is clinically desirable to have a mix of properties from multiple polymers. For biomedical applications, particularly as a bioresorbable depot for drug release, a polymer or copolymer using at least one of poly(L-lactic acid) (PLA), PCL, and PGA are generally preferred. The physical properties for some of these polymers are provided in Table 3 below.

TABLE 3

| | | | Elastic | Tensile | Tensile | Degradation |
|---|---|---|---|---|---|---|
| | Tg | Tm | Modulus | Strength | Elongation | Time |
| Materials | (° C.) | (° C.) | (GPa) | (MPa) | (%) | (months) |
| PLA | 45-60 | 150-162 | 0.35-3.5 | 21-60 | 2.5-6 | 12-16 |
| PLLA | 55-65 | 170-200 | 2.7-4.14 | 15.5-150 | 3-10 | >24 |
| PDLA | 50-60 | — | 1.0-3.45 | 27.6-50 | 2-10 | 6-12 |
| PLA/PGA | 40-50 | — | 1.0-4.34 | 41.4-55.2 | 2-10 | 3 |
| (50:50) | | | | | | |
| PGA | 35-45 | 220-233 | 6.0-7.0 | 60-99.7 | 1.5-20 | 6-12 |
| PCL | −60-−65 | 58-65 | 0.21-0.44 | 20.7-42 | 300-1000 | >24 |

In many embodiments, the polymer may include polyglycolide (PGA). PGA is one of the simplest linear aliphatic polyesters. It is prepared by ring opening polymerization of a cyclic lactone, glycolide. It is highly crystalline, with a crystallinity of 45-55%, and thus is not soluble in most organic solvents. It has a high melting point (220-225° C.), and a glass transition temperature of 35-40° C. (Vroman, L., et al., Materials, 2009, 2:307-44). Rapid in vivo degradation of PGA leads to loss of mechanical strength and a substantial local production of glycolic acid, which in substantial amounts may provoke an inflammatory response.

In many embodiments, the polymer may include polylactide (PLA). PLA is a hydrophobic polymer because of the presence of methyl (—CH3) side groups off the polymer backbone. It is more resistant to hydrolysis than PGA because of the steric shielding effect of the methyl side groups. The typical glass transition temperature for representative commercial PLA is 63.8° C., the elongation at break is 30.7%, and the tensile strength is 32.22 MPa (Vroman, 2009). Regulation of the physical properties and biodegradability of PLA can be achieved by employing a hydroxy acids co-monomer component or by racemization of D- and L-isomers (Vroman, 2009). PLA exists in four forms: poly(L-lactic acid) (PLLA), poly(D-lactic acid) (PDLA), meso-poly(lactic acid) and poly(D,L-lactic acid) (PDLLA), which is a racemic mixture of PLLA and PDLA. PLLA and PDLLA have been the most studied for biomedical applications.

Copolymerization of PLA (both L- and D,L-lactide forms) and PGA yields poly(lactide-co-glycolide) (PLGA), which is one of the most commonly used degradable polymers for biomedical applications. In many embodiments, the polymer may include PLGA. Since PLA and PGA have significantly different properties, careful choice of PLGA composition can enable optimization of performance in intended clinical applications. Physical property modulation is even more significant for PLGA copolymers. When a composition is comprised of 25-75% lactide, PLGA forms amorphous polymers which are very hydrolytically unstable compared to the more stable homopolymers. This is demonstrated in the degradation times of 50:50 PLGA, 75:25 PLGA, and 85:15 PLGA, which are 1-2 months, 4-5 months and 5-6 months, respectively. In some embodiments, the polymer may be an ester-terminated poly (DL-lactide-co-glycolide) in a molar ratio of 50:50 (DURECT Corporation).

In some embodiments, the polymer may include polycaprolactone (PCL). PCL is a semi-crystalline polyester with high organic solvent solubility, a melting temperature of 55-60° C., and glass transition temperature of −54° C. (Vroman, 2009). PCL has a low in vivo degradation rate and high drug permeability, thereby making it more suitable as a depot for longer term drug delivery. For example, Capronor® is a commercial contraceptive PCL product that is able to deliver levonorgestrel in vivo for over a year. PCL is often blended or copolymerized with other polymers like PLLA, PDLLA, or PLGA. Blending or copolymerization with polyethers expedites overall polymer erosion. Additionally, PCL has a relatively low tensile strength (-23 MPa), but very high elongation at breakage (4700%), making it a very good elastic biomaterial. PCL also is highly processable, which enables many potential form factors and production efficiencies.

Suitable bioresorbable polymers and copolymers for use in the present technology include, but are not limited to, poly(alpha-hydroxy acids), poly(lactide-co-glycolide)(PLGA or DLG), poly(DL-lactide-co-caprolactone) (DL-PLCL), polycaprolactone (PCL), poly(L-lactic acid) (PLA), poly(trimethylene carbonate) (PTMC), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(glycolide-co-carolactone) (PGCL), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (such as hyaluronic acid, chitosan and starch), proteins (such as gelatin and collagen) or PEG derivatives and copolymers thereof. Other suitable polymers or copolymers include polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAM (sucrose acetate isobutyrate)hydroxypropyl cellulose, hydroxypropyl methylcellulose, hydroxyethyl methylcellulose, carboxymethylcellulose or salts thereof, Carbopol®, poly(hydroxyethylmethacrylate), poly(methoxyethylmethacrylate), poly(methoxyethoxy-ethylmethacrylate), polymethylmethacrylate (PMMA), methylmethacrylate (MMA), gelatin, polyvinyl alcohols, propylene glycol, or combinations thereof.

In various embodiments, the molecular weight of the polymer can be a wide range of values. The average molecular weight of the polymer can be from about 1000 to about 10,000,000; or about 1,000 to about 1,000,000; or about 5,000 to about 500,000; or about 10,000 to about 100,000; or about 20,000 to 50,000.

As described above, it may be desirable in certain clinical applications using depots for controlled delivery of therapeutic agents to use copolymers comprising at least two of PGA, PLA, PCL, PDO, and PVA. These include, for example, poly(lactide-co-caprolactone) (PLCL) (e.g. having a PLA to PCL ratio of from 90:10 to 60:40) or its derivatives and copolymers thereof, poly(DL-lactide-co-caprolactone) (DL-PLCL) (e.g. having a DL-PLA to PCL ratio of from 90:10 to 50:50) or its derivatives and copolymers thereof, poly(glycolide-co-caprolactone) (PGCL) (e.g. having a PGA to PCL ratio of from 90:10 to 10:90) or its derivatives and copolymers thereof, or a blend of PCL and PLA (e.g. a ratio blend of PCL and PLA having a wt:wt ratio of 1:9 to 9:1). In one preferred embodiment, the bioresorbable polymer comprises a copolymer of polycaprolactone (PCL), poly(L-lactic acid) (PLA) and polyglycolide (PGA). In such a preferred embodiment, the ratio of PGA to PLA to PCL of the copolymer may be 5-60% PGA, 5-40% PLA and 10-90% PCL. In additional embodiments, the PGA:PLA:PCL ratio may be 40:40:20, 30:30:50, 20:20:60, 15:15:70, 10:10:80, 50:20:30, 50:25:25, 60:20:20, or 60:10:30. In some embodiments, the polymer is an ester-terminated poly (DL-lactide-co-glycolide-co-caprolactone) in a molar ratio of 60:30:10 (DURECT Corporation).

In some embodiments, a terpolymer may be beneficial for increasing the degradation rate and ease of manufacturing, etc.

To minimize the size of a bioresorbable depot, it is generally preferred to maximize the loading of therapeutic agent in the polymer to achieve the highest possible density of therapeutic agent. However, polymer carriers having high densities of therapeutic agent are more susceptible to burst release kinetics and, consequently, poor control over time release. As described above, one significant benefit of the depot structure described herein, and particularly the control region feature of the depot, is the ability to control and attenuate the therapeutic agent release kinetics even with therapeutic agent densities that would cause instability in other carriers. In certain embodiments, the therapeutic agent loading capacity includes ratios (wt:wt) of the therapeutic agent to bioresorbable polymer of approximately 1:3, 1:2, 1:1, 3:2, 2:1, 3:1, 4:1, 5:1, 6:1, 7:1, 8:1, 9:1, 10:1, 12:1, 14:1, or 16:1. In some embodiments, it may be desirable to increase the therapeutic effect or potency of the therapeutic agent released from the depot described herein while still maintaining the same or similar polymer to therapeutic agent ratio. This can be accomplished by using an essentially pure form of the therapeutic agent as opposed to a salt derivative. Additionally or alternatively, the therapeutic agent can be mixed with clonidine or epinephrine, which are known to increase the therapeutic effect of certain drugs.

In some embodiments, the bioresorbable polymer used in various layers of the depot may manifest as a layer of electrospun microfibers or nanofibers. Biocompatible electrospun microfibers/nanofibers are known in the art and may be used, for example, to manufacture implantable supports for the formation of replacement organs in vivo (U.S. Patent Publication No. 2014/0272225; Johnson; Nanofiber Solutions, LLC), for musculoskeletal and skin tissue engineering (R. Vasita and D.S. Katti, Int. J. Nanomedicine, 2006, 1:1, 15-30), for dermal or oral applications (PCT Publication No. 2015/189212; Hansen; Dermtreat APS) or for management of postoperative pain (U.S. Patent Publication No. 2013/0071463; Palasis et al.). As a manufacturing technique, electrospinning offers the opportunity for control over the thickness and the composition of the nano- or micro-fibers along with control of the porosity of the fiber meshes (Vasita and Katti, 2006). These electrospun scaffolds are three-dimensional and thus provide ideal supports for the culture of cells in vivo for tissue formation. Typically, these scaffolds have a porosity of 70-90% (U.S. Pat. No. 9,737,632; Johnson; Nanofiber Solutions, LLC). Suitable bioresorbable polymers and copolymers for the manufacture of electrospun microfibers include, but are not limited to, natural materials such as collagen, gelatin, elastin, chitosan, silk fibrion, and hyaluronic acid, as well as synthetic materials such as poly(c-caprolactone) (PCL), poly(glycolic acid) (PGA), poly(lactic-co-glycolic acid) (PLGA), poly(l-lactide-co-ε-caprolactone), and poly(lactic acid) (PLA).

Electrospun microfibers that are made from a bioresorbable polymer or copolymer and have been used in conjunction with a therapeutic agent are known in the art. For example, Johnson et al. have disclosed the treatment of joint inflammation and other conditions with an injection of biocompatible polymeric electrospun fiber fragments along with a carrier medium containing chitosan (U.S. Published Application No. 2016/0325015; Nanofiber Solutions, LLC). Weldon et al. reported the use of electrospun bupivacaine-eluting sutures manufactured from poly(lactic-co-glycolic acid) in a rat skin wound model, wherein the sutures provided local anesthesia at an incision site (J. Control Release, 2012, 161:3, 903-909). Similarly, Palasis et al. disclosed the treatment of postoperative pain by implanting electrospun fibers loaded with an opioid, anesthetic or a non-opioid analgesic within a surgical site (U.S. Patent Publication No. 2013/0071463; Palasis et al.). Electrospun microfibers suitable for use in the present technology may be obtained by the methods disclosed in the above cited references, which are herein incorporated in their entirety.

When implanted in a patient's body, the bioresorbable depot described above may be subject to articulation, pinching, or other movement or impinging forces throughout the duration of release. So as to avoid premature release of the therapeutic agent, it is desirable for the depot to have a threshold level of mechanical integrity and stability until most of the therapeutic agent has been released. While it may be desirable to maximize the loading of therapeutic agent in the bioresorbable depot, as described above, such maximization can typically be at the expense of mechanical integrity and stability of the depot. It is desirable for the depot described herein to have a high density loading of therapeutic agent while still maintaining sufficient mechanical integrity and stability in the body. The layered structure and, particularly, the presence of the control region provide some safeguard against the premature release of therapeutic agent. Moreover, the use of heat compression in the manufacturing process enables substantial loading of therapeutic agent into the therapeutic region while creating a thermal bond between the therapeutic region and control region, thereby preventing delamination, and a consequent uncontrolled release of drug, if and when the depot is subjected to mechanical stress in the body.

It is generally desirable that the implanted polymer fully degrade following complete delivery of the therapeutic agent. Full degradation is preferred because, unless the implanted polymer provides some structural function or support, the clinical practitioner would have to reconcile leaving in a foreign body with no functional purpose, which could be a source of inflammation or infection, or perform another surgery simply to remove the remaining polymer. As an alternative to full degradation, it would be desirable for any remaining polymer to be fully encapsulated by the body.

The degradation of an implanted polymer consists essentially of two sequential processes: diffusion of an aqueous solution (e.g., physiological fluids) followed by hydrolytic degradation. Degradation usually takes one of two forms: (1) surface erosion; and (2) bulk degradation. Surface erosion of a polymer occurs when the polymer erodes from the surface inward, where hydrolytic erosion at the surface is faster than the ingress of water into the polymer. Conversely, bulk degradation occurs throughout the entire polymer, where water penetrates and degrades the interior of the material faster than the surface can erode. Polymers such as PLA, PGA, PLGA and PCL all resorb into the body via bulk degradation.

The time necessary for complete degradation can vary greatly based on the material selected and the clinical performance requirements of the depot. For example, in treating some conditions, it may be desirable for the polymer depot to release a first therapeutic agent for anywhere from 5 to 30 days. In the case of treating or preventing other conditions, it may be desirable for the polymer depot to release a second therapeutic agent for anywhere from 2 to 4 months. Alternatively, even if the entire amount of therapeutic agent loaded into the polymer has been released, it may be desirable for the polymer to degrade over a longer period than the duration of drug release. For example, rapid degradation can often make the polymer brittle and fragile, thereby compromising mechanical performance, or provoking an inflammatory response from the body. In particular, it may be desirable, in certain clinical applications, to have an embodiment wherein degradation of the polymer commenced only after release of substantially all of the therapeutic agent.

In certain embodiments of the present technology, it may be desirable for the polymer to fully resorb into the body after substantially all therapeutic agent loaded therein is released. In certain embodiments, this degradation can be as short as 1 month. Alternatively, in other embodiments, full degradation could take as long as 2 months, 3 months, 4 months, 6 months, 9 months or 12 months. In some embodiments, the bioresorbable polymer substantially degrades in vivo within about one month, about two months, about three months, about four months, about five months or about six months. In some embodiments, it may be desirable for full degradation to be 6 months such that the mechanical properties of the implanted polymer are preserved for the first 2 months following implantation.

### Core Acidification

Traditional bioresorbable implants often lead to tissue inflammation due to a phenomenon known as “core acidification.” For example, as shown schematically in FIG. 49, polymer implants having a thickness greater than 1 mm degrade by bulk erosion (i.e., degradation occurs throughout the whole material equally; both the surface and the inside of the material degrade at substantially the same time). As the polymer degrades, lactate accumulates at an internal region of the implant. Eventually, because of the high pH in the internal region of the implant, the lactate becomes lactic acid. The accumulated lactic acid will invariably release into the body, thereby provoking an inflammatory response. FIG. 51, for example, is a scanning electron microscope (“SEM”) image of a polymer tablet of the prior art after 20 days of degradation. Inflammation in and around a prosthetic joint may be particularly concerning because of the risk of inflammation-induced osteolysis, which may cause a loosening of the newly implanted joint.

The degree of core acidification is determined in large part by the geometry and dimensions of the polymer implant. (See, e.g., Grizzi et al., Hydrolytic degradation of devices based on poly(dl-lactic acid) size-dependence, BIOMATERIALS, 1995, Vol. 16 No. 4, pp. 305-11; Fukuzaki et al., in vivo characteristics of high molecular weight copoly(l-lactide/glycolide) with S-type degradation pattern for application in drug delivery systems, Biomaterials 1991, Vol. 12 May, pp. 433-37; Li et al., Structure-property relationships in the case of degradation of massive alipathic poly-(α-hydroxy acids) in aqueous media, JOURNAL OF MATERIALS SCIENCE: MATERIALS IN MEDICINE I (1990), pp. 123-130.) For example, degradation in more massive monolithic devices (mm-size scales and greater) proceeds much more rapidly in their interior than on their surface, leading to an outer layer of slowly degrading polymer entrapping more advanced internal degradation products from interior zone autocatalysis (so-called “S-type” non-linear kinetic degradation profile.). In contrast to a thicker film, a thin film of less than 1 mm thickness will typically degrade via surface erosion, wherein the lactate resulting from degradation will not accumulate in the interior of the film. Thin films, because of their high surface area to volume ratios, are known to degrade uniformly and do not lead to core acidification. (See Grizzi et al.)

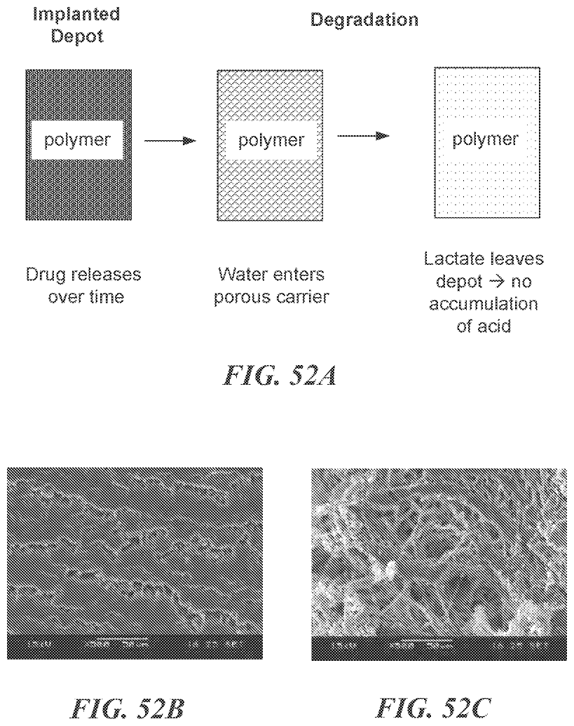

As shown schematically in FIG. 52A, the depots of the present technology may shed up to 50%, 60%, 70% or 80% of their individual mass (anesthetic and releasing agent) over the course of releasing the anesthetic (e.g., 5 days, 7 days, 10 days, 14 days, 20 days, 30 days, 60 days, 90 days, 180 days, etc.), resulting in a highly porous, mesh-like system that—at least for the purpose of degradation—behaves like a thin-film because of its high surface area to volume ratio. Body fluids will invade the highly porous polymer carrier to degrade the remaining polymer via surface erosion, thereby avoiding core acidification and the resulting inflammatory response. Without being bound by theory, it is believed that the drug core matrix of the therapeutic region becomes highly porous as degradation continues. For example, FIGS. 52B and 52C are scanning electron microscope (“SEM”) images showing the therapeutic region before and after elution, respectively. However, even after the release of therapeutic agent, there is still a clear porous structure left through which water and acid can diffuse effectively. Thus, depots 100 of the present technology having a thickness greater than about 1 mm degrade like a thin film, and surprisingly do not exhibit core acidification.

F. Releasing Agent

In many implantable drug eluting technologies, the depot provides an initial, uncontrolled burst release of drug followed by a residual release. These drug release kinetics may be desirable in certain clinical applications, but may be unavoidable even when undesirable. Hydrophilic drugs loaded in a polymer carrier will typically provide a burst release when exposed to physiologic fluids. This dynamic may present challenges, particularly when it is desirable to load a large volume of drug for controlled, sustained in vivo administration. For example, although it may be desirable to implant several days or weeks' worth of dosage to achieve a sustained, durable, in vivo pharmacological treatment, it is imperative that the therapeutic agent is released as prescribed, otherwise release of the entire payload could result in severe complications to the patient.

To achieve finer control over the release of the therapeutic agent when exposed to fluids, the depots 100 of the present technology may include a releasing agent. In some embodiments, both the therapeutic region 200 and the control region 300 include a releasing agent (or mix of releasing agents), which can be the same or different releasing agent (or mix of releasing agents) in the same or different amount, concentration, and/or weight percentage. In some embodiments, the control region 300 includes a releasing agent and the therapeutic region 200 does not include a releasing agent. In some embodiments, the therapeutic region 200 includes a releasing agent and the control region 300 does not include a releasing agent. At least as used in this section, “the releasing agent” applies to a releasing agent that may be used in the therapeutic region 200 and/or in the control region 300.

The type and/or amount of releasing agent within the therapeutic region 200 and/or control region 300 may be varied according to the desired release rate of the therapeutic agent into the surrounding biological fluids. For example, choosing releasing agents with different dissolution times will affect the rate of release. Also, the weight percentage of releasing agent in a region of polymer will influence the number and the size of the diffusion openings subsequently formed in the polymer, thereby affecting the rate of therapeutic agent release from the depot 100 (e.g., the greater the weight percentage of releasing agent, the faster the release). The presence of releasing agent in select regions also influences the release rate of therapeutic agent. For example, a depot with releasing agent in the control region 300 and/or therapeutic region 200 will generally release therapeutic agent at a higher rate compared to a depot with no releasing agent. Similarly, releasing agent in both the control region 300 and the therapeutic region 200 will generally release therapeutic agent at a higher rate than when releasing agent is in the control region alone.

In certain embodiments of the present technology, the layer-by-layer ratio of releasing agent to bioresorbable polymer can be adjusted to control the rate of therapeutic agent released from the depot 100. For example, in many embodiments of the present technology, the depot 100 includes a therapeutic region 200 having a weight percentage of releasing agent that is different than the weight percentage of the releasing agent in the control region 200. For example, the therapeutic region 200 may have a greater or lesser weight percentage of releasing agent than the control region 300. In some embodiments, the control region 300 may have a weight percentage of releasing agent that is at least 2 times greater than the weight percentage of the releasing agent in the therapeutic region 200. In some embodiments, the control region 300 may have a weight percentage of releasing agent that is at least 3-20 times greater, at least 4 times greater, at least 5 times greater, at least 6 times greater, at least 7 times greater, at least 8 times greater, at least 9 times greater, at least 10 times greater, at least 11 times greater, at least 12 times greater, at least 13 times greater, at least 14 times greater, at least 16 times greater, at least 17 times greater, at least 18 times greater, at least 19 times greater, at least 20 times greater, at least 25 times greater, at least 30 times greater, about 5 to 10 times greater, about 10 to 15 times greater, about 5 to 15 times greater, or about 15 to 25 times greater than the weight percentage of the releasing agent in the therapeutic region 200.

In many embodiments of the present technology, the releasing agent is a surfactant. Unlike the use as a releasing agent as described herein, surfactants are usually used to control the dispersions, flocculation and wetting properties of a drug or polymer. Fundamentally, surfactants operate on the interface between the polymer and drug or the interface between the drug and biological membrane. Depending on the type of formulation, surfactants typically play a role in several aspects of drug delivery: (1) solubilization or stabilization of hydrophobic drugs by lowering the entropic cost of solvating hydrophobic drug through complexation with drug molecules in solution (C. Bell and K. A. Woodrow, ANTIMICROB. AGENTS CHEMOTHER., 2014, 58:8, 4855-65); (2) improvement of the wetting of tablet or polymer for fast disintegration (M. Irfan, et al., SAUDI PHARM. J., 2016, 24, 537-46); (3) formation of colloidal drug delivery systems, such as reverse micelles, vesicles, liquid crystal dispersions, nanoemulsions and nanoparticles (M. Fanun, Colloids in Drug Delivery, 2010, p. 357); and (4) improvement the bioperformance of drugs by altering the permeability of biological membrane and consequently drug penetration/permeation profile (S. Jain, et al., Lipid Based Vesicular Drug Delivery Systems, 2014, Vol. 2014, Article ID 574673).

In order to illustrate the unique aspects of using a releasing agent in the polymeric control region to form diffusion openings and/or microchannels in the present technology, it is helpful to explain the more common approach of using hydrophilic molecules to enhance drug release. Conventionally, drug release is enhanced by creating a larger surface area in order to increase contact between the drug and the bodily fluid, thereby accelerating drug release. The most common mechanism for forming pores prior to implantation is to use non-surfactant hydrophilic molecules as pore-forming agents in polymer layers, either as a coating layer or a free-standing film (Kanagale, P., et al., AAPS PHARM. SCI.TECH., 2007; 8(3), E1-7). Usually, pores are pre-formed by blending hydrophilic molecules with polymer, then removing the hydrophilic molecules by contact with water. However, when hydrophilic molecules are blended with hydrophobic polymer, the molecules tend to form hydrophilic domains and hydrophobic domains, which are energetically favorable due to the increase in entropy. When the film contacts water, hydrophilic domains are removed and replaced with large pores. The rate of drug release in this case is solely controlled by the porosity of the film and the resulting increased total surface area. The typical drug release curve in this case has a high, uncontrolled initial burst followed with a very slow release of residual drug afterwards.

Previously, when non-surfactant hydrophilic molecules are mixed into the polymer and then removed, a film with a porous structure is created. This porous layer reduces mechanical strength and elasticity, making it less suitable for certain applications. Additionally, this structure does not withstand heat compression bonding of the film because the pores would collapse. The loss of porous structure during heat compression negates the original intent of using the hydrophilic molecule, thus resulting in a densely packed film without any enhanced therapeutic agent release capability.

Further, if the hydrophilic molecule remains in the polymer layer during heat compression, the dissolution of the hydrophilic molecule in vivo causes the formation of very large pores, approximately 3-10 μm in diameter. Such large pores provide a large surface area, thereby causing a burst release of drug. In contrast to the use of hydrophilic molecules, the use of a surfactant as a releasing agent in the present technology enables the formation of microchannels approximately 5-20 nanometers in diameter, which is two orders of magnitude smaller than the pores resulting from the use of hydrophilic molecules. This allows tight control of the drug release by diffusion and, if desirable, without an uncontrolled burst release upon implantation. Additionally, use of a surfactant as a releasing agent allows the agent to remain present in the polymer prior to use and no pre-formed pores are created. This approach is particularly advantageous because the polymer's mechanical properties are preserved, thereby allowing the polymer to be easily processed and worked into different configurations.

In the present technology, the releasing agent is pre-mixed into the bioresorbable polymer such that each layer of polymer is contiguous and dense. The depot 100 is then formed when these layers are bonded together via heat compression without any adverse impact to the functional capabilities of the film. When the densely packed film is ultimately implanted, the releasing agent dissolves to enable efficient, controlled release of the therapeutic agent.

In some embodiments, the releasing agent comprises a polysorbate. Polysorbate is commonly used in the pharmaceutical industry as an excipient and solubilizing agent. Polysorbate is a non-ionic surfactant formed by the ethoxylation of sorbitan followed by esterification by lauric acid. Polysorbate 20 [IUPAC name: polyoxyethylene(20)sorbitan monolaurate] contains a mixture of ethoxylated sorbitan with 20 repeat units of polyethylene glycol distributed among four different sites in the sorbitan molecule. Common commercial names include Tween™ and Tween 20™ (Croda International Plc, Goole, East Yorkshire, UK) and Alkest® TW 20 (Oxiteno, Houston, Tex.).

Polysorbate is often utilized to improve oral bioavailability of a poorly water-soluble/hydrophobic drug. For example, polysorbate was used to improve bioavailability of active molecules that possess low solubility and/or intestinal epithelial permeability and it was observed that the bioavailability of this poorly water-soluble drug was greatly enhanced in a formulation with polysorbate or similar surfactants. (WO2008/030425; Breslin; Merck.) Akbari, et al., observed that using the hydrophilic carrier polyethylene glycol (PEG) along with polysorbate leads to faster an oral enhanced drug release rate because the polysorbate brings the drug in close contact with the PEG. (Akbari, J., et al., ADV. PHARM. BULL., 2015, 5(3): 435-41.)

Polysorbate also functions as a water-soluble emulsifier that promotes the formation of oil/water emulsions. For example, the drug famotidine is known to have high solubility in water but low in vivo permeability. Polysorbate was used in an oral microemulsion formulation for enhancing the bioavailability of famotidine. (Sajal Kumar Jha, et al., IJDDR, 2011, 3(4): 336-43.) Polysorbate is also used as a wetting agent to achieve rapid drug delivery. For example, Ball et al., achieved rapid delivery of maraviroc via a combination of a polyvinylpyrrolidone (PVP) electrospun nanofiber and 2.5 wt % Tween 20, which allowed for the complete release of 28 wt % maraviroc in just six minutes. It was believed that use of Tween 20 as a wetting agent allowed water to penetrate the PVP nanofiber matrix more quickly, thereby increasing the rate of drug release. (Ball, C., et al., ANTIMICROB. AGENTS CHEMOTHERAPY, 2014, 58:8, 4855-65.)

As described above, in order to improve drug release in certain polymer carriers, hydrophilic polymers, such as polysorbate, have been added to these carriers to accelerate or to enhance drug release from biocompatible polymers such as polyethylene glycol (PEG) in oral formulations (Akbari, J., et al., ADV. PHARM. BULL., 2015, 5(3): 435-441). However, these formulations are intended to provide an immediate release of a hydrophobic drug into a hydrophilic environment (the in vivo physiologic fluid), not a variable or sustained controlled release as part of a control region.

In some embodiments, the releasing agent is polysorbate 20, commercially known as Tween 20™. Other releasing agents suitable for use in the present technology include polysorbates, such as Polysorbate 80, Polysorbate 60, Polysorbate 40, and Polysorbate 20; sorbitan fatty acid esters, such as sorbitan monostearate (Span 60), sorbitan tristearate (Span 65), sorbitane trioleate (Span 85), sorbitan monooleate (Span 80), sorbitan monopalmitate, sorbitan monostearate, sorbitan monolaurate, sorbitan monopalmitate, sorbitan trioleate, and sorbitan tribehenate; sucrose esters, such as sucrose monodecanoate, sucrose monolaurate, sucrose distearate, and sucrose stearate; castor oils such as polyethoxylated castor oil, polyoxyl hydrogenated castor oil, polyoxyl 35 castor oil, Polyoxyl 40 Hydrogenated castor oil, Polyoxyl 40 castor oil, Cremophor® RH60, and Cremophor® RH40; polyethylene glycol ester glycerides, such as Labrasol®, Labrifil® 1944; poloxamer; polyoxyethylene polyoxypropylene 1800; polyoxyethylene fatty acid esters, such as Polyoxyl 20 Stearyl Ether, diethylene glycol octadecyl ether, glyceryl monostearate , triglycerol monostearate, Polyoxyl 20 stearate, Polyoxyl 40 stearate, polyoxyethylene sorbitan monoisostearate, polyethylene glycol 40 sorbitan diisostearate; oleic acid; sodium desoxycholate; sodium lauryl sulfate; myristic acid; stearic acid; vitamin E-TPGS (vitamin E d-alpha-tocopherol polyethylene glycol succinate); saturated polyglycolized glycerides, such as Gelucire® 44/14 and Gelucire® 50/13; and polypropoxylated stearyl alcohols such as Acconon® MC-8 and Acconon® CC-6.

### Diffusion Openings

The channels or voids formed within the therapeutic region 200 and/or control region 300 by dissolution of the releasing agent may be in the form of a plurality of interconnected openings or pores and/or a plurality of interconnected pathways, referred to herein as “diffusion openings.” In some embodiments, one or more of the channels may be in the form of discrete pathways, channels, or openings within the respective therapeutic and/or control region. Depending on the chemical and material composition of the therapeutic and control regions, one or more of the formed channels may extend: (a) from a first end within the therapeutic region to a second end also within the therapeutic region; (b) from a first end within the therapeutic region to a second end at the interface of the therapeutic region and the control region; (c) from a first end within the therapeutic region to a second end within the control region; (d) from a first end within the therapeutic region through the control region to a second end at an outer surface of the control region; (e) from a first end at the interface between the therapeutic region and the control region through the control region to a second end within the control region; (f) from a first end at the interface between the therapeutic region and the control region to a second end at an outer surface of the control region; (g) from a first end within the control region to a second end also within the control region; and (h) from a first end within the control region to a second end at an outer surface of the control region. Moreover, one or more of the channels may extend between two or more microlayers of the therapeutic region and/or control region.

G. Constituent Ratios

In some embodiments, the ratio of the polymer in the control region 300 to the releasing agent in the control region 300 is at least 1:1. In some embodiments, the ratio may be at least 1.5:1, at least 2:1, at least 2.5:1, or at least 3:1.

In some embodiments, a ratio of the mass of the therapeutic agent in the depot 100 to the polymer mass of the depot is at least 1:1, at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, or at least 16:1.

In some embodiments, the ratio of releasing agent to polymer to therapeutic agent in the therapeutic region 200 is of from about 0.1:10:20 to about 2:10:20, and in some embodiments of from about 0.1:10:20 to about 1:10:20, and in some embodiments of from about 0.1:10:20 to about 0.5:10:20.

In some embodiments, the ratio of releasing agent to polymer in the control region 300 is of from about 1:2 to about 1:10. In some embodiments, one or more of the control regions may have a ratio of releasing agent to polymer of 1:2, and one or more of the other control regions may have a ratio of releasing agent to polymer of 1:10

H. Example Methods of Manufacture

The depots of the present technology may be constructed using various combinations of bioresorbable polymer layers, wherein these layers may include therapeutic agents, releasing agents, delayed release agents, etc., in varying combinations and concentrations in order to meet the requirements of the intended clinical application(s). In some embodiments, the polymer regions or layers may be constructed using any number of known techniques to form a multilayer film of a particular construction. For example, a bioresorbable polymer and a therapeutic agent can be solubilized and then applied to the film via spray coating, dip coating, solvent casting, and the like. In an alternative embodiment, a polymer layer for use as a control region and/or a therapeutic region can be constructed from electrospun nanofibers.

The depots 100 described herein may be constructed by placing therapeutic regions (and/or sub-regions) and/or control regions (and/or sub-regions) on top of one another in a desired order and heat compressing the resulting multilayer configuration to bond the layers together. Heat compression may be accomplished using any suitable apparatus known in the art. In one embodiment, the heat compression process consists of utilizing a heat compressor (Kun Shan Rebig Hydraulic Equipment Co. Ltd., China), and heat compressing the stacked assembly of therapeutic 200 and/or control regions 300 at a temperature that is above room temperature (e.g., at least 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., or 120° C., etc.) and a pressure of from about 0.01 MPa to about 1.0 MPa, or about 0.10 MPa to about 0.8 MPa, or about 0.2 MPa to about 0.6 MPa. The inventors have discovered that heating the therapeutic and control regions during compression (separately or after stacking) increases the therapeutic agent density in the depot 100. The inventors have also discovered that heat compression at lower pressures enable higher drug densities.

Depending on the therapeutic dosage needs, anatomical targets, etc., the depot 100 can be processed, shaped and otherwise engineered to produce form factors that can be administered to the patient by implantation in the body by a clinical practitioner. For example, various configurations of the film may be achieved by using a jig with a pre-shaped cutout, hand cutting the desired shape or both. Some of the form factors producible from the multilayer film for implantation into the body include: strips, ribbons, hooks, rods, tubes, patches, corkscrew-formed ribbons, partial or full rings, nails, screws, tacks, rivets, threads, tapes, woven forms, t-shaped anchors, staples, discs, pillows, balloons, braids, tapered forms, wedge forms, chisel forms, castellated forms, stent structures, suture buttresses, coil springs, and sponges. As described below with respect to FIG. 48C, in some embodiments a pellet-like or mini-cylindrical depot 100 can be punched or otherwise cut out of a sheet of a multilayer film. A depot 100 may also be processed into a component of the form factors mentioned above. For example, the depot 100 could be rolled and incorporated into tubes, screws tacks or the like. In the case of woven embodiments, the depot 100 may be incorporated into a multi-layer woven film wherein some of the filaments used are not the inventive device. In one example, the depot 100 is interwoven with Dacron, polyethylene or the like.

In some embodiments, one or more depots 100 can be cut into a desired shape or form factor using precision laser cutting. Various laser modalities may be used, for example infrared lasers, near-infrared lasers, deep ultraviolet lasers, or other suitable lasers for cutting depots 100 to the desired configurations. Such laser cutting can use continuous or pulsed, and the operating parameters (e.g., intensity, frequency, polarization, etc.) may be selected to achieve the desired cut. Using computer-controller laser-cutting can provide for a precise, repeatable manufacturing process that achieves consistent dimensions and release profiles. In some embodiments, the cut surfaces resulting from the laser-cut can be significantly smoother than those achieved using a mechanical stamp, jig, or punch to cut depots from a sheet of a multi-layer film. In some instances, the smoother cut surfaces can provide for improved release profiles, for example with more consistency among depots 100 manufactured according to this process.

In some embodiments, the therapeutic region 200 can be extruded into an elongated form (e.g., a cylindrical rod), after which the control region 300 may be spray- or dip-coated over the extruded therapeutic region 200. Portions of the extruded therapeutic region 200 may be masked to leave gaps in the control region 300, or alternatively portions of the control region 300 may be removed via etching, scraping, or other techniques to achieve any desired openings or thinning of the control region 300 in any desired portions. In some embodiments, an extruded cylinder having a lumen extending therethrough can be selectively filled with a therapeutic region 200 and/or a control region 300 along its length to form an elongated depot 100.

In some embodiments, a therapeutic region 200 in the shape of a cylindrical rod is formed by dissolving the therapeutic region composition (e.g., a mixture of polymer(s) and therapeutic agent) into acetone, and then loading the dissolved therapeutic region composition into a syringe (e.g., a 1 mL syringe) and attaching a needle thereto (e.g., a 19G needle). The therapeutic region solution is then injected into ethanol for polymer solidification. After waiting for the solution to harden (e.g., approximately 90 seconds), the resulting rod can be removed from the ethanol and air-dried. In another embodiment, the therapeutic region composition can be injected into a cross-linking solution to solidify the polymer.

The therapeutic region 200 may be spray- or dip-coated with a surrounding control region 300. Alternatively, in some embodiments, the therapeutic region 200 in elongated cylindrical form can be inserted into an inner lumen of a coaxial needle. The coaxial needle can include an inner needle disposed coaxially within the lumen of an outer needle. In one example, the inner needle can have an inner diameter of approximately 0.84 mm and an outer diameter of approximately 1.24 mm, and the outer needle can have an inner diameter of approximately 1.6 mm and an outer diameter of approximately 2.11 mm, though these dimensions can vary and be tailored to the desired dimensions of the therapeutic region 200 and control region 300. A control region composite (e.g., a mixture of polymer and releasing agent) can be dissolved in acetone, and then loaded into a syringe (e.g., a 1 mL syringe). The control region solution is then injected through the outer needle, surrounding the cylindrical therapeutic region disposed within the inner needle. The resulting depot 100 is a cylindrical form with a control region 300 substantially uniformly surrounding the inner cylindrical therapeutic region 200. In some embodiments, the resulting cylindrical form can be suitable for injecting using a needle, thereby providing for a convenient mechanism to deliver the depot to any number of different treatment sites. In other embodiments, a coaxial needle having three or more coaxial lumens can be used for the formation of multiple therapeutic and/or control regions, for example having a plurality of different therapeutic agents that can be configured to be released sequentially from the depot 100.

In some embodiments, an extruded depot 100 in the form an elongated columnar structure (e.g., a cylindrical rod, strip, etc.) can be pinched down at one or more positions along its length to be subdivided into discrete portions. For example, an elongated depot 100 may be pinched such that the depot is completely severed into discrete sections, or to provide a narrowed, weakened portion that can be susceptible to flexing and/or breaking.

FIG. 48C illustrates one method of manufacturing depots in the form of pellets as shown in FIGS. 48A and 48B. A sheet including a plurality of layered regions such as outer control regions 300 at least partially surrounding an inner therapeutic region 200 is provided. A punch 600 with a hollow blade can be used to cut out individual pellets from the sheet, for example by pressing the punch 600 through the sheet along an axis orthogonal to the surface of the sheet. In some embodiments, the resulting pellets each retain the layered regions of the sheet (e.g., a therapeutic region 200 sandwiched between first and second control regions 300). In such embodiments, the resulting pellet can have at least a portion of the therapeutic region 200 exposed through the control region(s) 300, for example with lateral sides of the pellet having exposed portions of the therapeutic region 200. Such exposed portions of the therapeutic region 200 can contribute to a higher initial release rate of the therapeutic agent.

In some embodiments, the punch 600 is heated before cutting the pellets from the sheet, for example by being heated in an oven to approximately 80° C., or to a suitable temperature to at least partially melt or deform the control region 300. The heated punch 600 can at least partially deform the top layer (e.g., partially melting the upper control region 300) causing it to wrap around the lateral edges of the therapeutic region 200. The resulting depot 100 may then take the form of a pellet 100 in which the inner therapeutic region 200 is completely or substantially completely surrounded by the control region(s) 300. In some embodiments, the motion of pressing the punch 600 can be varied to achieve the desired coverage of the control region(s) 300 over the therapeutic region 200. For example, in some embodiments, the punch 600 can be rotated while being pressed through the sheet, and in some embodiments the punch 600 can be moved more slowly or move quickly to allow varying degrees of deformation and flow of the control region(s) 300. In other embodiments, the punch 600 is not heated before being pressed through the sheet.

The dimensions of the depots 100 in the form of pellets or mini-cylinders can be controlled by varying the thickness of the sheet and by selecting the diameter or lumen cross-sectional dimensions of the punch 600. In some embodiments, the sheet can have a thickness of between about 0.5 and 2 mm (e.g., approximately 0.85 mm), and the punch 600 can have a circular lumen with a diameter of between about 0.5 mm and about 3 mm (e.g., approximately 1 mm). In other embodiments, the punch 600 can cut out depots 100 in other shapes, for example, square, rectangular, elliptical, star-shaped, wavy, irregular polygonal, or any other suitable cross-sectional shape. In some embodiments, a wavy or jagged shape can provide a larger surface area for the resulting pellets, thereby increasing a rate of release of therapeutic agent from the pellets. In some embodiments, the resulting depots 100 in the form of pellets or mini-cylinders are insertable through a needle or other suitable delivery shaft. For example, a plurality of approximately pellets having 1 mm diameters may be loaded coaxially into a 17-gauge needle and inserted subcutaneously to a treatment site in a patient. Smaller pellet-like depots 100 could be inserted through even smaller needles, for example 18- to 22-gauge needles. Such pellets or mini-cylinders can achieve a considerably high drug loading, as described elsewhere herein, for example at least 50% by weight of the therapeutic agent or more. In various embodiments, the depot 100 can be configured to be injected or inserted via a needle that is no greater than 14, 16, 18, 20, or 22 gauge in size.

In some embodiments, microbead and/or pellet-like depots (e.g., as in FIGS. 47-48) can be formed by providing an elongated structure (e.g., a cylindrical, columnar, or rod-shaped structure) having a therapeutic region 200 at least partially surrounded by a control region 300, and then cutting or otherwise dividing the structure into a plurality of pellets, particles, or microbeads along its length.

### II. Systems and Methods for Treating Type II Diabetes

Nearly half of all adults and approximately 8% of children (aged 5-17 years old) worldwide have one or more chronic conditions that can be treated or mitigated with existing medications. Unfortunately, the prevalence of medication non-adherence/non-compliance amongst patients has inhibited their effective treatment for years. In one estimate, approximately 50% of patients needing long-term therapy for chronic illnesses do not adhere to the prescribed frequency and/or dosage requirements for their medication. As a result, medication non-adherence leads to approximately 125,000 preventable deaths each year and $300 billion in avoidable healthcare costs. On a per patient basis in the United States, the annual adjusted disease-specific economic cost of non-adherence can range from approximately $1,000-$44,000. Moreover, as worldwide population continues to age, the negative effects of non-adherence continue to increase. Accordingly, medication non-adherence for chronic illnesses remains a major impediment to achieving high levels of effective patient treatment.

A variety of factors contributes to intentional and unintentional medication non-adherence, such as (1) the patient (e.g., age, cognitive impairment, lack of disease knowledge), (2) socio-economic conditions (e.g., lack of health insurance, medication cost, low health literacy), (3) the disease (e.g., lack or severity of symptoms, depression, psychotic disorders), and (4) the prescribed therapy (e.g., complexity of the medication regime, therapy duration, actual or perceived side effects). Each of these factors, and others, indicate the complexity associated with developing a solution to medication non-adherence. Moreover, the combination of these factors indicates that pharmacological development of new medications, by itself, is insufficient to cure the problems associated with non-adherence.

One of the biggest areas affected by medication non-adherence is diabetes, a worldwide chronic disease affecting millions of people. In the United States alone, it is estimated that more than 30 million people have diabetes, of which approximately 95% have type II diabetes (“T2D”). Additionally, over 80 million people in the United States have prediabetes and are at risk of becoming type II diabetics. T2D patients are unable to properly process insulin and/or do not produce enough insulin to regulate glucose levels. As a result, these patients have elevated blood sugar which causes debilitating symptoms that generally affect quality of life.

Currently, the predominant treatment for T2D is a pharmacological approach involving medications that are typically self-administered, either orally or via injection. These medications can include metformin, sulfonylureas, thiazolidinediones, dipeptidyl peptidase-IV inhibitors and insulin, amongst others. While each of these medications can provide immediate, short-term benefits to treating symptoms associated with T2D, they also have undesired and sometimes long-term side effects. For example, some of these medications (e.g., sulfonylureas, thiazolidinediones, and insulin) have been shown to cause hypoglycemia and/or weight gain, while others (e.g., metformin and dipeptidyl peptidase-IV) have been shown to increase the risk of cardiovascular disease, joint pain, and/or infection of the kidney, liver, pancreas or genitals. These medications are further limited by having a short time of action or half-life, usually lasting less than 1-4 hours in vivo in adults. As a result, T2D patients must often administer medication on a daily basis. The combination of a short half-life and self-administration of the medication has contributed to the previously described issues associated with medication non-adherence. Accordingly, these medications have been generally unable to provide optimal, long-term treatment for T2D patients.

Another medication commonly used to treat T2D is a type of non-insulin injectable known as glucagon-like peptide (GLP-1) receptor agonists. GLP-1 is naturally produced by the body, but is generally lower in T2D patients. Functionally, GLP-1 is normally released from the small intestine after eating, and signals the brain, pancreas, and stomach to decrease appetite, slow gastric emptying, and stimulate insulin secretion. Administered GLP-1 receptor agonists have a similar effect, helping T2D patients slow their digestion and lower blood sugar levels. Additionally, GLP-1 receptor agonists have been associated with weight loss and decreased risk of heart attacks and strokes, and have generally not been associated with some of the side effects (e.g., hypoglycemia and weight gain) previously described for other T2D medications. However, similar to the medications previously described, the half-life of GLP-1 receptor agonists are still relatively short. To increase their half-life, an array of extension strategies have been utilized, including (1) attachment of a fatty-acid, as done with medications like Victoza® (Novo Nordisk, Bagsvaerd, Denmark) and Ozempic® (Novo Nordisk, Bagsvaerd, Denmark), (2) fusion with albumin or monoclonal antibodies, as done with medications like Tanzeum® (GlaxoSmithKline, London, United Kingdom) and Trulicity® (Eli Lilly, Indianapolis, Ind.), and (3) sequential modification, as done with medications like Byetta® (AstraZeneca, Cambridge, United Kingdom) and Adlyxin® (Sanofi, Paris, France). While these half-life improvements to GLP-1 receptor agonists have decreased dosage rates to be less than once daily, the dosage rates still require patients to administer the medications multiple times per week or weekly. Accordingly, medication non-adherence issues associated with these extended half-life medications remain an issue.

In an effort to overcome these adherence issues, a number of injectable, sustained release systems have been developed, such as Bydureon® (AstraZeneca, Cambridge, United Kingdom) and Intarcia® (Intarcia Therapeutics, Boston, Mass.). The Bydureon® release system is a pen device that delivers dosages to T2D patients via self-administered subcutaneous injection. The dosages contain polylactic-co-glycolic acid (PLGA) based microspheres that degrade over time to provide a continuous release of the GLP-1 receptor agonist. The Bydureon® release system, however, has multiple drawbacks. For example, the injected microspheres have been unable to consistently deliver the GLP-1 receptor agonist at a steady rate over a period of time exceeding one week. Instead, the release rate of Bydureon® generally corresponds to a mountain-shaped plot, with the dosage rate rising to a peak, and then falling until depleted. The variation in delivered GLP-1 receptor agonist to the patient can cause the T2D symptoms to persist, or cause other side effects (e.g., nausea and/or vomiting) to occur. Yet another drawback of the Bydureon® system is that weekly administration is still required. As such, the non-adherence issues previously described persist.

Intarcia®, another sustained release system, is an osmotic mini-pump subcutaneously implanted in patients to continuously release GLP-1 receptor agonists over a maximum time period of 6 months. Intarcia® has been more successful relative to Bydureon® in that patients can administer it on a weekly or bi-weekly basis. However, Intarcia® does have other drawbacks. For example, the Intarcia mini-pump is made of a rigid, nonbiodegradable material, and thus must be removed from the patient once the medication is depleted.

Thus, a need exists for biocompatible implantable systems capable of providing a controlled, sustained release of medication to T2D patients. The depots 100 of the present technology may be used to treat a variety of symptoms (e.g., abnormal blood sugar levels) associated with T2D depending upon the nature of the therapeutic agent delivered as described above. The depots 100 of the present technology may be implanted in vivo proximate to the target tissue (i.e., bone, soft tissue, etc.) in the patient's body to provide a controlled, sustained release of a therapeutic agent for the treatment of a particular condition. For example, the depot can be configured to provide a sustained presence of the therapeutic agent to the treatment site over a predefined period of time. This implantation may be associated with a surgery or intervention for systemically treating the particular symptom or condition, whereby the depot enables chronic, sustained pharmacological treatment following completion of the surgery or intervention. The depot may be a standalone element, or may be coupled to or integrated as part of an implantable device or prosthesis associated with the intervention or surgery.

The amount of the therapeutic agent that will be effective in a patient in need thereof will depend on the specific nature of the condition, and can be determined by standard clinical techniques known in the art. In addition, in vitro or in vivo assays may optionally be employed to help identify optimal dosage ranges. The specific dose level for any particular individual will depend upon a variety of factors including the activity of the drug, the age, body weight, general physical and mental health, genetic factors, environmental influences, sex, diet, time of administration, location of administration, rate of excretion, and the severity of the particular problem being treated.

Some aspects of the present technology include a system comprising a plurality of depots (each of which could be any of the depots described herein) provided for implantation by a clinical practitioner. In this system, each depot may be configured for controlled release of therapeutic agent to tissue proximate to the implantation site of the depot. The depots in the system may be identical or may vary in several respects (e.g., form factor, therapeutic agent, release profile, etc.). For example, the system may be comprised of a depot having a release profile that provides for an immediate release of therapeutic agent and other depots comprised of a depot having a release profile that provides for a delayed release of therapeutic agent.

The depot 100 used to deliver the therapeutic agent(s) for treating select symptoms associated with T2D may comprise any of those depots 100 previously described herein, such as those described with reference to FIGS. 1-52C. For example, the depot 100 may comprise a substantially columnar shape, a substantially cylindrical shape, a rolled film configuration, and/or microdepots (e.g., beads, microspheres, microcyliners, etc.). The shape of the depot 100, the arrangement of the various regions of the depot 100, and the mechanical properties of the depot 100 can be optimized for ease of delivery (by the patient and/or clinician) and/or patient comfort. For example, the elongated depot configurations disclosed herein have a low-profile, thereby improving patient comfort and enabling injection/insertion through a lower diameter needle. In these and other embodiments described herein, many depots 100 of the present technology are sufficiently flexible and/or provide features for preferential bending such that the depot 100 can flex or deform relative to the delivery device and/or better adapt and conform to the patient's anatomy. The flexibility and low-profile configuration of the depots 100 herein thus provide less resistance to patient movement and/or unwanted protrusions or visible indications of the depot 100 on the patient's skin, making the depots 100 more conducive to long-term (e.g., a month or multi-month) wear.

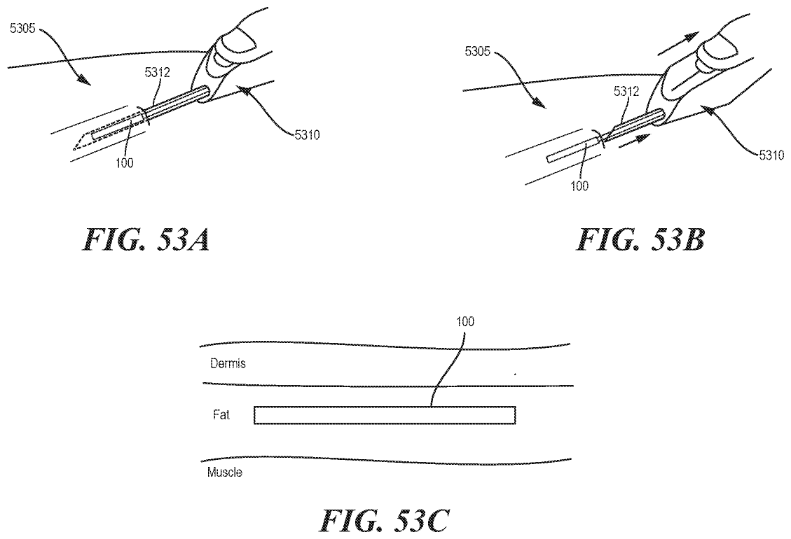

In some applications, the depot 100 may be delivered to the patient via subcutaneous or intramuscular (IM) administration, and/or positioned at or proximate to areas including the abdomen, deltoid, gluteal, arm, or thigh/femur, as well as other areas wherein the therapeutic agent delivered from the depot can be systemically absorbed into a patient's bloodstream. FIGS. 53A-C are partially schematic perspective views of a delivery system 5310 for subcutaneously delivering a depot 100 to a target site 5305 in accordance with some embodiments of the present technology. As previously described, the target site 5305 can include the abdomen, deltoid, gluteal, arm, or femur areas, as well as any other area that allows the therapeutic agent delivered from the depot to be systemically absorbed into the patient's bloodstream. As shown in FIG. 53A, the delivery system 5310 includes a needle 5312 inserted beneath a patient's skin, e.g., to engage subcutaneous tissue (e.g., the dermis, fat or muscle layers, or other layer beneath the muscle) of the patient. The needle can be 25 gauge, 24 gauge, 23 gauge, 23 gauge, 21 gauge, 20 gauge, 19 gauge, 18 gauge, 17 gauge, 16 gauge, 15 gauge, 14 gauge, 13 gauge, 12 gauge, 11 gauge, 10 gauge, 9 gauge, 8 gauge, 7 gauge, or other size depending on lateral dimensions of the depot 100 to be delivered to the patient. As shown in FIG. 53A, once the needle 5312 is inserted such that the length of the depot 100 is disposed beneath the patient's skin, the delivery system 5310 can be actuated, e.g., away from the depot 100, to cause the depot 100 to be released from the delivery system 5310 to the patient. In some embodiments, the depot 100 may be disposed within a sheath, and actuating the delivery system 5310 withdraws the sheath from the depot 100, which remains implanted in the patient. FIG. 53C illustrates the implanted depot 100 positioned in the fat layer between the dermis and muscle after the delivery system has been removed. In other embodiments, the implanted depot 100 can be implanted elsewhere, including between the fat layer and underlying muscle layer or beneath the muscle layer.

In some embodiments, the depot 100 (or a system of depots 100) is configured to release the therapeutic agent at a rate according to a particular profile as previously described, e.g., with reference to FIGS. 3A and 3B. For example, the depot 100 for treating symptoms associated with T2D can be configured to release the therapeutic agent in a substantially steady state manner (i.e., a zero-order release profile) for a desired period of time. Stated differently, the depot 100 is configured to release the therapeutic agent at a consistent rate for a period of time (e.g., from 1 day to 365 days, or as described herein in Sections I) post-implantation or post-immersion in a fluid. During such a period of time, the therapeutic agent may be released from the depot 100 to the surrounding area at a substantially consistent release rate such that about 20-30% of the therapeutic agent has been released when one quarter of the period of time has passed, 40-60% of the therapeutic agent has been released when one half of the period of time has passed, 70-80% of the therapeutic agent has been released after three quarters of the period of time has passed, at least 90% of the therapeutic agent has been released once the entire period of time has passed.

The zero-order release profile achieved by depots 100 of the present technology can be beneficial to patients across a variety of applications. For example, the consistent release of therapeutic agent may allow a more favorable pharmacokinetic profile in that the concentration of the therapeutic agent remains substantially steady over the period of treatment or release time. Compared to therapeutic agents that when delivered result in peaks and troughs of plasma drug levels, which can be associated with adverse events and symptom breakthrough, embodiments of the present technology can achieve more steady plasma drug levels on a daily, weekly, monthly, and/or yearly basis. The combination of the zero-order release profile and weekly, monthly, and/or yearly duration of release times in embodiments of the present technology offers a solution to the medication non-adherence issues previously described in that patients can receive the prescribed, steady-state dosage of the therapeutic agent via the implanted depot without bearing the burden of self-administering medication on a daily, weekly or monthly basis to maintain the prescribed plasma drug levels.

As previously described, the release profile of the depot may be tuned to release a therapeutic agent for particular durations and/or at particular release rates by adjusting the structure, composition, and the process by which the depot is manufactured. In some embodiments, including but not limited to those embodiments used to treat symptoms associated with T2D, the depot may be configured to release the therapeutic agent through the duration of release (i.e., the period of time) at a rate of from about 5 μg/day to about 10 mg/day, about 5 μg/day to about 5 mg/day, about 5 μg/day to about 1 mg/day, about 5 μg/day to about 900 μg/day, about 5 μg/day to about 800 μg/day, about 5 μg/day to about 700 μg/day, about 5 μg/day to about 600 μg/day, about 5 μg/day to about 500 μg/day, about 5 μg/day to about 400 μg/day, about 5 μg/day to about 300 μg/day, about 5 μg/day to about 200 μg/day, about 5 μg/day to about 150 μg/day, about 5 μg/day to about 100 μg/day, about 5 μg/day to about 90 μg/day, about 5 μg/day to about 80 μg/day, about 5 μg/day to about 70 μg/day, about 5 μg/day to about 60 μg/day, about 5 μg/day to about 50 μg/day, about 5 μg/day to about 40 μg/day, about 5 μg/day to about 30 μg/day, about 5 μg/day to about 20 μg/day, about 10 μg/day to about 20 μg/day, or any other incremental ranges therebetween.

In some embodiments, the depot is configured to release less than about 1000 μg/day, less than about 900 μg/day, less than about 800 μg/day, less than about 700 μg/day, less than about 600 μg/day, less than about 500 μg/day, less than about 450 μg/day, less than about 400 μg/day, less than about 350 μg/day, less than about 300 μg/day, less than about 250 μg/day, less than about 200 μg/day, less than about 175 μg/day, less than about 150 μg/day, less than about 125 μg/day, less than about 100 μg/day, less than about 90 μg/day, less than about 80 μg/day, less than about 70 μg/day, less than about 60 μg/day, less than about 50 μg/day, less than about 45 μg/day, less than about 40 μg/day, less than about 35 μg/day, less than about 30 μg/day, less than about 25 μg/day, less than about 20 μg/day, less than about 15 μg/day, less than about 10 μg/day, or less than about 5 μg/day, over the period of time.

In some embodiments, the depot may be configured to release the therapeutic agent throughout the duration of release at a rate of from about 1 nmol/day to about 1.2 μmol/day, about 10 nmol/day to about 1.0 μmol/day, about 10 nmol/day to about 900 nmol/day, about 10 nmol/day to about 800 nmol/day, about 10 nmol/day to about 700 nmol/day, about 10 nmol/day to about 600 nmol/day, about 10 nmol/day to about 500 nmol/day, about 10 nmol/day to about 400 nmol/day, about 10 nmol/day to about 300 nmol/day, about 10 nmol/day to about 200 nmol/day, about 10 nmol/day to about 150 nmol/day, about 10 nmol/day to about 100 nmol/day, about 10 nmol/day to about 90 nmol/day, about 10 nmol/day to about 80 nmol/day, about 20 nmol/day to about 80 nmol/day, about 30 nmol/day to about 80 nmol/day, about 40 nmol/day to about 80 nmol/day, about 50 nmol/day to about 80 nmol/day, about 50 nmol/day to about 70 nmol/day, or other incremental ranges therebetween.

In some embodiments, the depot is configured to release less than about 500 nmol/day, less than about 300 nmol/day, less than about 250 nmol/day, less than about 200 nmol/day, less than about 175 nmol/day, less than about 150 nmol/day, less than about 125 nmol/day, less than about 100 nmol/day, less than about 90 nmol/day, less than about 80 nmol/day, less than about 70 nmol/day, less than about 60 nmol/day, less than about 50 nmol/day, less than about 45 nmol/day, less than about 40 nmol/day, less than about 35 nmol/day, less than about 30 nmol/day, less than about 25 nmol/day, less than about 20 nmol/day, less than about 15 nmol/day, or less than about 10 nmol/day, over the period of time.

In addition to or in lieu of the therapeutic agents previously described, in some embodiments, the therapeutic agent may comprise systemic incretins, such as a GLP-1 receptor agonist. Suitable GLP-1 receptor agonists include, but are not limited to, exenatide, liraglutide, albiglutide, dulaglutide, lixisenatide, semaglutide, insulin degludec, insulin glargine, derivatives thereof. The therapeutic agent may comprise the pharmacologically active drug or a pharmaceutically acceptable salt thereof. Preferred therapeutic agents include exenatide and liraglutide. In some embodiments, the therapeutic agent includes sulfonylureas, biguanidines (e.g., metformin), thiazolidinediones, gliptins (e.g., dipeptidyl peptidase-4 inhibitors), combinations thereof, and combinations thereof with one or more GLP-1 receptor agonists. Typically, naturally produced GLP-1 stimulates insulin release and/or inhibits glucagon release, each of which can lower blood glucose. In addition to or in lieu of the therapeutic agents previously described, any chemical compound possessing such stimulatory or inhibitory properties is suitable for use in the present technology.

In some embodiments, the depot 100 may include other therapeutic agents in addition to GLP-1 agonists for combination therapy. One such therapeutic agent that may be beneficial for delivering alongside GLP-1 is metformin. Metformin ameliorates insulin resistance, reduces hyperinsulinemia, and counteracts weight gain. Other suitable adjunctive therapeutic agents may also be included. The different therapeutic agents may be released from the depot 100 at the same or different times, be contained within the same or different therapeutic regions of the depot 100, and/or be released for the same or different durations.

A particular challenge in sustained delivery of peptides in vivo (such as GLP-1 agonists) is preventing or reducing denaturing of the peptide in response to the local environment. Body temperature, endogenous enzymes, salts, local acidity, and other influences can denature the peptide, thereby reducing or altogether nullifying the therapeutic effects of delivery of the therapeutic agent. The depot(s) 100 of the present technology may include one or more control regions (such as those described herein) surrounding the therapeutic region to reduce or eliminate significant denaturation of the peptide. In some embodiments, the depot(s) 100 may include a thermal stabilizer, such as a sugar compound (e.g., trehalose), an antioxidant (e.g., methionine, ascorbic acid, sodium thiosulfate, catalase, platinum, ethylenediaminetetraacetic acid (EDTA), citric acid, cysteins, thioglycerol, thioglycolic acid, thiosorbitol, butylated hydroxanisol, butylated hydroxyltoluene, and propyl gallate) and/or a buffer (e.g., citrate, histidine, succinate and tris), e.g., to help improve stability of the peptide or other therapeutic agent. Sugars can make hydrogen bonds which provide more structure to the peptide, thereby increasing its half-life under adverse conditions. In some embodiments, the therapeutic agent comprises the thermal stabilizer, whereas in other embodiments, the sugar at least partially surrounds the therapeutic agent.

### III. Systems and Methods for Treating Mental Illness

As noted previously, patient non-adherence can be a significant concern for chronic conditions. Some of the biggest areas affected by medication non-adherence are mental illnesses such as depression, schizophrenia, and dementia. These and other mental illnesses have a widespread effect around the world, with approximately 350 million people (approximately 5% of the world population) suffering from depression, approximately 20 million people suffering from schizophrenia, and approximately 50 million people suffering from dementia. Moreover, the number of people affected by mental illnesses is growing as the worldwide population ages.

Currently, the predominant treatment for depression includes a number or orally administered antidepressant medications. These include selective serotonin reuptake inhibitors (SSRIs), serotonin-norepinephrine reuptake inhibitors (SNRIs), tricyclic antidepressants (TCAs), and monoamine oxidase inhibitors (MAOIs). Given the well-known issues of medication non-adherence for these drugs, a number of antidepressant release systems have been developed, including paroxetine (Paxil®, GlaxoSmithKline, Research Triangle Park, N.C.), venlafaxine (Effexor®, Pfizer, New York City, N.Y.), fluoxetine (Prozac®, Eli Lilly, Indianapolis, Ind.), and bupropion (Wellbutrin®, GlaxoSmithKline, Research Triangle Park, N.C.). Each of these medications, however, is administered orally by the patient and requires daily or weekly dosing. Accordingly, the predominant antidepressants do not provide an optimal solution to medication non-adherence issues.

The predominant treatment for schizophrenia includes first-generation antipsychotics (e.g., chlorpromazine, fluphenazine, haloperidol, and perphenazine) and second-generation antipsychotics. The second-generation antipsychotics include aripiprazole (Abilify®, Otsuka America Pharmaceutical, Tokyo, Japan), lurasidone (Latuda®, Sumitomo Dainippon Pharma, Chuo-ku, Japan), and risperidone, (Risperdal®, Janssen Pharmaceutical, Beerse, Belgium or Perseris®, Indivior, Richmond, Va.), amongst others. Extended release systems have been developed for many of the second-generation antipsychotics. For example, Perseris® and Ability® are long-acting injectables that can be subcutaneously administered on a bi-weekly basis. The extended release systems currently available, however, have drawbacks that stand in the way of an optimal treatment for patients. For example, these systems lack a true controlled release mechanism in that they typically provide a burst of drug upon contact with surrounding physiologic fluids followed by a residual release of drug. In order to improve drug release in certain polymer carriers, hydrophilic polymers, such as polysorbate, have been added to these carriers as wetting agents to accelerate or to enhance drug release from biocompatible polymers such polyethylene glycol (PEG) in oral formulations (Akbari, J., et al., ADV. PHARM. BULL., 2015, 5(3): 435-441). However, these formulations are intended to provide an immediate release of a hydrophobic drug into a hydrophilic environment (the in vivo physiologic fluid), where a substantial portion of the entire drug payload is immediately or aggressively released, not a variable or sustained control release. Accordingly, the predominant antipsychotic medications and their release systems do not provide an optimal solution to the issues associated with medication non-adherence.

Treatments available for dementia suffer from similar issues as those of schizophrenia. For example, while medications such as donepezil, galantamine and rivastigmine are generally available to treat dementia, the extended release systems for these medications prevent effective treatment for a number of patients. As previously stated, the release systems currently available suffer from a lack of a true controlled release mechanism in that they typically provide a burst of drug upon contact with surrounding physiologic fluids followed by a residual release of drug. Accordingly, the predominant dementia medications, including their release systems, do not provide an optimal treatment and/or solution to the issues associated with medication non-adherence.

Thus, a need exists for biocompatible implantable systems capable of providing a controlled release of medication to patients with symptoms associated with mental illnesses. The depots 100 of the present technology may be used to treat a variety of symptoms associated with a mental illness depending upon the nature of the therapeutic agent delivered as described above. The depots 100 of the present technology may be implanted in vivo proximate to the target tissue (i.e., bone, soft tissue, etc.) in the patient's body to provide a controlled, sustained release of a therapeutic agent for the treatment of a particular condition. For example, the depot can be configured to provide a sustained presence of the therapeutic agent to the treatment site over a predefined period of time. This implantation may be associated with a surgery or intervention for systemically treating the particular symptom or condition, whereby the depot enables chronic, sustained pharmacological treatment following completion of the surgery or intervention. The depot may be a standalone element, or may be coupled to or integrated as part of an implantable device or prosthesis associated with the intervention or surgery.

The amount of the therapeutic agent that will be effective in a patient in need thereof will depend on the specific nature of the condition, and can be determined by standard clinical techniques known in the art. In addition, in vitro or in vivo assays may optionally be employed to help identify optimal dosage ranges. The specific dose level for any particular individual will depend upon a variety of factors including the activity of the drug, the age, body weight, general physical and mental health, genetic factors, environmental influences, sex, diet, time of administration, location of administration, rate of excretion, and the severity of the particular problem being treated.

Some aspects of the present technology include a system comprising a plurality of depots 100 (each of which could be any of the depots described herein) provided for implantation by a clinical practitioner. In this system, each depot may be configured for controlled release of therapeutic agent to tissue proximate to the implantation site of the depot. The depots in the system may be identical or may vary in several respects (e.g., form factor, therapeutic agent, release profile, etc.). For example, the system may be comprised of a depot having a release profile that provides for an immediate release of therapeutic agent and other depots comprised of a depot having a release profile that provides for a delayed release of therapeutic agent.

The depot 100 used to deliver the therapeutic agent(s) for treating select symptoms associated with a mental illness may comprise any of those depots 100 previously described herein, such as those described with reference to FIGS. 1-52C. For example, the depot 100 may comprise a substantially columnar shape, a substantially cylindrical shape, a rolled film configuration, and/or microdepots (e.g., beads, microspheres, microcyliners, etc.). The shape of the depot 100, the arrangement of the various regions of the depot 100, and the mechanical properties of the depot 100 can be optimized for ease of delivery (by the patient and/or clinician) and/or patient comfort. For example, the elongated depot configurations disclosed herein have a low-profile, thereby improving patient comfort and enabling injection/insertion through a lower diameter needle. In these and other embodiments described herein, many depots 100 of the present technology are sufficiently flexible and/or provide features for preferential bending such that the depot 100 can flex or deform relative to the delivery device and/or better adapt and conform to the patient's anatomy. The flexibility and low-profile configuration of the depots 100 herein thus provide less resistance to patient movement and/or unwanted protrusions or visible indications of the depot 100 on the patient's skin, making the depots 100 more conducive to long-term (e.g., a month or multi-month) wear.

In some applications, the depot 100 may be delivered to the patient via subcutaneous or intramuscular (IM) administration, and/or positioned at or proximate to areas including the abdomen, deltoid, gluteal, arm, or thigh/femur, as well as other areas wherein the therapeutic agent delivered from the depot can be systemically absorbed into a patient's bloodstream. Such a depot can be delivered to a target site as described elsewhere herein with respect to FIGS. 53A-53C.

In some embodiments, the depot 100 (or a system of depots 100) is configured to release the therapeutic agent at a rate according to a particular profile, as previously described with reference to FIGS. 3A and 3B. For example, the depot 100 for treating symptoms associated with mental illness can be configured to release the therapeutic agent in a substantially steady state manner (i.e., a zero-order release profile) for a desired period of time. Stated differently, the depot 100 is configured to release the therapeutic agent at a consistent rate for a period of time (e.g., from 1 day to 365 days, or as described herein in Sections I) post-implantation or post-immersion in a fluid. During such a period of time, the therapeutic agent may be released from the depot 100 to the surrounding area at a substantially consistent release rate such that about 20-30% of the therapeutic agent has been released when one quarter of the period of time has passed, 40-60% of the therapeutic agent has been released when one half of the period of time has passed, 70-80% of the therapeutic agent has been released after three quarters of the period of time has passed, at least 90% of the therapeutic agent has been released once the entire period of time has passed. As noted previously, the zero-order release profile achieved by depots 100 of the present technology can be beneficial to patients across a variety of applications. For example, the consistent release of therapeutic agent may allow a more favorable pharmacokinetic profile in that the concentration of the therapeutic agent remains substantially steady over the period of treatment or release time.

As previously described, the release profile of the depot may be tuned to release a therapeutic agent for particular durations and/or at particular release rates by adjusting the structure, composition, and the process by which the depot is manufactured. In some embodiments, including but not limited to those embodiments used to treat symptoms associated with a mental illness, the depot may be configured to release the therapeutic agent through the duration of release (i.e., the period of time) at a rate of from about 0.1 mg/day to about 100 mg/day, about 0.1 mg/day to about 90 mg/day, about 0.1 mg/day to about 80 mg/day, about 0.1 mg/day to about 70 mg/day, about 0.1 mg/day to about 60 mg/day, about 0.1 mg/day to about 50 mg/day, about 0.1 mg/day to about 40 mg/day, about 0.1 mg/day to about 30 mg/day, about 1 mg/day to about 30 mg/day, about 1 mg/day to about 20 mg/day, about 5 mg/day to about 20 mg/day, about 10 mg/day to about 20 mg/day, or about 15 mg/day to about 20 mg/day, or any other incremental ranges therebetween.

In some embodiments, the depot may be configured to release the therapeutic agent through the duration of release at a rate no more than 100 mg/day, no more than 90 mg/day, no more than 80 mg/day, no more than 70 mg/day, no more than 60 mg/day, no more than 50 mg/day, no more than 40 mg/day, no more than 30 mg/day, no more than 20 mg/day, no more than 15 mg/day, no more than 10 mg/day, no more than 5 mg/day, or no more than 1 mg/day.

In addition to or in lieu of the therapeutic agents previously described, in some embodiments the therapeutic agent may comprise antidepressants such as selective serotonin reuptake inhibitors (SSRIs), serotonin-norepinephrine reuptake inhibitors (SNRIs), tricyclic antidepressants (TCAs), monoamine oxidase inhibitors (MAOIs), atypical antidepressants, and derivatives thereof. Examples of appropriate SSRIs can include citalopram, escitalopram, fluoxetine, fluvoxamine, fluvoxamine, paroxetine, and sertraline, as well as other chemical compounds configured to selectively block the reabsorption or reuptake of serotonin in the brain. Examples of appropriate SNRIs can include desvenlafaxine, duloxetine, venlafaxine, milnacipran, and levomilnacipran, as well as other chemical compounds configured to block the reabsorption of serotonin and/or norepinephrine in the brain. Examples of appropriate TCAs can include amitriptyline, desipramine, doxepine, imipramine, nortriptyline, amoxapine, clomipramine, maprotiline, trimipramine, and protriptyline, as well as other chemical compounds configured to block muscarinic M1, histamine H1, and/or α-adrenergic receptors. Examples of appropriate MAOIs can include phenelzine, selegiline, and tranylcypromine, as well as other chemical compounds configured to block the activity of monoamine oxidase. Examples of atypical antidepressants can include bupropion, mirtazapine, nefazodone, trazodone, vilazodone, and vortioxetine, as well as other chemical compounds that affect the level of dopamine, serotonin, and/or norepinephrine in the brain.

In addition to or in lieu of the therapeutic agents previously described, in some embodiments the therapeutic agent may comprise antipsychotics, including first and second generation antipsychotics, such as aripiprazole, aripirazole lauroxil, flupentixol, pipotiazine palmitate, haloperidol, asenapine, brexpiprazole, cariprazine, clozapine, iloperidone, lurasidone, olanzapine, paliperidone, quetiapine, risperidone, ziprasidone, chlorpromazine, fluphenazine, haloperidol, perphenazine, zuclopenthixol, derivatives and combinations thereof, as well as other chemical compounds configured to block dopamine receptors, 5-HT receptors, and/or dopaminergic pathways.

In addition to or in lieu of the therapeutic agents previously described, in some embodiments the therapeutic agent may comprise medications for treating dementia, such as donepezil, galantamine, rivastigmine, memantine, as well as other cholinesterase inhibitors and N-methyl-D-aspartate (NMDA) receptor agonists.

### IV. Systems and Methods for Treating Cardiovascular Disease

Additional conditions affected by medication non-adherence are hypertension and hypercholesterolemia, which represent some of the leading preventable risk factors of cardiovascular disease (CVD). It is estimated that there are more than 1 billion hypertensive patients worldwide, and more than 100 million adults in the United States alone that have cholesterol levels above what is considered to be a healthy level. Adherence to anti-hypertensive medicines is the cornerstone to blood pressure control and subsequent reductions in CVD related death and disability. However, worldwide adherence to anti-hypertensive medicines remains suboptimal at best.

The predominant classes of medications used to treat hypertension are thiazide-type diuretics, angiotensin converting enzyme (ACE) inhibitors, angiotensin II receptor blockers (ARBs), and calcium-channel blockers. The predominant classes of medications used to treat hypercholesterolemia are statins, bile-acid binding resins, and cholesterol absorption inhibitors. These medications are typically administered on a daily basis or multiple times per week, and can have side effects that include anemia, diarrhea or constipation, nausea or vomiting, and headaches. Due to the chronic nature of the treatment and possibility of these side effects, problems with medication non-adherence have become commonplace for symptoms associated with CVD, thereby leading to further cardiovascular complications.

To address these non-adherence issues, injectable medications with biweekly or monthly dosage rates have been developed. Two such medications are alirocumab (Praluent®, Sanofi, Paris, France), and evolocumab (Repatha®, Amgen Inc, Thousand Oaks, Calif.), both of which deliver proprotein convertase subtilisin-kexin type 9 (PCSK9) inhibitors that are configured to lower the amount of cholesterol circulating in a patient's bloodstream. While the biweekly and monthly dosage rates of these injectable medications are improvements over the more common orally administered daily dosage rates, these injectables still fail to provide an optimal solution to the non-adherence issues previously described. Most notably, the injectables currently available still lack a true controlled release mechanism in that they typically provide a burst of drug upon contact with surrounding physiologic fluids followed by a residual release of drug. Accordingly, the predominant cholesterol medications and their release systems do not provide an optimal treatment and/or solution to the issues associated with medication non-adherence.

Thus, a need exists for biocompatible implantable systems capable of providing a controlled release of medication to patients with symptoms associated with CVD. The depots 100 of the present technology may be used to treat a variety of symptoms and/or risk factors (e.g., abnormal blood pressure or cholesterol levels) associated with CVD depending upon the nature of the therapeutic agent delivered as described above. The depots 100 of the present technology may be implanted in vivo proximate to the target tissue (i.e., adipose tissue, bone, soft tissue, etc.) in the patient's body to provide a controlled, sustained release of a therapeutic agent for the treatment of a particular condition. For example, the depot can be configured to provide a sustained presence of the therapeutic agent to the treatment site over a predefined period of time. This implantation may be associated with a surgery or intervention for systemically treating the particular symptom or condition, whereby the depot enables chronic, sustained pharmacological treatment following completion of the surgery or intervention. The depot may be a standalone element, or may be coupled to or integrated as part of an implantable device or prosthesis associated with the intervention or surgery.

As noted elsewhere herein, the amount of the therapeutic agent that will be effective in a patient in need thereof will depend on the specific nature of the condition, and can be determined by standard clinical techniques known in the art. In addition, in vitro or in vivo assays may optionally be employed to help identify optimal dosage ranges. The specific dose level for any particular individual will depend upon a variety of factors including the activity of the drug, the age, body weight, general physical and mental health, genetic factors, environmental influences, sex, diet, time of administration, location of administration, rate of excretion, and the severity of the particular problem being treated.

Some aspects of the present technology include a system comprising a plurality of depots (each of which could be any of the depots described herein) provided for implantation by a clinical practitioner. In this system, each depot may be configured for controlled release of therapeutic agent to tissue proximate to the implantation site of the depot. The depots in the system may be identical or may vary in several respects (e.g., form factor, therapeutic agent, release profile, etc.). For example, the system may be comprised of a depot having a release profile that provides for an immediate release of therapeutic agent and other depots comprised of a depot having a release profile that provides for a delayed release of therapeutic agent.

The depot 100 used to deliver the therapeutic agent(s) for treating select symptoms or risk factors associated with CVD may comprise any of those depots 100 previously described herein, such as those described with reference to FIGS. 1-52C. For example, the depot 100 may comprise a substantially columnar shape, a substantially cylindrical shape, a dumpling configuration, a rolled film configuration, and/or microdepots (e.g., beads, microspheres, microcyliners, etc.). The shape of the depot 100, the arrangement of the various regions of the depot 100, and the mechanical properties of the depot 100 can be optimized for ease of delivery (by the patient and/or clinician) and/or patient comfort. For example, the elongated depot configurations disclosed herein have a low-profile, thereby improving patient comfort and enabling injection/insertion through a lower diameter needle. In these and other embodiments described herein, many depots 100 of the present technology are sufficiently flexible and/or provide features for preferential bending such that the depot 100 can flex or deform relative to the delivery device and/or better adapt and conform to the patient's anatomy. The flexibility and low-profile configuration of the depots 100 herein thus provide less resistance to patient movement and/or unwanted protrusions or visible indications of the depot 100 on the patient's skin, making the depots 100 more conducive to long-term (e.g., a month or multi-month) wear.

As described previously with respect to FIGS. 53A-53C, in some applications, the depot 100 may be delivered to the patient via subcutaneous or intramuscular (IM) administration, and/or positioned at or proximate to areas including the abdomen, deltoid, gluteal, arm, or thigh/femur, as well as other areas wherein the therapeutic agent delivered from the depot can be systemically absorbed into a patient's bloodstream.

In some embodiments, the depot 100 (or a system of depots 100) is configured to release the therapeutic agent at a rate according to a particular profile, as previously described with reference to FIGS. 3A and 3B. For example, the depot 100 for treating symptoms or risk factors associated with CVD can be configured to release the therapeutic agent in a substantially steady state manner (i.e., a zero-order release profile) for a desired period of time. Stated differently, the depot 100 is configured to release the therapeutic agent at a consistent rate for a period of time (e.g., from 1 day to 365 days, or as described herein in Sections I) post-implantation or post-immersion in a fluid. During such a period of time, the therapeutic agent may be released from the depot 100 to the surrounding area at a substantially consistent release rate such that about 20-30% of the therapeutic agent has been released when one quarter of the period of time has passed, 40-60% of the therapeutic agent has been released when one half of the period of time has passed, 70-80% of the therapeutic agent has been released after three quarters of the period of time has passed, at least 90% of the therapeutic agent has been released once the entire period of time has passed.

The zero-order release profile achieved by depots 100 of the present technology can be beneficial to patients across a variety of applications. For example, the consistent release of therapeutic agent may allow a more favorable pharmacokinetic profile in that the concentration of the therapeutic agent remains substantially steady over the period of treatment or release time. Compared to therapeutic agents that when delivered result in peaks and troughs of plasma drug levels, which can be associated with adverse events and symptom breakthrough, embodiments of the present technology can achieve more steady plasma drug levels on a daily, weekly, monthly, and/or yearly basis. The combination of the zero-order release profile and weekly, monthly, and/or yearly duration of release times in embodiments of the present technology offers a solution to the medication non-adherence issues previously described in that patients can receive the prescribed, steady-state dosage of the therapeutic agent via the implanted depot without bearing the burden of self-administering medication on a daily, weekly or monthly basis to maintain the prescribed plasma drug levels.

As previously described, the release profile of the depot may be tuned to release a therapeutic agent for particular durations and/or at particular release rates by adjusting the structure, composition, and the process by which the depot is manufactured. In some embodiments, including but not limited to those embodiments used to treat symptoms or risk factors associated with CVD, the depot may be configured to release the therapeutic agent through the duration of release (i.e., the period of time) at a rate of from about 1 mg/day to about 600 mg/day, about 1 mg/day to about 500 mg/day, about 1 mg/day to about 400 mg/day, about 1 mg/day to about 350 mg/day, about 1 mg/day to about 300 mg/day, about 1 mg/day to about 250 mg/day, about 1 mg/day to about 200 mg/day, about 1 mg/day to about 150 mg/day, about 1 mg/day to about 100 mg/day, about 1 mg/day to about 80 mg/day, about 1 mg/day to about 60 mg/day, about 1 mg/day to about 50 mg/day, about 1 mg/day to about 40 mg/day, about 1 mg/day to about 30 mg/day, about 1 mg/day to about 20 mg/day, about 10 mg/day to about 20 mg/day, or about 15 mg/day to about 20 mg/day, or other incremental ranges therebetween.

In some embodiments, the depot may be configured to release the therapeutic agent over the period of time at a rate no more than 600 mg/day, no more than 500 mg/day, no more than 450 mg/day, no more than 400 mg/day, no more than 350 mg/day, no more than 300 mg/day, no more than 250 mg/day, no more than 200 mg/day, no more than 175 mg/day, no more than 150 mg/day, no more than 125 mg/day, no more than 100 mg/day, no more than 90 mg/day, no more than 80 mg/day, no more than 70 mg/day, no more than 60 mg/day, no more than 50 mg/day, no more than 40 mg/day, no more than 30 mg/day, no more than 20 mg/day, no more than 15 mg/day, no more than 10 mg/day, no more than 5 mg/day, or no more than 1 mg/day.

In addition to or in lieu of the therapeutic agents previously described, in some embodiments the therapeutic agent may comprise antihypertensive agents such as thiazide-type diuretics, angiotensin converting enzyme (ACE) inhibitors, angiotensin II receptor blockers (ARBs), calcium-channel blockers, and derivatives thereof. Examples of appropriate thiazide-type diuretics include chlorthalidone, hydrochlorithiazide, indapamide, as well as other chemical compounds configured to reduce the ability of the kidneys to reabsorb salt and water from urine. Examples of appropriate ACE inhibitors benazepril, fosinopril, lisinopril, moexipril, perindopril, quinapril, ramipril, trandolapril, as well as other chemical compounds configured to decrease the activity of the ACE. Examples of appropriate ARBs can include azilsartan, candesartan, eprosartan, irbesartan, losartan, olmesartan, telmisartan, valsartan, as well as other chemical compounds configured to block or inhibit the effect of angiotensin II. Examples of appropriate calcium-channel blockers include dihydropyridine type blockers such as amlodipine, felodipine, isradipine, nicardipine, nifedipine, nisoldipine, bepridil, dilitiazem, nisoldipine, as well as nondihydropyridine type blockers such as diltiazem and verapamil.

In some embodiments, the therapeutic agent may be configured to treat hypercholesteremia or other cholesterol-related issues, and can include statins, cholesterol absorption inhibitors, proprotein convertase subtilisin-kexin type 9 (PCSK9) inhibitors, nicotinic acid, fibric acids, and omega-3-fatty acids, amongst others. Examples of appropriate statins include lovastatin, pravastatin, simvastatin, fluvastatin, atorvastatin, rosuvastatin, pitavastatin, as well as other chemical compounds configured to lower low-density lipoproteins (LDL) levels. Examples of appropriate cholesterol absorption inhibitors include exetimibe. Examples of appropriate PCSK9 inhibitors include evolocumab and alirocumab. Examples of fibric acids include gemfibrozil, bezafibrate, fenofibrate, fenofibric acid, as well as other chemical compounds configured to lower blood triglyceride levels or increase high-density lipoproteins (HDL) levels.

### V. Systems and Methods for Treating HIV and Malaria

HIV/Aids is yet another area of concern for medication non-adherence. In 2017 there were approximately 37 million people worldwide living with HIV/AIDS, of which 1.8 million were children less than 15 years old. Although there is currently no cure for HIV, effective treatment with antiretroviral therapy (ART) can control HIV to reduce the risk of transmitting the virus, as well as allow for better quality of life. HIV patients that reliably adhere to ART, for example, have a lifespan comparable to that of uninfected individuals. Additionally, ART may be used by high-risk uninfected individuals to prevent infection, an approach referred to as pre-expose prophylaxis (PrEP). Despite the clear benefits to ART, medication non-adherence has emerged as a key barrier to successful HIV treatment and prevention. These non-adherence issues are driven by a variety of factors, including access to affordable medications, the difficulty and frequency of administering pills, stigma about disease status, and side effects of ART, to name a few.

To overcome these challenges, simplified dosage regimens and long-acting injectables able to release drug for weeks after administration have been developed. However, even despite these breakthroughs, effective treatment and infection prevention of HIV is still missing. Specifically, the in vivo physiological environment quickly degrade the released drugs and result in a relatively short duration of release. As such, the ability for these drugs to actively treat HIV or prevent infection thereof is limited and does not occur over a sufficient period of time to cure non-adherence issues previously described. Moreover, the implants, injectables, extended release systems, and other means currently available for prolonging the release duration of HIV still lack a true controlled release mechanism. For example, the currently available means for delivering antiretrovirals typically provide a burst of drug upon contact with surrounding physiologic fluids, but lack an ability to release the drug in a consistent manner thereafter. It follows that current HIV treatment options are generally unable to provide for the consistent release of a drug over an extended period of time. Furthermore, many of the currently available means for delivering HIV medication are not biocompatible. As such, the delivery systems need to be removed, typically by a medical specialist.

Thus, there exists a need for a biocompatible delivery system able to deliver an antiretroviral agent over an extended period of time. The depots 100 of the present technology may be used to treat a variety of symptoms associated with HIV or malaria depending upon the nature of the therapeutic agent delivered as described above. The depots 100 of the present technology may be implanted in vivo proximate to the target tissue (i.e., adipose tissue, soft tissue, etc.) in the patient's body to provide a controlled, sustained release of a therapeutic agent for the treatment of a particular condition. For example, the depot can be configured to provide a sustained presence of the therapeutic agent to the treatment site over a predefined period of time. This implantation may be associated with a surgery or intervention for systemically treating the particular symptom or condition, whereby the depot enables chronic, sustained pharmacological treatment following completion of the surgery or intervention. The depot may be a standalone element, or may be coupled to or integrated as part of an implantable device or prosthesis associated with the intervention or surgery.

The amount of the therapeutic agent that will be effective in a patient in need thereof will depend on the specific nature of the condition, and can be determined by standard clinical techniques known in the art. In addition, in vitro or in vivo assays may optionally be employed to help identify optimal dosage ranges. The specific dose level for any particular individual will depend upon a variety of factors including the activity of the drug, the age, body weight, general physical and mental health, genetic factors, environmental influences, sex, diet, time of administration, location of administration, rate of excretion, and the severity of the particular problem being treated.

Some aspects of the present technology include a system comprising a plurality of depots (each of which could be any of the depots described herein) provided for implantation by a clinical practitioner. In this system, each depot may be configured for controlled release of therapeutic agent to tissue proximate to the implantation site of the depot. The depots in the system may be identical or may vary in several respects (e.g., form factor, therapeutic agent, release profile, etc.). For example, the system may be comprised of a depot having a release profile that provides for an immediate release of therapeutic agent and other depots comprised of a depot having a release profile that provides for a delayed release of therapeutic agent.

The depot 100 used to deliver the therapeutic agent(s) for treating select symptoms or risk factors associated with HIV or malaria may comprise any of those depots 100 previously described herein, such as those described with reference to FIGS. 1-52C. For example, the depot 100 may comprise a substantially columnar shape, a substantially cylindrical shape, a dumpling configuration, a rolled film configuration, and/or microdepots (e.g., beads, microspheres, microcyliners, etc.). The shape of the depot 100, the arrangement of the various regions of the depot 100, and the mechanical properties of the depot 100 can be optimized for ease of delivery (by the patient and/or clinician) and/or patient comfort. For example, the elongated depot configurations disclosed herein have a low-profile, thereby improving patient comfort and enabling injection/insertion through a lower diameter needle. In these and other embodiments described herein, many depots 100 of the present technology are sufficiently flexible and/or provide features for preferential bending such that the depot 100 can flex or deform relative to the delivery device and/or better adapt and conform to the patient's anatomy. The flexibility and low-profile configuration of the depots 100 herein thus provide less resistance to patient movement and/or unwanted protrusions or visible indications of the depot 100 on the patient's skin, making the depots 100 more conducive to long-term (e.g., a month or multi-month) wear.

As noted elsewhere herein with respect to FIGS. 53A-53C, in some applications, the depot 100 may be delivered to the patient via subcutaneous or intramuscular (IM) administration, and/or positioned at or proximate to areas including the abdomen, deltoid, gluteal, arm, or thigh/femur, as well as other areas wherein the therapeutic agent delivered from the depot can be systemically absorbed into a patient's bloodstream.

In some embodiments, a depot 100 for treating symptoms associated with HIV or malaria can be configured to release the therapeutic agent in a substantially steady state manner (i.e., a zero-order release profile) for a desired period of time. Stated differently, the depot 100 is configured to release the therapeutic agent at a consistent rate for a period of time (e.g., from 1 day to 365 days, or as described herein in Sections I) post-implantation or post-immersion in a fluid. During such a period of time, the therapeutic agent may be released from the depot 100 to the surrounding area at a substantially consistent release rate such that about 20-30% of the therapeutic agent has been released when one quarter of the period of time has passed, 40-60% of the therapeutic agent has been released when one half of the period of time has passed, 70-80% of the therapeutic agent has been released after three quarters of the period of time has passed, at least 90% of the therapeutic agent has been released once the entire period of time has passed.

As noted previously, the zero-order release profile achieved by depots 100 of the present technology can be beneficial to patients. For example, the consistent release of therapeutic agent may allow a more favorable pharmacokinetic profile in that the concentration of the therapeutic agent remains substantially steady over the period of treatment or release time. Compared to therapeutic agents that when delivered result in peaks and troughs of plasma drug levels, which can be associated with adverse events and symptom breakthrough, embodiments of the present technology can achieve more steady plasma drug levels on a daily, weekly, monthly, and/or yearly basis. The combination of the zero-order release profile and weekly, monthly, and/or yearly duration of release times in embodiments of the present technology offers a solution to the medication non-adherence issues previously described in that patients can receive the prescribed, steady-state dosage of the therapeutic agent via the implanted depot without bearing the burden of self-administering medication on a daily, weekly or monthly basis to maintain the prescribed plasma drug levels.

As previously described, the release profile of the depot may be tuned to release a therapeutic agent for particular durations and/or at particular release rates by adjusting the structure, composition, and the process by which the depot is manufactured. In some embodiments, including but not limited to those embodiments used to treat symptoms or risk factors associated with HIV, the depot may be configured to release the therapeutic agent through the duration of release (i.e., the period of time) at a rate of from about 100 μg/day to 50 mg/day, 100 μg/day to 40 mg/day, 100 μg/day to 30 mg/day, 100 μg/day to 20 mg/day, 100 μg/day to 10 mg/day, 100 μg/day to 5 mg/day, 100 μg/day to 1000 μg/day, 100 μg/day to 100 μg/day, 100 μg/day to 800 μg/day, 100 μg/day to 700 μg/day, 100 μg/day to 600 μg/day, 100 μg/day to 600 μg/day, 200 μg/day to 600 μg/day, 300 μg/day to 600 μg/day, 400 μg/day to 600 μg/day, or 400 μg/day to 500 μg/day, or any other incremental range therebetween (e.g., 500 μg/day to 700 μg/day). In some embodiments, the depot 100 may be configured to release the therapeutic agent through the duration of release at a rate no more than 10 mg/day, no more than 1 mg/day, no more than 500 μg/day, no more than 100 μg/day, no more than 90 μg/day, no more than 80 μg/day, no more than 70 μg/day, no more than 60 μg/day, no more than 50 μg/day. In some embodiments, the total payload (e.g., the total therapeutic agent) of the depot 100 is at least 10 mg, 50 mg, 100 mg, 200 mg, 300 mg, 400 mg, 500 mg, 600 mg, 700 mg, 800 mg, 900 mg, 1.0 g, 1.1 g, 1.2 g, 1.3 g, 1.4 g or 1.5 g. In some embodiments, the total payload of the depot 100 is from 10 mg to 1.5 g, 10 mg to 1.2 g, 10 mg to 1.1 g, 10 mg to 1.0 g, 10 mg to 900 mg, 10 mg to 800 mg, 100 mg to 800 mg, 200 mg to 800 mg, 300 mg to 800 mg, 400 mg to 800 mg, 600 mg to 800 mg, 700 mg to 800 mg, or any other incremental ranges therebetween (e.g., 600 mg to 1200 mg).

In some embodiments, including but not limited to those embodiments used to treat symptoms or risk factors associated with malaria, the depot may be configured to release the therapeutic agent through the duration of release (i.e., the period of time) at a rate of from about 10 mg/day to 400 mg/day, 10 mg/day to 350 mg/day, 10 mg/day to 300 mg/day, 10 mg/day to 250 mg/day, 50 mg/day to 250 mg/day, 100 mg/day to 250 mg/day, 150 mg/day to 250 mg/day, 200 mg/day to 250 mg/day, or any other incremental range therebetween (e.g., 300 mg/day to 700 mg/day). In some embodiments, the depot 100 may be configured to release the therapeutic agent through the duration of release at a rate no more than 400 mg/day, no more than 300 mg/day, no more than 200 mg/day, no more than 150 mg/day, no more than 100 mg/day, or no more than 50 mg/day. In some embodiments, the total payload (e.g., the total therapeutic agent) of the depot 100 is at least 10 mg, 50 mg, 100 mg, 200 mg, 300 mg, 400 mg, 500 mg, 600 mg, 700 mg, 800 mg, 900 mg, 1.0 g, 1.1 g, 1.2 g, 1.3 g, 1.4 g or 1.5 g. In some embodiments, the total payload of the depot 100 is at least 100 mg, 200 mg, 300 mg, 400 mg, 500 mg, 600 mg, 700 mg, 800 mg, 900 mg, 1.0 g, 1.5 g, 2.0 g, 3.0 g, 4.0 g, 5.0 g, 6.0 g, 7.0 g, 8.0 g, 9.0 g, or 10g of the therapeutic agent, or any other incremental ranges therebetween (e.g., 850 mg or 1.3 g).

In addition to or in lieu of the therapeutic agents previously described, in some embodiments, the therapeutic agent can be configured to treat or prevent infection of HIV. In such embodiments, the therapeutic agent includes antiretroviral, such as dolutegravir (DTG), cabotegravir (CAB), rilpivirine (RPV) and combinations thereof (e.g., DTG and RPV, DTG and CAB, and CAB and RPV). The therapeutic agent can also include entry inhibitors (e.g., enfuvirtide and maraviroc), pharmacokinetic enhancers (ritonavir and cobicistat), integrase inhibitors (e.g., raltegravir, dolutegravir and elvitegravir), nucleoside and nucleotide reverse transcriptase inhibitors (e.g., emtricitabine, lamivudine, zidovudine, didanosine, tenofovir, stavudine and abacavir), non-nuceloside reverse transcriptase inhibitors (e.g., rilpivirine, etravirine, delavirdine, doravirine, efavirenz and nevirapine), protease inhibitors (e.g., tipranavir, indinavir, saquinavir, lopinavir and norvir, fosamprenavir, darunavir, atazanavir and nelfinavir). The therapeutic agent can also include fixed dose combinations of sustiva, viread, emtriva, bictegravir, tenofovir alagenamide, edurant, pifeltro, epivir, vitekta, tybost, tivicay, retrovir and ziagen (e.g., sustiva, viread and emtriva; bictegravir, viread and tenofovir; edurant, viread and emtriva; and pifeltro, epivir and viread). The therapeutic agent can include the pharmaceutically acceptable salt of any of the therapeutic agents described herein. The therapeutic agents described herein, including their pharmaceutically acceptable salts, can be administered alone or in combination.

In some embodiments, the therapeutic agent can be configured to treat or prevent infection of malaria. In such embodiments, the therapeutic agent can include, but is not limited to, antimalarial agents, artemisinin-based combination therapies (ACTs), chemoprophylaxis, vaccines, and combinations thereof. The antimalarial agent can include quinine, chloroquine, amodiaquine, mefloquine, primaquine, sulfadoxine-pyrimethamine, intravenous artesunate, atovaquone-proguanil, azithromycin, ferroquine, artesunate, foxmidomycin, clindamycin, ozonide, piperaquine, sprioindolone, artesunate-amodiaquine, artesunate, coartem, eurartesim, pyramax, imidazolopiperazine, timidazole, tafenoquine, bulaquine, and/or combinations thereof. The vaccine can include RTS,S. In some embodiments, the therapeutic agent can be configurd to treat P. falciparum, or target infected red blood cells (iRBC).

In some embodiments, the therapeutic agents described herein may comprise or be combined with one or more adjunctive agents, including analgesics, chemotherapeutic agents, anti-inflammatory agents, antibiotics and/or antimicrobial agents, antifungal agents, agents that promote nerve regeneration, steroids, immunosuppressants, pharmaceutically acceptable salts thereof, and combinations thereof.

The analgesics include, but are not limited to, bupivacaine, ropivacaine, mepivacaine, etidocaine, levobupivacaine, trimecaine, carticaine, articaine, lidocaine, prilocaine, benzocaine, procaine, tetracaine, chloroprocaine, and combinations thereof.

The chemotherapeutic agents include, but are not limited to, antibodies, alkylating agents, angiogenesis inhibitors, antimetabolites, DNA cleavers, DNA crosslinkers, DNA intercalators, DNA minor groove binders, enediynes, heat shock protein 90 inhibitors, histone deacetylase inhibitors, immunomodulators, microtubule stabilizers, nucleoside (purine or pyrimidine) analogs, nuclear export inhibitors, proteasome inhibitors, topoisomerase (I or II) inhibitors, tyrosine kinase inhibitors, and serine/threonine kinase inhibitors. Specific therapeutic agents include, but are not limited to, adalimumab, ansamitocin P3, auristatin, bendamustine, bevacizumab, bicalutamide, bleomycin, bortezomib, busulfan, callistatin A, camptothecin, capecitabine, carboplatin, carmustine, cetuximab, cisplatin, cladribin, cytarabin, cryptophycins, dacarbazine, dasatinib, daunorubicin, docetaxel, doxorubicin, duocarmycin, dynemycin A, epothilones, etoposide, floxuridine, fludarabine, 5-fluorouracil, gefitinib, gemcitabine, ipilimumab, hydroxyurea, imatinib, infliximab, interferons, interleukins, beta-lapachone, lenalidomide, irinotecan, maytansine, mechlorethamine, melphalan, 6-mercaptopurine, methotrexate, mitomycin C, nilotinib, oxaliplatin, paclitaxel, procarbazine, suberoylanilide hydroxamic acid (SAHA), 6-thioguanidine, thiotepa, teniposide, topotecan, trastuzumab, trichostatin A, vinblastine, vincristine, vindesine, tamoxifen and combinations thereof.

The anti-inflammatory agents include, but are not limited to, prednisone, betamethasone, cortisone, dexamethasone, hydrocortisone, methylprednisolone, aspirin, Ibuprofen, naproxen sodium, diclofenac, diclofenac-misoprostol, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, mefenamic acid, COX-2 inhibitors, and combinations thereof.

The antibiotics and/or antimicrobial agents include, but are not limited to, amoxicillin, amoxicillin/clavulanate, cephalexin, ciprofloxacin, clindamycin, metronidazole, azithromycin, levofloxacin, sulfamethoxazole/trimethoprim, tetracycline(s), minocycline, tigecycline, doxycycline, rifampin, triclosan, chlorhexidine, penicillin(s), aminoglycides, quinolones, fluoroquinolones, vancomycin, gentamycin, cephalosporin(s), carbapenems, imipenem, ertapenem, antimicrobial peptides, cecropin-mellitin, magainin, dermaseptin, cathelicidin, α-defensins, α-protegrins, and combinations thereof.

The antifungal agents include, but are not limited to, ketoconazole, clortrimazole, miconazole, econazole, intraconazole, fluconazole, bifoconazole, terconazole, butaconazole, tioconazole, oxiconazole, sulconazole, saperconazole, voriconazole, terbinafine, amorolfine, naftifine, griseofulvin, haloprogin, butenafine, tolnaftate, nystatin, cyclohexamide, ciclopirox, flucytosine, terbinafine, amphotericin, and combinations thereof.

The steroids include, but are not limited to, prednisone, betamethasone, cortisone, dexamethasone, hydrocortisone, methylprednisolone, and combinations thereof.

The immunosuppressants include, but are not limited to, cyclosporine, pimecrolimus, sirolimus, tacrolimus, and combinations thereof.

VI. Systems and Methods for Treating or Preventing Infection Associated with Implantable Medical Devices

Implantable medical devices (IMD) are becoming increasingly common. While providing many benefits to patients, in some instances the implanted devices develop infections, which can be dangerous to the patient and extremely costly to treat. For example, it can cost more than $50,000 to treat infection following an arthroplasty procedure. Because infections typically develop at the interface between the IMD surface and the surrounding tissue, local delivery of antibiotics is preferable and more effective than systemic clinical treatments. A number of attempts have been made to provide antimicrobial films, coatings, cases, or other systems to deliver antibiotic and/or antimicrobial agents to medical devices prior to or after implantation within a patient. However, the currently available means for delivering antimicrobial and/or antibiotic agents typically fail to effectively provide for a controlled, sustained release over an extended period of time, instead typically providing a burst of drug upon contact with surrounding physiologic fluids. It follows that current options are generally unable to provide for the consistent release of antibiotic and/or antimicrobial agents to ward off infection of an implanted medical device over an extended period of time. Thus, a need exists for implantable systems capable of providing a controlled release of antibiotic and/or antimicrobial agents to treat or prevent infection associated with implanted medical devices.

Embodiments of the present technology relate to implants and insertables configured to be disposed at a surgical or interventional treatment site proximate an IMD for controlled release of a therapeutic agent over a period of time to treat or prevent infection. For example, the depot can be configured to provide a sustained presence of the therapeutic agent to the treatment site over a predefined period of time. As described in more detail below, in some embodiments the depots 100 described herein can be configured to at least partially surround or cover an implantable medical device, to be coupled to an exterior surface of an IMD, or to be integrated into an exterior surface of an IMD, and release one or more therapeutic agents configured to prevent or treat infection. The depot 100 may be tuned to meet the particular conditions of patients receiving the implant patients, e.g., by altering various factors (e.g., shape and/or configuration) of the depot 100 such that the depot 100 has a particular release profile, duration of release, and/or desired effect on the tissue adjacent to the implantable medical device.

The amount of the therapeutic agent that will be effective in a patient in need thereof will depend on the specific nature of the condition, and can be determined by standard clinical techniques known in the art. In addition, in vitro or in vivo assays may optionally be employed to help identify optimal dosage ranges. The specific dose level for any particular individual will depend upon a variety of factors including the activity of the drug, the age, body weight, general physical and mental health, genetic factors, environmental influences, sex, diet, time of administration, location of administration, rate of excretion, and the severity of the particular problem being treated.

Some aspects of the present technology include a system comprising a plurality of depots (each of which could be any of the depots described herein) provided for implantation by a clinical practitioner. In this system, each depot may be configured for controlled release of therapeutic agent to tissue proximate to the implantation site of the depot. The depots in the system may be identical or may vary in several respects (e.g., form factor, therapeutic agent, release profile, etc.). For example, the system may be comprised of a depot having a release profile that provides for an immediate release of therapeutic agent and other depots comprised of a depot having a release profile that provides for a delayed release of therapeutic agent.

Embodiments of the present technology can be applied any device configured to implanted, whether temporarily or permanently, within a patient. Examples of IMDs include, but are not limited to, intravascular IMDs (e.g., peripheral venous catheters, peripheral arterial catheters, midline catheters, central venous catheters, non-tunneled catheters, tunneled catheters, pulmonary artery catheters, totally implanted ports, and vascular access devices), cardiovascular IMDs (e.g., mechanical heart valves, implantable defibrillators and related devices, pacemakers, vascular grafts, ventricular assist devices, coronary stents, and implantable patient monitors), neurosurgical IMDs (e.g., ventricular shunts, Ommaya reservoirs, intracranial pressure devices, and implantable neurological stimulators), orthopedic IMDs (e.g., joint prosthesis and other reconstructive orthopedic implants, spinal implants, fracture-fixation devices), urological IMDs (e.g., inflatable penile implants), gynecological IMDs (e.g., IMDs), otolaryngological IMDs (e.g., cochlear implants, middle-ear implants), ophthalmological IMDs (e.g., intra-ocular lenses, glaucoma tubes), dental IMDs (e.g., dental implants, dental appliances). As described in more detail below, in various embodiments one or more depots 100 can be configured to be coupled to an IMD, for example partially or completely surrounding the IMD, affixed to an outer surface of an IMD, disposed adjacent to an IMD within the body, etc.

The therapeutic agent carried by the depots 100 of the present technology may be any biologically active substance (or combination of substances) that provide a therapeutic effect in a patient in need thereof. In some embodiments, the therapeutic agent include an antibiotic agent, and anti-biofilm agent, an anti-septic agent, an anti-fungal agent, or other agents effective to treat various conditions associated with implant surgery and recovery.

Antibiotic agents can be any substance with antibiotic and/or antimicrobial properties. Examples of antibiotic agents include, but are not limited to, amoxicillin, amoxicillin/clavulanate, ampicilline, cephalexin, cefixime, ceftriaxone, ciprofloxacin, clindamycin, cloxacillin, cotrimaxazole, metronidazole, clindamycin, azithromycin, erythromycin, and clarithromycin, levofloxacin, ofloxacin, sulfamethoxazole/trimethoprim, tetracycline(s), minocycline, tigecycline, doxycycline, rifampin, triclosan, chlorhexidine, penicillin(s), aminoglycides, quinolones, fluoroquinolones,beta-lactams, rifampicin, vancomycin, daptomycin, fostomycin, gentamycin, cephalosporin(s), carbapenems, imipenem, ertapenem, antimicrobial peptides, cecropin-mellitin, magainin, dermaseptin, cathelicidin, α-defensins, α-protegrins, and/or combinations thereof.

Anti-biofilm agents include therapeutic agents configured to inhibit formation of or promote dissolution of biofilms within the body. Example anti-biofilm agents include, but are not limited to, lactoferrin, ethylenediaminetetraacetic acid (EDTA), xylitol, gallium, dispersin B, farsenol, RNA-III inhibiting peptide (RIP), and furanone C30, lysostaphin, DNase I, V8 protease, apto-transferrin, ethylene glycol tetraacetic acid (EGTA), and 1,2,3,4,6-Penta-O-galloyl-beta-D-glucopyranose (PGG), Cis-2 decenoic acid (C2DA), diarylacrylonitriles, aryl ethyl ketones, and vinyl sulfones.

Anti-septic agents include, but are not limited to, N-acetyl-L-cysetine (NAC), ethanol, and chlorohexidine. Antifungal agents include, but are not limited to, ketoconazole, clortrimazole, miconazole, econazole, intraconazole, fluconazole, bifoconazole, terconazole, butaconazole, tioconazole, oxiconazole, sulconazole, saperconazole, voriconazole, terbinafine, amorolfine, naftifine, griseofulvin, haloprogin, butenafine, tolnaftate, nystatin, cyclohexamide, ciclopirox, flucytosine, terbinafine, and amphotericin B.

In some embodiments, the depot 100 can be configured to release the therapeutic agent at a rate of between about 0.1 g and about 10 g per day, for example between about 0.5 g and about 5 g per day. In some embodiments, the depot 100 can be configured to release the therapeutic agent at a rate of at least 100 mg per day, at least 200 mg per day, at least 300 mg per day, at least 400 mg per day, at least 500 mg per day, at least 600 mg per day, at least 700 mg per day, at least 800 mg per day, at least 900 mg per day, at least 1 g per day, at least 1.5 g per day, at least 2 g per day, at least 2.5 g per day, at least 3 g per day, at least 4 g per day, at least 5 g per day, at least 6 g per day, at least 7 g per day, at least 8 g per day, at least 9 g per day, or at least 10 g per day. In some embodiments, the depot 100 is configured to release the therapeutic agent at a rate of no more than 100 mg per day, no more than 200 mg per day, no more than 300 mg per day, no more than 400 mg per day, no more than 500 mg per day, no more than 600 mg per day, no more than 700 mg per day, no more than 800 mg per day, no more than 900 mg per day, no more than 1 g per day, no more than 1.5 g per day, no more than 2 g per day, no more than 2.5 g per day, no more than 3 g per day, no more than 4 g per day, no more than 5 g per day, no more than 6 g per day, no more than 7 g per day, no more than 8 g per day, no more than 9 g per day, or no more than 10 g per day. The particular rate of release can be tailored to the particular application and the particular therapeutic agent carried by the depot 100.

In some embodiments, the total payload (e.g., the total therapeutic agent or combination of therapeutic agent and any adjunctive agent) of the depot 100 may be at least 100 mg, at least 150 mg, at least 200 mg, at least 300 mg, at least 400 mg, at least 500 mg, at least 600 mg, at least 700 mg, at least 800 mg, at least 900 mg, at least 1 g, at least 2 g, at least 3 g, at least 4 g, at least 5 g, at least 6 g, at least 7 g, at least 8 g, at least 9 g, at least 10 g, at least 11 g, at least 12 g, at least 13 g, at least 14 g, at least 15 g, at least 16 g, at least 17 g, at least 18 g, at least 19 g, or at least 20 g.

In some embodiments, the depot 100 is configured to release the therapeutic agent through the duration of release at a rate of from about 0.1 mg/day to about 200 mg/day, about 0.1 mg/day to about 150 mg/day, about 0.1 mg/day to about 100 mg/day, about 0.1 mg/day to about 90 mg/day, about 0.1 mg/day to about 80 mg/day, about 0.1 mg/day to about 70 mg/day, about 0.1 mg/day to about 60 mg/day, about 0.1 mg/day to about 50 mg/day, about 0.1 mg/day to about 40 mg/day, about 0.1 mg/day to about 30 mg/day, about 1 mg/day to about 30 mg/day, about 1 mg/day to about 20 mg/day, about 5 mg/day to about 20 mg/day, about 10 mg/day to about 20 mg/day, or about 15 mg/day to about 20 mg/day, or any other incremental ranges therebetween (e.g., 50 mg/day to 100 mg/day, 150 mg/day to 175 mg/day, etc.).

As previously described, in some embodiments the depot 100 is configured to release the therapeutic agent over a varying period of time (i.e., duration of release). For those embodiments associated with treating or preventing infection associated with an IMD, the depot 100 can be configured to release the therapeutic agent(s) at the treatment site for no less than 1 day, no less than 2 days, no less than 3 days, no less than 4 days, no less than 5 days, no less than 6 days, no less than 7 days, no less than 8 days, no less than 9 days, no less than 10 days, no less than 11 days, no less than 12 days, no less than 13 days, no less than 14 days, no less than 15 days, no less than 16 days, no less than 17 days, no less than 18 days, no less than 19 days, no less than 20 days, no less than 21 days, no less than 22 days, no less than 23 days, no less than 24 days, no less than 25 days, no less than 26 days, no less than 27 days, no less than 28 days, no less than 29 days, no less than 30 days, no less than 40 days, no less than 50 days, no less than 60 days, no less than 70 days, no less than 90 days, no less than 100 days, no less than 150 days, no less than 200 days, no less than 300 days, or no less than 365 days.

As previously described, the depot 100 of the present technology can achieve a release profile or kinetics that suits the objectives of the intended therapy. For those embodiments directed to treating or preventing infection associated with an IMD, the release profile may be (a) zero-order such that release of the payload of therapeutic agent is at a substantially steady rate over the duration of release, (b) first-order such that release of the payload of the therapeutic agent increases in a substantially linear manner over the duration of release, or (c) a second-order such that release of the payload of the therapeutic agent occurs at a high, substantially linear rate for a first period of time and then at a lower, substantially linear rate for a second period of time over the duration of release.

Each of these release profiles can be advantageous for patient having implanted medical devices depending on their particular condition. For example, a zero-order release profile may be desired when the therapeutic agent is used predominantly to prevent infection. In such cases, release of the therapeutic agent in a substantially consistent manner over a duration of release can maximize the amount of time drug is released from the depot, thereby maximizing the amount of time that infection is actively prevented by the therapeutic agent. As another example, a second-order release profile may be desired when bacterial infection is already present at or adjacent to the treatment site. In such cases, release of therapeutic agent during a first period of time at the higher rate is used to first target the existing infection around the IMD, and a subsequent release of therapeutic agent during a second period of time at the lower rate is used to prevent recurrence of infection. Embodiments of the present technology enable the depot to be tuned according to the optimal treatment needed for each patient.

The depots 100 of the present technology previously described are generally applicable to use in conjunction with IMDs. In some embodiments, certain form factors may be particularly beneficial to achieve more effective treatment. For example, in some embodiments the depot 100 can be shaped, dimensioned, and configured to form a cover for an IMD, for example extending over a portion or all of the exterior surface of an IMD. In such embodiments, the depot 100 when used with IMDs can reduce or prevent infection as well as treat or prevent pain, inflammation, scarring or other indications or complications associated with IMDs.

In various embodiments, the depot 100 may be a cover formed separately from an IMD into which an IMD may be inserted, or the depot 100 may be a coating formed upon the surface of the IMD. FIG. 52 illustrates an IMD 700 partially surrounded by a depot 100. While the illustrated embodiment depicts the IMD 700 as an orthopedic hip implant, in various embodiments the IMD 700 may take other forms, for example other orthopedic implants, vascular implants, neurological implants, or any of the other IMDs noted previously herein or any other IMD. Although the illustrated embodiment depicts the depot 100 partially surrounding the IMD 700, in various embodiments the depot 100 may cover all or substantially all of the outer surface of the IMD 700. In some embodiments, the depot 100 covers only a relatively small portion of the IMD 700, for example forming a strip or patch that extends only partially over the surface of the IMD 700. Once implanted in the patient, the depot 100 will come into contact with physiological fluids and release therapeutic agent(s) to surrounding tissue as described previously herein.

As will be readily apparent to one of skill in the art, the size and shape of any particular IMD can vary widely. For example, orthopedic IMDs can include a femoral head implant which is semispherical shell, a bone fixation plate which is an elongated planar strip, and a bone anchor which can be an elongate threaded rod. For these and other IMDs, one or more depots 100 can be configured to have sizes, dimensions, and other properties (e.g., elasticity, flexural strength, etc.) suited to the particular IMD.

As noted above, the present invention is directed to a depot that covers at least a portion of an IMD. The depot 100 in the form of a cover has an appropriate form-fitting shape for the implant and may encase an implant in the operating room prior to its insertion during surgery. The depot 100 in the form of a cover can also be preassembled with the IMD and supplied for surgery as an IMD assembly, where the term “IMD assembly” also includes a depot 100 supplied for surgery in the form of a coated IMD.

In some embodiments, the depot 100 has substantially the same shape and size as the implant itself, and partially or completely covers the IMD 700. In some embodiments, the depot 100 is shaped to define an inner chamber with an opening, and the depot 100 may be sealed after the implant is inserted into the inner space of the depot 100 via the opening, for example by the depot 100 being shrunk around the IMD 700 to provide a closely fitting covering. The depot 100 can be useful to reduce infection, for example by delivering therapeutic agent(s) (e.g., antibiotics) into the surrounding tissue, to reduce and/or treat pain and/or other conditions, indications or complications associated with IMDs.

The depot 100 may have a substantially uniform thickness over the surface of the IMD 700. In other embodiments, the depot 100 can have a greater thickness in a first region than in a second region. In some embodiments, the implant is inserted into the depot 100 through a suitable aperture, which preferably may be a slit that can readily be overlapped to fully cover the IMD 700 so as to minimize and preferably prevent infection to an uncovered portion of the implant. Those of skill in the art can readily determine appropriate shapes, sizes and configurations for the apertures or slits for a given size and shape of IMD 700.

In some embodiments, the depot 100 may cover only relatively small portion of the IMD 700, and may be adhered to the outer surface using adhesive, barbs, hooks, tines, or other fixation mechanisms. In some embodiments, the depot 100 can be sutured or otherwise attached to a portion of the IMD 700 prior to surgery or after the IMD has already been surgically implanted within the patient.

In some embodiments, the depot 100 include a layered design, such as those depot embodiments comprising a therapeutic region including a first portion having a therapeutic agent, and a second portion having an adjunctive agent (e.g., an immunotherapeutic agent, anti-inflammatory agent, antibiotic agent and/or antifungal agent). Such embodiments can provide the combined release (e.g., simultaneous or sequential release) of the therapeutic agent and adjunctive agent. In some embodiments, the depot 100 can include a barrier region 400 configured to face towards the IMD, such that therapeutic agent released from the depot 100 preferentially is released away from the IMD.

In some embodiments, the depot 100 in the form of a cover may also be elastomeric so that it can be stretched around the IMD, or can be stretched and will shrink to fit tightly around the IMD. For example, the depot 100 can form a socket or pocket with at least one opening, or a sleeve or band with at least two opening. In some embodiments, so that for the socket can securely hold an IMD, the depot 100 made from the elastomeric material can be smaller than the IMD to be inserted into it when the depot 100 is in the relaxed state. This may result in the depot 100 securely holding the IMD by a resilient holding force generated from the elastomeric polymeric material that makes up the depot 100. In some embodiments, the depot 100 in the form of a socket (and hence the opening(s) defined by the depot 100) are stretchable to at least 1.1 times (e.g. from 1.2 times to 10 times) to allow for insertion of the IMD into the socket, and can recover to more than 80% to securely hold the IMD within the socket and prevent slippage or fall off.

In various embodiments, the depot 100 can form a socket, pocket, sleeve, or band configured to securely hold an IMD by surrounding the whole or part of the IMD (e.g. the socket may leave parts of the object uncovered to enable further connectivity of the IMD). In some embodiments, the depot 100 can be smaller than the IMD it is intended to hold and accomplishes the secure holding by its elastic nature of the depot 100, such that the depot 100 may be stretched to a size larger than the IMD and then recovers towards its original size once the IMD has been placed within the socket, pocket, sleeve, or band defined by the depot 100. As will be apparent, the depot 100 may include at least one opening to permit the IMD to be inserted within it. In various embodiments, the depot 100 may define one, two, or more openings configured to receive all or a portion of the IMD therethrough.

This may be accomplished by the resilient force applied to the object inserted into the socket by the elastomeric polymeric material that comprises the film. Thus, the socket or film can resiliently engage or resiliently hold a device inserted into a socket formed from the film after stretching. In addition or alternatively, the socket or film can stretch from its original size to an expanded size and return to its original size or to a size no greater than the expanded size minus (80% of the difference between expanded size and original size), optionally wherein the socket or film can stretch from its original size to an expanded size and return to its original size or to a size no greater than the expanded size minus (90% of the difference between expanded size and original size).

The controlled release antibiotic socket of the current invention provides enhanced stability of the object (e.g., the IMD) within the socket, reducing the possibility of the object falling out of the socket, and is able to effectively achieve that for a wide range of objects (e.g., IMDs of different sizes) with one size of socket. The selection of an elastic polymeric material is an intricate balance of elastic modulus and strain recovery. Certain polymers with high elasticity (low elastic modulus) have poor strain recovery, and would not be able to hold an object well. Other polymers that have relatively low elasticity (high elastic modulus) are not suitable for the construction of a sleeve that can securely hold different sizes of object. The design of the sleeve aids in enhancing the stability of the object (e.g., an IMD) within. Thus care need to be exercised in selecting the materials used to form the polymer film.

When used herein, the term “elastic” refers to a material or an assembly (e.g., a depot in the form of a multilayer thin film) that can resist a distorting influence or stress and can return to towards its original size and shape when the stress is removed. For example, an elastic depot 100 may be stretched up to 10 times its original size in any direction (e.g. from 1.1 times to 4 times its original size) and may then recover at least to 80%, such as at least 90% of its original size following release of the stretch. For example, when stretching a depot 100 to size B (a difference of size C) from size A results in the depot 100 returning to a maximum size of B−(0.8×C) following stretching and release, where C is B−A, such as a maximum size of B−(0.9×C). That is, if one stretches a depot 100 from 0.1 cm to 0.11 cm (difference of 0.01 cm), the resulting depot 100 will have maximum size of 0.11−(0.8×0.01)=0.102 cm if the depot 100 recovers at least to 80% of its original size or will have a maximum size of 0.101 cm if the depot 100 recovers to at least 90% of its original size following stretching. In some embodiments, the depot 100 may recover to its original size or almost to its original size.

After manufacture, the depot 100 in the form of a cover can be sterilized and packaged for assembly onto an IMD 700 immediately prior to surgery. Alternatively, the depot 100 of can be assembled onto the IMD 700, sterilized and packaged at the time of manufacture, so that a completed IMD assembly is delivered to the surgical suite. Sterile gloves and sterile and atraumatic instruments may be used when handling the coverings to provide a sterile IMD assembly that includes the IMD 700 and the depot 100. Once form-fitted with depot 100 as a cover or as a coated IMD 700, the implant assembly can be inserted into the subject using standard surgical techniques.

In some embodiments, one or more depots 100 can be provided with an IMD 700 as a kit. For example, the depot 100 in the kit may be size matched to the IMD 700 supplied with the kit. The kits may be sterile, and may contain instructions for inserting the accompanying IMD 700 into the depot 100 or for handling and surgically implanting the IMD assembly in the subject. In operation, the kits may be opened, and the IMD 700 may be inserted into the depot 100 before implantation in the patient. Alternatively, the kit can include an IMD assembly that comprises the depot 100 coupled to the IMD 700 (e.g., wrapped around the IMD 700), or an IMD 700 coated with the depot 100. As with the previously described kits, the depots 100 and IMDS of the assembly can be appropriately size-matched and sterile.

### VII. Systems and Methods for Treating Ocular Conditions

Ocular diseases such as diabetic retinopathy, glaucoma, and macular degeneration represent a significant burden on public health generally. In the United States alone, it is estimated that roughly 9 million adults have been diagnosed with at least one of these conditions. Moreover, as the majority of these diseases disproportionately affect elderly populations, their prevalence will likely increase in the coming years as the worldwide population continues to age.

Ocular diseases can be difficult to treat for a number of reasons. For example, treatment typically requires ocular drugs to be received at the posterior segment of the eye, which is impeded from the eye's anterior segment by both static barriers to penetration (e.g., the conjunctiva, sclera, choroid, retinal pigment epithelium and the cornea), as well as dynamic barriers to penetration (e.g., tear flow and nasolacrimal drainage, the subconjunctival-episcleral lymph and choroidal circulation). As a result, topical drug administration to anterior portions of the eye is rarely successful. Other factors such as poor patient adherence to daily medication dosing instructions, difficulties in accurately administering drug to the eye, and variable drug efficacy further decrease the effective treatment of ocular diseases via topical drug administration.

Other currently used delivery methods for ocular drugs include intravitreal injection, suprachoroidal injection, and sub-tenon's injection, amongst others. However, each of these methods also have significant drawbacks. For example, intravitreal injection can be ineffective due to the short half-life of ocular drugs after in vivo placement, and generally difficult because repeated intravitreal injections can cause vitreous hemorrhage, retinal detachment, and ocular trauma.

In an effort to overcome the invasive nature of repeated intravitreal injections (and other similar surgeries), a number of extended release systems have been developed for treating ocular diseases. However, the currently available release systems for ocular diseases have drawbacks that prevent effective treatment thereof. Specifically, the physiological environment of intrascleral, subretinal, suprachoroidal, punctal, subconjunctival, or intravitreal aspects of the eye can degrade drugs implanted therein in a relatively short timeframe. As such, the ability for these drugs to actively treat the underlying disease is limited and does not occur over a sufficient period of time. Moreover, the implants, injectables, extended release systems, and other means currently available to prolong the release duration of ocular drugs still lack a true controlled release mechanism. For example, the currently available means for delivering medication to ocular regions typically provide a burst of drug upon contact with surrounding physiologic fluids, but lack an ability to release the drug in a consistent manner thereafter. It follows that current treatment options for ocular diseases are generally unable to provide for the consistent release of a drug over an extended period of time. Furthermore, many of the currently available means for delivering medication to ocular regions are not biocompatible and thus need to removed (e.g., by a medical specialist).

Thus, there exists a need for a biocompatible ocular delivery system able to deliver a therapeutic agent over an extended period of time. The depots 100 of the present technology may be used to treat a variety of symptoms (e.g., abnormal intraocular pressure) associated with ocular conditions depending upon the nature of the therapeutic agent delivered as described above. The depots 100 of the present technology may be implanted in vivo proximate to the eye to provide a localized, controlled and sustained release of a therapeutic agent for the treatment of a particular condition. For example, the depot can be configured to provide a sustained presence of the therapeutic agent to the treatment site over a predefined period of time. This implantation may be associated with a surgery or intervention for treating the particular symptom or condition, whereby the depot enables chronic, sustained pharmacological treatment following completion of the surgery or intervention. The depot 100 may be a standalone element, or may be coupled to or integrated as part of an implantable device or prosthesis associated with the intervention or surgery.

The amount of the therapeutic agent that will be effective in a patient in need thereof will depend on the specific nature of the condition, and can be determined by standard clinical techniques known in the art. In addition, in vitro or in vivo assays may optionally be employed to help identify optimal dosage ranges. The specific dose level for any particular individual will depend upon a variety of factors including the activity of the drug, the age, genetic factors, environmental influences, sex, diet, time of administration, location of administration, and the severity of the particular problem being treated.

Some aspects of the present technology include a system comprising a plurality of depots (each of which could be any of the depots described herein) provided for implantation by a clinical practitioner. In such a system, each depot may be configured for controlled release of therapeutic agent to the eye. The depots in the system may be identical or may vary in several respects (e.g., form factor, therapeutic agent, release profile, etc.). For example, the system may be comprised of one or more depots having a release profile that provides for an immediate release of therapeutic agent and other depots comprised of a depot having a release profile that provides for a delayed release of therapeutic agent.

The depot 100 used to deliver the therapeutic agent(s) for treating select symptoms associated with ocular conditions may comprise any of those depots 100 previously described herein, such as those described with reference to FIGS. 1-52C. For example, the depot 100 may comprise a substantially columnar shape, pellets, disks, a substantially cylindrical shape, a dumpling configuration, a rolled film configuration, and/or microdepots (e.g., beads, microspheres, microcyliners, etc.). The shape of the depot 100, the arrangement of the various regions of the depot 100, and the mechanical properties of the depot 100 can be optimized for ease of delivery by the clinician. For example, the elongated depot configurations disclosed herein have a low-profile, thereby improving patient comfort and enabling injection/insertion through a lower diameter needle. In these and other embodiments described herein, many depots 100 of the present technology are sufficiently flexible and/or provide features for preferential bending such that the depot 100 can flex or deform relative to the delivery device and/or better adapt and conform to the anatomy of the eye. The flexibility and low-profile configuration of the depots 100 herein thus provide less resistance to eye movement, making the depots 100 more conducive to long-term (e.g., a month or multi-month) wear.

The depots 100 of the present technology described herein can be positioned proximate any region of the eye. For example, depots 100 of the present technology can be positioned at or proximate the conjunctiva, subconjunctival space, punctal space, cornea, sclera, pars plana, macula, vitreous cavity, choroid, suprachoroidal space, retina, posterior chamber, anterior chamber, and surgically-induced avascular regions of the eye. FIG. 55 is an anatomical cross-sectional illustration of the eye including multiple depots, in accordance with embodiments of the present technology. As shown in the illustrated embodiment, depot 100a is positioned proximate an outer surface of the cornea, depot 100b is positioned proximate the subconjunctival space, depot 100c is positioned proximate the conjunctiva or punctal space, depot 100d is positioned proximate the anterior sub-Tenon space, depot 100e is positioned at the vitreous cavity in vitreous gel, depot 100f is positioned proximate the choroid at a suprachoroidal space, depot 100g is positioned proximate the posterior sub-Tenon space and optic nerve, depot 100h is positioned proximate the retina at a subretinal space, and depot 100i proximate the sclera at a intrascleral space. The depots 100a-i and their positioning, as shown in FIG. 55, represent some embodiments of the present technology. In other embodiments, the depots may be positioned at other regions of the eye and/or include different depot configurations. For example, as illustrated in FIG. 55, depot 100e comprises a cylindrical configuration, but in other embodiments could include a microdepot, such as a microsphere or bead configuration.

Depots 100a-i can include any of the configurations or form factors previously described, though certain configurations and form factors may be particularly beneficial to achieve more effective treatment of ocular conditions. For example, depots 100b, 100d, 100e, 100f, 100h and 100i may beneficially include a thin film configuration, e.g., to enable the depot to be sandwiched between adjacent layers of the eye, such as the sclera, choroid and retina, and/or maximize the surface in contact with such layers. As another example, depots 100a, 100b, 100d, 100f, 100h and 100i may beneficially include a curved, bent, or rounded configuration, e.g., to increase or provide adequate contact with the curved surface area of a treatment site, such as the sclera, choroid, retina or cornea. As previously described, the curved configuration can be achieved after the depot 100 is deployed in vivo in the presence of physiological fluids and/or temperatures. Additionally, in some embodiments, elasticity of one or more layers (e.g., the control region) of the depot 100 can be adjusted tuned based at least in part of the target site. As another example, depots 100c and 100g may beneficially include a pellet configuration having a cylindrical, circular, elliptical, regular polygonal or irregular polygonal shape, e.g., to complement that of the target site (e.g., the conjunctiva or posterior sub-Tenon space). As another example, depots 100a, 100b, 100d, 100e, 100f, 100h and 100i may include a polymer composition that is more hydrophilic (e.g., PLGA) or less hydrophilic (PLA) than other regions of the depot to absorb more of less water or other fluids when implanted in vivo. Depending on the intended application, a hydrophilic or hydrophobic polymer composition of the depot 100 can prolong or decrease the duration of release of therapeutic region to the eye. For example, a hydrophilic polymer composition of depots 100a or 100e could enable a quicker release of therapeutic agent once implanted in vivo. As another example, depots 100a-i may include a fixation portion (e.g., a tab, a ridge, a hook, a barb, a protrusion, or a notch) configured to penetrate at least a portion of the thickness of the adjacent ocular structure (e.g., the sclera, choroid, retina, etc.) and thereby secure the depot at the eye.

In some embodiments, the depot 100 (or a system of depots 100) is configured to release the therapeutic agent at a rate according to a particular profile, as previously described, e.g., with reference to FIGS. 3A and 3B. For those embodiments directed to treating ocular conditions, the release profile may be (a) zero-order such that release of the payload of therapeutic agent is at a substantially steady rate over the duration of release, (b) first-order such that release of the payload of the therapeutic agent increases in a substantially linear manner over the duration of release, or (c) second-order such that release of the payload of the therapeutic agent occurs at a high, substantially linear rate for a first period of time and then at a lower, substantially linear rate for a second period of time over the duration of release.

Each of these release profiles can be advantageous depending on a particular ocular condition. For example, a zero-order release profile may be desired when a continuous and steady supply of the therapeutic agent is needed. For example, ocular hypertension is a risk factor for glaucoma and is treated by reducing intraocular pressure (TOP). Conventional methods for reducing IOP involve topically administering eye drops several times daily, the difficult nature of which leads to relatively high non-adherence rates amongst patients. Depots 100 of embodiments of the present technology having a zero-order release profile can be implanted at the ocular region to mitigate this non-adherence by releasing the therapeutic agent in a substantially consistent manner over an extended period of time. As such, the patient's burden of self-administering a therapeutic agent on a daily or weekly basis can be relieved.

As previously described, the release profile of the depot may be tuned to release a therapeutic agent for particular durations and/or at particular release rates by adjusting the structure, composition, and the process by which the depot is manufactured. In some embodiments, the total payload (e.g., the total therapeutic agent) of the depot 100 is at least 10 μg, 50 μg, 100 μg, 200 μg, 300 μg, 400 μg, 500 μg, 600 μg, 700 μg, 800 μg, 900 μg, 1 mg, 10 mg, 100 mg, or 150 mg. In some embodiments, the total payload of the depot 100 is from 10 μg to 10 mg, 10 μg to 1000 μg, 10 μg to 900 μg, 10 μg to 800 μg, 10 μg to 700 μg, 10 μg to 600 μg, 10 μg to 500 μg, 10 μg to 400 μg, 10 μg to 300 μg, 10 μg to 200 μg, 10 μg to 100 μg, 10 μg to 75 μg, 10 μg to 50 μg, or 10 μg to 20 μg, or any other incremental ranges therebetween (e.g., 20 μg to 60 μg).

In some embodiments, the depot 100 is configured to release the therapeutic agent through the duration of release at a rate of from about 10 ng/day to 900 μg/day, 10 ng/day to 700 μg/day, 10 ng/day to 500 μg/day, 10 ng/day to 400 μg/day, 10 ng/day to 300 μg/day, 10 ng/day to 200 μg/day, 10 ng/day to 100 μg/day, 10 ng/day to 10 μg/day, 10 ng/day to 900 ng/day, 10 ng/day to 800 ng/day, 10 ng/day to 700 ng/day, 10 ng/day to 600 ng/day, 10 ng/day to 500 ng/day, 10 ng/day to 400 ng/day, 10 ng/day to 300 ng/day, 10 ng/day to 200 ng/day, 10 ng/day to 100 ng/day, 10 ng/day to 75 ng/day, 10 ng/day to 50 ng/day, 10 ng/day to 25 ng/day, or any other incremental range therebetween (e.g., 1 μg/day to 5 μg/day or 40 μg/day to 100 μg/day).

In some embodiments, the depot 100 may be configured to release the therapeutic agent through the duration of release at a rate no more than 10 mg/day, no more than 1 mg/day, no more than 500 μg/day, no more than 100 μg/day, no more than 90 μg/day, no more than 80 μg/day, no more than 70 μg/day, no more than 60 μg/day, no more than 50 μg/day, no more than 40 μg/day, no more than 30 μg/day, no more than 20 μg/day, no more than 10 μg/day, no more than 5 μg/day, no more than 1 μg/day, no more than 900 ng/day, no more than 800 ng/day, no more than 700 ng/day, no more than 600 ng/day, no more than 500 ng/day, no more than 400 ng/day, no more than 300 ng/day, no more than 200 ng/day, no more than 100 ng/day, no more than 50 ng/day, no more than 40 ng/day, no more than 30 ng/day, no more than 20 ng/day, or no more than 10 ng/day.

As previously described, in some embodiments the depot 100 is configured to release the therapeutic agent over a varying period of time (i.e., duration of release). For those embodiments associated with treating an ocular condition, the depot 100 can be configured to release the therapeutic agent and/or adjunctive agents at the eye for no less than 1 day, no less than 2 days, no less than 3 days, no less than 4 days, no less than 5 days, no less than 6 days, no less than 7 days, no less than 8 days, no less than 9 days, no less than 10 days, no less than 11 days, no less than 12 days, no less than 13 days, no less than 14 days, no less than 15 days, no less than 16 days, no less than 17 days, no less than 18 days, no less than 19 days, no less than 20 days, no less than 21 days, no less than 22 days, no less than 23 days, no less than 24 days, no less than 25 days, no less than 26 days, no less than 27 days, no less than 28 days, no less than 29 days, no less than 30 days, no less than 40 days, no less than 50 days, no less than 60 days, no less than 70 days, no less than 90 days, no less than 100 days, no less than 150 days, no less than 200 days, no less than 300 days, or no less than 365 days.

The therapeutic agent carried by the depots 100 of the present technology may be any biologically active substance (or combination of substances) that provide a therapeutic effect in a patient in need thereof. In some embodiments, the therapeutic agent can be configured to treat ocular conditions including glaucoma, inflammation, macular degeneration, macular edema, cataracts, ocular hypertension, uveitis, and/or dry eye, amongst other conditions. In some embodiments, the therapeutic agent includes cholinergic agonists (e.g., pilocarpine and cevimeline), prostaglandins analogs (e.g., latanoprost, travoprost, bimatoprost, and unoprostine), carbonic anhydrase inhibitors (e.g., methazolamide, 5-acylimino- and related iminosubstituted analogs), alpha and/or beta adrenic agonists (e.g., brimonidine, brimonidine tartrate, apraclonidine, timolol, levobunalol, carteolol, metipranolol, betaxolol), antibodies (e.g., adalimumab, alefacept, basiliximab, bevacizumab, certolizumab, daclizumab, efalizumab, golimumab, infliximab, natalizumab, ranibizumab and rituximab), fusion proteins (e.g., abatacept, alefacept, anakinra, and etanercept), peptides (e.g., antimicrobial peptides, calcitonin gene-related peptide, cell penetrating peptides, fibronectin-derived peptides, neurotransmitters, substance P, tachykinins and vasoactive intestinal peptide), chemokines (e.g., C-C motif chemokine 22), interleukins (e.g., IL-2, TNF or IL-Iβ), neuroprotective agents (e.g., brain-derived neurotrophic factor, glial cell-line neurotrophic factor, nerve growth factor), as well as other agents (e.g., dipivefrin, carbachol, acetazolamide, dorzolamide, ethacrynic acid, mitomycin C, diclofenac, flurbiprofen, dexamethasone, coenzyme-Q10, ganciclovir, fluocinolone acetonide, triamcinolone acetonide, brinzolamide, hydroxypropylcellulose, albumin and immunoglobulin) configured to treat ocular conditions. The therapeutic agent can include the pharmaceutically acceptable salt of any of the therapeutic agents described herein. The therapeutic agents previously described, including their pharmaceutically acceptable salts, can be administered alone or in combination.

These therapeutic agents may be configured to treat one or more of the ocular conditions previously described. For example, (a) pilocarpine, ethacrynic acid, dorzolamide hydrochloride, timolol maleate, latanoprost, bimatoprost, travoprost, ciliary neurotrophic factor, and combinations thereof may be configured to treat glaucoma and/or ocular hypertension, (b) dexamethasone, triamcinolone acetonide, ganciclovir, fluocinolone acetonide, cyclosporine, corticosteroids, and combinations thereof may be configured to treat inflammation, (c) dexamethasone, triamcinolone acetonide, fluocinolone acetonide, ciliary neurotrophic factor, and combinations thereof may be configured to treat macular degeneration and/or macular edema, and (d) hydroxypropyl cellulose, cyclosporine, corticosteroids, cholinergics (e.g., pilocarpine or cevimeline) may be configured to treat dry eye.

In some embodiments, the therapeutic agents may comprise or be combined with one or more adjunctive agents, including analgesics, chemotherapeutic agents, anti-inflammatory agents, antibiotics and/or antimicrobial agents, antifungal agents, agents that promote nerve regeneration, steroids, immunosuppressants, pharmaceutically acceptable salts thereof, and combinations thereof.

The analgesics include, but are not limited to bupivacaine, ropivacaine, mepivacaine, etidocaine, levobupivacaine, trimecaine, carticaine, articaine, lidocaine, prilocaine, benzocaine, procaine, tetracaine, chloroprocaine, and combinations thereof.

The chemotherapeutic agents include, but are not limited to, antibodies, alkylating agents, angiogenesis inhibitors, antimetabolites, DNA cleavers, DNA crosslinkers, DNA intercalators, DNA minor groove binders, enediynes, heat shock protein 90 inhibitors, histone deacetylase inhibitors, immunomodulators, microtubule stabilizers, nucleoside (purine or pyrimidine) analogs, nuclear export inhibitors, proteasome inhibitors, topoisomerase (I or II) inhibitors, tyrosine kinase inhibitors, and serine/threonine kinase inhibitors. Specific therapeutic agents include, but are not limited to, adalimumab, ansamitocin P3, auristatin, bendamustine, bevacizumab, bicalutamide, bleomycin, bortezomib, busulfan, callistatin A, camptothecin, capecitabine, carboplatin, carmustine, cetuximab, cisplatin, cladribin, cytarabin, cryptophycins, dacarbazine, dasatinib, daunorubicin, docetaxel, doxorubicin, duocarmycin, dynemycin A, epothilones, etoposide, floxuridine, fludarabine, 5-fluorouracil, gefitinib, gemcitabine, ipilimumab, hydroxyurea, imatinib, infliximab, interferons, interleukins, beta-lapachone, lenalidomide, irinotecan, maytansine, mechlorethamine, melphalan, 6-mercaptopurine, methotrexate, mitomycin C, nilotinib, oxaliplatin, paclitaxel, procarbazine, suberoylanilide hydroxamic acid (SAHA), 6-thioguanidine, thiotepa, teniposide, topotecan, trastuzumab, trichostatin A, vinblastine, vincristine, vindesine, tamoxifen and combinations thereof.

The anti-inflammatory agents include, but are not limited to, prednisone, betamethasone, cortisone, dexamethasone, hydrocortisone, methylprednisolone, aspirin, Ibuprofen, naproxen sodium, diclofenac, diclofenac-misoprostol, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, mefenamic acid, COX-2 inhibitors, and combinations thereof.

The antibiotics and/or antimicrobial agents include, but are not limited to, amoxicillin, amoxicillin/clavulanate, cephalexin, ciprofloxacin, clindamycin, metronidazole, azithromycin, levofloxacin, sulfamethoxazole/trimethoprim, tetracycline(s), minocycline, tigecycline, doxycycline, rifampin, triclosan, chlorhexidine, penicillin(s), aminoglycides, quinolones, fluoroquinolones, vancomycin, gentamycin, cephalosporin(s), carbapenems, imipenem, ertapenem, antimicrobial peptides, cecropin-mellitin, magainin, dermaseptin, cathelicidin, α-defensins, α-protegrins, and combinations thereof.

The antifungal agents include, but are not limited to, ketoconazole, clortrimazole, miconazole, econazole, intraconazole, fluconazole, bifoconazole, terconazole, butaconazole, tioconazole, oxiconazole, sulconazole, saperconazole, voriconazole, terbinafine, amorolfine, naftifine, griseofulvin, haloprogin, butenafine, tolnaftate, nystatin, cyclohexamide, ciclopirox, flucytosine, terbinafine, amphotericin, and combinations thereof.

The steroids include, but are not limited to, prednisone, betamethasone, cortisone, dexamethasone, hydrocortisone, methylprednisolone, and combinations thereof.

The immunosuppressants include, but are not limited to, cyclosporine, pimecrolimus, sirolimus, tacrolimus, and combinations thereof.

As previously described, the aspects of the polymer, including its composition, thermal response, thickness and configuration, can be tuned to allow for a particular release rate and/or duration of release. For example, the hydrophilicity or hydrophobicity can prolong or decrease the duration of release of therapeutic region to the eye. Suitable polymers of the depot(s) 100 disclosed herein for treating an ocular condition (and/or the symptoms of an ocular condition) include, but are not limited to, poly(alpha-hydroxy acids), poly(lactide-co-glycolide)(PLGA or DLG), poly(DL-lactide-co-caprolactone) (DL-PLCL), polycaprolactone (PCL), poly(L-lactic acid) (PLA), poly(trimethylene carbonate) (PTMC), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(glycolide-co-carolactone) (PGCL), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (such as hyaluronic acid, chitosan and starch), proteins (such as gelatin and collagen) or PEG derivatives and copolymers thereof. Other suitable polymers or copolymers include polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAM (sucrose acetate isobutyrate)hydroxypropyl cellulose, hydroxypropyl methylcellulose, hydroxyethyl methylcellulose, carboxymethylcellulose or salts thereof, Carbopol®, poly(hydroxyethylmethacrylate), poly(methoxyethylmethacrylate), poly(methoxyethoxy-ethylmethacrylate), polymethylmethacrylate (PMMA), methylmethacrylate (MMA), gelatin, polyvinyl alcohols, propylene glycol, or combinations thereof.

In various embodiments, the molecular weight of the polymer can be a wide range of values. The average molecular weight of the polymer can be from about 1000 to about 10,000,000; or about 1,000 to about 1,000,000; or about 5,000 to about 500,000; or about 10,000 to about 100,000; or about 20,000 to 50,000.

### VIII. Systems and Methods for Treating Otolaryngologic Conditions

Otolaryngologic diseases such as rhinosinusitis, allergic rhinitis, nasal infection, and chronic nasal congestion are common ailments, affecting hundreds of millions of individuals worldwide. These conditions can be particularly prevalent among asthma patients. Symptoms associated with rhinosinusitis include headaches, nasal congestion, snot discharge, olfactory dysfunction, fever, chills, constipation, and aches and pain. In young children, rhinosinusitis is also associated with emesis, diarrhea and coughing. Symptoms of allergic rhinitis include telangiectasia (chronic dilation of the capillaries on the skin or mucous membranes), increased glandular secretion, and eosinophilic infiltration. If not treated, this can lead to flu-like symptoms, such as nasal itching, congestion, snot discharge, runny nose, etc. In some instances these conditions develop into rhinosinusitis, asthma or ear infections.

Conventional treatments for otolaryngologic conditions such as rhinosinusitis and allergic rhinitis include drugs delivered via nasal drop or spray. In many cases, intranasally administered drugs are unable to reach the target locations due to barriers of intervening tissue, often with a relatively small percentage of the emitted liquid drug reaching the intended anatomical targets. As a result, intranasal administration of drugs is often ineffective. As an alternative to intranasal sprays or drops, nasal irrigation may be used to deliver saline or liquid drugs into the nasal cavity. The typically short-lived drugs delivered with this approach are often unable to reach the frontal sinus and maxillary sinus, and accordingly fail to reach primary sites of inflammation. Accordingly, drugs delivered via nasal wash are unable to provide a long-term effective treatment.

In some instances, surgical intervention can be used to treat these conditions, for example removing tissue and/or bone to expand the ostium of the sinus, or using a nerve block or other intervention to reduce parasympathetic response to allergens. These operations, though, are expensive and introduce significant risks to patient safety.

In an effort to overcome the invasive nature of repeated intranasal administrations (e.g., sprays and nasal washes) or surgical interventions, a number of extended release systems have been developed for treating otolaryngologic diseases. However, the currently available release systems for otolaryngologic diseases have drawbacks that prevent effective treatment thereof. Specifically, the extended release systems currently available to prolong the release duration of otolaryngologic drugs still lack a true controlled release mechanism. For example, the currently available means for delivering medication to otolaryngologic regions typically provide a burst of drug upon contact with surrounding physiologic fluids, but lack an ability to release the drug in a consistent manner thereafter. It follows that current treatment options for otolaryngologic diseases are generally unable to provide for the consistent release of a drug over an extended period of time. Furthermore, many of the currently available means for delivering medication to otolaryngologic regions are not biocompatible and thus need to removed (e.g., by a medical specialist).

Thus, there exists a need for a biocompatible otolaryngologic delivery system able to deliver a therapeutic agent over an extended period of time. The depots 100 of the present technology may be used to treat a variety of symptoms (e.g., chronic rhinosinusitis) associated with otolaryngologic conditions depending upon the nature of the therapeutic agent delivered as described above. The depots 100 of the present technology may be implanted in vivo proximate to the nasal cavity to provide a localized, controlled and sustained release of a therapeutic agent for the treatment of a particular condition. For example, the depot can be configured to provide a sustained presence of the therapeutic agent to the treatment site over a predefined period of time. This implantation may be associated with a surgery or intervention for treating the particular symptom or condition, whereby the depot 100 enables chronic, sustained pharmacological treatment following completion of the surgery or intervention. The depot 100 may be a standalone element, or may be coupled to or integrated as part of an implantable device or prosthesis associated with the intervention or surgery.

The amount of the therapeutic agent that will be effective in a patient in need thereof will depend on the specific nature of the condition, and can be determined by standard clinical techniques known in the art. In addition, in vitro or in vivo assays may optionally be employed to help identify optimal dosage ranges. The specific dose level for any particular individual will depend upon a variety of factors including the activity of the drug, the age, genetic factors, environmental influences, sex, diet, time of administration, location of administration, and the severity of the particular problem being treated.

Some aspects of the present technology include a system comprising a plurality of depots (each of which could be any of the depots described herein) provided for implantation by a clinical practitioner. In this system, each depot may be configured for controlled release of therapeutic agent to tissue proximate to the implantation site of the depot. The depots in the system may be identical or may vary in several respects (e.g., form factor, therapeutic agent, release profile, etc.). For example, the system may be comprised of a depot having a release profile that provides for an immediate release of therapeutic agent and other depots comprised of a depot having a release profile that provides for a delayed release of therapeutic agent.

The depot 100 used to deliver the therapeutic agent(s) for treating select symptoms associated with otolaryngologic conditions may comprise any of those depots 100 previously described herein, such as those described with reference to FIGS. 1-52C. For example, the depot 100 may comprise a substantially columnar shape, pellets, disks, a substantially cylindrical shape, a rolled film configuration, a dumpling configuration, and/or microdepots (e.g., beads, microspheres, microcyliners, etc.). The shape of the depot 100, the arrangement of the various regions of the depot 100, and the mechanical properties of the depot 100 can be optimized for ease of delivery by the clinician. For example, the elongated depot configurations disclosed herein have a low-profile, thereby improving patient comfort and enabling injection/insertion through a lower diameter needle. In these and other embodiments described herein, many depots 100 of the present technology are sufficiently flexible and/or provide features for preferential bending such that the depot 100 can flex or deform relative to the delivery device and/or better adapt and conform to the anatomy of the nasal cavity or other treatment site. The flexibility and low-profile configuration of the depots 100 herein thus provide less resistance to movement within the nasal cavity, making the depots 100 more conducive to long-term (e.g., a month or multi-month) wear.

The depots 100 of the present technology described herein can be positioned proximate any region of the ear, nose, and throat. For example, depots 100 of the present technology can be positioned at or proximate the nasal cavity, the frontal sinus, the sphenoid sinus, the ethmoid sinus, the maxillary sinus, the superior nasal concha, the middle nasal concha, the inferior nasal concha, the vestibule, the nasopharynx, the superior turbinate, middle turbinate, inferior turbinate, eustachian tube orifice, Fossa of Rosenmuller, adenoid pad, or any areas adjacent thereto. [symbol] 56 illustrates anterior and lateral anatomical views of the nasal cavity and surrounding paranasal areas, and FIG. 57 illustrates a cross-sectional anatomical view of the nasal cavity with a plurality of depots 100a-100e disposed therein. As shown in the illustrated embodiment, depot 100a is positioned proximate the inferior turbinate, depot 100b is positioned in or proximate the frontal sinus, depot 100c is positioned in or proximate the superior turbinate, depot 100d is positioned in or proximate the sphernoid sinus, and depot 100e is positioned in or adjacent the adenoid pad. The depots 100a-e and their positioning, as shown in FIG. 57, represent some illustrative embodiments of the present technology. In other embodiments, the depots may be positioned at other regions of the nasal cavity and/or surrounding paranasal areas, and/or include different depot configurations. For example, as illustrated in FIG. 57, depot 100a assumes a bead or pellet configuration, but in other embodiments could include a cylindrical or rod-shaped depot. In various embodiments, one or more depots 100 can be positioned at any location within or around the nasal cavity, the frontal sinus, the sphenoid sinus, the ethmoid sinus, and/or the maxillary sinus. In some embodiments, one or more depots 100 can be positioned within the anterior portion of the nasal cavity, for example facilitating intranasal delivery and implantation.

The depot(s) 100 implanted within the nasal cavity or other treatment site of the ear, nose, and throat can include any of the configurations or form factors previously described, though certain configurations and form factors may be particularly beneficial to achieve more effective treatment of otolaryngologic conditions. For example, the depot 100 may beneficially include a thin film configuration, e.g., to enable the depot to be sandwiched between folds of the ethmoid sinus, or to facilitate insertion intranasally. As another example, the depot 100 may beneficially include a curved, bent, or rounded configuration, e.g., to increase or provide adequate contact with the curved surface area of a treatment site, such as the superior nasal concha. As previously described, the curved configuration can be achieved after the depot 100 is deployed in vivo in the presence of physiological fluids and/or temperatures. Additionally, in some embodiments, elasticity of one or more layers (e.g., the control region) of the depot 100 can be adjusted tuned based at least in part of the target site. As another example, the depot 100 may beneficially include a pellet configuration having a cylindrical, circular, elliptical, regular polygonal or irregular polygonal shape, e.g., to complement that of the target site. As another example, the depot 100 may include a polymer composition that is more hydrophilic (e.g., PLGA) or less hydrophilic (PLA) than other regions of the depot to absorb more of less water or other fluids when implanted in vivo. Depending on the intended application, a hydrophilic or hydrophobic polymer composition of the depot 100 can prolong or decrease the duration of release of therapeutic region to the nasal cavity. For example, a hydrophilic polymer composition of the depot 100 could enable a quicker release of therapeutic agent once implanted in vivo. As another example, the depot 100 may include a fixation portion (e.g., a tab, a ridge, a hook, a barb, a protrusion, or a notch) configured to penetrate at least a portion of the thickness of the adjacent otolaryngologic structure (e.g., at or adjacent to the superior nasal concha, the middle nasal concha, the inferior nasal concha, the vestibule, or the nasopharynx) and thereby secure the depot within the nasal cavity. In some embodiments, the depot 100 can be configured fold or compress to be fitted within a sinus or the nasal passage. Once positioned and released, the depot 100 can expand into apposition with adjacent tissue (e.g., within or superior to the nasal vestibule), thereby securing the depot 100 in place at or adjacent to the treatment site.

In some embodiments, the depot 100 (or a system of depots 100) is configured to release the therapeutic agent at a rate according to a particular profile, as previously described with reference to FIGS. 3A and 3B. For those embodiments directed to treating otolaryngologic conditions, the release profile may be (a) zero-order such that release of the payload of therapeutic agent is at a substantially steady rate over the duration of release, (b) first-order such that release of the payload of the therapeutic agent increases in a substantially linear manner over the duration of release, or (c) second-order such that release of the payload of the therapeutic agent occurs at a high, substantially linear rate for a first period of time and then at a lower, substantially linear rate for a second period of time over the duration of release.

Each of these release profiles can be advantageous depending on a particular otolaryngologic condition. For example, a zero-order release profile may be desired when a continuous and steady supply of the therapeutic agent is needed. For example, continuous delivery of steroids can effectively treat chronic rhinosinusitis. Depots 100 of the present technology having a zero-order release profile can be implanted in the nasal cavity to release the therapeutic agent in a substantially consistent manner over a duration of release.

As previously described, the release profile of the depot may be tuned to release a therapeutic agent for particular durations and/or at particular release rates by adjusting the structure, composition, and the process by which the depot is manufactured. In some embodiments, the total payload (e.g., the total therapeutic agent) of the depot 100 is at least 10 μg, 50 μg, 100 μg, 200 μg, 300 μg, 400 μg, 500 μg, 600 μg, 700 μg, 800 μg, 900 μg, 1 mg, 10 mg, 100 mg, or 150 mg. In some embodiments, the total payload of the depot 100 is from 10 μg to 10 mg, 10 μg to 1000 μg, 10 μg to 900 μg, 10 μg to 800 μg, 10 μg to 700 μg, 10 μg to 600 μg, 10 μg to 500 μg, 10 μg to 400 μg, 10 μg to 300 μg, 10 μg to 200 μg, 10 μg to 100 μg, 10 μg to 75 μg, 10 μg to 50 μg, or 10 μg to 20 μg, or any other incremental ranges therebetween (e.g., 20 μg to 60 μg).

In some embodiments, the depot 100 is configured to release the therapeutic agent through the duration of release at a rate of from about 10 ng/day to 900 μg/day, 10 ng/day to 700 μg/day, 10 ng/day to 500 μg/day, 10 ng/day to 400 μg/day, 10 ng/day to 300 μg/day, 10 ng/day to 200 μg/day, 10 ng/day to 100 μg/day, 10 ng/day to 10 μg/day, 10 ng/day to 900 ng/day, 10 ng/day to 800 ng/day, 10 ng/day to 700 ng/day, 10 ng/day to 600 ng/day, 10 ng/day to 500 ng/day, 10 ng/day to 400 ng/day, 10 ng/day to 300 ng/day, 10 ng/day to 200 ng/day, 10 ng/day to 100 ng/day, 10 ng/day to 75 ng/day, 10 ng/day to 50 ng/day, 10 ng/day to 25 ng/day, or any other incremental range therebetween (e.g., 1 μg/day to 5 μg/day or 40 μg/day to 100 μg/day).

In some embodiments, the depot 100 may be configured to release the therapeutic agent through the duration of release at a rate no more than 10 mg/day, no more than 1 mg/day, no more than 500 μg/day, no more than 100 μg/day, no more than 90 μg/day, no more than 80 μg/day, no more than 70 μg/day, no more than 60 μg/day, no more than 50 μg/day, no more than 40 μg/day, no more than 30 μg/day, no more than 20 μg/day, no more than 10 μg/day, no more than 5 μg/day, no more than 1 μg/day, no more than 900 ng/day, no more than 800 ng/day, no more than 700 ng/day, no more than 600 ng/day, no more than 500 ng/day, no more than 400 ng/day, no more than 300 ng/day, no more than 200 ng/day, no more than 100 ng/day, no more than 50 ng/day, no more than 40 ng/day, no more than 30 ng/day, no more than 20 ng/day, or no more than 10 ng/day.

As previously described, in some embodiments the depot 100 is configured to release the therapeutic agent over a varying period of time (i.e., duration of release). For those embodiments associated with treating an otolaryngologic condition, the depot 100 can be configured to release the therapeutic agent and/or adjunctive agents at the nasal cavity for no less than 1 day, no less than 2 days, no less than 3 days, no less than 4 days, no less than 5 days, no less than 6 days, no less than 7 days, no less than 8 days, no less than 9 days, no less than 10 days, no less than 11 days, no less than 12 days, no less than 13 days, no less than 14 days, no less than 15 days, no less than 16 days, no less than 17 days, no less than 18 days, no less than 19 days, no less than 20 days, no less than 21 days, no less than 22 days, no less than 23 days, no less than 24 days, no less than 25 days, no less than 26 days, no less than 27 days, no less than 28 days, no less than 29 days, no less than 30 days, no less than 40 days, no less than 50 days, no less than 60 days, no less than 70 days, no less than 90 days, no less than 100 days, no less than 150 days, no less than 200 days, no less than 300 days, or no less than 365 days.

The therapeutic agent carried by the depots 100 of the present technology may be any biologically active substance (or combination of substances) that provide a therapeutic effect in a patient in need thereof. In some embodiments, the therapeutic agent can be configured to treat otolaryngologic conditions including rhinosinusitis, allergic rhinitis, nasal infection, and chronic nasal congestion, amongst other conditions. In some embodiments, the therapeutic agent includes steroids, antibiotic agents, and/or anti-fungal agents configured to treat otolaryngologic conditions. The therapeutic agent can include the pharmaceutically acceptable salt of any of the therapeutic agents described herein. The therapeutic agents previously described, including their pharmaceutically acceptable salts, can be administered alone or in combination.

The steroids include, but are not limited to, mometasone furoate, triamcinolone, dexamethasone, fluticasone, prednisone, methylprednisolone, betamethasone, cortisone, hydrocortisone, methylprednisolone, ciclesonide, beclomethasone, budesonide, flunisolide, and combinations thereof.

The antibiotic and/or antimicrobial agents include, but are not limited to, amoxicillin, amoxicillin/clavulanate, cephalexin, ciprofloxacin, clindamycin, metronidazole, azithromycin, levofloxacin, sulfamethoxazole/trimethoprim, tetracycline(s), minocycline, metronidazole/cefalexin, metronidazole/cefuroxime, metronidazole/cefprozil, moxifloxacin, levofloxacin, metronidazole/cephalosporin, tigecycline, doxycycline, rifampin, triclosan, chlorhexidine, tobramycin, cefuroxime, ceftazidime, ofloxacin, gentamicin, mupirocin, penicillin(s), aminoglycides, quinolones, fluoroquinolones, vancomycin, gentamycin, cephalosporin(s), carbapenems, imipenem, ertapenem, antimicrobial peptides, cecropin-mellitin, magainin, dermaseptin, cathelicidin, α-defensins, α-protegrins, and combinations thereof.

The antifungal agents include, but are not limited to, amphotericin B, ketoconazole, clortrimazole, miconazole, econazole, intraconazole, fluconazole, bifoconazole, terconazole, butaconazole, tioconazole, oxiconazole, sulconazole, saperconazole, voriconazole, terbinafine, amorolfine, naftifine, griseofulvin, haloprogin, butenafine, tolnaftate, nystatin, cyclohexamide, ciclopirox, flucytosine, terbinafine, amphotericin, and combinations thereof.

These therapeutic agents may be configured to treat one or more of the otolaryngologic conditions previously described. Additional conditions may benefit from intranasal administration of therapeutic agents. For example, analgesics such as diamorphine hydrochloride and fentanyl citrate can be delivered using a depot 100 positioned intranasally. Other conditions that can be treated using intranasal depots 100 include migraines (e.g., sumatriptan, zolmitriptan), endometriosis (e.g., nafarelin acetate), nasal congestion (e.g., xylometazoline, oxymetazoline, azelastine, ephedrine, ipratropium bromide, neomycin sulfate, chlorhexidine dihydrochloride), perennial and seasonal allergic rhinitis (e.g., budesonide, beclomethasone dipropionate, mometasone furoate, triamcinolone acetonide, fluticasone propionate, fluticasone furoate, sodium cromiglicate), prostate carcinoma (e.g., buserelin acetate), nicotine withdrawal symptoms (e.g., nicotine), nocturia (e.g., desmopressin acetate), and vaccinations (e.g., influenza vaccine).

In some embodiments, the therapeutic agents may comprise or be combined with one or more adjunctive agents, including analgesics, chemotherapeutic agents, anti-inflammatory agents, antibiotics and/or antimicrobial agents, antifungal agents, agents that promote nerve regeneration, steroids, immunosuppressants, pharmaceutically acceptable salts thereof, and combinations thereof.

The analgesics include, but are not limited to bupivacaine, ropivacaine, mepivacaine, etidocaine, levobupivacaine, trimecaine, carticaine, articaine, lidocaine, prilocaine, benzocaine, procaine, tetracaine, chloroprocaine, and combinations thereof.

The chemotherapeutic agents include, but are not limited to, antibodies, alkylating agents, angiogenesis inhibitors, antimetabolites, DNA cleavers, DNA crosslinkers, DNA intercalators, DNA minor groove binders, enediynes, heat shock protein 90 inhibitors, histone deacetylase inhibitors, immunomodulators, microtubule stabilizers, nucleoside (purine or pyrimidine) analogs, nuclear export inhibitors, proteasome inhibitors, topoisomerase (I or II) inhibitors, tyrosine kinase inhibitors, and serine/threonine kinase inhibitors. Specific therapeutic agents include, but are not limited to, adalimumab, ansamitocin P3, auristatin, bendamustine, bevacizumab, bicalutamide, bleomycin, bortezomib, busulfan, callistatin A, camptothecin, capecitabine, carboplatin, carmustine, cetuximab, cisplatin, cladribin, cytarabin, cryptophycins, dacarbazine, dasatinib, daunorubicin, docetaxel, doxorubicin, duocarmycin, dynemycin A, epothilones, etoposide, floxuridine, fludarabine, 5-fluorouracil, gefitinib, gemcitabine, ipilimumab, hydroxyurea, imatinib, infliximab, interferons, interleukins, beta-lapachone, lenalidomide, irinotecan, maytansine, mechlorethamine, melphalan, 6-mercaptopurine, methotrexate, mitomycin C, nilotinib, oxaliplatin, paclitaxel, procarbazine, suberoylanilide hydroxamic acid (SAHA), 6-thioguanidine, thiotepa, teniposide, topotecan, trastuzumab, trichostatin A, vinblastine, vincristine, vindesine, tamoxifen and combinations thereof.

The anti-inflammatory agents include, but are not limited to, prednisone, betamethasone, cortisone, dexamethasone, hydrocortisone, methylprednisolone, aspirin, Ibuprofen, naproxen sodium, diclofenac, diclofenac-misoprostol, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, mefenamic acid, COX-2 inhibitors, and combinations thereof.

The immunosuppressants include, but are not limited to, cyclosporine, pimecrolimus, sirolimus, tacrolimus, and combinations thereof.

IX. Systems and Methods for Treating or Preventing Capsular Contracture or Other Conditions Associated with Breast Implants

Breast implants are increasingly popular procedures, and are typically performed either for reconstruction following mastectomy or for cosmetic augmentation. Various types of implants are available, covering a range of different shapes and sizes, surface textures (e.g., smooth or textured), and materials used to fill the implant (e.g., saline or silicone). Capsular contracture is a common complication of breast implant surgery. A surgically reconstructed or augmented breast should be soft, flexible, and drape naturally. While the causes of capsular contracture have not been definitively established, it is thought to be a local complication resulting from excessive fibrotic foreign body reaction to the implant. In particular, an inflammatory reaction causes fibrosis through the production of collagen, leading to excessively firm and painful breasts, often with visible signs of skin tightening, dimpling, or twisting. If severe enough, capsular contraction can require reoperation. Breast implants are particularly susceptible to capsular contraction if they have smooth exterior surfaces. In contrast, breast implants having a textured outer surface (e.g., having a surface roughness of 100-300 microns) cause less fibrosis, and micro-textured outer surfaces (e.g., surface roughness of 10 micron to 100 microns) are associated with a lower capsular contracture rate.

FIG. 58 illustrates a breast implant in which a capsule has formed, resulting in capsular contracture. As seen in FIG. 58, the breast implant is surrounded by a biofilm that encapsulates the implant. The capsule typically includes fibroblasts, macrophages, and lymphocytes. The capsule is thought to form when fibroblasts accumulate at the contact zone of the implant and the capsule as a result of an immunological reaction to the implant as a foreign body. The fibroblasts produce collagen fibers, which over time thicken and form cable-like bundles and result in hardening and painful swelling of the breast. Tumor growth factor beta (TGF-β) may act as a master switch in development of the condition, allowing a cascade of reactions which subsequently leads to formation of the capsule. Other immunological factors have also been shown to have a role in the development of capsular contracture. These include connective tissue growth factor and interleukins amongst others, all of which promote fibrosis. Although recent evidence appears to support the role of the immune system, historically it has been suggested that capsular contracture may result from an exaggerated inflammatory response caused by local bacterial infection.

The severity of capsular contracture may be classified using the Baker classification system. This is a subjective classification system that is based upon clinical findings in the implant patient by the physician. It is divided into four grades. In grade I, the breast is normally soft and appears natural. In grade II, the breast is slightly firm but appears normal. Grades I and II are not clinically significant. In grade III, the breast is firm and appears abnormal, with some visible distortion. And in grade IV, the breast is hard, painful, and looks abnormal, with more significant visible distortion. Studies have published incidence rates of capsular contracture ranging from 2.8% to 20.4%.

To treat the symptoms of and/or prevent the occurrence of capsular contracture, it may be useful to suppress fibrosis, prevent local infection, and/or to treat any local infection. Conventional treatments for capsular contracture include reoperation (e.g., capsulectomy or capsulotomy), as well as application of one or more medications, such as leukotriene receptors agonists (e.g., zafirlukast). However, the currently available means for delivering medication typically provide a burst of drug upon contact with surrounding physiologic fluids, but lack an ability to then release the drug in a controlled, sustained, consistent manner. It follows that current treatment options are generally unable to provide for the consistent release of a drug over an extended period of time.

Thus, a need exists for implantable systems capable of providing a controlled release of medication to treat capsular contracture following breast implant surgery. Embodiments of the present technology relate to implants and insertables configured to be disposed at a surgical or interventional treatment site proximate an implanted breast prosthesis for controlled release of a therapeutic agent over a period of time, e.g., to treat and/or reduce the effects of capsular contracture. For example, the depot can be configured to provide a sustained presence of the therapeutic agent to the treatment site over a predefined period of time. As described in more detail below, in some embodiments the depots 100 described herein can be configured to (a) at least partially surround or cover a breast implant, (b) be coupled to an exterior surface of a breast implant, and/or (c) be integrated into an exterior surface of a breast implant, and release one or more therapeutic agents configured to prevent or treat capsular contracture. The depot 100 of may be tuned to meet the particular conditions of breast implant patients, e.g., by altering various factors (e.g., shape and/or configuration) of the depot 100 such that the depot 100 has a particular release profile, duration of release, and/or desired effect on the tissue adjacent to the breast implant.

The amount of the therapeutic agent that will be effective in a patient in need thereof will depend on the specific nature of the condition, and can be determined by standard clinical techniques known in the art. In addition, in vitro or in vivo assays may optionally be employed to help identify optimal dosage ranges. The specific dose level for any particular individual will depend upon a variety of factors including the activity of the drug, the age, body weight, general physical and mental health, genetic factors, environmental influences, sex, diet, time of administration, location of administration, rate of excretion, and the severity of the particular problem being treated.

Some aspects of the present technology include a system comprising a plurality of depots 100 (each of which could be any of the depots 100 described herein) provided for implantation by a clinical practitioner. In this system, each depot 100 may be configured for controlled release of therapeutic agent to tissue proximate to the implantation site of the depot 100. The depots 100 in the system may be identical or may vary in several respects (e.g., form factor, therapeutic agent, release profile, etc.). For example, the system may be comprised of a depot having a release profile that provides for an immediate release of therapeutic agent and other depots 100 comprised of a depot 100 having a release profile that provides for a delayed release of therapeutic agent.

Embodiments of the present technology enable short and long-term treatment of capsular contracture in that therapeutic agents released from the depot 100 can immediately act to suppress fibrosis and/or to kill bacteria, as well as limit the recurrence of capsular contracture due to the continuous release of therapeutic agent(s) from the depot 100 over an extended duration of release. In doing so, patients can avoid reoperation and the side effects therefrom. Accordingly, embodiments of the present technology enable a comprehensive treatment of capsular contracture compared to conventional treatments.

The therapeutic agent carried by the depots 100 of the present technology may be any biologically active substance (or combination of substances) that provide a therapeutic effect in a patient in need thereof. In some embodiments, the therapeutic agent includes fibrosis-inhibiting agents, anti-scarring agents, leukotriene inhibitors or agonists, cell growth inhibitors, anti-inflammatory agents, antibiotic or antimicrobial agents, or other agents effective to treat various conditions associated with implant surgery and recovery. In some embodiments, the therapeutic agent(s) include one or more of leukotriene antagonists (e.g., zafirlukast, montelukast, pranlukast, zileuton, acitazanolast, iralukast, pranlukast, or verlukast) anti-adhesion barrier solution (AABS); therapeutic agents that target the TGF-β signaling pathway (e.g., halofuginone or tranilast); anti-inflammatory agents, and antimicrobial and/or antibiotic agents. As noted previously, such therapeutic agents may be useful in treating or preventing capsular contracture in breast implant patients, for example by disrupting the process of capsule formation around the breast implant, by reducing the thickness of the capsule, or otherwise.

The anti-inflammatory agents include, but are not limited to, pirfenidone, prednisone, betamethasone, cortisone, dexamethasone, hydrocortisone, methylprednisolone, aspirin, Ibuprofen, naproxen sodium, diclofenac, diclofenac-misoprostol, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, mefenamic acid, COX-2 inhibitors, and/or combinations thereof.

The antibiotics and/or antimicrobial agents include, but are not limited to, amoxicillin, amoxicillin/clavulanate, cephalexin, ciprofloxacin, clindamycin, metronidazole, azithromycin, levofloxacin, sulfamethoxazole/trimethoprim, tetracycline(s), minocycline, tigecycline, doxycycline, rifampin, triclosan, chlorhexidine, penicillin(s), aminoglycides, quinolones, fluoroquinolones, vancomycin, gentamycin, cephalosporin(s), carbapenems, imipenem, ertapenem, antimicrobial peptides, cecropin-mellitin, magainin, dermaseptin, cathelicidin, α-defensins, α-protegrins, and/or combinations thereof.

In some embodiments, the total payload (e.g., the total therapeutic agent or combination of therapeutic agent and adjunctive agent) of the depot 100 may be at least 100 mg, at least 150 mg, at least 200 mg, at least 300 mg, at least 400 mg, at least 500 mg, at least 600 mg, at least 700 mg, at least 800 mg, at least 900 mg, or at least 1000mg.

In some embodiments, the depot 100 is configured to release the therapeutic agent through the duration of release at a rate of from about 0.1 mg/day to about 200 mg/day, about 0.1 mg/day to about 150 mg/day, about 0.1 mg/day to about 100 mg/day, about 0.1 mg/day to about 90 mg/day, about 0.1 mg/day to about 80 mg/day, about 0.1 mg/day to about 70 mg/day, about 0.1 mg/day to about 60 mg/day, about 0.1 mg/day to about 50 mg/day, about 0.1 mg/day to about 40 mg/day, about 0.1 mg/day to about 30 mg/day, about 1 mg/day to about 30 mg/day, about 1 mg/day to about 20 mg/day, about 5 mg/day to about 20 mg/day, about 10 mg/day to about 20 mg/day, or about 15 mg/day to about 20 mg/day, or any other incremental ranges therebetween (e.g., 50 mg/day to 100 mg/day, 150 mg/day to 175 mg/day, etc.).

In some embodiments, the depot 100 may be configured to release the therapeutic agent through the duration of release at a rate no more than 100 mg/day, no more than 90 mg/day, no more than 80 mg/day, no more than 70 mg/day, no more than 60 mg/day, no more than 50 mg/day, no more than 40 mg/day, no more than 30 mg/day, no more than 20 mg/day, no more than 15 mg/day, no more than 10 mg/day, no more than 5 mg/day, no more than 1 mg/day, no more than 0.5 mg/day, no more than 0.1 mg/day, no more than 75 μg/day, no more than 50 μg/day, no more than 25 μg/day, or no more than 10 μg/day.

As previously described, in some embodiments the depot 100 is configured to release the therapeutic agent over a varying period of time (i.e., duration of release). For those embodiments associated with treating capsular contracture, the depot 100 can be configured to release the therapeutic agent(s) at the treatment site for no less than 1 day, no less than 2 days, no less than 3 days, no less than 4 days, no less than 5 days, no less than 6 days, no less than 7 days, no less than 8 days, no less than 9 days, no less than 10 days, no less than 11 days, no less than 12 days, no less than 13 days, no less than 14 days, no less than 15 days, no less than 16 days, no less than 17 days, no less than 18 days, no less than 19 days, no less than 20 days, no less than 21 days, no less than 22 days, no less than 23 days, no less than 24 days, no less than 25 days, no less than 26 days, no less than 27 days, no less than 28 days, no less than 29 days, no less than 30 days, no less than 40 days, no less than 50 days, no less than 60 days, no less than 70 days, no less than 90 days, no less than 100 days, no less than 150 days, no less than 200 days, no less than 300 days, or no less than 365 days.

In one example, the depot 100 can be configured to release the therapeutic agent, such as a leukotriene receptor agonist (e.g., zafirlukast, montelukast), at a rate of approximately 5 mg/kg of patient body weight per day for a period of time (e.g., any of the periods of time noted described herein). For example, a depot 100 can be provided with a payload of a leukotriene receptor agonist of between about 1 g and about 5 g and be configured to release the therapeutic agent continuously for at least 7 days. In another example, the depot 100 can be configured to contain a payload of approximately 100 mg/kg of tranilast, and can be configured to release the therapeutic agent over a period of between 1-3 months. In another example, a depot 100 can be configured to contain a payload of between about 20-60 mg, or approximately 40 mg, of triamcinolone and be configured to release the therapeutic agent continuously over a period of between about 2-8 weeks, or approximately 4 weeks. In another example, a depot 100 can be configured to contain a payload of between about 50 mg and about 500 mg, or approximately 150 mg, of pirfenidone and be configured to release the therapeutic agent continuously over a period of between about 2-8 weeks, or approximately 4 weeks. In yet another example, the depot 100 can be configured to release 0.1 mL of AABS over a period of approximately 1-3 months.

As previously described, the depot 100 of the present technology can achieve a release profile or kinetics that suits the objectives of the intended therapy. For those embodiments directed to treating capsular contracture associated with breast implant surgery, the release profile may be (a) zero-order such that release of the payload of therapeutic agent is at a substantially steady rate over the duration of release, (b) first-order such that release of the payload of the therapeutic agent increases in a substantially linear manner over the duration of release, or (c) a second-order such that release of the payload of the therapeutic agent occurs at a high, substantially linear rate for a first period of time and then at a lower, substantially linear rate for a second period of time over the duration of release.

Each of these release profiles can be advantageous for capsular contracture patients depending on their particular condition. For example, a zero-order release profile may be desired when the therapeutic agent is used predominantly to prevent fibrosis. In such cases, release of the therapeutic agent in a substantially consistent manner over a duration of release can maximize the amount of time drug is released from the depot, thereby maximizing the amount of time that fibrosis is actively limited by the therapeutic agent. As another example, a second-order release profile may be desired when excessive fibrous tissue has already formed and capsular contracture has developed. In such cases, release of therapeutic agent during a first period of time at the higher rate is used to first target the existing fibrous connective tissue around the implant, and a subsequent release of therapeutic agent during a second period of time at the lower rate is used to prevent recurrence of fibrotic tissue. Embodiments of the present technology enable the depot to be tuned according to the optimal treatment needed for each patient.

The depots 100 of the present technology previously described are generally applicable to treating capsular contracture and other conditions associated with breast implant surgery. In some embodiments, certain form factors may be particularly beneficial to achieve more effective treatment. For example, in some embodiments the depot 100 can be shaped, dimensioned, and configured to form a cover for a breast implant, for example extending over a portion or all of the exterior surface of a breast implant. In such embodiments, the depot 100 when used with breast implants can reduce or prevent capsular contracture as well as treat or prevent infection, pain, inflammation, scarring or other indications or complications associated with breast augmentation or breast reconstruction.

In various embodiments, the depot 100 may be a cover formed separately from a breast implant into which a breast implant may be inserted, or the depot 100 may be a coating formed upon the surface of the breast implant. FIG. 59 illustrates a breast implant 800 encapsulated by a depot 100 and positioned within a patient's breast. Although the illustrated embodiment depicts the depot 100 completely surrounding the implant 800, in various embodiments the depot 100 may cover only a portion of the outer surface of the implant 800, for example forming a strip or patch that extends only partially over the surface of the implant 800. Once implanted in the patient, the depot 100 will come into contact with physiological fluids and release therapeutic agent(s) to surrounding tissue as described previously herein.

As noted above, in some embodiments the depot 100 covers at least a portion of the breast implant 800. The depot 100 in the form of a cover has an appropriate form-fitting shape for the implant and may encase an implant 800 in the operating room prior to its insertion during surgery. The depot 100 in the form of a cover can also be preassembled with the breast implant 800 and supplied for surgery as a breast implant assembly, where the term “breast implant assembly” also includes a depot 100 supplied for surgery in the form of a coated breast implant.

In some embodiments, the depot 100 has substantially the same shape and size as the implant 800 itself (e.g., round, teardrop, anatomical, etc.), and partially or completely covers the implant 800. In some embodiments, the depot 100 is shaped to define an inner chamber with an opening, and the depot 100 may be sealed after the implant is inserted into the inner space of the depot 100 via the opening, for example by the depot 100 being shrunk around the implant 800 to provide a closely fitting covering. The depot 100 can be useful to reduce capsular contracture, for example by delivering therapeutic agent(s) into the surrounding tissue, to reduce and/or prevent infection, pain, and/or other conditions, indications or complications associated with breast implants.

The depot 100 may have a substantially uniform thickness over the breast implant wall. In some embodiments, the implant 800 is inserted into the depot 100 through a suitable aperture, which preferably may be a slit that can readily be overlapped to fully cover the implant 800 so as to minimize and preferably prevent infection to an uncovered portion of the implant. Those of skill in the art can readily determine appropriate shapes, sizes and configurations for the apertures or slits for a given size and shape of breast implant 800.

In some embodiments, the depot 100 may cover only relatively small portions of the breast implant 800, and may be adhered to the outer surface using adhesive, barbs, hooks, tines, or other fixation mechanisms. In some embodiments, the depot 100 can be sutured or otherwise attached to a portion of the breast implant 800 prior to or after the implant has been surgically positioned within the patient.

In some embodiments, the outer layer of the depot 100 can provide a textured surface, for example having a surface roughness of at least 10 microns, at least 100 microns, at least 200 microns, at least 300 microns, or least 500 microns. Varying the surface roughness can help reduce the risk of capsular contracture. In some embodiments, the depot 100 includes a layered design, such as those depot embodiments comprising a therapeutic region including a first portion having a therapeutic agent, and a second portion having an adjunctive agent (e.g., an immunotherapeutic agent, anti-inflammatory agent, antibiotic agent and/or antifungal agent). Such embodiments can provide the combined release (e.g., simultaneous or sequential release) of the therapeutic agent and adjunctive agent as previously described herein.

In some embodiments, the depot 100 can be configured to have similar mechanical properties to those of the implant 800. For example, at body temperature, both the outer shell of the implant 800 and the depot 100 may be soft and pliable. The depot 100 in the form of a cover may also be elastomeric so that it can be stretched around the implant 800, or can be stretched and will shrink to fit tightly around the implant 800.

After manufacture, the depot 100 in the form of a cover can be sterilized and packaged for assembly onto a breast implant 800 immediately prior to surgery. Alternatively, the depot 100 of can be assembled onto the breast implant 800, sterilized and packaged at the time of manufacture, so that a completed breast implant assembly is delivered to the surgical suite. Sterile gloves and sterile and atraumatic instruments may be used when handling the coverings to provide a sterile breast implant assembly that includes the implant 800 and the depot 100. Once form-fitted with depot 100 as a cover or as a coated implant 800, the implant assembly can be inserted into the subject using standard breast reconstructive or augmentative surgical techniques.

In some embodiments, one or more depots 100 can be provided with a breast implant 800 as a kit. For example, the depot 100 in the kit may be size matched to the implant 800 supplied with the kit. The kits may be sterile, and may contain instructions for inserting the accompanying implant into the depot 100 or for handling and surgically implanting the breast implant assembly in the subject. In operation, the kits may be opened, and the implant 800 may be inserted into the depot 100 before implantation in the patient. Alternatively, the kit can include a breast implant assembly that comprises the depot 100 coupled to the breast implant 800 (e.g., wrapped around the breast implant 800), or a breast implant coated with the depot 100. As with the previously described kits, the depots 100 and implants of the assembly are appropriately size-matched and sterile.

### X. Conclusion

Although many of the embodiments are described above with respect to systems, devices, and methods for treating various conditions, the technology is applicable to other applications and/or other approaches. For example, the depots of the present technology may be used to treat other conditions than those described herein. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 2-59.

The above detailed descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

Unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. Additionally, all ranges disclosed herein are to be understood to encompass any and all subranges subsumed therein. For example, a range of “1 to 10” includes any and all subranges between (and including) the minimum value of 1 and the maximum value of 10, i.e., any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. For example, reference to “a therapeutic agent” includes one, two, three or more therapeutic agents.

The headings above are not meant to limit the disclosure in any way. Embodiments under any one heading may be used in conjunction with embodiments under any other heading.

1. A depot for the treatment of symptoms associated with type II diabetes, comprising:
a therapeutic region comprising a therapeutic agent, the therapeutic agent including a glucagon-like peptide-1 (GLP-1) receptor agonist; and
a control region comprising a bioresorbable polymer and a releasing agent mixed with the polymer, wherein the releasing agent is configured to dissolve when the depot is placed in vivo to form diffusion openings in the control region,
wherein the depot is configured to be implanted in vivo and, while implanted, release the GLP-1 receptor agonist for a period of time.

2. The depot of claim 1, wherein the GLP-1 receptor agonist comprises at least one of exenatide, liraglutide, albiglutide, dulaglutide, or lixisenatide, semaglutide, derivatives thereof, or combinations thereof.

3. The depot of claim 1, wherein the therapeutic agent is a first therapeutic agent, the therapeutic region further comprising a second therapeutic agent comprising metformin.

4. The depot of claim 3, wherein the first therapeutic agent is released before or after the second therapeutic agent.

5. The depot of claim 3, wherein the first and second therapeutic agents are released substantially simultaneously.

6. The depot of claim 1, wherein the GLP-1 receptor agonist in the therapeutic region comprises at least 50% of the total weight of the depot.

7. The depot of claim 1, wherein the period of time is no less than 1 month.

8. The depot of claim 1, wherein the depot is biodegradable and/or bioerodible.

9. The depot of claim 1, wherein about 40% to about 60% of the GLP-1 receptor agonist in the therapeutic region is released in the first half of the period of time.

10. The depot of claim 1, wherein the depot is configured to release about 2 μg/day to about 10 mg/day of the GLP-1 receptor agonist.

11. The depot of claim 1, wherein the GLP-1 receptor agonist is released at a substantially steady state rate throughout the period of time.

12. The depot of claim 1, wherein the depot further comprises a thermal stabilizer.

13. The depot of claim 12, wherein the thermal stabilizer comprises at least one of a sugar, antioxidant or buffer.

14. The depot of claim 13, wherein the sugar comprises at least one of trehalose, raffinose or mannitol.

15. The depot of claim 13, wherein the antioxidant comprises at least one of methionine, ascorbic acid, sodium thiosulfate, catalase, platinum ethylenediaminetetraacetic acid (EDTA), citric acid, cysteins, thioglycerol, thioglycolic acid, thiosorbitol, butylated hydroxyanisol, butylated hydroxyltoluene, or propyl gallate.

16. The depot of claim 13, wherein the buffer comprises at least one of citrate, histidine, succinate or tris.

17. The depot of claim 1, wherein the depot further comprises a chemical compound configured to inhibit denaturing of the therapeutic agent in vivo.

18. A system for delivering a therapeutic agent to a patient, the system comprising:
a needle having a lumen;
a syringe operatively coupled to the needle; and
a depot disposed within the lumen and configured to be emitted from the needle via activation of the syringe, the depot comprising:
a therapeutic region comprising the therapeutic agent, the therapeutic agent including glucagon-like peptide-1 (GLP-1) receptor agonist; and
a control region at least partially surrounding the therapeutic region and elongated along the first axis, the control region comprising a bioresorbable polymer and a releasing agent mixed with the polymer, wherein the releasing agent is configured to dissolve when the depot is placed in vivo to form diffusion openings in the control region;
wherein the depot is configured to be implanted in vivo and, while implanted, release the therapeutic agent for a period of time.

19. The system of claim 18, wherein the depot comprises the depot of claim 1.

20. The system of claim 18, wherein the needle has a lumen that is no greater than 22 gauge in size.
